(19) United States
(12) Patent Application Publication
(10) Pub. No.: US 2004/0186098 A1
(43) Pub. Date: Sep. 23, 2004

(54) Method for preventing and treating hearing loss using sensorineurotrophic compounds

(75) Inventor: Ella Magal, Thousand Oaks, CA (US)

Correspondence Address: AMGEN, INC., U.S. Patent Operations/JWB, Dept. 4300, M/S 27-4-A, One Amgen Center Drive, Thousand Oaks, CA, 91320-1799, US

(73) Assignee: Amgen Inc.

(21) Appl. No.: 10/813,081

(22) Filed: Mar. 29, 2004

Related U.S. Application Data
(63) Continuation of application No. 09/159,105, filed on Sep. 23, 1998, now abandoned.
(60) Provisional application No. 60/059,905, filed on Sep. 24, 1997.
(60) Provisional application No. 60/059,963, filed on Sep. 25, 1997.

(51) Int. Cl. A61K 31/55; A61K 31/54; A61K 31/537; A61K 31/496; A61K 31/445; A61K 31/40; A61K 31/426; A61K 31/421

(52) U.S. Cl. 514/217.12; 514/227.2; 514/237.5; 514/252.12; 514/317; 514/365; 514/374; 514/400; 514/408

(57) ABSTRACT

The present invention relates generally to methods for preventing and/or treating injury or degeneration of cochlear hair cells and spiral ganglion neurons by administering sensorineurotrophic compounds described below. The invention relates more specifically to methods for treating sensorineural hearing loss as well as vestibular disorders and tinnitus.

### BACKGROUND OF THE INVENTION

The invention relates generally to methods for preventing and/or treating hearing loss due to variety of causes. The present invention relates more specifically to methods for preventing and/or treating injury or degeneration of inner ear sensory cells, such as hair cells and auditory neurons, by administering a sensorineurotrophic compound to a patient in need thereof.

A. Neuroimmunophilins

The peptidyl-prolyl isomerases (“PPIases”) are a family of ubiquitous enzymes which catalyze the interconversion of cis and trans amide bond rotamers adjacent to proline residues in peptide substrates. See, for example, Galat, A., Eur. J. Biochem. (1993) 216:689-707 and Kay, J. E., Biochem. J. (1996) 314:361-385. The PPIases have been referred to as “immunophilins” because of their interaction with certain immunosuppressant drugs. Schreiber, S. L., Science (1991) 251:283-287; Rosen, M. K. and Schreiber, S. L., Angew. Chem. Intl. Ed. Engi. (1992) 31:384-400.

The PPIase, cyclophilin A, was found to be the intracellular protein target for the potent immunosuppressant drug cyclosporin A. Subsequently, the structurally unrelated macrolide immunosuppressant FK506 was discovered to bind to a different PPIase enzyme which was named FK506-binding protein, or FKBP. Rapamycin, another macrolide drug which is a structural analogue of FK506, also interacts with FKBP.

All three of these drugs bind to their respective immunophilins and inhibit the respective PPIase activities. However, inhibition of immunophilin enzymatic activity is not the cause of the observed immunosuppressive effects. Binding of the drugs to the immunophilins results in the formation of “activated complexes”, which interact with downstream proteins to inhibit proliferation of T-lymphocytes. Schreiber, supra; Rosen, et al., supra. In the case of FK506, binding to FKBP results in a drug-protein complex which is a potent inhibitor of the calcium-calmodulin-dependent protein phosphatase, calcineurin. Bierer, B. E., Mattila, P. S., Standaert, R. F., Herzenberg, L. A., Burakoff, S. J., Crabtree, G., Schreiber, S. L., Proc. Natl. Acad. Sci. USA (1990) 87:9231-9235; Liu, J., Farmer, J. D., Lane, W. S., Friedman, J., Weissman, I., Schreiber, S. L.; Cell (1991) 66:807-815.

Neither FK506 or FKBP alone appreciably inhibits calcineurin's activity. Inhibiting calcineurin blocks the signaling pathway by which the activated T-cell receptor causes transcription of the gene for interleukin-2, inhibiting the immune response. Despite the structural dissimilarity between FK506 and cyclosporin A (and cyclophilin and FKBP), the cyclosporin A-cyclophilin complex also inhibits calcineurin, and thus cyclosporin A and FK506 have the same mechanism of action.

On the other hand, while rapamycin and FK506 have similar structures and bind to the same immunophilin (FKBP), rapamycin's mechanism of action is different from that of FK506. The complex of FKBP12 with rapamycin interacts with a protein called FRAP, or RAFT, and in so doing blocks the signal pathway leading from the IL-2 receptor on the surface of T-cells to promotion of entry into the cell cycle in the nucleus. Sabatini, D. M., Erdjument-Bromage, H., Lui, M.; Tempst, P., Snyder, S. H., Cell (1994) 78:35-43; Brown, E. J., Albers, M. W., Shin, T. B., Ichikawa, K., Keith, C. T., Lane, W. S., Schreiber, S. L. Nature (1994) 369:756-758; Brown, E. J., Beal, P. A., Keith, C. T., Chen, J., Shin, T. B., Schreiber, S. L., Nature (1995) 377:441-446.

Thus, all three drugs produce the same effect—suppression of T-cell proliferation—but do so by inhibiting distinct signal transduction pathways. The introduction of cyclosporin (“CsA”) marked a breakthrough in organ transplantation, and the drug became a major pharmaceutical product. The subsequent discovery of rapamycin (“Rapa”) and FK506 further fueled interest in the cellular basis of the actions of these drugs. The discovery of the interaction of the immunophilins with CsA, FK506 and Rapa led to research on the mechanistic basis of immunophilin-mediated immunosuppression.

### Immunophilins and the Nervous System

Because the initial interest in the immunophilins was largely driven by their role in the mechanism of action of the immunosuppressant drugs, most of the original studies of these proteins and their actions focused on the tissues of the immune system. In 1992, it was reported that levels of FKBP12 in the brain were 30 to 50 times higher than in the immune tissues. Steiner, J. P., Dawson, T. M., Fotuhi, M., Glatt, C. E., Snowman, A. M., Cohen, N., Snyder, S. H., Nature (1992) 358:584-587. This finding suggested a role for the immunophilins in the functioning of the nervous system. Both FKBP and cyclophilin were widely distributed in the brain and were found almost exclusively within neurons. The distribution of the immunophilins in the brain closely resembled that of calcineurin, suggesting a potential neurological link. Steiner, J. P., Dawson, T. M., Fotuhi, M., Glatt, C. E., Snowman, A. M., Cohen, N., Snyder, S. H., Nature (1992) 358:584-587; Dawson, T. M., Steiner, J. P., Lyons, W. E., Fotuhi, M., Blue, M., Snyder, S. H., Neuroscience (1994) 62:569-580.

Subsequent work demonstrated that the phosphorylation levels of several known calcineurin substrates were altered in the presence of FK506. Steiner, J. P., Dawson, T. M., Fotuhi, M., Glatt, C. E., Snowman, A. M., Cohen, N., Snyder, S. H., Nature (1992) 358:584-587. One of the proteins affected by FK506 treatment, GAP-43, mediates neuronal process elongation. Lyons, W. E., Steiner, J. P., Snyder, S. H., Dawson, T. M., J. Neurosci. (1995) 15:2985-2994. This research revealed that FKBP12 and GAP-43 were upregulated in damaged facial or sciatic nerves in rats. Also, FKBP12 was found in very high levels in the growth cones of neonatal neurons. FK506 was tested to determine whether or not it might have an effect on nerve growth or regeneration. In cell culture experiments with PC12 cells or sensory neurons from dorsal root ganglia, FK506 promoted process (neurite) extension with subnanomolar potency. Lyons, W. E., George, E. B., Dawson, T. M., Steiner, J. P., Snyder, S. H., Proc. Natl. Acad. Sci. USA (1994) 91:3191-3195. Gold et al. demonstrated that FK506 functioned as a neurotrophic agent in vivo. In rats with crushed sciatic nerves, FK506 accelerated nerve regeneration and functional recovery. Gold, B. G., Storm-Dickerson, T., Austin, D. R., Restorative Neurol. Neurosci., (1994) 6:287; Gold, B. G., Katoh, K., Storm-Dickerson, T. J, Neurosci. (1995) 15:7509-7516. See, also, Snyder, S. H., Sabatini, D. M., Nature Medicine (1995) 1:32-37 (regeneration of lesioned facial nerves in rats augmented by FK506).

Besides FK506, rapamycin and cyclosporin also produced potent neurotrophic effects in vitro in PC12 cells and chick sensory neurons. Steiner, J. P., Connolly, M. A., Valentine, H. L., Hamilton, G. S., Dawson, T. M., Hester, L., Snyder, S. H., Nature Medicine (1997) 3:421-428. As noted above, the mechanism for immunosuppression by rapamycin is different than that of FK506 or cyclosporin. The observation that rapamycin exerted neurotrophic effects similar to FK506 and cyclosporin suggested that the nerve regenerative effects of the compounds are mediated by a different mechanism than that by which they suppress T-cell proliferation.

Analogues of FK506, rapamycin, and cyclosporin which bind to their respective immunophilins, but are devoid of immunosuppressive activity, are known in the art. Thus, the FK506 analogue L-685,818 binds to FKBP but does not interact with calcineurin, and is therefore nonimmunosuppressive. Dumont, F. J., Staruch, M. J., Koprak, S. L., J. Exp. Med. (1992) 176:751-760.

Similarly, 6-methyl-alanyl cyclosporin A (6-[Me]-ala-CsA) binds to cyclophilin but likewise lacks the ability to inhibit calcineurin. The rapamycin analogue WAY-124,466 binds FKBP but does not interact with RAFT, and is likewise nonimmunosuppressive. Ocain, T. D., Longhi, D., Steffan, R. J., Caccese, R. G., Sehgal, S. N., Biochem. Biophys. Res. Commun. (1993) 192:1340-1346; Sigal, N. H., Dumont, F., Durette, P., Siekierka, J. J., Peterson, L., Rich, D., J. Exp. Med. (1991) 173:619-628. These nonimmunosuppressive compounds were shown to be potent neurotrophic agents in vitro, and one compound, L-685,818, was as effective as FK506 in promoting morphological and functional recovery following sciatic nerve crush in rats. Steiner, J. P., Connolly, M. A., Valentine, H. L., Hamilton, G. S., Dawson, T. M., Hester, L., Snyder, S. H., Nature Medicine (1997) 3:421-428. These results demonstrated that the neurotrophic properties of the immunosuppressant drugs could be functionally dissected from their immune system effects.

Published work by researchers studying the mechanism of action of FK506 and similar drugs had shown that the minimal FKBP-binding domain of FK506 (as formulated by Holt et al., BioMed. Chem. Lett. (1994) 4:315-320) possessed good affinity for FKBP. Hamilton et al. proposed that the neurotrophic effects of FK506 resided within the immunophilin binding domain, and synthesized a series of compounds which were shown to be highly effective in promoting neurite outgrowth from sensory neurons, often at picomolar concentrations. Hamilton, G. S., Huang, W., Connolly, M. A., Ross, D. T., Guo, H., Valentine, H. L., Suzdak, P. D., Steiner, J. P., BioMed. Chem. Lett. (1997). These compounds were shown to be effective in animal models of neurodegenerative disease.

### FKBP12 Inhibitors/Ligands

A number of researchers in the early 1990s explored the mechanism of immunosuppression by FK506, cyclosporin and rapamycin, and sought to design second-generation immunosuppressant agents that lacked the toxic side effects of the original drugs. A pivotal compound, 506BD (for “FK506 binding domain”—see Bierer, B. E., Somers, P. K., Wandless, T-J., Burakoff, S. J., Schreiber, S. L., Science (1990) 250:556-559), retained the portion of FK506 which binds FKBP12 in an intact form, while the portion of the macrocyclic ring of FK506 which extends beyond FKBP12 in the drug-protein complex was significantly altered. The finding that 506BD was a high-affinity ligand for, and inhibitor of, FK506, but did not suppress T-cell proliferation was the first demonstration that the immunosuppressant effects of FK506 were not simply caused by rotamase activity inhibition.

In addition to various macrocyclic analogues of FK506 and rapamycin, simplified compounds which represent the excised FKBP binding domain of these drugs were synthesized and evaluated. Non-macrocyclic compounds with the FKBP-binding domain of FK506 excised possess lower affinity for FKBP12 than the parent compounds. Such structures still possess nanomolar affinity for the protein. See, e.g., Hamilton, G. S., Steiner, J. P., Curr. Pharm. Design (1997) 3:405-428; Teague, S. J., Stocks, M. J., BioMed. Chem. Lett., (1993) 3:1947-1950; Teague, S. J., Cooper, M. E., Donald, D. K., Furber, M., BioMed. Chem. Lett. (1994) 4:1581-1584.

Holt et al. published several studies of simple pipecolate FKBP12 inhibitors which possessed excellent affinity for FKBP12. In initial studies, replacement of the pyranose ring of FK506 mimetics demonstrated that simple alkyl groups such as cyclohexyl and dimethylpentyl worked well in this regard. Holt et al., BioMed. Chem. Lett. (1994) 4:315-320. Simple compounds possessed good affinity for FKBP12 (Kᵢ values of 250 and 25 nM, respectively). These structures demonstrated that these simple mimics of the binding domain of FK506 bound to the immunophilin in a manner nearly identical to that of the corresponding portion of FK506. Holt, D. A., Luengo, J. I., Yamashita, D. S., Oh, H. J., Konialian, A. L., Yen, H. K., Rozamus, L. W., Brandt, M., Bossard, M. J., Levy, M. A., Eggleston, D. S., Liang, J., Schultz, L. W.; Stout, T. J.; Clardy, I., J. Am. Chem. Soc. (1993) 115:9925-9938.

Armistead et al. also described several pipecolate FKBP12 inhibitors. X-ray structures of the complexes of these molecules with FKBP also demonstrated that the binding modes of these simple structures were related to that of FK506. Armistead, D. M., Badia, M. C., Deininger, D. D., Duffy, J. P., Saunders, J. O., Tung, R. D., Thomson, J. A.; DeCenzo, M. T.; Futer, O., Livingston, D. J., Murcko, M. A., Yamashita, M. M., Navia, M. A., Acta Cryst. (1995) D51:522-528.

As expected from the noted effector-domain model, FKBP12 ligands lacking an effector element were inactive as immunosuppressant agents, failing to suppress lymphocyte proliferation both in vitro and in vivo.

### Neuroprotective/Neuroregenerative Effects of FKBP12 Ligands

Steiner et al., U.S. Pat. No. 5,696,135 (issued Dec. 9, 1997) describe the neurotrophic actions of a large number of compounds such as those described above. Cultured chick sensory neurons were used as an in vitro assay to measure the ability of compounds to promote neurite outgrowth (fiber extension) in neurons. Compounds were also tested for their ability to bind to FKBP12 and inhibit its enzymatic (rotamase) activity. As the data demonstrate, many of these compounds were found to be extremely potent nerve growth agents, promoting fiber extension from cultured neurons with half-maximal effects seen in some cases at picomolar concentrations. The effects of these simple FKBP12 ligands on nervous tissue are comparable to, or in some cases more potent than, FK506 itself.

Some of the compounds were also shown to promote regrowth of damaged peripheral nerves in vivo. Steiner, J. P., Connolly, M. A., Valentine, H. L., Hamilton, G. S., Dawson, T. M., Hester, L., Snyder, S. H., Nature Medicine (1997) 3:421-428. In whole-animal experiments in which the sciatic nerves of rats were crushed with forceps and animals treated with these compounds subcutaneously, there was found significant regeneration of damaged nerves relative to control animals, resulting in both more axons in drug-treated animals and axons with a greater degree of myelination. Lesioning of the animals treated only with vehicle caused a significant decrease in axon number (50% decrease compared to controls) and degree of myelination (90% decrease compared to controls). Treatment with the FKBP12 ligands resulted in reduction in the decrease of axon number (25% and 5% reduction, respectively, compared to controls) and in the reduction of myelination levels (65% and 50% decrease compared to controls). Similar results were subsequently reported by Gold et al. Gold, B. G., Zeleney-Pooley, M., Wang, M. S., Chaturvedi, P.; Armistead, D. M., Exp. Neurobiol. (1997) 147:269-278.

Several of these compounds were shown to promote recovery of lesioned central dopaminergic neurons in an animal model of Parkinson's Disease. Hamilton, G. S., Huang, W., Connolly, M. A., Ross, D. T., Guo, H., Valentine, H. L., Suzdak, P. D., Steiner, J. P., BioMed. Chem. Lett. (1997). N-Methyl-4-phenyl-1,2,3,6-tetrahydropyridine (“MPTP”) is a neurotoxin which selectively destroys dopaminergic neurons. Gerlach, M., Riederer, P., Przuntek, H., Youdim, M. B., Eur. J. Pharmacol. (1991) 208:273-286. The nigral-striatal dopaminergic pathway in the brain is responsible for controlling motor movements.

Parkinson's Disease is a serious neurodegenerative disorder resulting from degeneration of this motor pathway. Lesioning of the nigral-striatal pathway in animals with MPTP has been utilized as an animal model of Parkinson's Disease. In mice treated with MPTP and vehicle, a substantial loss of 60-70% of functional dopaminergic terminals was observed as compared to non-lesioned animals. Lesioned animals receiving FKBP12 ligands concurrently with MPTP showed a striking recovery of TH-stained striatal dopaminergic terminals, as compared with controls, suggesting that FKBP12 ligands may possess potent neuroprotective and neuro-regenerative effects on both peripheral as well as central neurons.

Other compounds which have an affinity for FKBP12 may also possess neurotrophic activities similar to those described above. For example, one skilled in the art is referred to the following patents and patent applications for their teaching of neurotrophic compounds which are lacking immunosuppressive activity:

Hamilton et al., U.S. Pat. No. 5,614,547 (Mar. 25, 1997);

Steiner et al., U.S. Pat. No. 5,696,135 (Dec. 9, 1997);

Hamilton et al., U.S. Pat. No. 5,721,256 (Feb. 24, 1998);

Hamilton et al., U.S. Pat. No. 5,786,378 (Jul. 28, 1998);

Hamilton et al., U.S. Pat. No. 5,795,908 (Aug. 18, 1998);

Steiner et al., U.S. Pat. No. 5,798,355 (Aug. 25, 1998);

Steiner et al., U.S. Pat. No. 5,801,197 (Sep. 1, 1998)

Li et al., U.S. Pat. No. 5,801,187 (Sep. 1, 1998)

These molecules are effective ligands for, and inhibitors of, FKBP12 and are also potent neurotrophic agents in vitro, promoting neurite outgrowth from cultured sensory neurons at nanomolar or subnanolar dosages.

Additionally, as noted, compounds which possess immunosuppressive activity, for example, FK506, CsA and Rapa, among others, also may possess a significant level of neurotrophic activity. Thus, to the extent that such compounds additionally may possess activities, including neurotrophic activities, such compounds are intended to be included within the term “sensorineurotrophic compound” as used herein. The following publications provide disclosures of compounds which presumably possess immunosuppressive activities, as well as possibly other activities, and are likewise intended to be included within the term “sensorineurotrophic compound” as used herein:

Armistead et al., U.S. Pat. No. 5,192,773 (Mar. 9, 1993);

Armistead et al., U.S. Pat. No. 5,330,993 (Jul. 19, 1994);

Armistead et al., U.S. Pat. No. 5,516,797 (May 14, 1996);

Armistead et al., U.S. Pat. No. 5,620,971 (Apr. 15, 1997);

Armistead et al., U.S. Pat. No. 5,622,970 (Apr. 22, 1997);

Armistead et al., U.S. Pat. No. 5,665,774 (Sep. 9, 1997);

Zelle et al., U.S. Pat. No. 5,780,484 (Jul. 14, 1998)

The neuroregenerative and neuroprotective effects of FKBP12 ligands are not limited to dopaminergic neurons in the central nervous system. In rats treated with para-chloro-amphetamine (“PCA”), an agent which destroys neurons which release serotonin as a neurotransmitter, treatment with an FKBP ligand was reported to exert a protective effect. Steiner, J. P., Hamilton, G. S., Ross, D. T., Valentine, H. L., Guo, H., Connolly, M. A., Liang, S., Ramsey, C., Li, J. H., Huang, W., Howorth, P.; Soni, R., Fuller, M., Sauer, H., Nowotnick, A., Suzdak, P. D., Proc. Natl. Acad. Sci. USA (1997) 94:2019-2024. In rats lesioned with PCA, cortical density of serotonin fibers was reduced 90% relative to controls. Animals receiving the ligand showed a greater serotonin innervation in the cortex—serotonergic innervation in the somatosensory cortex was increased more than two-fold relative to lesioned, non-drug treated animals.

Similarly, such ligands have been shown to induce sprouting of residual cholinergic axons following partial transection of the fimbria fornix in rats. Guo, H., Spicer, D. M., Howorth, P., Hamilton, G. S., Suzdak, P. D, Ross, D. T., Soc. Neurosci. Abstr. (1997) 677.12. The transection produced a 75-80% deafferentiation of the hippocampus. Subcutaneous administration of the FBKP12 ligand produced a four-fold sprouting of spared residual processes in the CA1, CA3 and dentate gyrus regions of the hippocampus, resulting in significant recovery of cholinergic innervation in all three regions as quantitated by choline acetyltransferase (ChAT) density.

Taken together, the data in the noted references indicate that certain ligands for FKBP 12, preferably those which are non-immuno-suppressive, comprise a class of potent active neurotrophic compounds which have been referred to as “neuroimmunophilins” or “neuroimmunophilin ligands” with potential for therapeutic utility in the treatment or prevention of neurodegenerative diseases. Thus, in the context of the present invention, a sensorineurotrophic compound is meant to encompass those compounds which have been designated as neuroimmunophilins and which also may have, but are not required to have, binding affinity for an FKBP. The ultimate mechanism of action and whether or not such compounds also possess other activity such as, for example, immunosuppressive activity, is not determinative of whether the compound is a “sensorineurotrophic” compound for purposes of the invention as long as the compound in question possesses the desired effect on sensory cells of the ear.

Until the present invention, none of the prior work, disclosed the use of the disclosed sensorineurotrophic compounds in the treatment or prevention of hearing loss and associated diseases. As described in more detail below, the present invention is directed to such uses.

B. Hearing Loss

To better understand the invention, the following discussion on hearing loss is provided. The epithelial hair cells in the organ of Corti of the inner ear, transduce sound into neural activity, which is transmitted along the cochlear division of the eighth cranial nerve. This nerve consists of fibers from three types of neurons (Spoendlin, H. H., in Friedmann, I. Ballantyne, J., eds. “Ultrastructural Atlas of the Inner Ear”, London, Butterworth, pp. 133-164, (1984)) 1) afferent neurons, which lie in the spiral ganglion and connect the cochlea to the brainstem; 2) efferent olivocochlear neurons, which originate in the superior olivary complex; and, 3) autonomic adrenergic neurons, which originate in the cervical sympathetic trunk and innervate the cochlea. In the human, there are approximately 30,000 afferent cochlear neurons, with myelinated axons, each consisting of about 50 lamellae, and 4-6 μm in diameter. This histologic structure forms the basis of uniform conduction velocity, which is an important functional feature. Throughout the length of the auditory nerve, there is a trophic arrangement of afferent fibers, with ‘basal’ fibers wrapped over the centrally placed ‘apical’ fibers in a twisted rope-like fashion. Spoendlin (Spoendlin, H. H. in Naunton, R. F., Fernadex, C. eds., “Evoked Electrical Activity in the Auditory Nervous System”, London, Academic Press, pp. 21-39, (1978)) identified two types of afferent neurons in the spiral ganglion on the basis of morphologic differences: type I cells (95%) are bipolar and have myelinated cell bodies and axons that project to the inner hair cells. Type II cells (5%) are monopolar with unmyelinated axons and project to the outer hair cells of the organ of Corti. Each inner hair cell is innervated by about 20 fibers, each of which synapses on only one cell. In contrast, each outer hair cell is innervated by approximately six fibers, and each fiber branches to supply approximately 10 cells. Within the cochlea, the fibers divide into: 1) an inner spiral group, which arises primarily ipsilaterally and synapses with the afferent neurons to the inner hair cells, and 2) a more numerous outer radial group, which arises mainly contralaterally and synapses directly with outer hair cells. There is a minimal threshold at one frequency, the characteristic or best frequency, but the threshold rises sharply for frequencies above and below this level (Pickles, J. O. in “Introduction to the Physiology of Hearing”, London, Academic Press, pp. 71-106, (1982)). Single auditory nerve fibers therefore appear to behave as band-pass filters. The basilar membrane vibrates preferentially to different frequencies, at different distances along its length, and the frequency selectivity of each cochlear nerve fiber is similar to that of the inner hair cell to which the fiber is connected. Thus, each cochlear nerve fiber exhibits a tuning curve covering a different range of frequencies from its neighboring fiber (Evans, E. F. in Beagley H. A. ed., “Auditory investigation: The Scientific and Technological basis”, New York, Oxford University Press, (1979)). By this mechanism, complex sounds are broken down into component frequencies (frequency resolution) by the filters of the inner ear.

Hearing loss of a degree sufficient to interfere with social and job-related communications is among the most common chronic neural impairments in the U.S. population. On the basis of health-interview data (Vital and health statistics. Series 10. No. 176. Washington, D.C. (DHHS publication no. (PHS) 90-1504)), it is estimated that approximately 4 percent of people under 45 years of age and about 29 percent of those 65 years or over have a handicapping loss of hearing. It has been estimated that more than 28 million Americans have hearing impairment and that as many as 2 million of this group are profoundly deaf (“A Report Of The Task Force On The National Strategic Plan”, Bethesda, Md., National Institute of Health, (1989)). The prevalence of hearing loss increases dramatically with age. Approximately 1 per 1000 infants has a hearing loss sufficiently severe to prevent the unaided development of spoken language (Gentile, A., et al., “Characteristics Of Persons With Impaired Hearing”, United States, 1962-1963. Series 10. No. 35. Washington, D.C., Government printing office, (1967) (DHHS publication no. (PHS) 1000)) (“Human Communication And Its Disorders: An Overview”, Bethesda, Md., National Institutes of health, (1970)). More than 360 per 1000 persons over the age of 75 have a handicapping hearing loss (Vital and health statistics. Series 10. No. 176. Washington, D.C. (DHHS publication no. (PHS) 90-1504).

It has been estimated that the cost of lost productivity, special education, and medical treatment may exceed $30 billion per year for disorders of hearing, speech and language (“1990 Annual Report Of The National Deafness And Other Communication Disorders Advisory Board”, Washington, D.C., Government Printing Office, 1991. (DHHS publication no. (NIH) 91-3189)). The major common causes of profound deafness in childhood are genetic disorders and meningitis, constituting approximately 13 percent and 9 percent of the total, respectively (Hotchkiss, D., Demographic Aspects Of Hearing Impairment: Questions And Answers”, 2nd ed., Washington, D.C., Gallaudet University Press, (1989)). In approximately 50 percent of the cases of childhood deafness, the cause is unknown, but is likely due to genetic causes or predisposition (Nance W., Otolaryngol. Clin. North Am. (1975), 8:19-48).

Impairment anywhere along the auditory pathway, from the external auditory canal to the central nervous system, may result in hearing loss. The auditory apparatus can be subdivided into the external and middle ear, inner ear and auditory nerve and central auditory pathways. Auditory information in humans is transduced from a mechanical signal to a neurally conducted electrical impulse by the action of approximately 15,000 epithelial cells (hair cells) and 30,000 first-order neurons (spiral ganglion cells) in the inner ear. All central fibers of spiral ganglion neurons form synapses in the cochlear nucleus of the pontine brainstem. The number of neurons involved in hearing increases dramatically from the cochlea to the auditory brain stem and the auditory cortex. All auditory information is transduced by only 15,000 hair cells, of which the so-called inner hair cells, numbering 3500, are critically important, since they form synapses with approximately 90 percent of the 30,000 primary auditory neurons. Thus, damage to a relatively few cells in the auditory periphery can lead to substantial hearing loss. Hence, most causes of sensorineural loss can be ascribed to lesions in the inner ear (Nadol, J. B., New England Journal of Medicine, (1993), 329:1092-1102).

Hearing loss can be on the level of conductivity, sensorineural and central level. Conductive hearing loss is caused by lesions involving the external or middle ear, resulting in the destruction of the normal pathway of airborne sound amplified by the tympanic membrane and the ossicles to the inner ear fluids. Sensorineural hearing loss is caused by lesions of the cochlea or the auditory division of the eighth cranial nerve. Central hearing loss is due to lesions of the central auditory pathways. These consist of the cochlear and dorsal olivary nucleus complex, inferior colliculi, medial geniculate bodies, auditory cortex in the temporal lobes and interconnecting afferent and efferent fiber tracts (Adams R. D. and Maurice, V., eds., in “Principles of Neurology”, (1989), McGraw-Hill Information Services Company, pp. 226-246).

As mentioned previously, at least 50 percent of cases of profound deafness in childhood have genetic causes (Brown, K. S., Med. Clin. North Am. (1969), 53: 741-72). If one takes into consideration the probability that genetic predisposition is a major causative factor in presbycusis—or age-related hearing loss—which affects one third of the population over 75 years of age (Nadol, J. B. Beasley, D. S., Davis G. A., eds., “Aging: Communication Processes and Disorders”, New York: Grune & Stratton, (1981), pp. 63-85), genetic and hereditary factors are probably the single most common cause of hearing loss. Genetic anomalies are much more commonly expressed as sensorineural hearing loss than as conductive hearing loss. Genetically determined sensorineural hearing loss is clearly a major, if not the main cause of sensorineural loss, particularly in children (Nance W. E., Sweeney A., Otolaryngol. Clin. North Am. (1975) 8:19-48). Among the most common syndromal forms of sensorineural loss are Waardenburg's syndrome, Alport's syndrome and Usher's syndrome.

A variety of commonly used drugs have ototoxic properties. The best known are the aminoglycoside antibiotics (Lerner, S. A., et al., eds., “Aminoglycoside Ototoxicity”, Boston: Little, Brown, (1981); Smith, C. R., et al., N. Engl. J. Med. (1980), 302:1106-9), loop diuretics (Bosher, S. K., Acta Otolaryngol. (Stockh) (1980), 90:4-54), salicylates (Myers, E. N., et al., N. Engl. J. Med. (1965), 273:587-90) and antineoplastic agents such as cisplatin (Strauss, M., et al., Laryngoscope (1983), 143:1263-5). Ototoxicity has also been described during oral or parenteral administration of erythromycin (Kroboth, P. D., et al., Arch. Intern. Med., (1983), 1:169-79; Achweitzer, V. G., Olson, N., Arch. Otolaryngol. (1984), 110:258-60).

Most ototoxic substances cause hearing loss by damaging the cochlea, particularly the auditory hair cells, auditory neurons and the stria vascularis, a specialized epithelial organ within the inner ear, responsible for the homeostasis of fluids and electrolytes (Nadol, J. B., New England J. Med., (1993), 329:1092-1102). Secondary neural degeneration may occur many years after an ototoxic event affecting the hair cells. There is evidence that some ototoxic substances may be selectively concentrated within the inner ear, resulting in progressive sensorineural loss despite the discontinuation of systemic administration (Federspil, P., et al., J. Infect. Dis., (1976), 134, Suppl: S200-S205)).

Trauma due to acoustic overstimulation is another leading cause of deafness. There is individual susceptibility to trauma from noise. Clinically important sensorineural hearing loss may occur in some people exposed to high-intensity noise, even below levels approved by the Occupational Safety and Health Agency (Osguthorpe, J. D., ed., Washington D.C., American Academy of Otolaryngology-Head and Neck Surgery Foundation, (1988)).

Demyelinating processes, such as multiple sclerosis, may cause sensorineural hearing loss (Noffsinger, D., et al., Acta Otolaryngol. Suppl. (Stockh.) (1972), 303:1-63). More recently, a form of immune-mediated sensorineural hearing loss has been recognized (McCabe, B. F., Ann. Otol. Rhinol. Laryngol. (1979), 88:585-9). The hearing loss is usually bilateral, is rapidly progressive (measured in weeks and months), and may or may not be associated with vestibular symptoms.

A variety of tumors, both primary and metastatic, can produce either a conductive hearing loss, or a sensorineural hearing loss, by invading the inner ear or auditory nerve (Houck, J. R., et al., Otolaryngol. Head Neck Surg. (1992), 106:92-7). A variety of degenerative disorders of unknown cause can produce sensorineural hearing loss. Meniere's syndrome (Nadol, J. B., ed., “Meniere's Disease: Pathogenesis, Pathophysiology, Diagnosis, And Treatment,” Amsterdam: Kugler & Ghedini (1989)), characterized by fluctuating sensorineural hearing loss, episodic vertigo, and tinnitus, appears to be caused by a disorder of fluid homeostasis within the inner ear, although the pathogenesis remains unknown. Sudden idiopathic sensorineural hearing loss (Wilson, W. R., et al., Arch. Otolaryngol. (1980), 106:772-6), causing moderate-to-severe sensorineural deafness, may be due to various causes, including inner ear ischemia and viral labyrinthitis.

Presbycusis, the hearing loss associated with aging, affects more than one third of persons over the age of 75 years. The most common histopathological correlate of presbycusis is the loss of epithelial (hair) cells, neurons, and the stria vascularis of the peripheral auditory system (Schuknecht, H. F., “Pathology of the Ear”, Cambridge, Mass., Harvard University Press, (1974), pp. 388-403). Presbycusis is best understood as resulting from the cumulative effects of several noxious influences during life, including noise trauma, ototoxicity and genetically influenced degeneration.

Regardless of the cause, there exists a need to prevent or treat sensorineural hearing loss. The present invention provides such a method.

### SUMMARY OF THE INVENTION

In particular, the present invention provides methods for treating sensorineural hearing loss comprising administering to a patient in need thereof, particularly a patient having a lesion in the inner ear, a therapeutically effective amount of a sensorineurotrophic compound. By way of example, the hearing loss may be associated with injury or degeneration of epithelial hair cells (cochlear hair cells) or spiral ganglion neurons in the inner ear.

The present invention is based on the discovery that hair cells respond to a sensorineurotrophic compound by resisting the toxic effects of ototoxins, such as cisplatin and neomycin or exposure to other damaging environmental conditions, for example, noise. Thus, a therapeutically effective amount of a sensorineurotrophic compound may be administered to promote the protection, survival or regeneration of hair cells and spiral ganglion neurons.

Similar to a defect in the hair cells in the cochlea, a lesion or disturbance to the hair cells of the vestibular apparatus may result in dizziness, vertigo or loss of balance. Such lesions or disturbances in a patient may also be treated in accordance with the invention by administering to said patient a therapeutically effective amount of a sensorineurotrophic compound as defined herein.

According to the invention, a sensorineurotrophic compound may be administered parenterally at a dose ranging from about 1 ng/ear/day to about 10 ng/ear/day, typically at a dose of about 1 μg/ear/day to about 10 μg/ear/day, and usually at a dose of about 5 mg/kg/day to about 20 mg/kg/day. It is also contemplated that, depending on the individual patient's needs and route of administration, the sensorineurotrophic compound may be given at a lower frequency such as monthly, weekly or several times per week, rather than daily. It is further contemplated that the sensorineurotrophic compound may be administered topically, for example in the form of ear drops, orally, for example in the form of tablets or pills, parenterally, such as by subcutaneous or intramuscular injection, or directly into the middle ear or the inner ear. One skilled in the art will appreciate that with direct administration a smaller amount of the desired compound may be used. For example, one may administer directly to the middle ear or inner-ear a dose in the range of about 1 ng/ear to about 10 ng/ear in a single dose or in a multiple administration regimen.

It is further contemplated that the sensorineurotrophic compound may be administered separately, sequentially, or simultaneously in combination or conjunction with an effective amount of a second therapeutic agent, such as GDNF, BDNF and NT-3, or any other agent useful for the treatment of the ear.

The invention also provides for the use of a sensorineurotrophic compound in the manufacture of a medicament or pharmaceutical composition for the treatment of injury or degeneration of hair cells and auditory neurons resulting from various causes of sensorineural hearing loss. Such pharmaceutical compositions include topical, systemic, oral or middle and inner ear sensorineurotrophic compound formulations, optionally in combination with cochlear implants.

### BRIEF DESCRIPTION OF THE DRAWINGS

FIG. 1 shows the protective effect of sensorineurotrophic compound I in cochlear explant cultures treated with cisplatin.

FIG. 2 shows the protective effect of sensorineurotrophic compound I in cochlear explant cultures treated with neomycin.

FIG. 3A shows the protection against neomycin induced outer hair cell loss by administering neuroimmunophilin compound I in an in vivo model.

FIGS. 3B and 3C show the protection against neomycin induced outer hair cell loss by administering sensorineurotrophic compound I at 10 ng and 1 ng dosages, respectively.

FIGS. 4A and 4B show the protection by Compound I (10 ng and 1 ng, respectively) against inner ear hair cell loss induced by treatment with neomycin.

FIG. 5 shows the protection against inner ear hair cell loss when sensorineurotrophic compound I is administered systemically.

FIG. 6 shows the location of hair cells protected by systemic administration of Compound I when the inner ear is treated with neomycin.

FIG. 7 shows the percentage of animals retaining a Preyer's reflex when treated with cisplatin and sensorineurotrophic compound XXV relative to treatment with cisplatin and vehicle alone.

FIG. 8 shows the percentage loss in outer hair cells when treated with neomycin and sensorineurotrophic compound XVI (10 ng) or vehicle applied to the round window.

FIG. 9 shows the protection against loss in outer hair cells when treated with neomycin or neomycin and compound XVI, depending on location in the cochlea.

FIG. 10 shows the protection against outer hair cell loss in animals treated with neomycin compared to neomycin and compound XVI together.

FIGS. 11 and 12 show the protective effect of the administration of a variety of sensorineurotrophic compounds at 1 pM and 10 pM, respectively, in cochlear explant cultures treated with neomycin.

### DETAILED DESCRIPTION OF THE INVENTION

The present invention provides a method for preventing and/or treating sensorineural hearing loss by administering to a patient a therapeutically effective amount of a sensorineurotrophic compound. According to one aspect of the invention, methods are provided for treating damaged hair cells and auditory neurons by administering a therapeutically effective amount of a sensorineurotrophic compound by means of a pharmaceutical composition.

The present invention is based on the discovery that a sensorineurotrophic compound protects hair cells from ototoxin-induced cell death in explant cultures of rat's cochlea and in an animal model (guinea pig) of deafness. It is contemplated that administration of exogenous sensorineurotrophic compound will protect hair cells and spiral ganglion neurons from traumatic damage, for example damage caused by noise trauma, acute or chronic treatment with cisplatin and aminoglycoside antibiotics or from damage resulting from a lack of neurotrophic factors resulting from interruption of transport of the factors from the axon to the cell body. Such treatment is expected to allow hair cells and/or auditory neurons to tolerate intermittent insults from either environmental noise trauma or treatment with ototoxins, and to slow down, prevent or reverse the progressive degeneration of the auditory neurons and hair cells which is responsible for hearing loss in pathological conditions such as presbycusis (age-related hearing loss), inherited sensorineural degeneration, and post-idiopathic hearing losses and to preserve the functional integrity of the inner ear. Such treatment will also support the auditory neurons for better and longer performance of cochlear implants.

According to the invention, the sensori-neurotrophic compound may be administered systemically at a dose ranging from about 1 to about 10 mg/kg/day or into the middle ear at a dose ranging from about 1 ng/ear/day to about 10 ng/ear/day, typically at a dose of about 1 μg ear/day to about 10 μg/ear/day, and usually at a dose of about 5 μg/ear/day to about 20 μg/ear/day. The sensorineurotrophic compound may be administered directly into the inner ear in cases where invasion of the inner ear has already occurred such as in surgical procedures for inserting a cochlear implant or other surgeries of the inner ear. In such cases, a smaller amount of sensorineurotrophic compound may be administered, for example, from about 0.1 ng/ear to about 1 ng/ear in a single injection or in multiple injections. The sensorineurotrophic compound can be prepared and administered in the form of ear-drops which will penetrate the tympanic membrane. It is further contemplated that the sensorineurotrophic compound may be administered with an effective amount of a second therapeutic agent for the treatment of auditory neuron degeneration, including GDNF, BDNF and NT-3 as well as other factors or drugs used currently or in the future for the treatment of various inner and middle ear pathologies. A variety of pharmaceutical formulations and different delivery techniques are described in further detail below.

C. Sensorineurotrophic Compound Pharmaceutical Compositions

Sensorineurotrophic compound pharmaceutical compositions typically include a therapeutically effective amount of a sensorineurotrophic compound described herein in admixture with one or more pharmaceutically and physiologically acceptable formulation materials. Suitable formulation materials include, but are not limited to, antioxidants, preservatives, coloring, flavoring and diluting agents, emulsifying agents, suspending agents, solvents, fillers, bulking agents, buffers, delivery vehicles, diluents, excipients and/or pharmaceutical adjuvants. For example, a suitable vehicle may be water for injection, physiological saline solution, or artificial perilymph, possibly supplemented with other materials common in compositions for parenteral administration. Neutral buffered saline or saline mixed with serum albumin are further exemplary vehicles.

The primary solvent in a vehicle may be either aqueous or non-aqueous in nature. In addition, the vehicle may contain other pharmaceutically-acceptable excipients for modifying, modulating or maintaining the pH, osmolarity, viscosity, clarity, color, sterility, stability, rate of dissolution, or odor of the formulation. Similarly, the vehicle may contain still other pharmaceutically-acceptable excipients for modifying or maintaining the rate of release of the therapeutic product(s), or for promoting the absorption or penetration of the therapeutic product(s) across the tympanic membrane. Such excipients are those substances usually and customarily employed to formulate dosages for middle-ear administration in either unit dose or multi-dose form.

Once the therapeutic composition has been formulated, it may be stored in sterile vials as a solution, suspension, gel, emulsion, solid, or dehydrated or lyophilized powder. Such formulations may be stored either in a ready to use form or in a form, e.g., lyophilized, requiring reconstitution prior to administration.

The optimal pharmaceutical formulations will be determined by one skilled in the art depending upon considerations such as the route of administration and desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the present therapeutic agents of the invention.

Other effective administration forms, such as middle-ear slow-release formulations, inhalant mists, or orally active formulations are also envisioned. For example, in a sustained release formulation, the sensorineurotrophic compound may be bound to or incorporated into particulate preparations of polymeric compounds (such as polylactic acid, polyglycolic acid, etc.) or liposomes. Hylauronic acid may also be used, and this may have the effect of promoting sustained duration in the circulation. The sensorineuro-trophic compound pharmaceutical composition also may be formulated for middle-ear administration, e.g., by tympanic membrane infusion or injection, and may also include slow-release or sustained circulation formulations. Such middle-ear administered therapeutic compositions are typically in the form of a pyrogen-free, middle-ear acceptable aqueous solution comprising the sensorineurotrophic compound in a pharmaceutically acceptable vehicle. One preferred vehicle is sterile distilled water.

Certain formulations containing a sensorineurotrophic compound may be administered orally. A sensorineurotrophic compound which is administered in this fashion may be encapsulated and may be formulated with or without those carriers customarily used in the compounding of solid dosage forms. The capsule may be designed to release the active portion of the formulation at the point in the gastrointestinal tract when bioavailability is maximized and pre-systemic degradation is minimized. Additional excipients may be included to facilitate absorption of sensorineurotrophic compound. Diluents, flavorings, low melting point waxes, vegetable oils, lubricants, suspending agents, tablet disintegrating agents, and binders may also be employed.

The formulation of topical ear preparations, including middle-ear solutions, suspensions and ointments is well known to those skilled in the art (see, for example, “Remington's Pharmaceutical Sciences”, 18th Edition, Chapter 86, pp. 1581-1592, Mack Publishing Company, 1990). Other modes of administration are available, including injections to the middle ear. Methods and means for producing middle-ear preparations suitable for such modes of administration are also well known.

As used in this application, “middle-ear” refers to the space between the tympanic membrane and the inner ear. This location is external to all inner ear tissue and an invasive procedure might not be required to access this region if a formulation capable of penetrating through the tympanic membrane is administered. Otherwise, the material will be introduced to the middle ear by injection through the tympanic membrane or, in case repeated administrations are needed, a hole can be made in the tympanic membrane. An opening in the tympanic membrane is a frequent procedure, performed on an office-visit basis, in cases such as infections of the middle ear (usually in children). The opening generally closes spontaneously after a few days. Examples of systems for administering the therapeutic agent to these regions include inserts and “topically” applied drops, gels or ointments.

In the treatment of inner ear disease or injury it is also advantageous that a topically applied formulation include an agent to promote the penetration or transport of the therapeutic agent into the middle and inner ear. Such agents are known in the art.

Inner-ear systems include those tissue compartments within, between or around the tissue layers of the inner-ear, such as the cochlea and vestibular organ. These locations include the different structures of the cochlea such as the stria vascularis, Reissner's membrane, organ of Corti, spiral ligament and the cochlear neurons. An invasive procedure might not be required to access those structures since it has been shown that even proteins, let alone small molecules, do penetrate the membrane of the round window into the perilymph of the inner ear.

A particularly suitable vehicle for introducing the sensorineurotrophic compound into the inner ear by penetration through the round window membrane is artificial perilymph. This solution consists of 10 mM D-glucose, 1.5 mM CaCl, 1.5 mM MgCl in a 1.0% solution of Dulbecco's phosphate-buffered saline in deionized water at 280-300 mOsm and pH of 7.2. Yet another preparation may involve the formulation of the sensorineurotrophic compound with an agent, such as injectable microspheres or liposomes into the middle ear, that provides for the slow or sustained release of the molecules which may then be delivered as a depot injection. Other suitable means for the inner-ear introduction of sensorineurotrophic compound include implantable drug delivery devices which contain the sensorineurotrophic compound, or a cochlear-implant including a tunnel through which the sensorineurotrophic compound can be continuously delivered to the inner ear.

The ear-treatment preparations of the present invention, particularly topical preparations, may include other components, for example middle-ear acceptable preservatives, tonicity agents, cosolvents, complexing agents, buffering agents or other pH controlling agents, antimicrobials, antioxidants and surfactants, as are well known in the art. For example, suitable tonicity enhancing agents include alkali metal halides (preferably sodium or potassium chloride), mannitol, sorbitol and the like. Sufficient tonicity enhancing agent is advantageously added so that the formulation to be instilled into the ear is compatible with the osmolarity of the endo- and perilymph. Suitable preservatives include, but are not limited to, benzalkonium chloride, thimerosal, phenethyl alcohol, methylparaben, propylparaben, chlorhexidine, sorbic acid and the like. Hydrogen peroxide may also be used as preservative. Suitable cosolvents include, but are not limited to, glycerin, propylene glycol and polyethylene glycol. Suitable complexing agents include caffeine, polyvinyl-pyrrolidone, β-cyclodextrin or hydroxypropyl-β-cyclodextrin. The buffers can be conventional buffers such as borate, citrate, phosphate, bicarbonate, or tris-HCl.

The formulation components are present in a concentration and form that is acceptable to the middle or inner ear. For example, buffers are used to maintain the composition at physiological pH or at slightly lower pH, typically within a pH range of from about 5 to about 8.

Additional formulation components may include materials which prolong the residence in the middle ear of the administered therapeutic agent, particularly to maximize the topical contact and promote absorption of the therapeutic agent through the round window membrane. Suitable materials may include polymers or gel forming materials which increase the viscosity of the middle-ear preparation. The suitability of the formulations of the instant invention for controlled release (e.g., sustained and prolonged delivery) can be determined by various procedures known in the art. Yet another ear preparation may involve an effective quantity of sensorineurotrophic compound in admixture with non-toxic middle-ear treatment acceptable excipients. For example, the sensorineurotrophic compound may be prepared in tablet form. By dissolving the tablets in sterile water, or other appropriate vehicle, middle-ear treatment solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia.

Administration/Delivery of Sensorineurotrophic Compound

The sensorineurotrophic compound may be administered parenterally via a subcutaneous, intramuscular, intravenous, transpulmonary, transdermal, intrathecal or intracerebral route. For the treatment of inner-ear conditions, the sensorineurotrophic compound may be administered orally, systemically, or directly into the middle-ear (or directly into the inner-ear, especially in those situations where an invasive surgical procedure has already taken place), by topical application, inserts, injection or implants. For example, slow-releasing implants containing the molecules embedded in a biodegradable polymer matrix can be used to deliver the sensorineurotrophic compound. As noted, the sensorineurotrophic compound may be administered in the middle or inner ear, or it may be administered on top of the tympanic membrane in connection with one or more agents capable of promoting penetration or transport of the sensorineurotrophic compound across the membranes of the ear. The frequency of dosing will depend on the pharmacokinetic parameters of the sensorineurotrophic compound as formulated, and the route of administration.

The specific dose may be calculated according to considerations of body weight, body surface area or organ size. Further refinement of the calculations necessary to determine the appropriate dosage for treatment involving each of the above mentioned formulations is routinely made by those of ordinary skill in the art and is within the ambit of tasks routinely performed, especially in light of the dosage information and assays disclosed herein. Appropriate dosages may be determined using established assays in conjunction with appropriate dose-response data. One skilled in the art will appreciate that the dosage used in inner-ear formulations of the invention normally will be smaller as compared to that used in a systemic injection or oral administration.

The final dosage regimen involved in a method for treating the above-described conditions will be determined by the attending physician, considering various factors which modify the action of drugs, e.g., the age, condition, body weight, sex and diet of the patient, the severity of the condition, time of administration and other clinical factors familiar to one skilled in the art.

It is envisioned that the continuous administration or sustained delivery of sensorineurotrophic compound may be advantageous for a given condition. While continuous administration may be accomplished via a mechanical means, such as with an infusion pump, it is contemplated that other modes of continuous or near continuous administration may be practiced. For example, such administration may be by subcutaneous or muscular injections as well as oral pills and ear drops.

Techniques for formulating a variety of other sustained- or controlled-delivery means, such as liposome carriers, bio-erodible particles or beads and depot injections, are also known to those skilled in the art.

The compounds described in Formulas I-LXVII, below, possess asymmetric centers and thus can be produced as mixtures of stereoisomers or as individual R- and S-stereoisomers. The individual stereoisomers may be obtained by using an optically active starting material, by resolving a racemic or non-racemic mixture of an intermediate at some appropriate stage of the synthesis, or by resolving the compounds of Formulas I-LXLVII. It is understood that the compounds of Formulas I-LXVII encompass individual stereoisomers as well as mixtures (racemic and non-racemic) of stereoisomers. Preferably, S-stereoisomers are used in the pharmaceutical compositions and methods of the present invention.

The term “carbocyclic”, as used herein, refers to an organic cyclic moiety in which the cyclic skeleton is comprised of only carbon atoms whereas the term “heterocyclic” refers to an organic cyclic moiety in which the cyclic skeleton contains one or more heteroatoms selected from nitrogen, oxygen, or sulfur and which may or may not include carbon atoms. Carbocyclic or heterocyclic includes within its scope a single ring system, multiple fused rings (for example, bi- or tricyclic ring systems) or multiple condensed ring systems. One skilled in the art, therefore, will appreciate that in the context of the present invention, a cyclic structure formed by A and B (or A′ and B′) as described herein may comprise bi- or tri-cyclic or multiply condensed ring systems.

“Heterocycle” or “heterocyclic”, as used herein, refers to a saturated, unsaturated or aromatic carbocyclic group having a single ring, multiple fused (for example, bi- or tri-cyclic ring systems) rings or multiple condensed rings, and having at least one hetero atom such as nitrogen, oxygen or sulfur within at least one of the rings. This term also includes “Heteroaryl” which refers to a heterocycle in which at least one ring is aromatic.

In the context of the invention, useful carbo- and heterocyclic rings include, for example and without limitation, phenyl, benzyl, naphthyl, indenyl, azulenyl, fluorenyl, anthracenyl, indolyl, isoindolyl, indolinyl, benzofuranyl, benzothiophenyl, indazolyl, benzimidazolyl, benzthiazolyl, tetrahydrofuranyl, tetrahydropyranyl, pyridyl, pyrrolyl, pyrrolidinyl, pyridinyl, pyrimidinyl, purinyl, quinolinyl, isoquinolinyl, tetrahydroquinolinyl, quinolizinyl, furyl, thiophenyl, imidazolyl, oxazolyl, benzoxazolyl, thiazolyl, isoxazolyl, isotriazolyl, oxadiazolyl, triazolyl, thiadiazolyl, pyridazinyl, pyrimidinyl, pyrazinyl, triazinyl, trithianyl, indolizinyl, pyrazolyl, pyrazolinyl, pyrazolidinyl, thienyl, tetrahydroisoquinolinyl, cinnolinyl, phthalazinyl, quinazolinyl, quinoxalinyl, naphthyridinyl, pteridinyl, carbazolyl, acridinyl, phenazinyl, phenothiazinyl, and phenoxazinyl.

“Aryl” or “aromatic” refers to an aromatic carbocyclic or heterocyclic group having a single ring, for example, a phenyl ring, multiple rings, for example, biphenyl, or multiple condensed rings in which at least one ring is aromatic, for example, naphthyl, 1,2,3,4,-tetrahydronaphthyl, anthryl, or phenanthryl, which can be unsubstituted or substi-tuted. The substituents attached to a phenyl ring portion of an aryl moiety in the compounds of the invention may be configured in the ortho-, meta- or para-orientations, with the para-orientation being preferred.

Examples of typical aryl moieties included in the scope of the present invention may include, but are not limited to, the following:

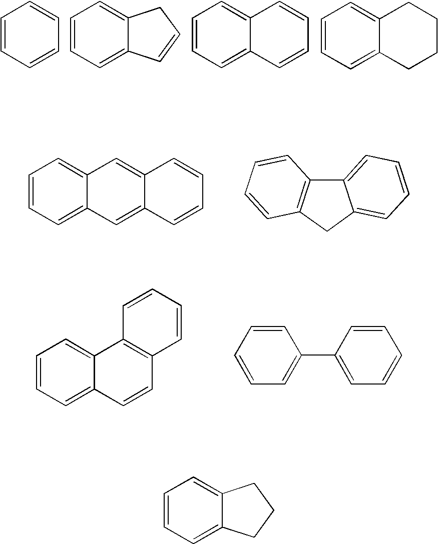

Examples of heterocyclic or heteroaryl moieties included in the scope of the present invention may include, but are not limited to, the following:

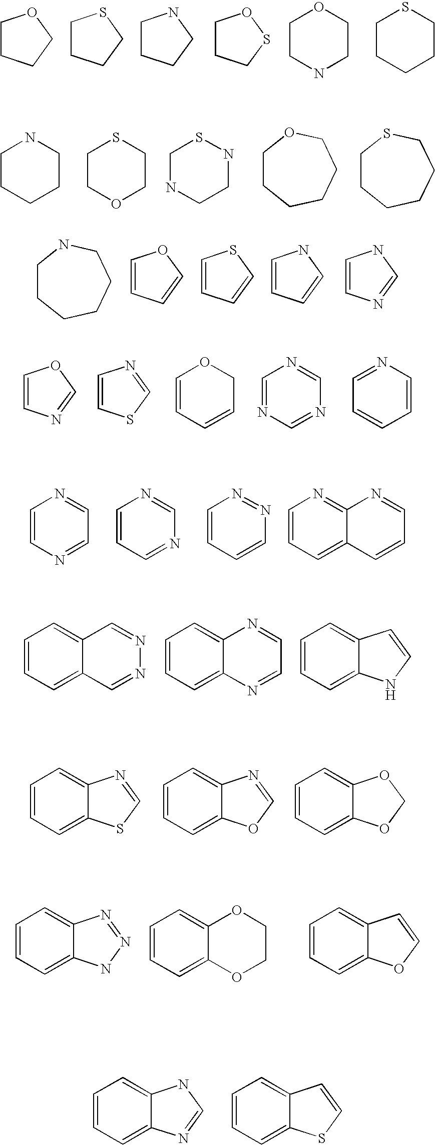

As one skilled in the art will appreciate such heterocyclic moieties may exist in several isomeric forms, all of which are to be encompassed by the present invention. For example, a 1,3,5-triazine moiety is isomeric to a 1,2,4-triazine group. Such positional isomers are to be considered within the scope of the present invention. Likewise, the heterocyclic or heteroaryl groups can be bonded to other moieties in the compounds of the invention. The point(s) of attachment to these other moieties is not to be construed as limiting on the scope of the invention. Thus, by way of example, a pyridyl moiety may be bound to other groups through the 2-, 3-, or 4-position of the pyridyl group. All such configurations are to be construed as within the scope of the present invention.

As used herein, “warm-blooded animal” includes a mammal, including a member of the human, equine, porcine, bovine, murine, canine or feline species. In the case of a human, the term “warm-blooded animal” may also be referred to as a “patient”. Further, as used herein, “a warm blooded animal in need thereof” refers to a warm-blooded animal which is susceptible to hearing loss due to genetic or environmental conditions or predispositions. This term also refers to a warm blooded animal which has already suffered some degree of sensorineural hearing loss because of genetic or environmental conditions to which the animal has been exposed or to which it has been predisposed. Environmental conditions can include the treatment with a therapeutic compound, such as an ototoxic substance, as well as other types of injury or insult such as noise or other factors contributing to hearing loss.

“Pharmaceutically acceptable salt”, as used herein, refers to an organic or inorganic salt which is useful in the treatment of a warm-blooded animal in need thereof. Such salts can be acid or basic addition salts, depending on the nature of the sensorineurotrophic agent compound to be used.

In the case of an acidic moiety in a sensorineurotrophic agent of the invention, a salt may be formed by treatment of the sensorineurotrophic agent with a basic compound, particularly an inorganic base. Preferred inorganic salts are those formed with alkali and alkaline earth metals such as lithium, sodium, potassium, barium and calcium. Preferred organic base salts include, for example, ammonium, dibenzylammonium, benzylammonium, 2-hydroxyethylammonium, bis(2-hydroxyethyl)ammonium, phenylethylbenzylamine, dibenzylethylenediamine, and the like salts. Other salts of acidic moieties may include, for example, those salts formed with procaine, quinine and N-methylglucosamine, plus salts formed with basic amino acids such as glycine, ornithine, histidine, phenylglycine, lysine and arginine. An especially preferred salt is a sodium or potassium salt of a sensorineurotrophic compound used in the invention.

With respect to basic moieties, a salt is formed by the treatment of the desired sensori-neurotrophic compound with an acidic compound, particularly an inorganic acid. Preferred inorganic salts of this type may include, for example, the hydrochloric, hydrobromic, hydroiodic, sulfuric, phosphoric or the like salts. Preferred organic salts of this type, may include, for example, salts formed with formic, acetic, succinic, citric, lactic, maleic, fumaric, palmitic, cholic, pamoic, mucic, d-glutamic, d-camphoric, glutaric, glycolic, phthalic, tartaric, lauric, stearic, salicyclic, methanesulfonic, benzenesulfonic, para-toluenesulfonic, sorbic, puric, benzoic, cinnamic and the like organic acids. An especially preferred salt of this type is a hydrochloride or sulfate salt of the desired sensorineurotrophic compound. Also, the basic nitrogen-containing groups can be quarternized with such agents as: 1) lower alkyl halides, such as methyl, ethyl, propyl, and butyl chloride, bromides and iodides; 2) dialkyl sulfates like dimethyl, diethyl, dibutyl and diamyl sulfates; 3) long chain alkyls such as decyl, lauryl, myristyl and stearyl substituted with one or more halide such as chloride, bromide and iodide; and 4) aralkyl halides like benzyl and phenethyl bromide and others.

Also encompassed in the scope of the present invention are pharmaceutically acceptable esters of a carboxylic acid or hydroxyl containing group, including a metabolically labile ester or a prodrug form of a compound of Formula (I′). A metabolically labile ester is one which may produce, for example, an increase in blood levels and prolong the efficacy of the corresponding non-esterified form of the compound. A prodrug form is one which is not in an active form of the molecule as administered but which becomes therapeutically active after some in vivo activity or biotransformation, such as metabolism, for example, enzymatic or hydrolytic cleavage. Esters of a compound of Formula (I′), may include, for example, the methyl, ethyl, propyl, and butyl esters, as well as other suitable esters formed between an acidic moiety and a hydroxyl containing moiety. Metabolically labile esters, may include, for example, methoxymethyl, ethoxymethyl, iso-propoxymethyl, α-methoxyethyl, groups such as α-((C₁-C₄)alkyloxy)ethyl; for example, methoxyethyl, ethoxyethyl, propoxyethyl, iso-propoxyethyl, etc.; 2-oxo-1,3-dioxolen-4-ylmethyl groups, such as 5-methyl-2-oxo-1,3,dioxolen-4-ylmethyl, etc.; C₁-C₃ alkylthiomethyl groups, for example, methylthio-methyl, ethylthiomethyl, isopropylthio-methyl, etc.; acyloxymethyl groups, for example, pivaloyloxy-methyl, α-acetoxymethyl, etc.; ethoxycarbonyl-1-methyl; or α-acyloxy-α-substituted methyl groups, for example α-acetoxyethyl.

Further, the compounds of the invention may exist as crystalline solids which can be crystal-lized from common solvents such as ethanol, N,N-dimethyl-formamide, water, or the like. Thus, crystalline forms of the compounds of the invention may exist as solvates and/or hydrates of the parent compounds or their pharmaceutically acceptable salts. All of such forms likewise are to be construed as falling within the scope of the invention.

“Alkyl” means a branched or unbranched saturated hydrocarbon chain comprising a designated number of carbon atoms. For example, C₁-C₆ straight or branched alkyl hydrocarbon chain contains 1 to 6 carbon atoms, and includes but is not limited to substituents such as methyl, ethyl, propyl, iso-propyl, butyl, iso-butyl, tert-butyl, n-pentyl, n-hexyl, and the like.

“Alkenyl” means a branched or unbranched unsaturated hydrocarbon chain comprising a designated number of carbon atoms. For example, C₂-C₆ straight or branched alkenyl hydrocarbon chain contains 2 to 6 carbon atoms having at least one double bond, and includes but is not limited to substituents such as ethenyl, propenyl, iso-propenyl, butenyl, iso-butenyl, tert-butenyl, n-pentenyl, n-hexenyl, and the like.

“Alkoxy” means the group —OR wherein R is alkyl as herein defined. Preferably, R is a branched or unbranched saturated hydrocarbon chain containing 1 to 6 carbon atoms.

“Aryl, heteroaryl, carbocycle, or heterocycle” includes but is not limited to cyclic or fused cyclic ring moieties and includes a mono-, bi- or tricyclic, carbo- or heterocyclic ring, wherein the ring is either unsubstituted or substituted in one or more position(s) with hydroxy, carbonyl, amino, amido, cyano, isocyano, nitro, nitroso, nitrilo, isonitrilo, imino, azo, diazo, sulfonyl, sulfhydryl, sulfoxy, thio, thiocarbonyl, thiocyano, formanilido, thioformamido, sulfhydryl, halo, halo-(C₁-C₆)-alkyl, trifluoromethyl, (C₁-C₆)-alkoxy, (C₂-C₆)-alkenoxy, (C₁-C₆)-alkylaryloxy, aryloxy, aryl-(C₁-C₆)-alkyloxy, (C₁-C₆)-alkylamino, amino-(C₁-C₆)-alkyl, thio-(C₁-C₆)-alkyl, C₁-C₆-alkylthio, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl and carbocyclic and heterocyclic moieties; wherein the individual ring sizes are 5-8 members; wherein the heterocyclic ring contains 1-4 heteroatom(s) selected from the group consisting of O, N, or S; wherein aromatic or tertiary alkyl amines are optionally oxidized to a corresponding N-oxide.

Examples of preferred carbocyclic and heterocyclic moieties include, without limitation, phenyl, benzyl, naphthyl, indenyl, azulenyl, fluorenyl, anthracenyl, indolyl, isoindolyl, indolinyl, benzofuranyl, benzothiophenyl, indazolyl, benzimidazolyl, benzthiazolyl, tetrahydrofuranyl, tetrahydropyranyl, pyridyl, pyrrolyl, pyrrolidinyl, pyridinyl, pyrimidinyl, purinyl, quinolinyl, isoquinolinyl, tetrahydroquinolinyl, quinolizinyl, furyl, thiophenyl, imidazolyl, oxazolyl, benzoxazolyl, thiazolyl, isoxazolyl, isotriazolyl, oxadiazolyl, triazolyl, thiadiazolyl, pyridazinyl, pyrimidinyl, pyrazinyl, triazinyl, trithianyl, indolizinyl, pyrazolyl, pyrazolinyl, pyrazolidinyl, thienyl, tetrahydroisoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, and adamantyl.

“Halo” means at least one fluoro, chloro, bromo, or iodo moiety.

“Stereoisomers” are isomers that differ only in the way the atoms are arranged in space.

“Isomers” are different compounds that have the same molecular formula and includes cyclic isomers such as (iso)indole and other isomeric forms of cyclic moieties.

“Enantiomers” are a pair of stereoisomers that are non-superimposable mirror images of each other.

“Diastereoisomers” are stereoisomers which are not mirror images of each other.

“Racemic mixture” means a mixture containing equal parts of individual enantiomers. “Non-racemic mixture” is a mixture containing unequal parts of individual enantiomers or stereoisomers.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN, wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carbocyclic and heterocyclic isosteres contemplated by this invention.

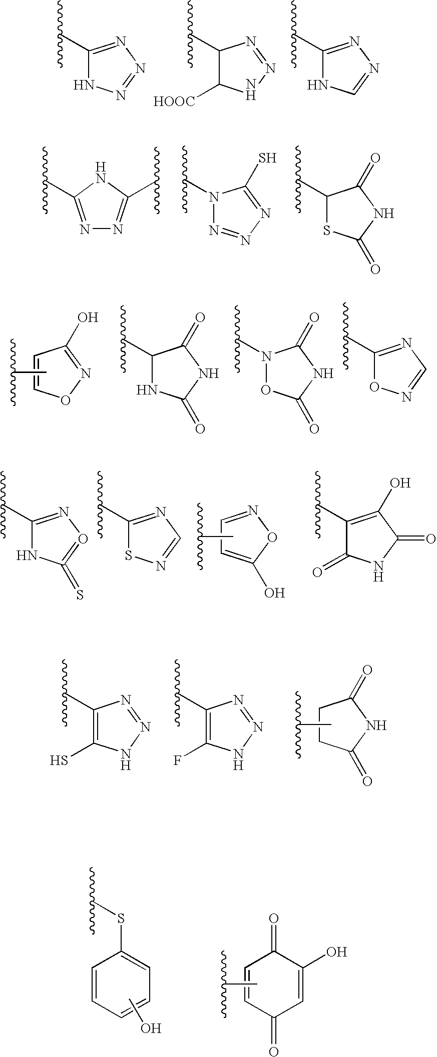

and —COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN, wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl and where the atoms of said ring structure may be optionally substituted at one or more positions with R₁, as defined herein. The present invention contemplates that when chemical substituents are added to a carboxylic isostere then the inventive compound retains the properties of a carboxylic isostere.

The present invention contemplates that when a carboxylic isostere is optionally substituted with one or more moieties selected from R³, as defined herein, then the substitution cannot eliminate the carboxylic acid isosteric properties of the inventive compound. The present invention contemplates that the placement of one or more R³ substituents upon a carbocyclic or heterocyclic carboxylic acid isostere shall not be permitted at one or more atom(s) which maintain(s) or is/are integral to the carboxylic acid isosteric properties of the inventive compound, if such substituent(s) would destroy the carboxylic acid isosteric properties of the inventive compound.

Other carboxylic acid isosteres not specifically exemplified or described in this specification are also contemplated by the present invention.

Further, as used throughout the teaching of the invention, a designation of:

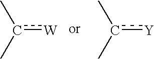

wherein W or Y is H₂, or similar designations, is meant to denote that two hydrogen atoms are attached to the noted carbon and that the bonds to each hydrogen are single bonds.

The sensorineurotrophic compounds useful in the invention comprise a variety of structural families. As noted, the primary consideration is that the compounds possess the desired sensorineurotrophic activity described herein. By way of description and not limitation, therefore, the following structural formulae are provided as exemplary of the sensorineurotrophic compound compounds useful in the treatment and prevention of sensorineural degeneration resulting in hearing loss:

In its broadest sense, the invention provides a method for the prevention or treatment of sensorineural hearing loss which comprises administering to a warm-blooded animal a compound of formula (I′):

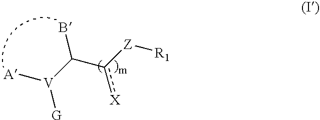

wherein

A′ is hydrogen, C₁ or C₂ alkyl, or benzyl;

B′ is C₁-C₄ straight or branched chain alkyl, benzyl or cyclohexylmethyl; or,

A′ and B′, taken together with the atoms to which they are attached, form a 5-7 membered saturated, unsaturated or aromatic heterocylic or carbocyclic ring which contains one or more additional O, C(R₁)₂, S(O)ₚ, N, NR₁, or NR₅ atoms;

V is CH, S, or N;

G is

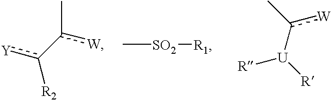

each R₁, independently, is hydrogen, C₁-C₉ straight or branched chain alkyl, or C₂-C₉ straight or branched chain alkenyl or alkynyl, C₃-C₉ cycloalkyl, C₅-C₇ cycloalkenyl, a carboxylic acid or carboxylic acid isostere, N(R₄)ₙ, Ar₁, Ar₄ or K-L wherein said alkyl, cycloalkyl, cycloalkenyl, alkynyl, alkenyl, Ar₁ or Ar₄ is optionally substituted with one or more substituent(s) independently selected from the group consisting of:

2-furyl, 2-thienyl, pyridyl, phenyl, C₃-C₆ cycloalkyl wherein said furyl, thienyl, pyridyl, phenyl or cycloalkyl group optionally is substituted with C₁-C₄ alkoxy, (Ar₁)ₙ, halo, halo-C₁-C₆-alkyl, carbonyl, thiocarbonyl, C₁-C₆ thioester, cyano, imino, COOR₆ in which R₆ is C₁-C₉ straight or branched chain alkyl or alkenyl, hydroxy, nitro, trifluoromethyl, C₁-C₆ alkoxy, C₂-C₄ alkenyloxy, C₁-C₆ alkylaryloxy C₁-C₆ aryloxy, aryl-(C₁-C₆)-alkyloxy, phenoxy, benzyloxy, thio-(C₁-C₆)-alkyl, C₁-C₆-alkylthio, sulfhydryl, sulfonyl, amino, (C₁-C₆)-mono- or di-alkylamino, amino-(C₁-C₆)-alkyl, aminocarboxy, C₃-C₈ cycloalkyl, C₁-C₆ straight or branched chain alkyl, C₂-C₆ straight or branched chain alkenyl optionally substituted with (Ar₁)ₙ, C₃-C₈ cycloalkyl, C₁-C₆ straight or branched chain alkyl, C₂-C₆ straight or branched chain alkenyl substituted with C₃-C₈ cycloalkyl, C₃-C₈ cycloalkyl, and Ar₂, and, wherein any carbon atom of an alkyl or alkenyl group may optionally replaced with O, NR₅, or S(O)_{P}; or,

R₁ is a moiety of the formula:

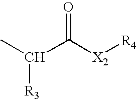

wherein:

R₃ is C₁-C₉ straight or branched chain alkyl which is optionally substituted with C₃-C₈ cycloalkyl or Ar₁;

X₂ is O or NR₆, wherein R₆ is selected from the group consisting of hydrogen, C₁-C₆ straight or branched chain alkyl, and C₂-C₆ straight or branched chain alkenyl;

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;

R₂ is C₁-C₉ straight or branched chain alkyl, C₂-C₉ straight or branched chain alkenyl, C₃-C₈ cycloalkyl, C₀-C₇ cycloalkenyl or Ar₁, wherein said alkyl, alkenyl, cycloalkyl, or cycloalkenyl is optionally substituted with one or more substituents selected from the group consisting of C₁-C₆ straight or branched chain alkyl, C₂-C₆ straight or branched chain alkenyl, C₃-C₈ cycloalkyl, C₅-C₇ cycloalkenyl, (Ar₁)ₙ and hydroxy; or,

R₂ is either hydrogen or P; Y is either oxygen or CH-P, provided that if R₂ is hydrogen, then Y is CH-P, or if Y is oxygen then R₂ is P;

P is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar₅, or Ar₅

Ar₁ or Ar₂, independently, is an alicyclic or aromatic, mono-, bi- or tricyclic, carbo- or heterocyclic ring, wherein the ring is optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₃-C₈ cycloalkyl, C₅-C₇ cycloalkenyl, C₁-C₄ alkoxy, C₂-C₄ alkenyloxy, phenoxy, benzyloxy, and amino; wherein the individual ring contains 5-8 members; and wherein the heterocyclic ring contains 1-6 heteroatom(s) independently selected from the group consisting of O, N, and S, and, wherein any aromatic or tertiary alkylamine is optionally oxidized to a corresponding N-oxide;

m is 0 or 1

n is 1 or 2;

p is 0, 1, or 2;

t is 0, 1, 2, 3, or 4;

X is O, CH₂ or S;

W and Y, independently, are O, S, CH₂ or H₂;

Z is C(R₁)₂, O, S, a direct bond or NR₁; or, Z-R₁ is J-K-L,

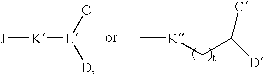

wherein:

C and D are, independently, hydrogen, Ar₄, Ar₁, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, Ar₁ and Ar₄; wherein said alkyl, alkenyl, cycloalkyl or cycloalkenyl is optionally substituted with C₁-C₆ alkyl, C₂-C₆ alkenyl, hydroxy, amino, halo, halo-(C₁-C₆)-alkyl, thiocarbonyl, C₁-C₆ ester, C₁-C₆ thioester, C₁-C₆ alkoxy, C₂-C₆ alkenoxy, cyano, nitro, imino, C₁-C₆ alkylamino, amino-(C₁-C₆)alkyl, sulfhydryl, thio-(C₁-C₆)alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NR₅, or (SO)ₚ;

C′ and D′ are independently hydrogen, Ar₅, C₁-C₆ straight or branched chain alkyl, or C₂-C₆ straight or branched chain alkenyl, wherein said alkyl or alkenyl is optionally substituted with C₅-C₇ cycloalkyl, C₅-C₇ cycloalkenyl, or Ar₅, wherein, one or two carbon atom(s) of said alkyl or alkenyl may be substituted with one or two heteroatom(s) independently selected from the group consisting of oxygen, sulfur, SO, and SO₂ in chemically reasonable substitution patterns, or

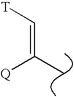

wherein

Q is hydrogen, C₁-C₆ straight or branched chain alkyl, or C₂-C₆ straight or branched chain alkenyl; and

T is Ar₅ or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl J is O, NR₁, S, or (CR₁)₂;

K is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl or Ar₃₁ is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl or Ar₃₁ is optionally replaced with O, NR′″, or S(O)ₚ;

K′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, C₁-C₆-ester, thio-C₁-C₆-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NR₅, S(O)_{P};

K″ is C(R₁)₂, 0 S, a direct bond or NR₁;

R′″ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₃ group;

L is an aromatic amine or a tertiary amine oxidized to a corresponding N-oxide; said aromatic amine being selected from the group consisting of pyridyl, pyrimidyl, quinolinyl, and isoquinolinyl, said aromatic amine being optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; and wherein said tertiary amine is NRₓR_{y}R_{z}, wherein Rₓ, R_{y}, and R_{z} are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl, or Ar₃ is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar₃ is optionally replaced with O, NR′, S(O)ₚ;

L′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NR₅, S(O)ₚ

Ar₃ is selected from the group consisting of pyrrolidinyl, pyridyl, pyrimidyl, pyrazyl, pyridazyl, quinolinyl, and isoquinolinyl; or,

Ar₄ is an alicyclic or aromatic, mono-, bi- or tricyclic, carbo- or heterocyclic ring, wherein the ring is optionally substituted with one or more substituent(s) independently selected from the group consisting of alkylamino, amido, amino, amino-(C₁-C₆)-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-(C₁-C₆)-alkyl, hydroxy, imino, isocyano, isonitrilo, nitrilo, nitro, nitroso, phenoxy, sulfhydryl, sulfonylsulfoxy, thio, thio-(C₁-C₆)-alkyl, thiocarbonyl, thiocyano, thio-C₁-C₆-ester, thioformamido, trifluoromethyl, and carboxylic and heterocyclic moieties; wherein the individual alicyclic or aromatic ring contains 5-8 members and wherein said heterocyclic ring contains 1-6 heteroatom(s) independently selected from the group consisting of O, N, and S; and wherein any aromatic or tertiary alkyl amine is optionally oxidized to a corresponding N-oxide;

Ar₅ is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar₅ optionally contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;

R₅ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₄ or Ar₁ group;

U is either O or N, provided that:

when U is O, then R′ is a lone pair of electrons and R″ is selected from the group consisting of Ar₄, C₃-C₈ cycloalkyl, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; and

when U is N, then R′ and R″ are, independently, selected from the group consisting of hydrogen, Ar₄, C₃-C₁₀ cycloalkyl, a C₇-C₁₂ bi- or tri-cyclic carbocycle, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; or R′ and R″ are taken together to form a heterocyclic 5- or 6-membered ring selected from the group consisting of pyrrolidine, imidazolidine, pyrazolidine, piperidine, and piperazine; or, a pharmaceutically acceptable salt, ester or solvate thereof.

Additionally, the invention provides a method for the prevention or treatment injury or degeneration of inner ear sensory cells by administering a sensorineurotrophic compound of Formula (I′) to a patient in need thereof.

Also provided are a compound of Formula (I′) for use in the preparation of a medicament for the treatment or prevention of hearing loss. Additionally, there is provided a compound of Formula (I′) for use in the preparation of a medicament for the treatment or prevention of injury or degeneration of inner ear sensory cells. In this aspect of the invention, there are also provided a formulation comprising a compound of Formula (I′) for use in the preparation of a medicament for the treatment or prevention of hearing loss, as well as a formulation comprising a compound of Formula (I′) for use in the preparation of a medicament for the treatment or prevention of injury or degeneration of inner ear sensory cells.

Additionally, there is provided a formulation adapted for use in the treatment of hearing loss which comprises a compound of Formula (I′) associated with a pharmaceutically acceptable carrier, diluent or excipient therefor, as well as a formulation adapted for use in the treatment or prevention of injury or degeneration of inner ear sensory cells which comprises a compound of Formula (I′) associated with a pharmaceutically acceptable carrier, diluent or excipient therefor.

More specifically, the invention provides methods, uses, and formulations described above which comprise the use of any of the compounds described below,

I. Heterocyclic Thioesters and Ketones

### Formula I

In particular, the sensorineurotrophic agent may be a compound of formula I:

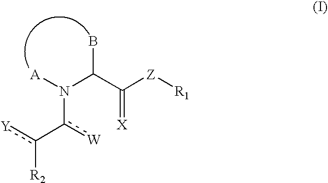

or a pharmaceutically acceptable salt, ester, or solvate thereof, wherein:

A and B, together with the nitrogen and carbon atoms to which they are respectively attached, form a 5-7 membered saturated or unsaturated heterocyclic ring containing one or more heteroatom(s) independently selected from the group consisting of O, S, SO, SO₂, N, NH, and NR₂;

X is either O or S;

Z is either S, CH₂, CHR₁ or CR₁R₃;

W and Y are independently O, S, CH₂ or H₂;

R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxy; and

Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

### Formula II

The sensorineurotrophic agent may also be a compound of formula II:

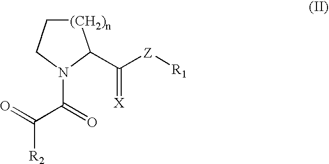

or a pharmaceutically acceptable salt, ester, or solvate thereof, wherein:

n is 1 or 2;

X is O or S;

Z is selected from the group consisting of S, CH₂, CHR₁, and CR₁R₃;

R₁ and R₃ are independently selected from the group consisting of C₁-C₅ straight or branched chain alkyl, C₂-C₅ straight or branched chain alkenyl, and Ar₁, wherein said alkyl, alkenyl or Ar₁ is unsubstituted or substituted with one or more substituent(s) independently selected from the group consisting of halo, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, and Ar₁;

R₂ is selected from the group consisting of C₁-C₉ straight or branched chain alkyl, C₂-C₉ straight or branched chain alkenyl, C₃-C₈ cycloalkyl, C₅-C₇ cycloalkenyl, and Ar₁; and

Ar₁ is phenyl, benzyl, pyridyl, fluorenyl, thioindolyl or naphthyl, wherein said Ar₁ is unsubstituted or substituted with one or more substituent(s) independently selected from the group consisting of halo, trifluoromethyl, hydroxy, nitro, C₁-C₆ straight or branched chain alkyl, C₂-C₆ straight or branched chain alkenyl, C₁-C₄ alkoxy, C₂-C₄ alkenyloxy, phenoxy, benzyloxy, and amino.

Preferred compounds of formula II are presented in TABLE I.

TABLE I

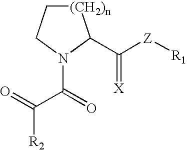

| No | n | X | Z | R₁ | R₂ |
|---|---|---|---|---|---|
| 1 | 1 | O | CH₂ | 3-Phenylpropyl | 1,1-Dimethylpropyl |
| 2 | 1 | O | CH₂ | 3-(3-Pyridyl)propyl | 1,1-Dimethylpropyl |
| 3 | 1 | O | CH₂ | 3-Phenylpropyl | tert-Butyl |
| 4 | 1 | O | CH₂ | 3-(3-Pyridyl)propyl | tert-Butyl |
| 5 | 1 | O | CH₂ | 3-(3-Pyridyl)propyl | Cyclohexyl |
| 6 | 1 | O | CH₂ | 3-(3-Pyridyl)propyl | Cyclopentyl |
| 7 | 1 | O | CH₂ | 3-(3-Pyridyl)propyl | Cycloheptyl |
| 8 | 1 | O | CH₂ | 2-(9-Fluorenyl)ethyl | 1,1-Dimethylpropyl |
| 9 | 1 | O | S | 2-Phenethyl | 1,1-Dimethylpropyl |
| 10 | 2 | O | S | 2-Phenethyl | 1,1-Dimethylpropyl |
| 11 | 1 | O | S | Methyl(2-thioindole) | 1,1-Dimethylpropyl |
| 12 | 1 | O | S | 2-Phenethyl | Cyclohexyl |
| 13 | 2 | O | S | 2-Phenethyl | tert-Butyl |
| 14 | 2 | O | S | 2-Phenethyl | Phenyl |
| 15 | 1 | O | CH₂ | 3-(4-Methoxyphenyl)propyl | 1,1-Dimethylpropyl |
| 16 | 2 | O | CH₂ | 4-(4-Methoxyphenyl)butyl | 1,1-Dimethylpropyl |
| 17 | 2 | O | CH₂ | 4-Phenylbutyl | 1,1-Dimethylpropyl |
| 18 | 2 | O | CH₂ | 4-Phenylbutyl | Phenyl |
| 19 | 2 | O | CH₂ | 4-Phenylbutyl | Cyclohexyl |
| 20 | 1 | S | CH₂ | 3-Phenylpropyl | 1,1-Dimethylpropyl |
| 21 | 1 | S | S | 2-Phenethyl | 1,1-Dimethylpropyl |
| 22 | 2 | S | CH₂ | 3-Phenylpropyl | 1,1-Dimethylpropyl |
| 23 | 2 | S | S | 2-Phenethyl | 1,1-Dimethylpropyl |
| 24 | 2 | O | CHR₁ | 3-Phenylpropyl | 1,1-Dimethylpropyl |
| 25 | 2 | O | CHR₁ | 3-Phenylpropyl | Cyclohexyl |
| 26 | 2 | O | CHR₁ | 3-Phenylpropyl | Phenyl |
| 27 | 2 | O | CHR₁ | 3-Phenylpropyl | 3,4,5- |
| | | | | | Trimethoxyphenyl |
| 28 | 1 | O | S | 2-Phenethyl | Cyclopentyl |
| 29 | 2 | O | S | 3-Phenylpropyl | tert-Butyl |
| 30 | 1 | O | S | 3-Phenylpropyl | 1,1-Dimethylpropyl |
| 31 | 1 | O | S | 3-(3-Pyridyl)propyl | 1,1-Dimethylpropyl |
| 32 | 1 | O | S | 3-Phenylpropyl | Cyclohexyl |
| 33 | 1 | O | S | 4-Phenylbutyl | Cyclohexyl |
| 34 | 1 | O | S | 4-Phenylbutyl | 1,1-Dimethylpropyl |
| 35 | 1 | O | S | 3-(3-Pyridyl)propyl | Cyclohexyl |
| 36 | 1 | O | S | 3,3-Diphenylpropyl | 1,1-Dimethylpropyl |
| 37 | 1 | O | S | 3,3-Diphenylpropyl | Cyclohexyl |
| 38 | 1 | O | S | 3-(4-Methoxyphenyl)propyl | 1,1-Dimethylpropyl |
| 39 | 2 | O | S | 4-Phenylbutyl | tert-Butyl |
| 40 | 2 | O | S | 1,5-Diphenylpentyl | 1,1-Dimethylpropyl |
| 41 | 2 | O | S | 1,5-Diphenylpentyl | Phenyl |
| 42 | 2 | O | S | 3-(4-Methoxyphenyl)propyl | 1,1-Dimethylpropyl |
| 43 | 2 | O | S | 3-(4-Methoxyphenyl)propyl | Phenyl |
| 44 | 2 | O | S | 3-(1-Naphthyl)propyl | 1,1-Dimethylpropyl |
| 45 | 1 | O | S | 3,3-Di(4-fluoro)phenyl- | 1,1-Dimethylpropyl |
| | | | | propyl | |
| 46 | 1 | O | S | 4,4-Di(4- | 1,1-Dimethylpropyl |
| | | | | fluoro)phenylbutyl | |
| 47 | 1 | O | S | 3-(1-Naphthyl)propyl | 1,1-Dimethylpropyl |
| 48 | 1 | O | S | 2.2-Diphenylethyl | 1,1-Dimethylpropyl |
| 49 | 2 | O | S | 2,2-Diphenylethyl | 1,1-Dimethylpropyl |
| 50 | 2 | O | S | 3,3-Diphenylpropyl | 1,1-Dimethylpropyl |
| 51 | 1 | O | S | 3-(4- | 1,1-Dimethylpropyl |
| | | | | {Trifluoromethyl}phenyl)propyl | |
| 52 | 1 | O | S | 3-(2-Naphthyl)propyl | 1,1-Dimethylpropyl |
| 53 | 2 | O | S | 3-(1-Naphthyl)propyl | 1,1-Dimethylpropyl |
| 54 | 1 | O | S | 3-(3-Chloro)phenylpropyl | 1,1-Dimethylpropyl |
| 55 | 1 | O | S | 3-(3- | 1,1-Dimethylpropyl |
| | | | | {Trifluoromethyl}phenyl)propyl | |
| 56 | 1 | O | S | 3-(2-Biphenyl)propyl | 1,1-Dimethylpropyl |
| 57 | 1 | O | S | 3-(2-Fluorophenyl)propyl | 1,1-Dimethylpropyl |
| 58 | 1 | O | S | 3-(3-Fluorophenyl)propyl | 1,1-Dimethylpropyl |
| 59 | 2 | O | S | 4-Phenylbutyl | 1,1-Dimethylpropyl |
| 60 | 2 | O | S | 3-Phenylpropyl | 1,1-Dimethylpropyl |
| 61 | 1 | O | S | 3-(2-Chloro)phenylpropyl | 1,1-Dimethylpropyl |
| 62 | 2 | O | S | 3-(3-Chloro)phenylpropyl | 1,1-Dimethylpropyl |
| 63 | 2 | O | S | 3-(2-Fluoro)phenylpropyl | 1,1-Dimethylpropyl |
| 64 | 2 | O | S | 3-(3-Fluoro)phenylpropyl | 1,1-Dimethylpropyl |
| 65 | 1 | O | S | 3-(2,5- | 1,1-Dimethylpropyl |
| | | | | Dimethoxyphenyl)propyl | |
| 66 | 1 | O | CH₂ | 3-Phenylpropyl | Cyclohexyl |
| 67 | 1 | O | CH₂ | 3-Phenylethyl | tert-Butyl |
| 68 | 2 | O | CH₂ | 4-Phenylbutyl | Cyclohexyl |
| 69 | 2 | O | CHR₁ | 2-Phenylethyl | tert-Butyl |
| 70 | 1 | O | CH₂ | 3,3-Di(4- | 1,1-Dimethylpropyl |
| | | | | fluorophenyl)propyl | |
| 71 | 2 | O | CH₂ | 3-Phenylpropyl | 1,1-Dimethylpropyl |

Preferred compounds of TABLE I are named as follows:

1 (2S)-2-({1-Oxo-5-phenyl}-pentyl-1-(3,3-dimethyl-1,2-dioxopentyl)pyrrolidine

2 3,3-Dimethyl-1-[(2S)-2-(5-(3-pyridyl)pentanoyl)-1-pyrrolidine]-1,2-pentanedione

3 (2S)-2-({1-Oxo-4-phenyl}-butyl-1-(3,3-dimethyl-1,2-dioxobutyl)pyrrolidine

9 2-Phenyl-1-ethyl (2S)-1-(3,3-dimethyl-1,2-dioxopentyl)-2-pyrrolidinecarbothioate

10 2-Phenyl-1-ethyl 1-(3,3-dimethyl-1,2-dioxopentyl)-2-piperidinecarbothioate

11 (3-Thioindolyl)methyl (2S)-1-(3,3-dimethyl-1,2-dioxopentyl)-2-pyrrolidinecarbothioate

12 2-Phenyl-1-ethyl (2S)-1-(2-cyclohexyl-1,2-dioxoethyl)-2-pyrrolidinecarbothioate

14 2-Phenyl-1-ethyl 1-(2-phenyl-1,2-dioxoethyl)-2-piperidinecarbothioate

28 2-Phenyl-1-ethyl (2S)-1-(1-cyclopentyl-1,2-dioxoethyl)-2-pyrrolidinecarbothioate

29 3-Phenyl-1-propyl 1-(3,3-dimethyl-1,2-dioxobutyl)-2-piperidinecarbothioate

30 3-Phenyl-1-propyl (2S)-1-(3,3-dimethyl-1,2-dioxopentyl)-2-pyrrolidinecarbothioate

31 3-(3-Pyridyl)-1-propyl (2S)-1-(3,3-dimethyl-1,2-dioxopentyl)-2-pyrrolidinecarbothioate

32 3-Phenyl-1-propyl (2S)-1-(2-cyclohexyl-1,2-dioxoethyl)-2-pyrrolidinecarbothioate

33 4-Phenyl-1-butyl (2S)-1-(2-cyclohexyl-1,2-dioxoethyl)-2-pyrrolidinecarbothioate

34 4-Phenyl-1-butyl (2S)-1-(3,3-dimethyl-1,2-dioxopentyl)-2-pyrrolidinecarbothioate

35 3-(3-Pyridyl)-1-propyl (2S)-1-(2-cyclohexyl-1,2-dioxoethyl)-2-pyrrolidinecarbothioate

36 3,3-Diphenyl-1-propyl (2S)-1-(3,3-dimethyl-1,2-dioxopentyl)-2-pyrrolidinecarbothioate

37 3,3-Diphenyl-1-propyl (2S)-1-(2-cyclohexyl-1,2-dioxoethyl)-2-pyrrolidinecarbothioate

38 3-(para-Methoxyphenyl)-1-propyl (2S)-1-(3,3-dimethyl-1,2-dioxopentyl)-2-pyrrolidine-carbothioate

39 4-Phenyl-1-butyl 1-(1,2-dioxo-3,3-dimethylbutyl)-2-piperidinecarbothioate

40 1,5-Diphenyl-3-pentyl 1-(3,3-dimethyl-1,2-dioxopentyl)-2-piperidinecarbothioate

41 1,5-Diphenyl-3-mercaptopentyl 1-(3-phenyl-1,2-dioxoethyl)-2-piperidinecarbothioate

42 3-(para-Methoxyphenyl)-1-propyl 1-(1,2-dioxo-3,3-dimethylpentyl)piperidine-2-carbothioate

43 3-(para-Methoxyphenyl)-1-propyl 1-(2-phenyl-1,2-dioxoethyl)piperidine-2-carbothioate

44 3-(1-Naphthyl)-1-propyl 1-(3,3-dimethyl-1,2-dioxopentyl)piperidine-2-carbothioate

45 3,3-Di(para-fluoro)phenyl-1-propyl (2S)-1-(3,3-dimethyl-1,2-dioxopentyl)-2-pyrrolidine-carbothioate

46 4,4-Di(para-fluorophenyl)butyl 1-(3,3-dimethyl-2-oxopentanoyl)-2-pyrrolidinecarbothioate

47 3-(1-Naphthyl)propyl (2S)-1-(3,3-dimethyl-2-oxopentanoyl)-2-pyrrolidinecarbothioate

48 2,2-Diphenylethyl (2S)-1-(3,3-dimethyl-2-oxopentanoyl)tetrahydro-1H-2-pyrrolidinecarbothioate

49 2,2-Diphenylethyl (2S)-1-(3,3-dimethyl-2-oxopentanoyl)-2-piperidinecarbothioate

50 3,3-Diphenylpropyl 1-(3,3-dimethyl-2-oxopentanoyl)-2-piperidinecarbothioate

51 3-[4-(Trifluoromethyl)phenyl]propyl (2S)-1-(3,3-dimethyl-2-oxopentanoyl)-2-pyrrolidine-carbothioate

52 3-(2-Naphthyl)propyl (2S)-1-(3,3-dimethyl-2-oxopentanoyl)-2-pyrrolidinecarbothioate

53 3-(2-Naphthyl)propyl (2R,S)-1-(3,3-dimethyl-2-oxopentanoyl)-2-piperidinecarbothioate

54 3-(3-Chlorophenyl)propyl (2S)-1-(3,3-dimethyl-2-oxopentanoyl)-2-pyrrolidinecarbothioate

55 3-[3-(Trifluoromethyl)phenyl]propyl (2S)-1-(3,3-dimethyl-2-oxopentanoyl)-2-pyrrolidine-carbothioate

56 3-(1-Biphenyl)propyl (2S)-1-(3,3-dimethyl-2-oxopentanoyl)-2-pyrrolidinecarbothioate

57 3-(2-Fluorophenyl)propyl (2S)-1-(3,3-dimethyl-2-oxopentanoyl)-2-pyrrolidinecarbothioate

58 3-(3-Fluorophenyl)propyl (2S)-1-(3,3-dimethyl-2-oxopentanoyl)-2-pyrrolidinecarbothioate

59 4-Phenylbutyl 1-(3,3-dimethyl-2-oxopentanoyl)-2-piperidinecarbothioate

60 3-Phenylpropyl 1-(3,3-dimethyl-2-oxopentanoyl)-2-piperidinecarbothioate

61 3-(2-Chlorophenyl)propyl (2S)-1-(3,3-dimethyl-2-oxopentanoyl)-2-pyrrolidinecarbothioate

62 3-(2-Chlorophenyl)propyl 1-(3,3-dimethyl-2-oxopentanoyl)-2-piperidinecarbothioate

63 3-(2-Fluorophenyl)propyl 1-(3,3-dimethyl-2-oxopentanoyl)-2-piperidinecarbothioate

64 3-(3-Fluorophenyl)propyl 1-(3,3-dimethyl-2-oxopentanoyl)-2-piperidinecarbothioate

65 3-(3,4-Dimethoxyphenyl)propyl (2S)-1-(3,3-dimethyl-2-oxopentanoyl)-2-pyrrolidinecarbothioate

66 (2S)-2-({1-Oxo-4-phenyl}-butyl-1-(2-Cyclohexyl-1,2-dioxoethyl)pyrrolidine

67 2-({1-Oxo-4-phenyl}-butyl-1-(3,3-dimethyl-1,2-dioxobutyl)pyrrolidine

68 2-({1-Oxo-6-phenyl}-hexyl-1-(2-Cyclohexyl-1,2-dioxoethyl)piperidine

69 2-({1-Oxo-[2-{2′-phenyl}ethyl]-4-phenyl}-butyl-1-(3,3-dimethyl-1,2-dioxobutyl)piperidine

70 1-{(2S)-2-[5,5-di(4-Fluorophenyl)pentanoyl]-2-pyrrolidine}-3,3-dimethyl-1,2-pentanedione

71 3,3-Dimethyl-1-[2-(4-phenylpentanoyl)piperidino]-1,2-pentanedione

### Formula III

Furthermore, the sensorineurotrophic agent may be a compound of formula III:

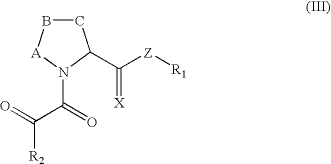

or a pharmaceutically acceptable salt, ester, or solvate thereof, wherein:

A, B, and C are independently CH₂, O, S, SO, SO₂, NH or NR₂;

X is O or S;

Z is S, CH₂, CHR₁ or CR₁R₃;

R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and

Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

Preferred compounds of formula III are presented in TABLE II:

TABLE II

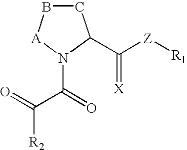

| No. | A | B | C | X | Z | R₁ | R₂ |
|---|---|---|---|---|---|---|---|
| 72 | CH₂ | S | CH₂ | O | S | 2-phenethyl | 1,1-dimethylpropyl |
| 73 | CH₂ | S | CH₂ | O | CH₂ | 3-phenylpropyl | 1,1-dimethylpropyl |
| 74 | CH₂ | CH₂ | NH | O | S | 2-phenethyl | 1,1-dimethylpropyl |
| 75 | CH₂ | S | CH₂ | S | S | 2-phenethyl | 1,1-dimethylpropyl |

### Formula IV

Alternatively, the sensorineurotrophic agent may be a compound of formula IV:

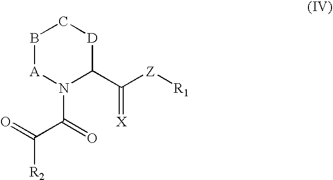

or a pharmaceutically acceptable salt, ester, or solvate thereof, wherein:

A, B, C and D are independently CH₂, O, S, SO, SO₂, NH or NR₂;

X is O or S;

Z is S, CH₂, CHR₁ or CR₁R₃;

R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₃-C₈ cycloalkyl, C₁-C₄ straight or branched chain alkyl, C₂-C₄ straight or branched chain alkenyl, and hydroxyl; and

Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoro-methyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

Preferred compounds of formula IV are presented in TABLE III.

TABLE III

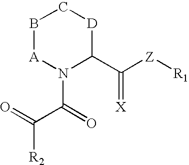

| No. | A | B | C | D | X | Z | R₁ | R₂ |
|---|---|---|---|---|---|---|---|---|
| 76 | CH₂ | CH₂ | O | CH₂ | O | CH₂ | 3-phenylpropyl | 1,1-dimethylpropyl |
| 77 | CH₂ | CH₂ | O | CH₂ | O | S | 2-phenethyl | 1,1-dimethylpropyl |
| 78 | CH₂ | CH₂ | S | CH₂ | O | CH₂ | 3-phenylpropyl | 1,1-dimethylpropyl |
| 79 | CH₂ | CH₂ | S | CH₂ | O | S | 2-phenethyl | 1,1-dimethylpropyl |

### Formula V

The sensorineurotrophic agent may further be a compound of formula V:

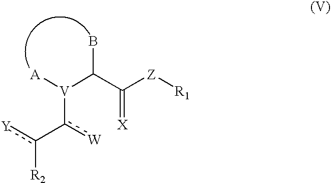

or a pharmaceutically acceptable salt, ester, or solvate thereof, wherein:

V is CH, N, or S;

A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR₄;

R₄ is either C₁-C₉ straight or branched chain alkyl, C₂-C₉ straight or branched chain alkenyl, C₃-C₉ cycloalkyl, C₅-C₇ cycloalkenyl, or Ar₃, wherein R₄ is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;

Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and

R₁, R₂, W, X, Y, and Z are as defined in Formula I above.

II. Heterocyclic Esters and Amides

### Formula VI

Additionally, the sensorineurotrophic agent may be a compound of formula VI:

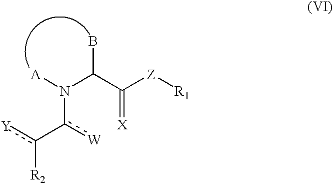

or a pharmaceutically acceptable salt, ester, or solvate thereof, wherein:

A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₁;

X is O or S;

Z is O, NH or NR₁;

W and Y are independently O, S, CH₂ or H₂;

R₁ is C₁-C₆ straight or branched chain alkyl or C₂-C₆ straight or branched chain alkenyl, which is substituted with one or more substituent(s) independently selected from the group consisting of (Ar₁)ₙ, C₁-C₆ straight or branched chain alkyl or C₂-C₆ straight or branched chain alkenyl substituted with (Ar₁)ᵣ, C₃-C₈ cycloalkyl, C₁-C₆ straight or branched chain alkyl or C₂-C₆ straight or branched chain alkenyl substituted with C₃-C₈ cycloalkyl, and Ar₂;

n is 1 or 2;

R₂ is either C₁-C₉ straight or branched chain alkyl, C₂-C₉ straight or branched chain or alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and

Ar₁ and Ar₂ are independently an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

Suitable carbo- and heterocyclic rings include without limitation naphthyl, indolyl, furyl, thiazolyl, thienyl, pyridyl, quinolinyl, isoquinolinyl, fluorenyl and phenyl.

### Formula VII

The sensorineurotrophic agent may also be a compound of formula VII:

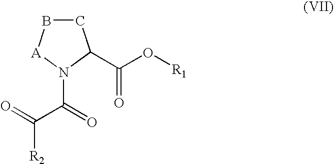

or a pharmaceutically acceptable salt, ester, or solvate thereof, wherein:

A, B and C are independently CH₂, O, S, SO, SO₂, NH or NR₁;

R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl, or Ar₁; and

Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

A preferred compound of formula VII is:

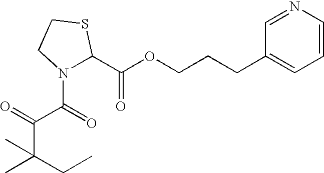

In a particularly preferred embodiment of formula VII compounds:

A is CH₂;

B is CH₂ or S;

C is CH₂ or NH;

R₁ is selected from the group consisting of 3-phenylpropyl and 3-(3-pyridyl)propyl; and

R₂ is selected from the group consisting of 1,1-dimethylpropyl, cyclohexyl, and tert-butyl.

Specific examples of this embodiment are presented in TABLE IV:

TABLE IV

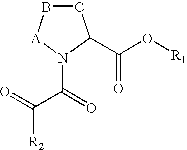

| No. | A | B | C | R₁ | R₂ |
|---|---|---|---|---|---|
| 80 | CH₂ | S | CH₂ | 3-phenylpropyl | 1,1-dimethylpropyl |
| 81 | CH₂ | S | CH₂ | 3-(3-pyridyl)propyl | 1,1-dimethylpropyl |
| 82 | CH₂ | S | CH₂ | 3-phenylpropyl | cyclohexyl |
| 83 | CH₂ | S | CH₂ | 3-phenylpropyl | tert-butyl |
| 84 | CH₂ | CH₂ | NH | 3-phenylpropyl | 1,1-dimethylpropyl |
| 85 | CH₂ | CH₂ | NH | 3-phenylpropyl | cyclohexyl |
| 86 | CH₂ | CH₂ | NH | 3-phenylpropyl | tert-butyl |

### Formula VIII

In a further embodiment of this invention, the sensorineurotrophic agent may be a compound of formula VIII:

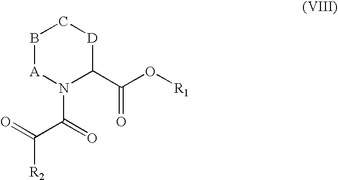

or a pharmaceutically acceptable salt, ester, or solvate thereof, wherein:

A, B, C and D are independently CH₂, O, S, SO, SO₂, NH or NR₁;

R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl, or Ar₁; and

Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

In a particularly preferred embodiment of formula VIII compounds:

A is CH₂;

B is CH₂;

C is S, O or NH;

D is CH₂;

R₁ is selected from the group consisting of 3-phenylpropyl and (3,4,5-trimethoxy)phenylpropyl; and

R₂ is selected from the group consisting of 1,1-dimethylpropyl, cyclohexyl, tert-butyl, phenyl, and 3,4,5-trimethoxyphenyl.

Specific examples of this embodiment are presented in TABLE V.

TABLE V

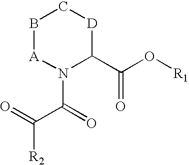

| No. | A | B | C | D | R₁ | R₂ |
|---|---|---|---|---|---|---|
| 87 | CH₂ | CH₂ | S | CH₂ | 3-phenylpropyl | 1,1-dimethylpropyl |
| 88 | CH₂ | CH₂ | O | CH₂ | 3-phenylpropyl | 1,1-dimethylpropyl |
| 89 | CH₂ | CH₂ | S | CH₂ | 3-phenylpropyl | cyclohexyl |
| 90 | CH₂ | CH₂ | O | CH₂ | 3-phenylpropyl | cyclohexyl |
| 91 | CH₂ | CH₂ | S | CH₂ | 3-phenylpropyl | phenyl |
| 92 | CH₂ | CH₂ | O | CH₂ | 3-phenylpropyl | phenyl |
| 93 | CH₂ | CH₂ | NH | CH₂ | 3-phenylpropyl | 1,1-dimethylpropyl |
| 94 | CH₂ | CH₂ | NH | CH₂ | 3-phenylpropyl | phenyl |

### Formula IX

Additionally, the sensorineurotrophic agent may be a compound of formula IX:

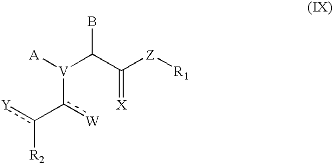

or a pharmaceutically acceptable salt, ester, or solvate thereof, wherein:

V is CH, N, or S;

A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;

R is either C₁-C₉ straight or branched chain alkyl, C₂-C₉ straight or branched chain alkenyl, C₃-C₉ cycloalkyl, C₅-C₇ cycloalkenyl, or Ar₃, wherein R is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;

Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and

R₁, R₂, W, X, Y, and Z are as defined in Formula VI above.

III. N-Oxides of Heterocyclic Esters, Amides, Thio-Esters and Ketones

### Formula X

The sensorineurotrophic agent may further be a compound of formula X:

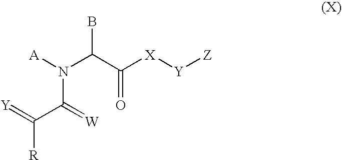

or a pharmaceutically acceptable salt, ester, or solvate thereof, wherein:

A and B, together with the nitrogen and carbon atoms to which they are respectively attached, form a 5-7 membered saturated or unsaturated heterocyclic ring containing one or more heteroatom(s) independently selected from the group consisting of CH, CH₂, O, S, SO, SO₂, N, NH, and NR₁;

W is O, S, CH₂, or H₂;

R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₂;

Ar₁ and Ar₂ are independently selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;

X is O, NH, NR₁, S, CH, CR₁, or CR₁R₃;

Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is an aromatic amine or a tertiary amine oxidized to a corresponding N-oxide;

said aromatic amine is selected from the group consisting of pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

said tertiary amine is NR₄R₅R₆, wherein R₄, R₅, and R₆ are independently selected from the group consisting of C₁-C₆ straight or branched chain alkyl or C₂-C₆ straight or branched chain alkenyl optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;

Ar is selected from the group consisting of pyrrolidinyl, pyridyl, pyrimidyl, pyrazyl, pyridazyl, quinolinyl, and isoquinolinyl; and

R₁ and R₃ are independently hydrogen, C₁-C₄ straight or branched chain alkyl, C₃-C₄ straight or branched chain alkenyl or alkynyl, or Y-Z.

### Formula XI

Moreover, the sensorineurotrophic agent may be a compound of formula XI:

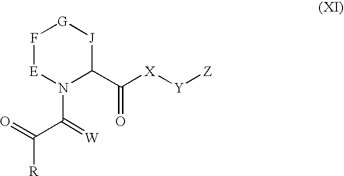

or a pharmaceutically acceptable salt, ester, or solvate thereof, wherein:

E, F, G and J are independently CH₂, O, S, SO, SO₂, NH or NR₁;

W is O, S, CH₂, or H₂;

R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;

Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl, and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;

X is O, NH, NR₁, S, CH, CR₁, or CR₁R₃;

Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is an aromatic amine or a tertiary amine oxidized to a corresponding N-oxide;

said aromatic amine is pyridyl, pyrimidyl, quinolinyl, and isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;

Ar is selected from the group consisting of pyrrolidinyl, pyridyl, pyrimidyl, pyrazyl, pyridazyl, quinolinyl, and isoquinolinyl; and

R₁ and R₃ are independently hydrogen, C₁-C₄ straight or branched chain alkyl, C₃-C₄ straight or branched chain alkenyl or alkynyl, or Y-Z.

### Formula XII

Furthermore, the sensorineurotrophic agent may be a compound of formula XII:

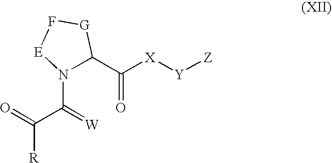

or a pharmaceutically acceptable salt, ester, or solvate thereof, wherein:

E, F, and G are independently CH₂, O, S, SO, SO₂, NH or NR₁;

W is O, S, CH₂, or H₂;

R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;

Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;

X is O, NH, NR₁, S, CH, CR₁, or CR₁R₃;

Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is an aromatic amine or a tertiary amine oxidized to a corresponding N-oxide;

said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;

Ar is selected from the group consisting of pyrrolidinyl, pyridyl, pyrimidyl, pyrazyl, pyridazyl, quinolinyl, and isoquinolinyl; and

R₁ and R₃ are independently hydrogen, C₁-C₄ straight or branched chain alkyl, C₃-C₄ straight or branched chain alkenyl or alkynyl, or Y-Z.

### Formula XIII

The sensorineurotrophic agent may also be a compound of formula XIII:

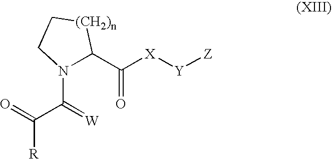

or a pharmaceutically acceptable salt, ester, or solvate thereof, wherein:

n is 1, 2, or 3, forming a 5-7 member heterocyclic ring;

W is O, S, CH₂, or H₂;

R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;

Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;

X is O, NH, NR₁, S, CH, CR₁, or CR₁R₃;

Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is an aromatic amine or a tertiary amine oxidized to a corresponding N-oxide;

said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;

Ar is selected from the group consisting of pyrrolidinyl, pyridyl, pyrimidyl, pyrazyl, pyridazyl, quinolinyl, and isoquinolinyl; and

R₁ and R₃, independently, are hydrogen, C₁-C₄ straight or branched chain alkyl, C₃-C₄ straight or branched chain alkenyl or alkynyl, or Y-Z.

Examples of the compounds of formula XIII when W is O are presented in TABLE VI:

TABLE VI

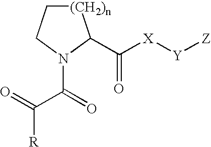

| No. | n | X | Y | Z | R |
|---|---|---|---|---|---|
| 95 | 1 | O | (CH₂)₃ | 3-Pyridyl N-oxide | 1,1-dimethylpropyl |
| 96 | 1 | O | (CH₂)₃ | 2-Pyridyl N-oxide | 1,1-dimethylpropyl |
| 97 | 1 | O | (CH₂)₃ | 4-Pyridyl N-oxide | 1,1-dimethylpropyl |
| 98 | 1 | O | (CH₂)₃ | 2-Quinolyl N-oxide | 1,1-dimethylpropyl |
| 99 | 1 | O | (CH₂)₃ | 3-Quinolyl N-oxide | 1,1-dimethylpropyl |
| 100 | 1 | O | (CH₂)₃ | 4-Quinolyl N-oxide | 1,1-dimethylpropyl |

Preferred compounds of formula XIII may be selected from the group consisting of:

3-(2-Pyridyl)-1-propyl(2S)-1-(1,1-Dimethyl-1,2-dioxopentyl)-2-pyrrolidinecarboxylate, N-oxide;

3-(3-Pyridyl)-1-propyl(2S)-1-(1,1-Dimethyl-1,2-dioxopentyl)-2-pyrrolidinecarboxylate, N-oxide;

3-(4-Pyridyl)-1-propyl(2S)-1-(1,1-Dimethyl-1,2-dioxopentyl)-2-pyrrolidinecarboxylate, N-oxide;

3-(2-Quinolyl)-1-propyl(2S)-1-(1,1-Dimethyl-1,2-dioxopentyl)-2-pyrrolidinecarboxylate, N-oxide;

3-(3-Quinolyl)-1-propyl(2S)-1-(1,1-Dimethyl-1,2-dioxopentyl)-2-pyrrolidinecarboxylate, N-oxide;

3-(4-Quinolyl)-1-propyl(2S)-1-(1,1-Dimethyl-1,2-dioxopentyl)-2-pyrrolidinecarboxylate, N-oxide; and

pharmaceutically acceptable salts, esters, and solvates thereof.

### Formula XIV

Additionally, the sensorineurotrophic agent may be a compound of formula XIV:

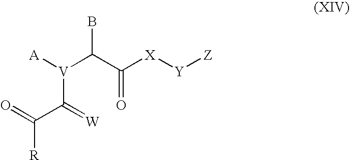

or a pharmaceutically acceptable salt, ester, or solvate thereof, wherein:

V is CH, N, or S;

A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR₇;

R₇ is either C₁-C₉ straight or branched chain alkyl, C₂-C₉ straight or branched chain alkenyl, C₃-C₉ cycloalkyl, C₅-C₇ cycloalkenyl, or Ar₃, wherein R₇ is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;

Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and

R, W, X, Y, and Z are as defined in Formula X above.

IV. N-Linked Ureas and Carbamates of Heterocyclic Thioesters

The sensorineurotrophic agent may further be a compound of formula XV:

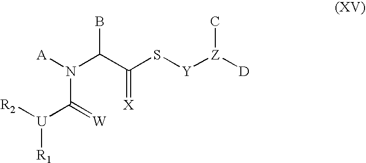

or a pharmaceutically acceptable salt, ester, or solvate thereof, wherein:

A and B, together with the nitrogen and carbon atoms to which they are respectively attached, form a 5-7 membered saturated or unsaturated heterocyclic ring containing, in addition to the nitrogen atom, one or more additional heteroatom(s) independently selected from the group consisting of O, S, SO, SO₂, N, NH, and NR₃;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;

W is O or S; and

U is either O or N, provided that:

when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and

when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

In a preferred embodiment of formula XV, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

### Formula XVI

Moreover, the sensorineurotrophic agent may be a compound of formula XVI:

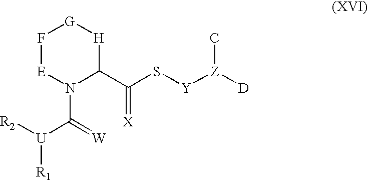

or a pharmaceutically acceptable salt, ester, or solvate thereof, wherein:

E, F, G and J are independently CH₂, O, S, SO, SO₂, NH, or NR₃;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;

W is O or S; and

U is either O or N, provided that:

when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and

when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

In a preferred embodiment of formula XVI, Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

### Formula XVII

The sensorineurotrophic agent may also be a compound of formula XVII:

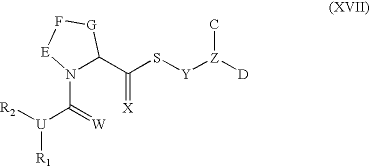

or a pharmaceutically acceptable salt, ester, or solvate thereof, wherein:

E, F, and G are independently CH₂, O, S, SO, SO₂, NH, and NR₃;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;

W is O or S; and

U is either O or N, provided that:

when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and

when U is N, then R₁ and R₂ are, independently, selected from the group consisting of hydrogen, Ar, C₃-C₈ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

In a preferred embodiment of formula XVII, Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

### Formula XVIII

The sensorineurotrophic agent may further be a compound of formula XVIII:

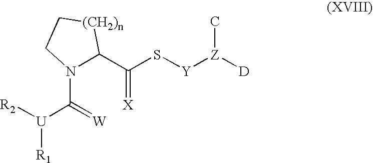

or a pharmaceutically acceptable salt, ester, or solvate thereof, wherein:

n is 1, 2 or 3;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;

W is O or S; and

U is either O or N, provided that:

when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain or alkenyl, wherein said alkyl or alkenyl is optionally substituted with one or more substituent(s) independently selected from the group consisting of Ar and C₃-C₈ cycloalkyl; and

when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

In a preferred embodiment of formula XVIII, Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

Exemplary compounds in which U is N and X is O of formula XVIII are presented in TABLE VII.

TABLE VII

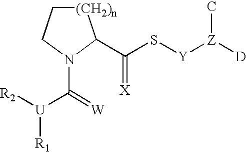

| No. | n | W | Y | Z | C | D | R₁ | R₂ |
|---|---|---|---|---|---|---|---|---|
| 101 | 1 | O | (CH₂)₂ | CH | 3-Pyridyl | H | H | 2-Methylbutyl |
| 102 | 1 | O | (CH₂)₂ | CH | 3-Pyridyl | H | H | 1,1- |
| | | | | | | | | dimethylpropyl |
| 103 | 1 | O | (CH₂)₂ | CH | 4- | H | H | 1,1- |
| | | | | | Methoxyphenyl | | | dimethylpropyl |
| 104 | 1 | O | CH₂ | CH | Phenyl | H | H | 1,1- |
| | | | | | | | | dimethylpropyl |
| 105 | 1 | S | (CH₂)₂ | CH | 4- | H | H | Cyclohexyl |
| | | | | | Methoxyphenyl | | | |
| 106 | 1 | O | (CH₂)₂ | CH | 3-Pyridyl | H | H | Cyclohexyl |
| 107 | 1 | S | (CH₂)₂ | CH | 3-Pyridyl | H | H | Cyclohexyl |
| 108 | 1 | S | (CH₂)₂ | CH | 3-Pyridyl | H | H | 1-Adamantyl |
| 109 | 1 | S | (CH₂)₂ | CH | 3-Pyridyl | H | H | 1,1- |
| | | | | | | | | dimethylpropyl |
| 110 | 1 | O | (CH₂)₂ | CH | Phenyl | Phenyl | H | 1,1- |
| | | | | | | | | dimethylpropyl |
| 111 | 2 | O | (CH₂)₂ | CH | Phenyl | H | H | 1,1- |
| | | | | | | | | dimethylpropyl |
| 112 | 2 | O | (CH₂)₂ | CH | Phenyl | H | H | Phenyl |
| 113 | 2 | O | Direct | CH | 2-Phenylethyl | 2- | H | Phenyl |
| | | | bond | | | Phenylethyl | | |
| 114 | 2 | O | Direct | CH | 2-Phenylethyl | 2- | H | Cyclohexyl |
| | | | bond | | | Phenylethyl | | |
| 115 | 2 | S | Direct | CH | 2-Phenylethyl | 2- | H | Cyclohexyl |
| | | | bond | | | Phenylethyl | | |
| 116 | 2 | O | (CH₂)₂ | CH | 4- | H | H | Cyclohexyl |
| | | | | | Methoxyphenyl | | | |

The most preferred compounds of formula XVIII are selected from the group consisting of:

3-(3-Pyridyl)-1-propyl-2S-1-[(2-methylbutyl) carbamoyl]pyrrolidine-2-carboxylate;

3-(3-Pyridyl)-1-propyl-2S-1-[(1′,1′-Dimethylpropyl) carbamoyl]pyrrolidine-2-carboxylate;

3-(3-Pyridyl)-1-propyl-2S-1-[(cyclohexyl) thiocarbamoyl]pyrrolidine-2-carboxylate; and

pharmaceutically acceptable salts, esters, and solvates thereof.

### Formula XIX

Additionally, the sensorineurotrophic agent may be a compound of formula XIX:

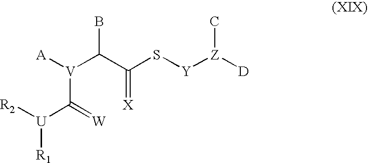

or a pharmaceutically acceptable salt, ester, or solvate thereof, wherein:

V is CH, N, or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂; and

A, B, R₁, R₂, U, W, and X are as otherwise defined in formula XV.

V. N-Linked Sulfonamides of Heterocyclic Thioesters

### Formula XX

The sensorineurotrophic agent may further be a compound of formula XX:

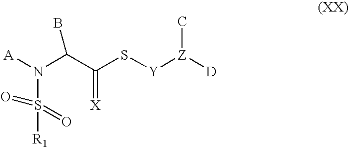

a pharmaceutically acceptable salt, ester, or solvate thereof, wherein:

A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₂;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and

R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₂, S, SO, or SO₂.

In a preferred embodiment of formula XX, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

In another preferred embodiment of formula XX, A and B, together with the nitrogen and carbon atoms to which they are respectfully attached, form a 6 membered saturated or unsaturated heterocyclic ring; and R₂ is C₄-C₇ branched chain alkyl, C₄-C₇ cycloalkyl, phenyl, or 3,4,5-trimethoxyphenyl.

In the most preferred embodiment of formula XX, the compound is selected from the group consisting of:

3-(para-Methoxyphenyl)-1-propylmercaptyl(2S)-N-(benzenesulfonyl)pyrrolidine-2-carboxylate;

3-(para-Methoxyphenyl)-1-propylmercaptyl(2S)-N-(α-toluenesulfonyl)pyrrolidine-2-carboxylate;

3-(para-Methoxyphenyl)-1-propylmercaptyl(2S)-N-(α-toluenesulfonyl)pyrrolidine-2-carboxylate;

1,5-Diphenyl-3-pentylmercaptyl N-(para-toluenesulfonyl)pipecolate; and

pharmaceutically acceptable salts, esters, and solvates thereof.

### Formula XXI

Moreover, the sensorineurotrophic agent may be a compound of formula XXI:

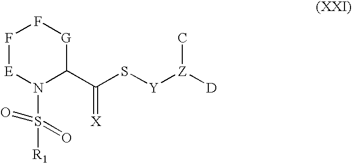

or a pharmaceutically acceptable salt, ester, or solvate thereof, wherein:

E, F, G and J are independently CH₂, O, S, SO, SO₂, NH or NR₂;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and

R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₂, S, SO, or SO₂.

In a preferred embodiment of formula XXI, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

### Formula XXII

The sensorineurotrophic agent may also be a compound of formula XXII:

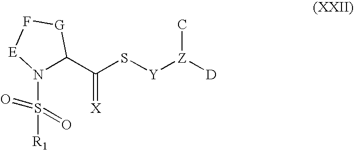

or a pharmaceutically acceptable salt, ester, or solvate thereof, wherein:

E, F, and G are independently CH₂, O, S, SO, SO₂, NH or NR₂;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and

R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₂, S, SO, or SO₂.

In a preferred embodiment of formula XXII, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

### Formula XXIII

Additionally, the sensorineurotrophic agent may be a compound of formula XXIII:

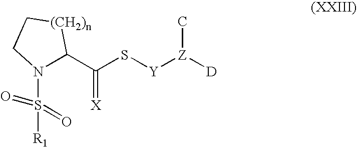

or a pharmaceutically acceptable salt, ester, or solvate thereof, wherein:

n is 1, 2 or 3;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;

C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and

R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₃, S, SO, or SO₂.

In a preferred embodiment of formula XXIII, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

Exemplary compounds of formula XXIII are presented in TABLE VIII:

TABLE VIII

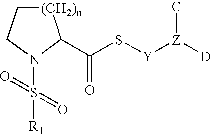

| No. | n | Y | Z | C | D | R₁ |
|---|---|---|---|---|---|---|
| 117 | 1 | CH₂ | CH | Phenyl | H | Phenyl |
| 118 | 1 | CH₂ | CH | Phenyl | H | α- |
| | | | | | | Methylphenyl |
| 119 | 1 | CH₂ | CH | Phenyl | H | 4- |
| | | | | | | Methylphenyl |
| 120 | 1 | (CH₂)₂ | CH | p-Methoxyphenyl | H | Phenyl |
| 121 | 1 | (CH₂)₂ | CH | p-Methoxyphenyl | H | α- |
| | | | | | | Methylphenyl |
| 122 | 1 | (CH₂)₂ | CH | p-Methoxyphenyl | H | 4- |
| | | | | | | Methylphenyl |
| 123 | 1 | (CH₂)₂ | CH | Phenyl | Phenyl | Phenyl |
| 124 | 1 | (CH₂)₂ | CH | Phenyl | Phenyl | α- |
| | | | | | | Methylphenyl |
| 125 | 1 | (CH₂)₂ | CH | Phenyl | Phenyl | 4- |
| | | | | | | Methylphenyl |
| 126 | 2 | (CH₂)₃ | CH | Phenyl | H | Phenyl |
| 127 | 2 | (CH₂)₃ | CH | Phenyl | H | α- |
| | | | | | | Methylphenyl |
| 128 | 2 | (CH₂)₃ | CH | Phenyl | H | 4- |
| | | | | | | Methylphenyl |
| 129 | 2 | (CH₂)₃ | CH | Phenyl | H | 3,4,5- |
| | | | | | | trimethoxyphenyl |
| 130 | 2 | (CH₂)₃ | CH | Phenyl | H | Cyclohexyl |
| 131 | 2 | Direct | CH | 3-Phenylpropyl | 3- | Phenyl |
| | | bond | | | Phenylpropyl | |
| 132 | 2 | Direct | CH | 3-Phenylpropyl | 3- | α- |
| | | bond | | | Phenylpropyl | Methylphenyl |
| 133 | 2 | Direct | CH | 3-Phenylpropyl | 3- | 4- |
| | | bond | | | Phenylpropyl | Methylphenyl |
| 134 | 2 | Direct | CH | 3-Phenylethyl | 3-Phenylethyl | 4- |
| | | bond | | | | Methylphenyl |
| 135 | 2 | Direct | CH | 3-(4- | 3- | 4- |
| | | bond | | Methoxyphenyl)propyl | Phenylpropyl | Methylphenyl |
| 136 | 2 | Direct | CH | 3-(2- | 3- | 4- |
| | | bond | | Pyridyl)propyl | Phenylpropyl | Methylphenyl |

The most preferred compounds of formula XXIII are selected from the group consisting of:

3-(para-Methoxyphenyl)-1-propylmercaptyl(2S)-N-(benzenesulfonyl)pyrrolidine-2-carboxylate;

3-(para-Methoxyphenyl)-1-propylmercaptyl(2S)-N-α-toluenesulfonyl)pyrrolidine-2-carboxylate;

3-(para-Methoxyphenyl)-1-propylmercaptyl(2S)-N-α-toluenesulfonyl)pyrrolidine-2-carboxylate;

1,5-Diphenyl-3-pentylmercaptyl N-(para-toluenesulfonyl)pipecolate; and

pharmaceutically acceptable salts, esters, and solvates thereof.

### Formula XXIV

Moreover, the sensorineurotrophic agent may be a compound of formula XXIV:

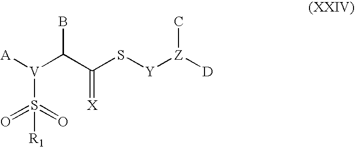

or a pharmaceutically acceptable salt, ester, or solvate thereof, wherein:

V is CH, N, or S;

A, B, C, D, R₁, X, Y, and Z are as defined in formula XX above.

VI. Pyrrolidine Derivatives

### Formula XXV

The sensorineurotrophic agent may also be a compound of formula XXV:

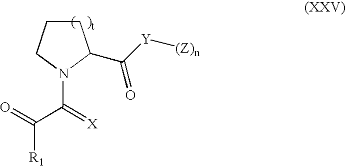

or a pharmaceutically acceptable salt, ester, or solvate thereof, wherein:

R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;

Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

X is O, S, CH₂ or H₂;

Y is O or NR₂, wherein R₂ is a direct bond to a Z, hydrogen or C₁-C₆ alkyl; and

each Z, independently, is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment

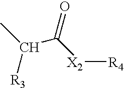

wherein:

R₃ is C₁-C₉ straight or branched chain alkyl which is unsubstituted or substituted with C₃-C₈ cycloalkyl or Ar₁;

X₂ is O or NR₅, wherein R₅ is selected from the group consisting of hydrogen, C₁-C₆ straight or branched chain alkyl, and C₂-C₆ straight or branched chain alkenyl;

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;

n is 1 or 2, and;

t is 1, 2 or 3.

In a preferred embodiment of formula XXV, Z and R₁ are lipophilic.

In a more preferred embodiment of formula XXV, the compound is selected from the group consisting of:

3-phenyl-1-propyl (2S)-1-(3,3-dimethyl-1,2-dioxopentyl)-2-pyrrolidinecarboxylate;

3-phenyl-1-prop-2-(E)-enyl (2S)-1-(3,3-dimethyl-1,2-dioxopentyl)-2-pyrrolidinecarboxylate;

3-(3,4,5-trimethoxyphenyl)-1-propyl (2S)-1-(3,3-dimethyl-1,2-dioxopentyl)-2-pyrrolidine-carboxylate;

3-(3,4,5-trimethoxyphenyl)-1-prop-2-(E)-enyl (2S)-1-(3,3-dimethyl-1,2-dioxopentyl)-2-pyrrolidinecarboxylate;

3-(4,5-dichlorophenyl)-1-propyl (2S)-1-(3,3-dimethyl-1,2-dioxopentyl)-2-pyrrolidinecarboxylate;

3-(4,5-dichlorophenyl)-1-prop-2-(E)-enyl (2S)-1-(3,3-dimethyl-1,2-dioxopentyl)-2-pyrrolidine-carboxylate;

3-(4,5-methylenedioxyphenyl)-1-propyl (2S)-1-(3,3-dimethyl-1,2-dioxopentyl)-2-pyrrolidine-carboxylate;

3-(4,5-methylenedioxyphenyl)-1-prop-2-(E)-enyl (2S)-1-(3,3-dimethyl-1,2-dioxopentyl)-2-pyrrolidinecarboxylate;

3-cyclohexyl-1-propyl (2S)-1-(3,3-dimethyl-1,2-dioxopentyl)-2-pyrrolidinecarboxylate;

3-cyclohexyl-1-prop-2-(E)-enyl (2S)-1-(3,3-dimethyl-1,2-dioxopentyl)-2-pyrrolidinecarboxylate;

(1R)-1,3-diphenyl-1-propyl (2S)-1-(3,3-dimethyl-1,2-dioxopentyl)-2-pyrrolidinecarboxylate;

(1R)-1,3-diphenyl-1-prop-2-(E)-enyl (2S)-1-(3,3-dimethyl-1,2-dioxopentyl)-2-pyrrolidine-carboxylate;

(1R)-1-cyclohexyl-3-phenyl-1-propyl (2S)-1-(3,3-dimethyl-1,2-dioxopentyl)-2-pyrrolidine-carboxylate;

(1R)-1-cyclohexyl-3-phenyl-1-prop-2-(E)-enyl (2S)-1-(3,3-dimethyl-1,2-dioxopentyl)-2-pyrrolidinecarboxylate;

(1R)-1-(4,5-dichlorophenyl)-3-phenyl-1-propyl (2S)-1-(3,3-dimethyl-1,2-dioxopentyl)-2-pyrrolidine-carboxylate;

3-phenyl-1-propyl (2S)-1-(1,2-dioxo-2-cyclohexyl)ethyl-2-pyrrolidinecarboxylate;

3-phenyl-1-propyl (2S)-1-(1,2-dioxo-4-cyclohexyl)butyl-2-pyrrolidinecarboxylate;

3-phenyl-1-propyl (2S)-1-(1,2-dioxo-2-[2-furanyl])ethyl-2-pyrrolidinecarboxylate;

3-phenyl-1-propyl (2S)-1-(1,2-dioxo-2-[2-thienyl])ethyl-2-pyrrolidinecarboxylate;

3-phenyl-1-propyl (2S)-1-(1,2-dioxo-2-[2-thiazolyl])ethyl-2-pyrrolidinecarboxylate;

3-phenyl-1-propyl (2S)-1-(1,2-dioxo-2-phenyl)ethyl-2-pyrrolidinecarboxylate;

1,7-diphenyl-4-heptyl (2S)-1-(3,3-dimethyl-1,2-dioxopentyl)-2-pyrrolidinecarboxylate;

3-phenyl-1-propyl (2S)-1-(3,3-dimethyl-1,2-dioxo-4-hydroxybutyl)-2-pyrrolidinecarboxylate;

3-phenyl-1-propyl (2S)-1-(3,3-dimethyl-1,2-dioxopentyl)-2-pyrrolidinecarboxamide;

1-[1-(3,3-dimethyl-1,2-dioxopentyl)-L-proline]-L-phenylalanine ethyl ester;

1-[1-(3,3-dimethyl-1,2-dioxopentyl)-L-proline]-L-leucine ethyl ester;

1-[1-(3,3-dimethyl-1,2-dioxopentyl)-L-proline]-L-phenylglycine ethyl ester;

1-[1-(3,3-dimethyl-1,2-dioxopentyl)-L-proline]-L-phenylalanine phenyl ester;

1-[1-(3,3-dimethyl-1,2-dioxopentyl)-L-proline]-L-phenylalanine benzyl ester;

1-[1-(3,3-dimethyl-1,2-dioxopentyl)-L-proline]-L-isoleucine ethyl ester; and

pharmaceutically acceptable salts, esters, and solvates thereof.

### Formula XXVI

Additionally, the sensorineurotrophic agent may be a compound of formula XXVI:

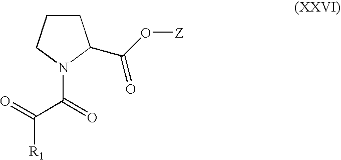

or a pharmaceutically acceptable salt, ester, or solvate thereof, wherein:

R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;

Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

Z is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment

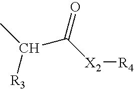

wherein:

R₃ is C₁-C₉ straight or branched chain alkyl which is unsubstituted or substituted with C₃-C₈ cycloalkyl or Ar₁;

X₂ is O or NR₅, wherein R₅ is selected from the group consisting of hydrogen, C₁-C₆ straight or branched chain alkyl, and C₂-C₆ straight or branched chain alkenyl; and

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl.

In a preferred embodiment of formula XXVI, R₁ is selected from the group consisting of C₁-C₉ straight or branched chain alkyl, 2-cyclohexyl, 4-cyclohexyl, 2-furanyl, 2-thienyl, 2-thiazolyl, and 4-hydroxybutyl.

In another preferred embodiment of formula XXVI, Z and R₁ are lipophilic.

### Formula XXVII

Furthermore, the sensorineurotrophic agent may be a compound of formula XXVII:

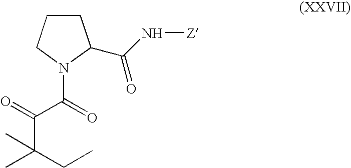

or a pharmaceutically acceptable salt, ester, or solvate thereof, wherein:

Z′ is the fragment

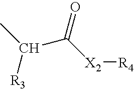

wherein:

R₃ is C₁-C₉ straight or branched chain alkyl or unsubstituted Ar₁, wherein said alkyl is unsubstituted or substituted with C₃-C₈ cycloalkyl or Ar₁;

X₂ is O or NR₅, wherein R₅ is selected from the group consisting of hydrogen, C₁-C₆ straight or branched chain alkyl, and C₂-C₆ straight or branched chain alkenyl;

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and

Ar₁ is as defined in formula XXVI.

In a preferred embodiment of formula XXVII, Z′ is lipophilic.

### Formula XXVIII

The sensorineurotrophic agent may also be a compound of formula XXVIII:

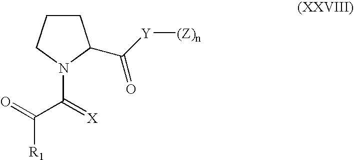

wherein:

R₁ is C₁-C₆ straight or branched chain alkyl, C₂-C₆ straight or branched chain alkenyl, C₃-C₆ cycloalkyl or Ar₁, wherein said alkyl or alkenyl is unsubstituted or substituted with C₃-C₆ cycloalkyl or Ar₂;

Ar₁ and Ar₂ are independently selected from the group consisting of 2-furyl, 2-thienyl, and phenyl;

X is selected from the group consisting of oxygen and sulfur;

Y is oxygen or NR₂, wherein R₂ is a direct bond to a Z, hydrogen or C₁-C₆ alkyl;

z is hydrogen, C₁-C₆ straight or branched chain alkyl, or C₂-C₆ straight or branched chain alkenyl, wherein said Z is substituted with one or more substituent(s) independently selected from the group consisting of 2-furyl, 2-thienyl, C₃-C₆ cycloalkyl, pyridyl, and phenyl, each having one or more substituent(s) independently selected from the group consisting of hydrogen and C₁-C₄ alkoxy; and n is 1 or 2.

In a preferred embodiment of formula XXVIII, Z and R₁ are lipophilic.

In another preferred embodiment of formula XXVIII, the compound is selected from the group consisting of:

3-(2,5-dimethoxyphenyl)-1-propyl (2S)-1-(3,3-dimethyl-1,2-dioxopentyl)-2-pyrrolidinecarboxylate;

3-(2,5-dimethoxyphenyl)-1-prop-2-(E)-enyl (2S)-1-(3,3-dimethyl-1,2-dioxopentyl)-2-pyrrolidine-carboxylate;

2-(3,4,5-trimethoxyphenyl)-1-ethyl (2S)-1-(3,3-dimethyl-1,2-dioxopentyl)-2-pyrrolidinecarboxylate;

3-(3-pyridyl)-1-propyl (2S)-1-(3,3-dimethyl-1,2-dioxopentyl)-2-pyrrolidinecarboxylate;

3-(2-pyridyl)-1-propyl (2S)-1-(3,3-dimethyl-1,2-dioxopentyl)-2-pyrrolidinecarboxylate;

3-(4-pyridyl)-1-propyl (2S)-1-(3,3-dimethyl-1,2-dioxopentyl)-2-pyrrolidinecarboxylate;

3-phenyl-1-propyl (2S)-1-(2-tert-butyl-1,2-dioxoethyl)-2-pyrrolidinecarboxylate;

3-phenyl-1-propyl (2S)-1-(2-cyclohexylethyl-1,2-dioxoethyl)-2-pyrrolidinecarboxylate;

3-(3-pyridyl)-1-propyl (2S)-1-(2-cyclohexylethyl-1,2-dioxoethyl)-2-pyrrolidine-carboxylate;

3-(3-pyridyl)-1-propyl (2S)-1-(2-tert-butyl-1,2-dioxoethyl)-2-pyrrolidinecarboxylate;

3,3-diphenyl-1-propyl (2S)-1-(3,3-dimethyl-1,2-dioxopentyl)-2-pyrrolidinecarboxylate;

3-(3-pyridyl)-1-propyl (2S)-1-(2-cyclohexyl-1,2-dioxoethyl)-2-pyrrolidinecarboxylate;

3-(3-pyridyl)-1-propyl (2S)-N-([2-thienyl]glyoxyl)pyrrolidinecarboxylate;

3,3-diphenyl-1-propyl (2S)-1-(3,3-dimethyl-1,2-dioxobutyl)-2-pyrrolidinecarboxylate;

3,3-diphenyl-1-propyl (2S)-1-cyclohexylglyoxyl-2-pyrrolidinecarboxylate;

3,3-diphenyl-1-propyl (2S)-1-(2-thienyl)glyoxyl-2-pyrrolidinecarboxylate; and

pharmaceutically acceptable salts, esters, and solvates thereof.

In a more preferred embodiment of formula XXVIII, the compound is selected from the group consisting of:

3-(3-pyridyl)-1-propyl (2S)-1-(3,3-dimethyl-1,2-dioxopentyl)-2-pyrrolidinecarboxylate;

3-(2-pyridyl)-1-propyl (2S)-1-(3,3-dimethyl-1,2-dioxopentyl)-2-pyrrolidinecarboxylate;

3-(3-pyridyl)-1-propyl (2S)-1-(2-cyclohexyl-1,2-dioxoethyl)-2-pyrrolidinecarboxylate; and

pharmaceutically acceptable salts, esters, and solvates thereof.

In the most preferred embodiment of formula XXVIII, the compound is 3-(3-pyridyl)-1-propyl (2S)-1-(3,3-dimethyl-1,2-dioxopentyl)-2-pyrrolidine-carboxylate, and pharmaceutically acceptable salts, esters, and solvates thereof.

### Formula XXIX

Additionally, the sensorineurotrophic agent may be a compound of formula XXIX:

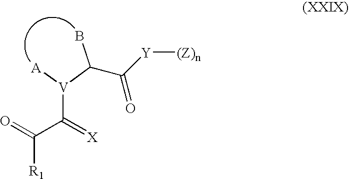

or a pharmaceutically acceptable salt, ester, or solvate thereof, wherein:

V is CH, N, or S;

A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;

R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo-(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, alkylthio, sulfhydryl, amino, (C₁-C₆)-alkylamino, amino-(C₁-C₆)-alkyl, aminocarboxyl, and Ar₂;

R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;

Ar₁ and Ar₂ are independently an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S;

X is O, S, CH₂ or H₂;

Y is O or NR₂, wherein R₂ is a direct bond to a Z, hydrogen or C₁-C₆ alkyl; and

Z is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment

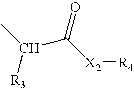

wherein:

R₃ is C₁-C₉ straight or branched chain alkyl which is unsubstituted or substituted with C₃-C₈ cycloalkyl or Ar₁;

X₂ is O or NR₅, wherein R₅ is selected from the group consisting of hydrogen, C₁-C₆ straight or branched chain alkyl, and C₂-C₆ straight or branched chain alkenyl; and

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and,

n is 1 or 2.

Other compounds which are sensorineurotrophic agents within the scope of the present invention are those compounds which may possess immunosuppressive, non-immunosuppressive or other activities as long as they also are useful in the treatment or prevention of hearing loss or other neurodegenerative diseases of the ear. For example, such compounds may include, but are not limited to those below:

### Compound 167

Ocain et al., Biochemical and Biophysical Research Communications (1993) 3:192, incorporated herein by reference, discloses an exemplary pipecolic acid derivative represented by Formula XXX. This compound is prepared by reacting 4-phenyl-1,2,4-triazoline-3,5-dione with rapamycin.

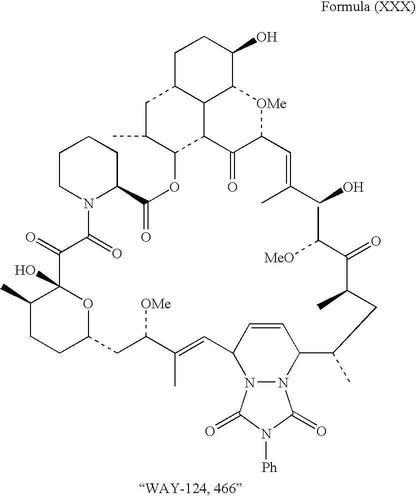

### Compound 168

Chakraborty et al., Chemistry and Biology (1995) 2:157-161, incorporated herein by reference, discloses an exemplary pipecolic acid derivative represented by Formula XXXI.

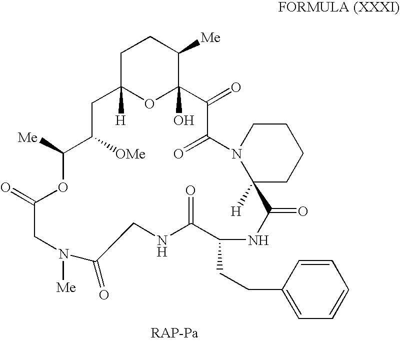

### Compounds 169-171

Ikeda et al., J. Am. Chem. Soc. (1994) 116:4143-4144, incorporated herein by reference, discloses exemplary pipecolic acid derivatives represented by Formula XXXII and Table XII.

TABLE XII
Formula (XXXII)

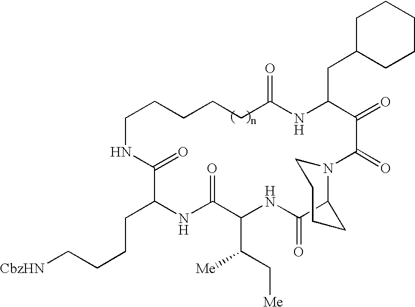

| Compound | Structure |
|---|---|
| 169 | n = 1 |
| 170 | n = 2 |
| 171 | n = 3 |

### Compounds 172-175

Wang et al., Bioorganic & Medicinal Chemistry Letters (1994) 4:1161-1166, 9, incorporated herein by reference, discloses exemplary pipecolic acid derivatives represented by Formula XXXIII and Table XIII.

TABLE XIII
FORMULA (XXXIII)

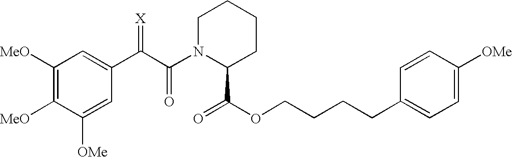

| Compound | Structure |
|---|---|
| 172 | X = H, H |
| 173 | X = CH₂ |
| 174 | X = H, CH₃ |
| 175 | X = O |

### Compound 176

Birkenshaw et al., Bioorganic & Medicinal Chemistry Letters (1994) 4(21):2501-2506, incorporated herein by reference, discloses an exemplary pipecolic acid derivative represented by Formula XXXIV:

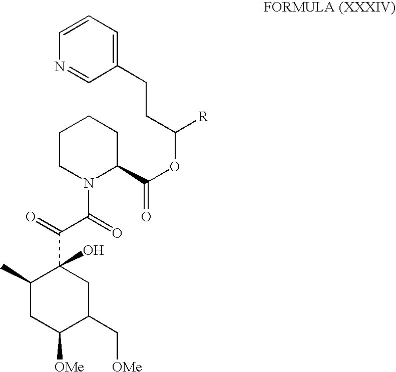

### Compounds 177-187

Holt et al., J. Am. Chem. Soc. (1993) 115:9925-9938, incorporated herein by reference, discloses exemplary pipecolic acid derivatives represented by Formula XXXV and Tables XIV and XV.

TABLE XV

| | |
|---|---|
| | FORMULA (XXXIII) |
| 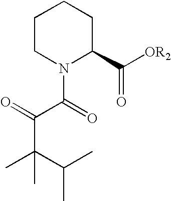 | |

| Compound | R₂ |
|---|---|
| 177 | 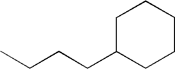 |
| 178 | 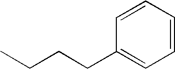 |
| 179 | 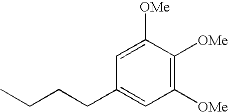 |
| 180 | 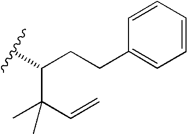 |
| 181 | 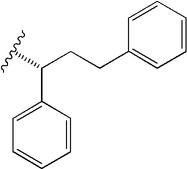 |
| 182 | 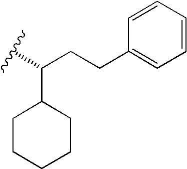 |
| 183 | 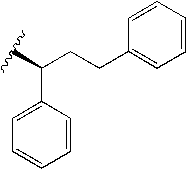 |
| 184 | 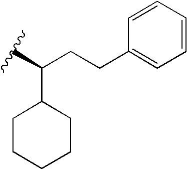 |

TABLE XV

| Compound | Structure |
|---|---|
| 185 | 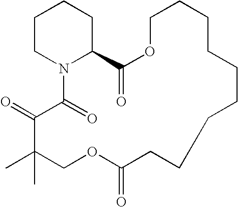 |
| 186 | 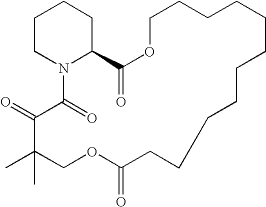 |
| 187 | 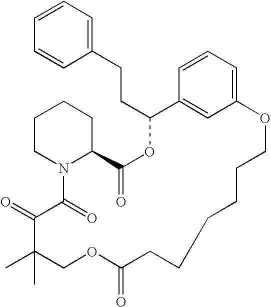 |

### Compounds 188-196

Caffery et al., Bioorganic & Medicinal Chemistry Letters (1994) 4(21):2507-2510, incorporated herein by reference, discloses exemplary pipecolic acid derivatives represented by Formulas XXXVI-XXXVIII and Tables XVI-XVIII.

TABLE XVI

| | |
|---|---|
| | FORMULA XXXVI |
| 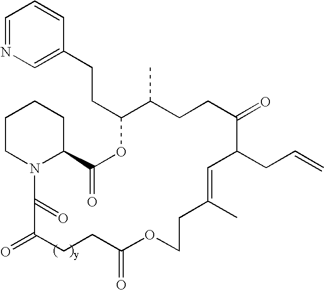 | |

| Compound | Structure |
|---|---|
| 188 | y = 1 |
| 189 | y = 2 |
| 190 | y = 3 |

TABLE XVII
FORMULA XXXVII

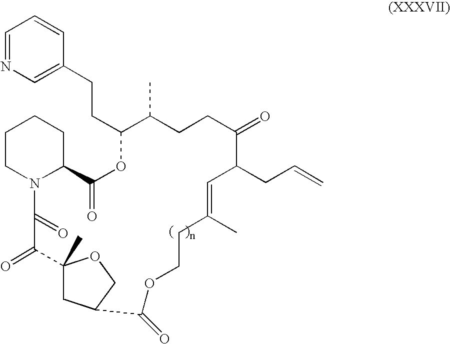

| Compound | Structure |
|---|---|
| 191 | n = 1 |
| 192 | n = 2 |
| 193 | n = 3 |

TABLE XVIII
FORMULA XXXVIII

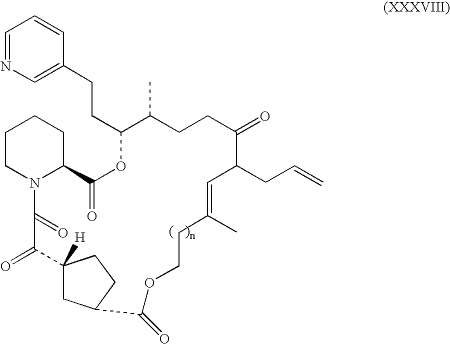

| Compound | Structure |
|---|---|
| 194 | n = 1 |
| 195 | n = 2 |
| 196 | n = 3 |

### Compound 197

Teague et al., Bioorganic & Medicinal Chemistry Letters (1993) 3(10):1947-1950, incorporated herein by reference, discloses an exemplary pipecolic acid derivative represented by Formula XXXIX.

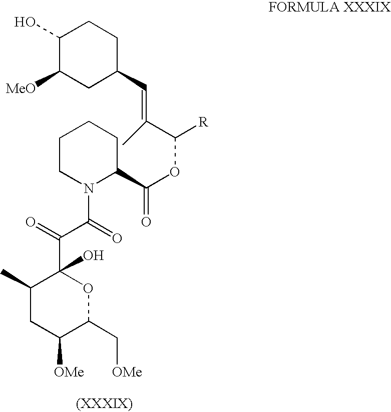

### Compounds 198-200

Yamashita et al., Bioorganic & Medicinal Chemistry Letters (1994) 4(2):325-328, incorporated herein by reference, discloses exemplary pipecolic acid derivatives represented by Formula XL and Table XIX.

TABLE XIX
FORMULA XL

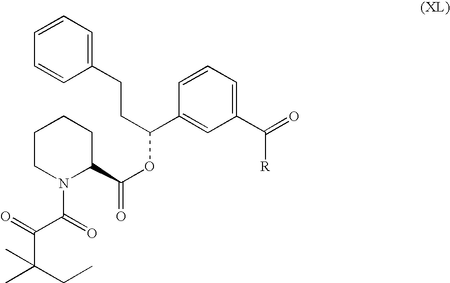

| Compound | Structure |
|---|---|
| 198 | R = phenyl |
| 199 | R = N(allyl)₂ |
| 200 | 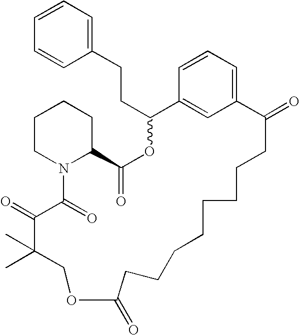 |

### Compounds 201-221

Holt et al., Bioorganic & Medicinal Chemistry Letters (1994) 4(2):315-320, incorporated herein by reference, discloses exemplary pipecolic acid derivatives represented by Formula XLI and Tables XX-XXII.

TABLE XX
FORMULA XLI

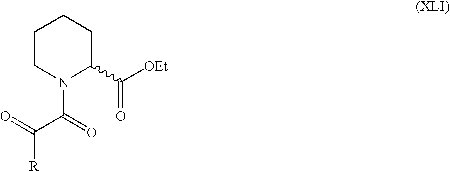

| Compound No. | R |
|---|---|
| 201 | 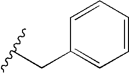 |
| 202 | 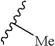 |
| 203 | 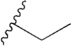 |
| 204 | 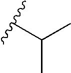 |
| 205 | 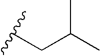 |
| 206 | 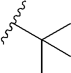 |
| 207 | 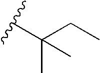 |
| 208 | 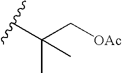 |
| 209 | 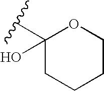 |
| 210 | 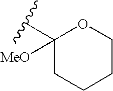 |
| 211 | 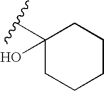 |
| 212 | 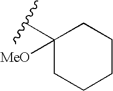 |
| 213 | 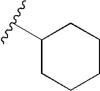 |
| 214 | 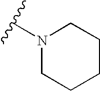 |
| 215 | 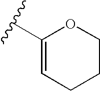 |
| 216 | 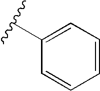 |

TABLE XXI

| Compound No. | Structure |
|---|---|
| 217 | 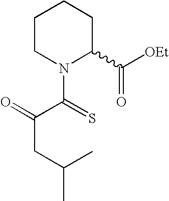 |
| 218 | 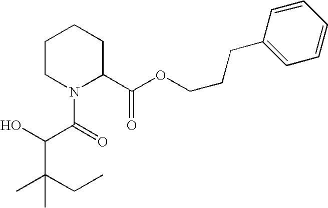 |
| 219 | 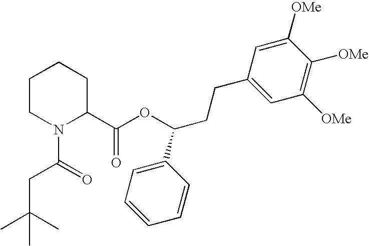 |

TABLE XXII

| Compound No. | Structure |
|---|---|
| 220 | 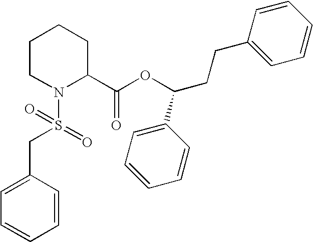 |
| 221 | 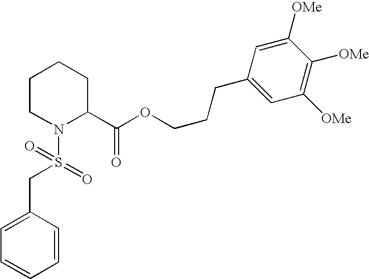 |

### Compounds 222-234

Holt et al., Bioorganic & Medicinal Chemistry Letters (1993) 3(10):1977-1980, incorporated herein by reference, discloses exemplary pipecolic acid derivatives represented by Formulas XLII and XLIII and Tables XXIII-XXV.

TABLE XXIII
FORMULA XLII

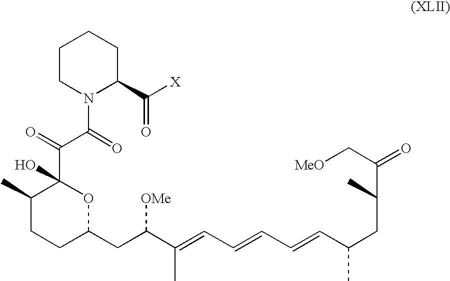

| Compound | Structure |
|---|---|
| 222 | X = OH |
| 223 | X = OMe |
| 224 | X = O-iso-Pr |
| 225 | X = OBn |
| 226 | X = OCH (Me)Ph |
| 227 | X = OCH₂CHCHPh |
| 228 | X = OCH₂CH₂CH₂(3,4-OMe₂)Ph |
| 229 | X = NHBn |
| 230 | X = NHCH₂CH₂CH₂Ph |

TABLE XXIV

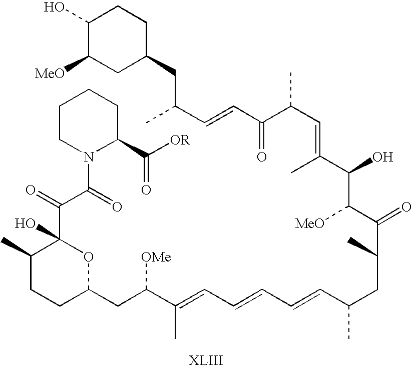

FORMULA XLIII

| Compound | Structure |
|---|---|
| 231 | R = Me |
| 232 | R = Bn |

TABLE XXV

| Compound | Structure |
|---|---|
| 233 | 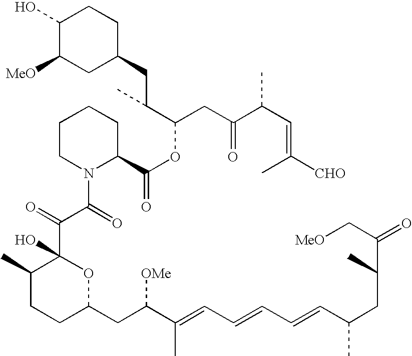 |
| 234 | 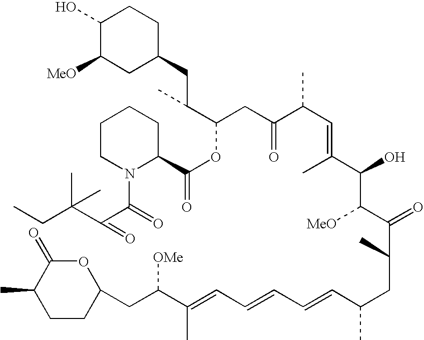 |

### Compounds 235-249

Hauske et al., J. Med. Chem. (1992) 35:4284-4296, incorporated herein by reference, discloses exemplary pipecolic acid derivatives represented by Formulas XLIV-XLVII and Tables XXVI-XXIX.

TABLE XXVI

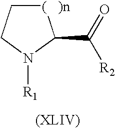

FORMULA XLIV

| Compound | Structure |
|---|---|
| 235 | n = 2 |
| | R₁ = |
| | 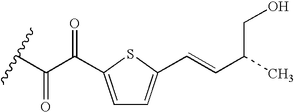 |
| | R₂ = Phe-O-tert-butyl |
| 236 | n = 2 |
| | R₁ = |
| | 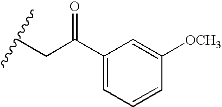 |
| | R₂ = Phe-O-tert-butyl |

TABLE XXVII

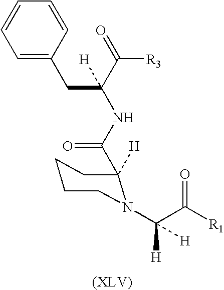

FORMULA XLV

| Compound | Structure |
|---|---|
| 237 | R₁ = m-OCH₃Ph |
| | R₃ = Val-O-tert-butyl |
| 238 | R₁ = m-OCH₃Ph |
| | R₃ = Leu-O-tert-butyl |
| 239 | R₁ = m-OCH₃Ph |
| | R₃ = Ileu-O-tert-butyl |
| 240 | R₁ = m-OCH₃Ph |
| | R₃ = hexahydro-Phe-O-tert-butyl |
| 241 | R₁ = m-OCH₃Ph |
| | R₃ = allylalanine-O-tert-butyl |
| 242 | R₁ = β-naphthyl |
| | R₃ = Val-O-tert-butyl |

TABLE XXVIII

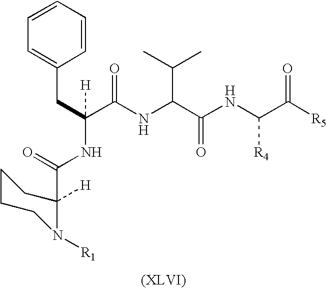

FORMULA XLVI

| Compound | Structure |
|---|---|
| 243 | R₁ = CH₂(CO)-m-OCH₃Ph |
| | R₄ = CH₂Ph |
| | R₅ = OCH₃ |
| 244 | R₁ = CH₂(CO)-β-naphthyl |
| | R₄ = CH₂Ph |
| | R₅ = OCH₃ |

TABLE XXIX

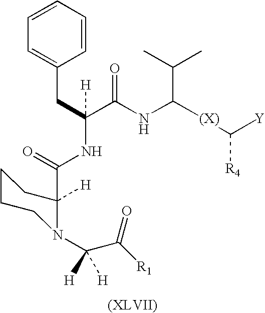

FORMULA XLVII

| Compound | Structure |
|---|---|
| 245 | R₁ = m-OCH₃Ph |
| | X = trans-CH═CH— |
| | R₄ = H |
| | Y = OC(O)Ph |
| 246 | R₁ = m-OCH₃Ph |
| | X = trans-CH═CH |
| | R₄ = H |
| | Y = OC(O)CF₃ |
| 247 | R₁ = m-OCH₃Ph |
| | X = trans-CH═CH— |
| | R₄ = — |
| | Y = — |
| 248 | R₁ = m-OCH₃Ph |
| | X = trans-CH═CH— |
| | R₄ = H |
| | Y = OCH₂CH═CH₂ |
| 249 | R₁ = m-OCH₃Ph |
| | X = C═O |
| | R₄ = H |
| | Y = Ph |

### Compound 250

Teague et al., Bioorganic & Med. Chem. Letters (1994) 4(13):1581-1584, incorporated herein by reference, discloses an exemplary pipecolic acid derivative represented by Formula XLVIII.

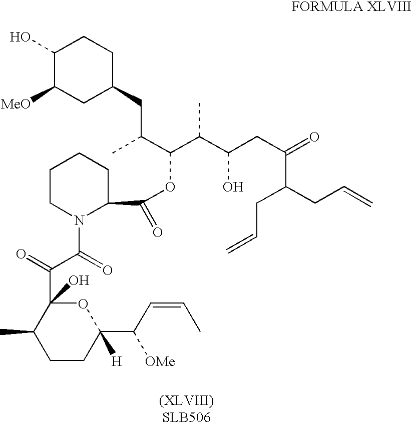

### Compounds 251-254

Stocks et al., Bioorganic & Med. Chem. Letters (1994) 4(12):1457-1460, incorporated herein by reference, discloses exemplary pipecolic acid derivatives represented by Formula XLIX and Tables XXX and XXXI.

TABLE XXX

| Compound No. | Structure |
|---|---|
| 251 | 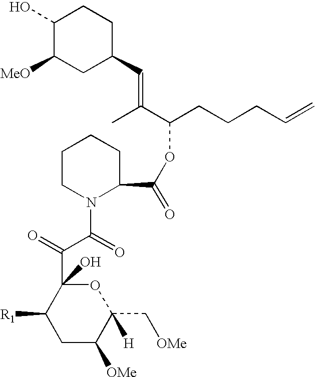 |

TABLE XXXI

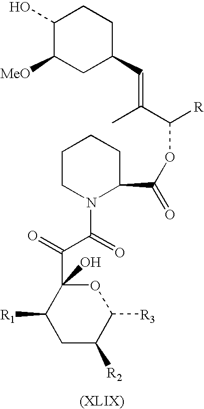

FORMULA XLIX

| Compound | Structure |
|---|---|
| 252 | R₁ = H |
| | R₂ = OMe |
| | R₃ = CH₂Ome |
| 253 | R₁ = H |
| | R₂ = H |
| | R₃ = H |
| 254 | R₁ = Me |
| | R₂ = H |
| | R₃ = H |

### Compounds 255-276

Additional exemplary pipecolic acid derivatives are represented by Formulas L-LIV and Tables XXXII-XXXVI.

TABLE XXXII

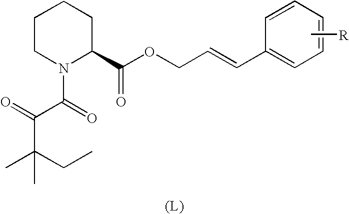

FORMULA L

| Compound | Structure |
|---|---|
| 255 | R = 3,4-dichloro |
| 256 | R = 3,4,5-trimethoxy |
| 257 | R = H |
| 258 | R = 3-(2,5-Dimethoxy)phenylpropyl |
| 259 | R = 3-(3,4-Methylenedioxy)phenylpropyl |

TABLE XXXIII

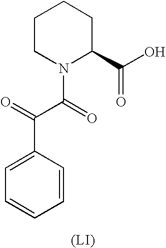

FORMULA LI

| Compound | Structure |
|---|---|
| 260 | R = 4-(p-Methoxy)butyl |
| 261 | R = 3-Phenylpropyl |
| 262 | R = 3-(3-Pyridyl)propyl |

TABLE XXXIV
FORMULA LII

| | |
|---|---|
| | (LII) |
| 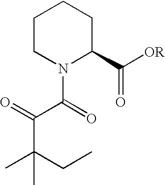 | |

| Compound | Structure |
|---|---|
| 263 | R = 3-(3-Pyridyl)propyl |
| 264 | R = 1,7-Diphenyl-4-heptyl |
| 265 | R = 4-(4-Methoxy)butyl |
| 266 | R = 1-Phenyl-6-(4-methoxyphenyl)-4-hexyl |
| 267 | R = 3-(2,5-Dimethoxy)phenylpropyl |
| 268 | R = 3-(3,4-Methylenedioxy)phenylpropyl |
| 269 | R = 1,5-Diphenylpentyl |

TABLE XXXV
FORMULA LIII

| | |
|---|---|
| | (LIII) |
| 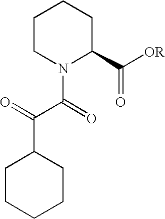 | |

| Compound | Structure |
|---|---|
| 270 | R = 4-(4-Methoxy)butyl |
| 271 | R = 3-Cyclohexylpropyl |
| 272 | R = 3-Phenylpropyl |

TABLE XXXVI
FORMULA LIV

| | |
|---|---|
| | (LIV) |
| 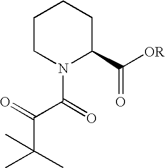 | |

| Compound | Structure |
|---|---|
| 273 | R = 3-Cyclohexylpropyl |
| 274 | R = 3-Phenylpropyl |
| 275 | R = 4-(4-Methoxy)butyl |
| 276 | R = 1,7-Diphenyl-4-heptyl |

The names of some of the compounds identified above are provided below in Table XXXVII.

TABLE XXXVII

| Compound | Name of Species |
|---|---|
| 172 | 4-(4-methoxyphenyl)butyl (2S)-1-[2-(3,4,5- |
| | trimethoxyphenyl)acetyl]hexahydro-2- |
| | pyridinecarboxylatel |
| 173 | 4-(4-methoxyphenyl)butyl (2S)-1-[2-(3,4,5- |
| | trimethoxyphenyl)acryloyl]hexahydro-2- |
| | pyridinecarboxylatel |
| 174 | 4-(4-methoxyphenyl)butyl (2S)-1-[2-(3,4,5- |
| | trimethoxyphenyl)propanoyl]hexahydro-2- |
| | pyridinecarboxylatel |
| 175 | 4-(4-methoxyphenyl)butyl (2S)-1-[2-oxo-2- |
| | (3,4,5-trimethoxyphenyl)acetyl]hexahydro-2- |
| | pyridinecarboxylatel |
| 177 | 3-cyclohexylpropyl (2S)-1-(3,3-dimethyl-2- |
| | oxopentanoyl)hexahydro-2- |
| | pyridinecarboxylatel |
| 178 | 3-phenylpropyl (2S)-1-(3,3-dimethyl-2- |
| | oxopentanoyl)hexahydro-2- |
| | pyridinecarboxylatel |
| 179 | 3-(3,4,5-trimethoxyphenyl)propyl (2S)-1- |
| | (3,3-dimethyl-2-oxopentanoyl)hexahydro-2- |
| | pyridinecarboxylatel |
| 180 | (1R)-2,2-dimethyl-1-phenethyl-3-butenyl |
| | (2S)-1-(3,3-dimethyl-2-oxopentanoyl) |
| | hexahydro-2-pyridinecarboxylatel |
| 181 | (1R)-1,3-diphenylpropyl (2S)-1-(3,3- |
| | dimethyl-2-oxopentanoyl)hexahydro-2- |
| | pyridinecarboxylatel |
| 182 | (1R)-1-cyclohexyl-3-phenylpropyl (2S)-1- |
| | (3,3-dimethyl-2-oxopentanoyl)hexahydro-2- |
| | pyridinecarboxylatel |
| 183 | (1S)-1,3-diphenylpropyl (2S)-1-(3,3- |
| | dimethyl-2-oxopentanoyl)hexahydro-2- |
| | pyridinecarboxylatel |
| 184 | (1S)-1-cyclohexyl-3-phenylpropyl (2S)-1- |
| | (3,3-dimethyl-2-oxopentanoyl)hexahydro-2- |
| | pyridinecarboxylatel |
| 185 | (22aS)-15,15-dimethylperhydropyrido[2,1- |
| | c][1,9,4]dioxazacyclononadecine-1,12,16,17- |
| | tetraonel |
| 186 | (24aS)-17,17-dimethylperhydropyrido[2,1- |
| | c][1,9,4]dioxazacyclohenicosine-1,14,18,19- |
| | tetraonel |
| 201 | ethyl 1-(2-oxo-3-phenylpropanoyl)-2- |
| | piperidinecarboxylatel |
| 202 | ethyl 1-pyruvoyl-2-piperidinecarboxylatel |
| 203 | ethyl 1-(2-oxobutanoyl)-2-piperidinecarboxylatel |
| 204 | ethyl 1-(3-methyl-2-oxobutanoyl)-2- |
| | piperidinecarboxylatel |
| 205 | ethyl 1-(4-methyl-2-oxopentanoyl)-2- |
| | piperidinecarboxylatel |
| 206 | ethyl 1-(3,3-dimethyl-2-oxobutanoyl)-2- |
| | piperidinecarboxylatel |
| 207 | ethyl 1-(3,3-dimethyl-2-oxopentanoyl)-2- |
| | piperidinecarboxylatel |
| 208 | 4-[2-(ethyloxycarbonyl)piperidino]-2,2- |
| | dimethyl-3,4-dioxobutyl acetatel |
| 209 | ethyll1-[2-(2-hydroxytetrahydro-2H-2- |
| | pyranyl)-2-oxoacetyl]-2- |
| | piperidinecarboxylatel |
| 210 | ethyll1-[2-(2-methoxytetrahydro-2H-2- |
| | pyranyl)-2-oxoacetyl]-2- |
| | piperidinecarboxylatel |
| 211 | ethyl 1-[2-(1-hydroxycyclohexyl)-2- |
| | oxoacetyl]-2-piperidinecarboxylatel |
| 212 | ethyl 1-[2-(1-methoxycyclohexyl)-2- |
| | oxoacetyl]-2-piperidinecarboxylatel |
| 213 | ethyl 1-(2-cyclohexyl-2-oxoacetyl)-2- |
| | piperidinecarboxylatel |
| 214 | ethyl 1-(2-oxo-2-piperidinoacetyl)-2- |
| | piperidinecarboxylatel |
| 215 | ethyl 1-[2-(3,4-dihydro-2H-6-pyranyl)-2- |
| | oxoacetyl)-2-piperidinecarboxylatel |
| 216 | ethyl 1-(2-oxo-2-phenylacetyl)-2- |
| | piperidinecarboxylatel |
| 217 | ethyl 1-(4-methyl-2-oxo-1-thioxopentyl)-2- |
| | piperidinecarboxylatel |
| 218 | 3-phenylpropyl 1-(2-hydroxy-3,3-dimethyl- |
| | pentanoyl)-2-piperidinecarboxylatel |
| 219 | (1R)-1-phenyl-3-(3,4,5-trimethoxyphenyl) |
| | propyl 1-(3,3-dimethylbutanoyl)-2- |
| | piperidinecarboxylatel |
| 220 | (1R)-1,3-diphenylpropyl 1-(benzylsulfonyl)- |
| | 2-piperidinecarboxylatel |
| 221 | 3-(3,4,5-trimethoxyphenyl)propyl 1- |
| | (benzylsulfonyl)-2-piperidinecarboxylate |
| 222 | 1-(2-[(2R,3R,6S)-6-[(2S,3E,5E,7E,9S,11R)- |
| | 2,13-dimethoxy-3,9,11-trimethyl-12-oxo- |
| | 3,5,7-tridecatrienyl]-2-hydroxy-3- |
| | methyltetrahydro-2H-2-pyranyl)-2-oxoacetyl)- |
| | 2-piperidinecarboxylic acidl |
| 223 | methyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylatel |
| 224 | isopropyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylatel |
| 225 | benzyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylatel |
| 226 | 1-phenylethyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylatel |
| 227 | (Z)-3-phenyl-2-propenyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyltetrahydro-2H-2-pyranyl)-2- |
| | oxoacetyl)-2-piperidinecarboxylatel |
| 228 | 3-(3,4-dimethoxyphenyl)propyl 1-(2- |
| | [(2R,3R,6S)-6-[(2S,3E,5E,7E,9S,11R)-2,13- |
| | dimethoxy-3,9,11-trimethyl-12-oxo-3,5,7- |
| | tridecatrienyl]-2-hydroxy-3-methyltetrahydro- |
| | 2H-2-pyranyl)-2-oxoacetyl)-2- |
| | piperidinecarboxylatel |
| 229 | N2-benzyl-1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylatel |
| 230 | N2-(3-phenylpropyl)-1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hyroxy-3-methyltetrahydro-2H-2-pyranyl)-2- |
| | oxoacetyl)-2-piperidinecarboxylate.l |
| 231 | (E)-3-(3,4-dichlorophenyl)-2-propenyl 1- |
| | (3,3-dimethyl-2-oxopentanoyl)-2-piperidinecarboxylatel |
| 232 | (E)-3-(3,4,5-trimethoxyphenyl)-2-propenyl 1- |
| | (3,3-dimethyl-2-oxopentanoyl)-2-piperidinecarboxylatel |
| 233 | (E)-3-phenyl-2-propenyl 1-(3,3-dimethyl-2- |
| | oxo-pentanoyl)-2-piperidinecarboxylatel |
| 234 | (E)-3-((3-(2,5-dimethoxy)-phenylpropyl)- |
| | phenyl)-2-propenyl 1-(3,3-dimethyl-2- |
| | oxopentanoyl)-2-piperidinecarboxylatel |
| 235 | (E)-3-(1,3-benzodioxol-5-yl)-2-propenyl 1- |
| | (3,3-dimethyl-2-oxopentanoyl)-2-piperidinecarboxylatel |
| 236 | 4-(4-methoxyphenyl)butyl 1-(2-oxo-2- |
| | phenylacetyl)-2-piperidinecarboxylatel |
| 237 | 3-phenylpropyl 1-(2-oxo-2-phenylacetyl)-2- |
| | piperidinecarboxylatel |
| 238 | 3-(3-pyridyl)propyl 1-(2-oxo-2- |
| | phenylacetyl)-2-piperidinecarboxylatel |
| 239 | 3-(3-pyridyl)propyl 1-(3,3-dimethyl-2- |
| | oxopentanoyl)-2-piperidinecarboxylatel |
| 240 | 4-phenyl-1-(3-phenylpropyl)butyl 1-(3,3- |
| | dimethyl-2-oxopentanoyl)-2-piperidinecarboxylatel |
| 241 | 4-(4-methoxyphenyl)butyl 1-(3,3-dimethyl-2- |
| | oxopentanoyl)-2-piperidinecarboxylatel |
| 242 | 1-(4-methoxyphenethyl)-4-phenylbutyl 1-(3,3- |
| | dimethyl-2-oxopentanoyl)-2-piperidinecarboxylatel |
| 243 | 3-(2,5-dimethoxyphenyl)propyl 1-(3,3- |
| | dimethyl-2-oxopentanoyl)-2- |
| | piperidinecarboxylatel |
| 244 | 3-(1,3-benzodioxol-5-yl)propyl 1-(3,3- |
| | dimethyl-2-oxopentanoyl)-2-piperidinecarboxylatel |
| 245 | 1-phenethyl-3-phenylpropyl 1-(3,3-dimethyl- |
| | 2-oxopentanoyl)-2-piperidinecarboxylatel |
| 246 | 4-(4-methoxyphenyl)butyl 1-(2-cyclohexyl-2- |
| | oxoacetyl)-2-piperidinecarboxylatel |
| 247 | 3-cyclohexylpropyl 1-(2-cyclohexyl-2- |
| | oxoacetyl)-2-piperidinecarboxylatel |
| 248 | 3-phenylpropyl 1-(2-cyclohexyl-2-oxoacetyl)- |
| | 2-piperidinecarboxylatel |
| 249 | 3-cyclohexylpropyl 1-(3,3-dimethyl-2- |
| | oxobutanoyl)-2-piperidinecarboxylatel |
| 250 | 3-phenylpropyl 1-(3,3-dimethyl-2- |
| | oxobutanoyl)-2-piperidinecarboxylatel |
| 251 | 4-(4-methoxyphenyl)butyl 1-(3,3-dimethyl-2- |
| | oxobutanoyl)-2-piperidinecarboxylatel |
| 252 | 4-phenyl-1-(3-phenylpropyl)butyl 1-(3,3- |
| | dimethyl-2-oxobutanoyl)-2-piperidinecarboxylate |

In yet a further embodiment, there is provided a method for treating or preventing hearing loss which comprises administering to a patient a compound of formula LV:

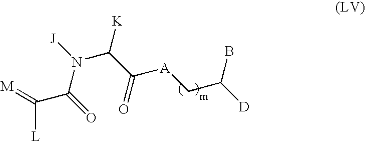

or a pharmaceutically acceptable salt, ester, or solvate thereof, wherein:

m is 0-3;

A is CH₂, O, NH, or N—(C₁-C₄ alkyl);

B and D are independently hydrogen, Ar, C₅-C₇ cycloalkyl substituted C₁-C₆ straight or branched chain alkyl or C₂-C₆ straight or branched chain alkenyl, C₅-C₇ cycloalkenyl substituted C₁-C₆ straight or branched chain alkyl or C₂-C₆ straight or branched chain alkenyl, or Ar substituted C₁-C₆ straight or branched chain alkyl or C₂-C₆ straight or branched chain alkenyl, wherein in each case, one or two carbon atom(s) of said alkyl or alkenyl may be substituted with one or two heteroatom(s) independently selected from the group consisting of oxygen, sulfur, SO, and SO₂ in chemically reasonable substitution patterns, or

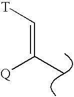

wherein Q is hydrogen, C₁-C₆ straight or branched chain alkyl, or C₂-C₆ straight or branched chain alkenyl; and

T is Ar or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl;

Ar is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), 0-(C₂-C₄ straight or branched chain alkenyl), O-benzyl, O-phenyl, amino, 1,2-methylenedioxy, carbonyl, and phenyl;

L is either hydrogen or U; M is either oxygen or CH-U, provided that if L is hydrogen, then M is CH-U, or if M is oxygen then L is U;

U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar, or Ar;

J is hydrogen, C₁ or C₂ alkyl, or benzyl; K is C₁-C₄ straight or branched chain alkyl, benzyl or cyclohexylmethyl; or J and K are taken together to form a 5-7 membered heterocyclic ring which is substituted with oxygen, sulfur, SO, or SO₂. Representative species of Formula LV are presented in Table XXXVIII:

TABLE XXXVIII

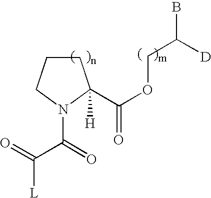

| Cpd. | n | m | B | D | L |
|---|---|---|---|---|---|
| 253 | 2 | 0 | 3-Phenylpropyl | 3-(3-Pyridyl)propyl | Phenyl |
| 254 | 2 | 0 | 3-Phenylpropyl | 3-(2-Pyridyl)propyl | Phenyl |
| 255 | 2 | 0 | 3-Phenylpropyl | 2-(4-Methoxyphenyl)ethyl | Phenyl |
| 256 | 2 | 0 | 3-Phenylpropyl | 3-Phenylpropyl | Phenyl |
| 257 | 2 | 0 | 3-Phenylpropyl | 3-Phenylpropyl | 3,4,5- |
| | | | | | Trimethoxyphenyl |
| 258 | 2 | 0 | 3-Phenylpropyl | 2-(3-Pyridyl)propyl | 3,4,5- |
| | | | | | Trimethoxyphenyl |
| 259 | 2 | 0 | 3-Phenylpropyl | 3-(2-Pyridyl)propyl | 3,4,5- |
| | | | | | Trimethoxyphenyl |
| 260 | 2 | 0 | 3-Phenylpropyl | 3-(4-Methoxyphenyl)propyl | 3,4,5- |
| | | | | | Trimethoxyphenyl |
| 261 | 2 | 0 | 3-Phenylpropyl | 3-(3-Pyridyl)propyl | 3-iso-propoxyphenyl |

### Formula (LVI)

U.S. Pat. No. 5,330,993, incorporated herein by reference, discloses an exemplary pipecolic acid derivative of Formula LVI:

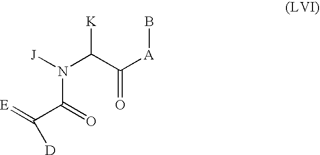

or a pharmaceutically acceptable salt, ester, or solvate thereof, wherein:

A is O, NH, or N—(C₁-C₄ alkyl);

B is hydrogen, CHL-Ar, C₁-C₆ straight or branched chain alkyl, C₂-C₆ straight or branched chain alkenyl, C₅-C₇ cycloalkyl, C₅-C₇ cycloalkenyl, Ar substituted C₁-C₆ alkyl or C₂-C₆ alkenyl, or

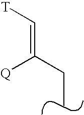

wherein L and Q are independently hydrogen, C₁-C₆ straight or branched chain alkyl, or C₂-C₆ straight or branched chain alkenyl; and

T is Ar or C₅-C₇ cyclohexyl substituted at positions 3 and 4 with substituents independently selected from the group consisting of hydrogen, hydroxy, O—(C₁-C₄ alkyl), O—(C₂-C₄ alkenyl), and carbonyl;

Ar is selected from the group consisting of 1-napthyl, 2-napthyl, 2-furyl, 3-furyl, 2-thienyl, 2-pyridyl, 3-pyridyl, 4-pyridyl and phenyl having 1-3 substituent(s) independently selected from the group consisting of hydrogen, halo, hydroxy, nitro, CF₃, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, and phenyl.

D is hydrogen or U; E is oxygen or CH-U, provided that if D is hydrogen, then E is CH-U, or if E is oxygen, then D is U;

U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇-cycloalkyl, C₅-C₇ cycloalkenyl substituted with C₁-C₄ straight or branched chain alkyl or C₂-C₄ straight or branched chain alkenyl, 2-indolyl, 3-indolyl, (C₁-C₄ alkyl or C₂-C₄ alkenyl)-Ar, or Ar;

J is hydrogen, C₁ or C₂ alkyl, or benzyl; K is C₁-C₄ straight or branched chain alkyl, benzyl or cyclohexylethyl; or J and K are taken together to form a 5-7 membered heterocyclic ring which is substituted with oxygen, sulfur, SO, or SO₂.

### Formula LVII

A preferred pipecolic acid derivative is a compound of Formula LVII:

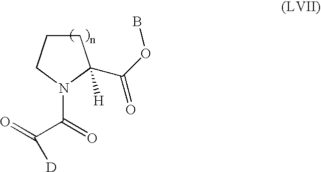

or a pharmaceutically acceptable salt, ester, or solvate thereof, wherein:

n is 2;

D is phenyl, methoxy, 2-furyl, or 3,4,5-trimethoxyphenyl; and

B is benzyl, 3-phenylpropyl, 4-(4-methoxyphenyl)butyl, 4-phenylbutyl, phenethyl, 3-cyclohexylpropyl, 4-cyclohexylbutyl, 3-cyclopentylpropyl, 4-cyclohexylbutyl, 3-phenoxybenzyl, 3-(3-indolyl)propyl, or 4-(4-methoxyphenyl)butyl;

provided that:

when D is phenyl, then B is benzyl, 3-phenylpropyl, 4-(4-methoxyphenyl)butyl, 4-phenylbutyl, phenethyl, or 4-cyclohexylbutyl; when D is methoxy, B is benzyl, 4-cyclohexylbutyl, 3-cyclohexylpropyl, or 3-cyclopentylpropyl;

when D is 2-furyl, then B is benzyl; and when D is 3,4,5-trimethoxyphenyl, then B is 4-cyclohexylbutyl, 3-phenoxybenzyl, 4-phenylbutyl, 3-(3-indolyl)propyl, or 4-(4-methoxyphenyl)butyl.

Representative species of Formula LVII are presented in Table XXXIX.

TABLE XXXIX

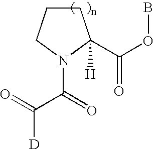

| Cpd. | B | D | n |
|---|---|---|---|
| 262 | Benzyl | Phenyl | 2 |
| 263 | 3-Phenylpropyl | Phenyl | 2 |
| 264 | 4-(4-Methoxyphenyl)butyl | Phenyl | 2 |
| 265 | 4-Phenylbutyl | Phenyl | 2 |
| 266 | Phenethyl | Phenyl | 2 |
| 267 | 4-Cyclohexylbutyl | Phenyl | 2 |
| 268 | Benzyl | Methoxy | 2 |
| 269 | 4-Cyclohexylbutyl | Methoxy | 2 |
| 269 | 3-Cyclohexylpropyl | Methoxy | 2 |
| 270 | 3-Cyclopentylpropyl | Methoxy | 2 |
| 271 | Benzyl | 2-Furyl | 2 |
| 272 | 4-Cyclohexylbutyl | 3,4,5-Trimethoxyphenyl | 2 |
| 273 | 3-Phenoxybenzyl | 3,4,5-Trimethoxyphenyl | 2 |
| 274 | 4-Phenylbutyl | 3,4,5-Trimethoxyphenyl | 2 |
| 275 | 3-(3-Indolyl)propyl | 3,4,5-Trimethoxyphenyl | 2 |
| 276 | 4-(4-Methoxyphenyl)butyl | 3,4,5-Trimethoxyphenyl | 2 |

### Formula LVIII

The pipecolic acid derivative may also be a compound of formula LVIII:

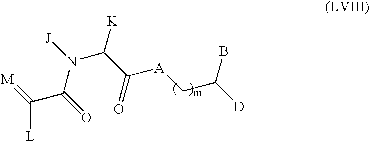

or a pharmaceutically acceptable salt, ester, or solvate thereof, wherein:

V is CH, N, or S;

J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;

R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;

Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;

A, B, D, L, M, and m are as defined in Formula LV, above.

In an additional embodiment of the invention, there is provided a method for the treatment or prevention of hearing loss or neurodegeneration in the ear which comprises administering to a warm-blooded animal a compound of the following formulae:

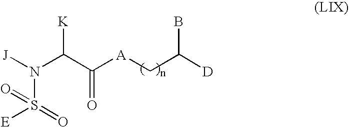

or a pharmaceutically acceptable salt, ester or solvate thereof, wherein:

A is CH₂, O, NH, or N—(C₁-C₄ alkyl);

B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or

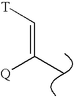

wherein Q is hydrogen, C₁-C₆ straight or branched chain alkyl, or C₂-C₆ straight or branched chain alkenyl; and

T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;

provided that both B and D are not hydrogen;

Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;

E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar;

J is hydrogen, C₁ or C₂ alkyl, or benzyl; K is C₁-C₄ straight or branched chain alkyl, benzyl, or cyclohexylmethyl; or J and K are taken together to form a 5-7 membered heterocyclic ring which is substituted with O, S, SO, or SO₂;

n is 0 to 3; and

the stereochemistry at carbon positions 1 and 2 is R or S.

### Formula LX

In a preferred embodiment of Formula I, J and K are taken together and the small molecule sulfonamide is a compound of Formula II:

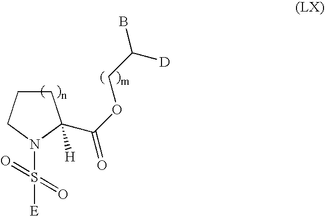

or a pharmaceutically acceptable salt thereof, wherein:

n is 1 or 2; and

m is 0 or 1.

In a more preferred embodiment, B is selected from the group consisting of hydrogen, benzyl, 2-phenylethyl, and 3-phenylpropyl;

D is selected from the group consisting of phenyl, 3-phenylpropyl, 3-phenoxyphenyl, and 4-phenoxyphenyl; and

E is selected from the group consisting of phenyl, 4-methylphenyl, 4-methoxyphenyl, 2-thienyl, 2,4,6-triisopropylphenyl, 4-fluorophenyl, 3-methoxyphenyl, 2-methoxyphenyl, 3,5-dimethoxyphenyl, 3,4,5-trimethoxyphenyl, methyl, 1-naphthyl, 8-quinolyl, 1-(5-N,N-dimethylamino)-naphthyl, 4-iodophenyl, 2,4,6-trimethylphenyl, benzyl, 4-nitrophenyl, 2-nitrophenyl, 4-chlorophenyl, and E-styrenyl.

### Formula LXI

Another exemplary small molecule sulfonamide is a compound of Formula III:

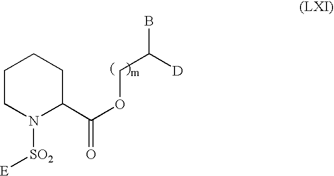

or a pharmaceutically acceptable salt thereof, wherein:

B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or

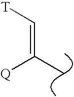

wherein Q is hydrogen, C₁-C₆ straight or branched chain alkyl, or C₂-C₆ straight or branched chain alkenyl; and

T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;

provided that both B and D are not hydrogen;

Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;

E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and

m is 0 to 3.

A further exemplary small molecule sulfonamide is a compound of Formula (LXII):

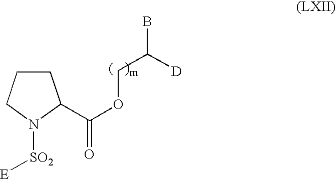

or a pharmaceutically acceptable salt thereof, wherein:

B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl, or Ar, and wherein one or two carbon atom(s) of said alkyl or alkenyl may be substituted with one or two heteroatom(s) independently selected from the group consisting of O, S, SO, and SO₂ in chemically reasonable substitution patterns, or

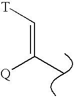

wherein Q is hydrogen, C₁-C₆ straight or branched chain alkyl, or C₂-C₆ straight or branched chain alkenyl; and

T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl; provided that both B and D are not hydrogen;

Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;

E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and

m is 0 to 3.

A further exemplary small molecule sulfonamide is a compound of Formula LXIII:

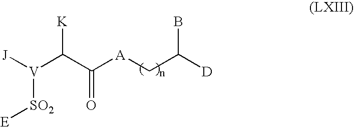

or a pharmaceutically acceptable salt, ester, or solvate thereof, wherein:

V is CH, N, or S;

J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;

R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;

Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;

A, B, D, E, and n are as defined in Formula I above.

Representative species of Formulas LIX-LXIII are presented in Table XL.

TABLE XL

| Cpd. | Structure and name |
|---|---|
| 278 | 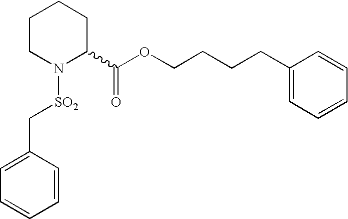 |
| | 4-phenyl-1-butyl-1-(benzylsulfonyl)-(2R, S)-2- |
| | pipecolinate |
| 279 | 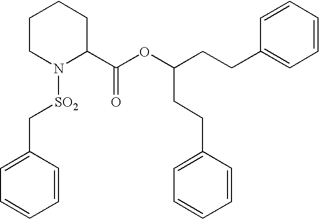 |
| | 1,5-diphenyl-3-pentyl-N-(a-toluenesulfonyl)- |
| | pipecolate |
| 280 | 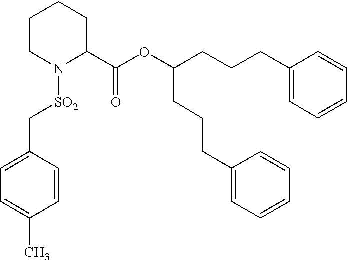 |
| | 1,7-diphenyl-4-heptyl-N-(para-toluene- |
| | sulfonyl)pipecolate |
| 281 | 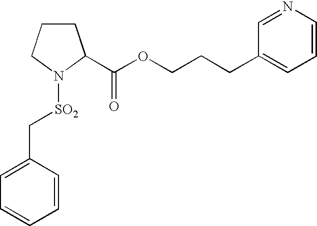 |
| | 3-(3-pyridyl)-1-propyl-(2S)-N-(a- |
| | toluenesulfonyl)-pyrrolidine-2-carboxylate |
| 282 | 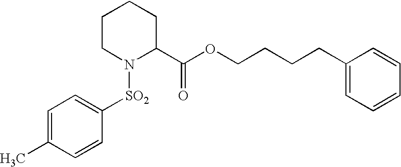 |
| | 4-phenyl-1-butyl-N-(para- |
| | toluenesulfonyl)pipecolate |
| 283 | 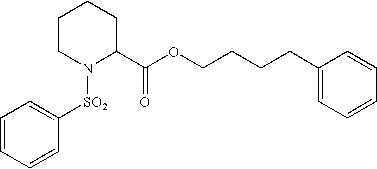 |
| | 4-phenyl-1-butyl-N-(benzenesulfonyl)-pipecolate |
| 284 | 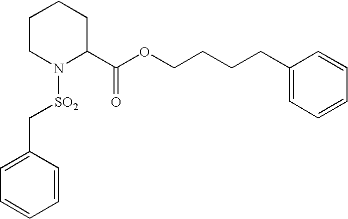 |
| | 4-phenyl-1-butyl-N-(a-toluenesulfonyl)pipecolate |

VII. Carboxylic Acid Isosteres as Sensorineuro-trophic Compounds

Another especially preferred embodiment of the invention is a compound of formula (LXIV):

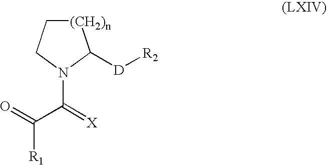

in which:

n is 1-3;

X is either O or S;

R₁ is selected from the group consisting of C₁-C₉ straight or branched chain alkyl, C₂-C₉ straight or branched chain alkenyl, aryl, heteroaryl, carbocycle, or heterocycle;

D is a bond, or a C₁-C₁₀ straight or branched chain alkyl, C₂-C₁₀ alkenyl or C₂-C₁₀ alkynyl; and

R₂ is a carboxylic acid or a carboxylic acid isostere;

or a pharmaceutically acceptable salt, ester, or solvate thereof;

Preferred embodiments of this invention are where R₂ is a carbocycle or heterocycle containing any combination of CH₂, O, S, or N in any chemically stable oxidation state, where any of the atoms of said ring structure are optionally substituted in one or more positions with R³.

Especially preferred embodiments of this invention are where R₂ is selected from the group below:

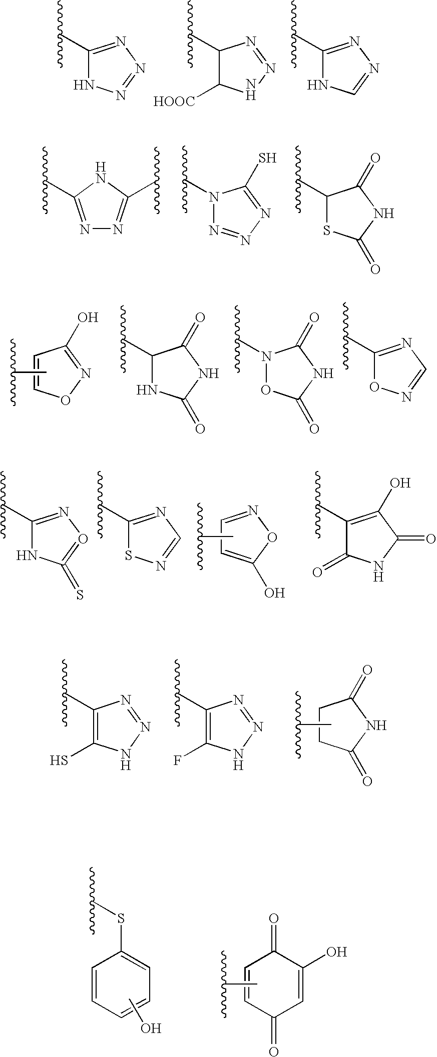

where the atoms of said ring structure may be optionally substituted at one or more positions with R³.

Another preferred embodiment of this invention is where R₂ is selected from the group consisting of —COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl.

Preferred embodiments of this invention are: (2S)-1-(1,2-dioxo-3,3-dimethylpentyl)-2-hydroxymethyl pyrrolidine; (2S)-1-(1,2-dioxo-3,3-dimethylpentyl)-2-pyrrolidinetetrazole; (2S)-1-(1,2-dioxo-3,3-dimethylpentyl)-2-pyrrolidinecarbonitrile; and (2S)-1-(1,2-dioxo-3,3-dimethylpentyl)-2-aminocarbonyl piperidine.

A compound of the present invention, especially formula LXIV, wherein n is 1, X is O, D is a bond, R₁ is 1,1,dimethylpropyl, and R₂ is —CN, is named (2S)-1-(1,2-dioxo-3,3-dimethylpentyl)-2-pyrrolidine-carbonitrile.

Specific embodiments of the inventive compounds are presented in Tables XLI, XLII, and XLIII. The present invention contemplates employing the compounds of Tables XLI, XLII and XLIII, below.

TABLE XLI

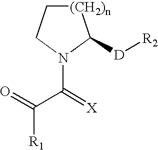

when D is a bond and R2 is COOH,

| No. | X | n | R₁ |
|---|---|---|---|
| 285 | O | 1 | 3,4,5-trimethylphenyl |
| 286 | O | 2 | 3,4,5-trimethylphenyl |
| 287 | O | 1 | tert-butyl |
| 287 | O | 3 | tert-butyl |
| 288 | O | 1 | cyclopentyl |
| 289 | O | 2 | cyclopentyl |
| 290 | O | 3 | cyclopentyl |
| 291 | O | 1 | cyclohexyl |
| 292 | O | 2 | cyclohexyl |
| 293 | O | 3 | cyclohexyl |
| 294 | O | 1 | cycloheptyl |
| 295 | O | 2 | cycloheptyl |
| 296 | O | 3 | cycloheptyl |
| 297 | O | 1 | 2-thienyl |
| 298 | O | 2 | 2-thienyl |
| 299 | O | 3 | 2-thienyl |
| 300 | O | 1 | 2-furyl |
| 301 | O | 2 | 2-furyl |
| 302 | O | 3 | 2-furyl |
| 303 | O | 3 | phenyl |
| 304 | O | 1 | 1,1-dimethylpentyl |
| 305 | O | 2 | 1,1-dimethylhexyl |
| 306 | O | 3 | ethyl |
| 307 | | | |

TABLE XLII

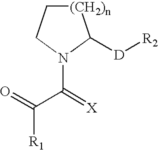

| No. | X | n | R₁ | D | R₂ |
|---|---|---|---|---|---|
| 308 | S | 1 | 1,1-dimethyl propyl | CH₂ | COOH |
| 309 | S | 1 | 1,1-dimethyl propyl | bond | COOH |
| 310 | O | 1 | 1,1-dimethyl propyl | CH₂ | OH |
| 311 | O | 1 | 1,1-dimethyl propyl | bond | SO₃H |
| 312 | O | 1 | 1,1-dimethyl propyl | CH₂ | CN |
| 313 | O | 1 | 1,1-dimethyl propyl | bond | CN |
| 314 | O | 1 | 1,1-dimethyl propyl | bond | tetrazolyl |
| 315 | S | 1 | phenyl | (CH₂)₂ | COOH |
| 316 | S | 1 | phenyl | (CH₂)₃ | COOH |
| 317 | S | 2 | phenyl | CH₂ | COOH |
| 318 | O | 1 | 1,1-dimethyl propyl | bond | CONH₂ |
| 319 | O | 2 | 1,1-dimethyl propyl | bond | CONH₂ |
| 320 | S | 2 | 2-furyl | bond | PO₃H₂ |
| 321 | O | 2 | propyl | (CH₂)₂ | COOH |
| 322 | O | 1 | propyl | (CH₂)₃ | COOH |
| 323 | O | 1 | tert-butyl | (CH₂)₄ | COOH |
| 324 | O | 1 | methyl | (CH₂)₅ | COOH |
| 325 | O | 2 | phenyl | (CH₂)₆ | COOH |
| 326 | O | 2 | 3,4,5-trimethoxy- | CH₂ | COOH |
| | | | phenyl | | |
| 327 | O | 2 | 3,4,5-trimethoxy- | CH₂ | tetrazolyl |
| | | | phenyl | | |

TABLE XLIII

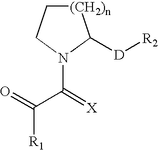

| No. | n | X | D | R₂ | R₁ |
|---|---|---|---|---|---|
| 328 | 1 | S | bond | COOH | Phenyl |
| 329 | 1 | O | bond | COOH | a-MethylBenzyl |
| 330 | 2 | O | bond | COOH | 4-MethylBenzyl |
| 331 | 1 | O | bond | Tetrazole | Benzyl |
| 332 | 1 | O | bond | SO₃N | a-MethylBenzyl |
| 333 | 1 | O | CH₂ | COOH | 4-MethylBenzyl |
| 334 | 1 | O | bond | SO₂HNMe | Benzyl |
| 335 | 1 | O | bond | CN | a-MethylBenzyl |
| 336 | 1 | O | bond | PO₃H₂ | 4-MethylBenzyl |
| 337 | 2 | O | bond | COOH | Benzyl |
| 338 | 2 | O | bond | COOH | a-MethylBenzyl |
| 339 | 2 | O | bond | COOH | 4-MethylBenzyl |
| 340 | 2 | S | bond | COOH | 3,4,5- |
| | | | | | trimethoxyphenyl |
| 341 | 2 | O | bond | COOH | Cyclohexyl |
| 342 | 2 | O | bond | PO₂HEt | i-propyl |
| 343 | 2 | O | bond | PO₂HPropyl | ethyl |
| 344 | 2 | O | bond | PO₃(Et)₂ | Methyl |
| 345 | 2 | O | bond | OMe | tert-butyl |
| 346 | 1 | O | bond | OEt | n-pentyl |
| 347 | 2 | O | bond | OPropyl | n-hexyl |
| 348 | 1 | O | bond | OButyl | Cyclohexyl |
| 349 | 1 | O | bond | OPentyl | cyclopentyl |
| 350 | 1 | O | bond | OHexyl | n-heptyl |
| 351 | 1 | O | bond | SMe | n-octyl |
| 352 | 1 | O | bond | SEt | n-nonyl |
| 353 | 2 | O | bond | SPropyl | 2-indolyl |
| 354 | 2 | O | bond | SButyl | 2-furyl |
| 355 | 2 | O | bond | NHCOMe | 2-thiazolyl |
| 356 | 2 | O | bond | NHCOEt | 2-thienyl |
| 357 | 1 | O | CH₂ | N(Me)₂ | 2-pyridyl |
| 358 | 1 | O | (CH₂)₂ | N(Me)Et | 1,1- |
| | | | | | dimethylpropyl |
| 359 | 1 | O | (CH₂)₃ | CON(Me)₂ | 1,1- |
| | | | | | dimethylpropyl |
| 360 | 1 | O | (CH₂)₄ | CONHMe | 1,1- |
| | | | | | dimethylpropyl |
| 361 | 1 | O | (CH₂)₅ | CONHEt | 1,1-dimethylpropyl |
| 362 | 1 | O | (CH₂)₆ | CONHPropyl | 1,1-dimethylpropyl |
| 363 | 1 | O | bond | CONH(O)Me | Benzyl |
| 364 | 1 | O | bond | CONH(O)Et | a-Methylphenyl |
| 365 | 1 | O | bond | CONH(O)Propyl | 4-Methylphenyl |
| 366 | 1 | O | (CH₂)₂ | COOH | Benzyl |
| 367 | 1 | O | bond | COOH | a-Methylphenyl |
| 368 | 1 | O | bond | COOH | 4-Methylphenyl |
| 369 | 1 | O | CH₂ | COOH | 1,1-dimethylpropyl |
| 370 | 1 | O | (CH₂)₂ | COOH | 1,1-dimethylbutyl |
| 371 | 1 | O | (CH₂)₃ | COOH | 1,1-dimethylpentyl |
| 372 | 1 | O | (CH₂)₄ | COOH | 1,1-dimethylhexyl |
| 373 | 1 | O | (CH₂)₅ | COOH | 1,1-dimethylethyl |
| 374 | 1 | O | (CH₂)₆ | COOH | iso-propyl |
| 375 | 1 | O | (CH₂)₇ | COOH | tert-butyl |
| 376 | 1 | O | (CH₂)₈ | COOH | 1,1-dimethylpropyl |
| 377 | 1 | O | (CH₂)₉ | COOH | benzyl |
| 378 | 1 | O | (CH₂)₁₀ | COOH | 1,1-dimethylpropyl |
| 379 | 1 | O | C₂H₂ | COOH | cyclohexylmethyl |
| 380 | 1 | O | 2-OH, Et | COOH | 1,1-dimethylpropyl |
| 381 | 1 | O | 2-butylene | COOH | 1,1-dimethylpropyl |
| 382 | 1 | S | i-Pro | COOH | 1,1-dimethylpropyl |
| 383 | 2 | S | t-Bu | COOH | phenyl |
| 384 | 2 | O | 2-NO₂-hexyl | COOH | 1,1-dimethylpropyl |
| 385 | 1 | O | (CH₂)₂ | CN | 1,1-dimethylpropyl |
| 386 | 1 | O | (CH₂)₃ | CN | 1,1-dimethylpropyl |
| 387 | 3 | O | bond | CONHNHSO₂Me | Benzyl |
| 388 | 3 | O | bond | CONHNHSO₂Et | a-Methylphenyl |
| 389 | 3 | O | bond | CONHSO₂Me | 4-Methylphenyl |
| 390 | 1 | O | bond | CONHNHSO₂Et | Phenyl |
| 391 | 2 | O | bond | CON(Me)CN | a-Methylphenyl |
| 392 | 1 | O | bond | CON(Et)CN | 4-Methylphenyl |
| 393 | 1 | O | (CH₂)₂ | COOH | methyl |
| 394 | 1 | O | (CH₂)₃ | COOH | ethyl |
| 395 | 1 | O | (CH₂)₄ | COOH | n-propyl |
| 396 | 1 | O | (CH₂)₅ | COOH | t-butyl |
| 397 | 1 | O | (CH₂)₆ | COOH | Pentyl |
| 398 | 1 | O | (CH₂)₇ | COOH | Hexyl |
| 399 | 1 | O | (CH₂)₈ | COOH | Heptyl |
| 400 | 1 | O | (CH₂)₉ | COOH | Octyl |
| 401 | 1 | O | C₂H₂ | COOH | Cyclohexyl |
| 402 | 2 | O | bond | 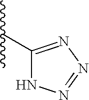 | 1,1-dimethylpropyl |
| 403 | 1 | O | bond | 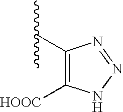 | 1,1-dimethylpropyl |
| 404 | 1 | O | bond | 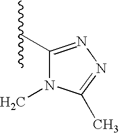 | 1,1-dimethylpropyl |
| 405 | 1 | O | bond | 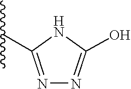 | 1,1-dimethylpropyl |
| 406 | 1 | O | bond | 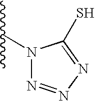 | 1,1-dimethylpropyl |
| 407 | 1 | O | bond | 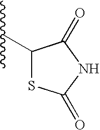 | 1,1-dimethylpropyl |
| 408 | 1 | O | bond | 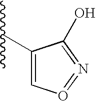 | 1,1-dimethylpropyl |
| 409 | 1 | O | bond | 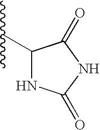 | 1,1-dimethylpropyl |
| 410 | 1 | O | bond | 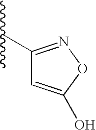 | 1,1-dimethylpropyl |
| 411 | 1 | O | bond | 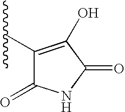 | 1,1-dimethylpropyl |
| 412 | 1 | O | bond | 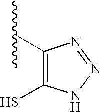 | 1,1-dimethylpropyl |
| 413 | 1 | O | bond | 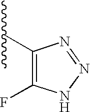 | 1,1-dimethylpropyl |
| 414 | 1 | O | bond | 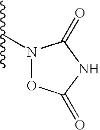 | 1,1-dimethylpropyl |
| 415 | 1 | O | bond | 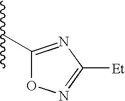 | 1,1-dimethylpropyl |
| 416 | 1 | O | bond | 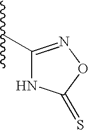 | 1,1-dimethylpropyl |
| 417 | 1 | O | bond | 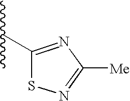 | 1,1-dimethylpropyl |
| 418 | 1 | O | bond | 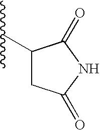 | 1,1-dimethylpropyl |
| 419 | 1 | O | bond | 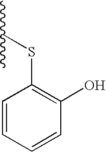 | 1,1-dimethylpropyl |
| 420 | 1 | O | bond | 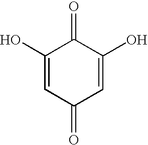 | 1,1-dimethylpropyl |
| 421 | 1 | O | bond | COOH | 1,1-dimethylpropyl |
| 422 | 2 | O | bond | COOH | 1,1-dimethylpropyl |

Another preferred embodiment of this aspect of the invention is the use for treating or preventing sensorineural hearing loss of a compound of the formula (LXV):

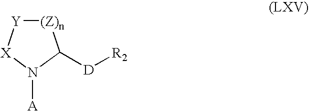

in which

X, Y, and Z are independently selected from the group consisting of C, O, S, or N, provided that X, Y, and Z are not all C;

n is 1-3;

A is selected from the group consisting of L₁, L₂, L₃, or L₄, in which

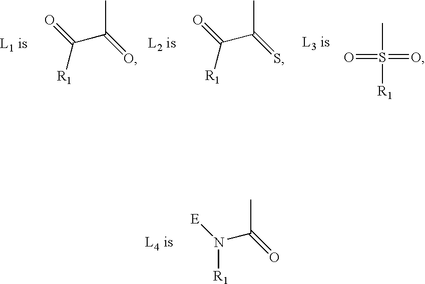

and R₁ and E, independently, are selected from the group consisting of hydrogen, C₁-C₉ straight or branched chain alkyl, C₂-C₉ straight or branched chain alkenyl, aryl, heteroaryl, carbocycle, and heterocycle;

R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where

R³ is hydrogen, hydroxy, halo, halo(C₁-C₆)-alkyl, thiocarbonyl, (C₁-C₆)-alkoxy, (C₂-C₆)-alkenoxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;

or a pharmaceutically acceptable salt, ester, or solvate thereof;

Preferred embodiments of this embodiment of the invention are those in which R₂ is a carbocycle or heterocycle containing any combination of CH₂, O, S, or N in any chemically stable oxidation state, where any of the atoms of said ring structure are optionally substituted in one or more positions with R³.

Especially preferred embodiments of this aspect of the invention are the use of those compounds in which R₂ is selected from the group below:

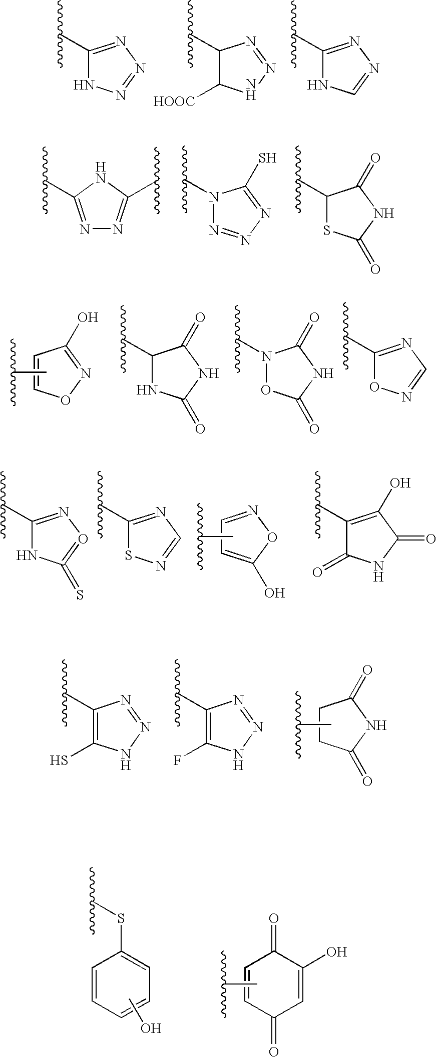

where the atoms of said ring structure may be optionally substituted at one or more positions with R³.

Another preferred embodiment of this invention is where R₂ is selected from the group consisting of —COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN.

Preferred embodiments of this embodiment are the sensorineurotrophic compounds (2S)-1-(phenylmethyl) carbamoyl-2-hydroxymethyl (4-thiazolidine), (2S)-1-(1,1-dimethyl propyl)carbamoyl-2-(4-thiazolidine)tetrazole and (2S)-1-(phenylmethyl) carbamoyl-2-(4-thiazolidine) carbonitrile.

The following structures are non-limiting examples of preferred carbocyclic and heterocyclic isosteres contemplated by this aspect of the invention:

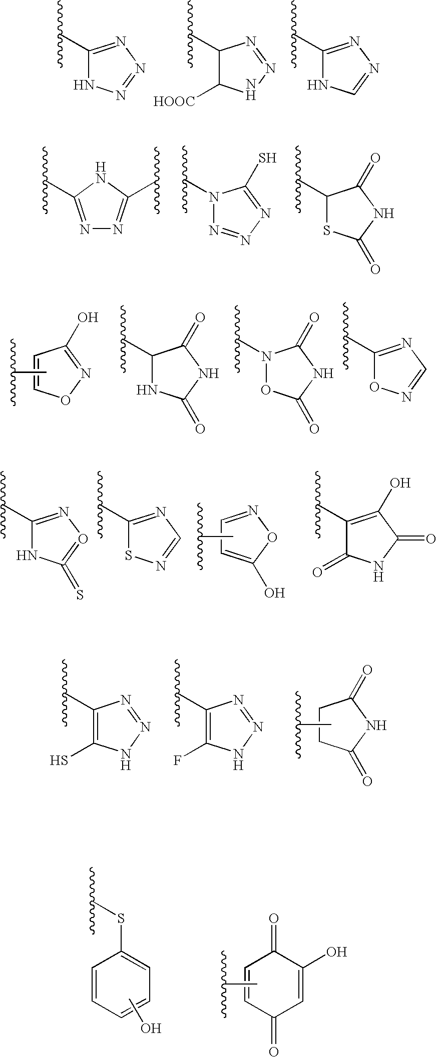

in which the atoms of said ring structure may be optionally substituted at one or more positions with R³ wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl. The present invention contemplates that when chemical substituents are added to a carboxylic isostere then the compound retains the properties of a carboxylic isostere. Particularly, the present invention contemplates that when a carboxylic isostere is optionally substituted with one or more moieties selected from R³, then the substitution cannot eliminate the carboxylic acid isosteric properties of the compound. The present invention contemplates that the placement of one or more R³ substituents upon a carbocyclic or heterocyclic carboxylic acid isostere shall not be at an atom(s) which maintains or is integral to the carboxylic acid isosteric properties of the inventive compound if such a substituent(s) would destroy the carboxylic acid isosteric properties of the inventive compound.

Other carboxylic acid isosteres not specifically exemplified or described in this specification are also contemplated by the present invention.

A compound for use in the present invention, especially formula LXV, wherein n is 1, X is O, D is a bond, R₁ is 1,1,dimethylpropyl, and R₂ is —CN, is named (2S)-1-(1,2-dioxo-3,3-dimethylpentyl)-2-pyrrolidinecarbonitrile.

Specific embodiments of the inventive compounds are presented in Tables XLIV, XLV, and XLVI. The present invention contemplates employing the compounds of Tables XLIV, XLV, and XLVI, below, for use in compositions and methods of the invention.

TABLE XLIV

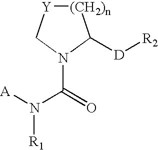

| No. | n | D | R₂ | A | Y | R₁ |
|---|---|---|---|---|---|---|
| 423 | 1 | bond | COOH | H | S | Benzyl |
| 424 | 1 | bond | COOH | H | S | a-MethylBenzyl |
| 425 | 1 | bond | COOH | H | S | 4-MethylBenzyl |
| 426 | 1 | bond | Tetrazole | H | S | Benzyl |
| 427 | 1 | bond | SO₃H | H | O | a-MethylBenzyl |
| 428 | 1 | CH₂ | COOH | H | O | 4-MethylBenzyl |
| 429 | 1 | bond | SO₂HNMe | H | O | Benzyl |
| 430 | 1 | bond | CN | H | N | a-MethylBenzyl |
| 431 | 1 | bond | PO₃H₂ | H | N | 4-MethylBenzyl |
| 432 | 2 | bond | COOH | H | N | Benzyl |
| 433 | 2 | bond | COOH | H | S | a-MethylBenzyl |
| 434 | 2 | bond | COOH | H | S | 4-MethylBenzyl |
| 435 | 2 | bond | COOH | H | S | 3,4,5-trimethoxy-phenyl |
| 436 | 2 | bond | COOH | H | S | Cyclohexyl |
| 437 | 2 | bond | PO₂HEt | H | O | i-propyl |
| 438 | 2 | bond | PO₃HPropyl | H | O | ethyl |
| 439 | 2 | bond | PO₃(Et)2 | H | N | Methyl |
| 440 | 2 | bond | OMe | H | S | tert-butyl |
| 441 | 2 | bond | OEt | H | S | n-pentyl |
| 442 | 2 | bond | OPropyl | H | S | n-hexyl |
| 443 | 1 | bond | OButyl | H | O | Cyclohexyl |
| 444 | 1 | bond | OPentyl | H | N | cyclopentyl |
| 445 | 1 | bond | OHexyl | H | S | n-heptyl |
| 446 | 1 | bond | SMe | H | S | n-octyl |
| 447 | 1 | bond | SEt | H | O | n-nonyl |
| 448 | 2 | bond | SPropyl | H | N | 2-indolyl |
| 449 | 2 | bond | SButyl | H | O | 2-furyl |
| 450 | 2 | bond | NHCOMe | H | S | 2-thiazolyl |
| 451 | 2 | bond | NHCOEt | H | S | 2-thienyl |
| 452 | 1 | CH₂ | N(Me)₂ | H | N | 2-pyridyl |
| 453 | 1 | (CH₂)₂ | N(Me)Et | H | S | 1,1-dimethylpropyl |
| 454 | 1 | (CH₂)₃ | CON(Me)₂ | H | O | 1,1-dimethylpropyl |
| 455 | 1 | (CH₂)₄ | CONHMe | H | N | 1,1-dimethylpropyl |
| 456 | 1 | (CH₂)₅ | CONHEt | H | S | 1,1-dimethylpropyl |
| 457 | 1 | (CH₂)₆ | CONHPropyl | H | S | 1,1-dimethylpropyl |

TABLE XLV

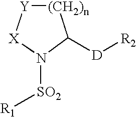

| No. | n | D | R₂ | Y | R₁ |
|---|---|---|---|---|---|
| 458 | | bond | CONH(O)Me | S | Benzyl |
| 459 | | bond | CONH(O)Et | S | a-Methylphenyl |
| 460 | 1 | bond | CONH(O)Propyl | S | 4-Methylphenyl |
| 461 | 2 | bond | COOH | S | Benzyl |
| 462 | 2 | bond | COOH | O | a-Methylphenyl |
| 463 | 2 | bond | COOH | O | 4-Methylphenyl |
| 464 | 1 | CH₂ | COOH | N | benzyl |
| 465 | 1 | (CH₂)₂ | COOH | N | benzyl |
| 466 | 1 | (CH₂)₃ | COOH | N | benzyl |
| 467 | 1 | (CH₂)₄ | COOH | S | benzyl |
| 468 | 1 | (CH₂)₅ | COOH | S | benzyl |
| 469 | 1 | (CH₂)₆ | COOH | S | benzyl |
| 470 | 1 | (CH₂)₇ | COOH | S | benzyl |
| 471 | 1 | (CH₂)₈ | COOH | O | benzyl |
| 472 | 1 | (CH₂)₉ | COOH | O | benzyl |
| 473 | 1 | (CH₂)₁₀ | COOH | O | benzyl |
| 474 | 1 | C₂H₂ | COOH | N | benzyl |
| 475 | 1 | 2-OH,Et | COOH | N | benzyl |
| 476 | 1 | 2butylene | COOH | S | benzyl |
| 477 | 1 | i-Pro | COOH | S | benzyl |
| 478 | 1 | tert-Bu | COOH | S | benzyl |
| 479 | 1 | 2-nitro | COOH | S | benzyl Hexyl |
| 480 | 3 | (CH₂)₂ | CN | S | benzyl |
| 481 | 1 | (CH₂)₃ | CN | S | benzyl |
| 482 | 3 | bond | CONHNHSO₂Me | N | Benzyl |
| 483 | 3 | bond | CONHNHSO₂Et | N | a-Methylphenyl |
| 484 | 3 | bond | CONHSO₂Me | N | 4-Methylphenyl |
| 485 | 2 | bond | CONHNHSO₂Et | N | Phenyl |
| 486 | 2 | bond | CON(Me)CN | O | a-Methylphenyl |
| 487 | 2 | bond | CON(Et)CN | O | 4-Methylphenyl |
| 488 | 1 | (CH₂)₂ | COOH | O | methyl |
| 489 | 1 | (CH₂)₃ | COOH | O | ethyl |
| 490 | 1 | (CH₂)₄ | COOH | N | n-propyl |
| 491 | 1 | (CH₂)₅ | COOH | N | t-butyl |
| 492 | 1 | (CH₂)₆ | COOH | N | Pentyl |
| 493 | 1 | (CH₂)₇ | COOH | S | Hexyl |
| 494 | 1 | (CH₂)₈ | COOH | S | Heptyl |
| 495 | 1 | (CH₂)₉ | COOH | S | Octyl |
| 496 | 1 | (CH₂)₁₀ | COOH | S | Nonyl |
| 497 | 1 | C₂H₂ | COOH | S | Cyclohexyl |

TABLE XLVI

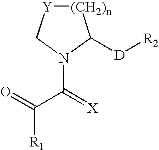

| No. | n | X | D | R₂ | Y | R₁ |
|---|---|---|---|---|---|---|
| 498 | 1 | O | bond | 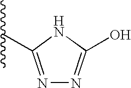 | O | 1,1-dimethylpropyl |
| 499 | 1 | O | bond | 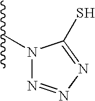 | S | 1,1-dimethylpropyl |
| 500 | 1 | O | bond | 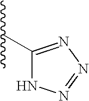 | S | 1,1-dimethylpropyl |
| 501 | 1 | O | bond | 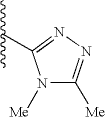 | O | 1,1-dimethylpropyl |
| 502 | 1 | O | bond | 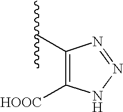 | N | 1,1-dimethylpropyl |
| 503 | 1 | O | bond | 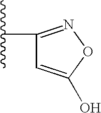 | S | 1,1-dimethylpropyl |
| 504 | 1 | O | bond | 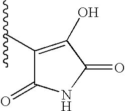 | N | 1,1-dimethylpropyl |
| 505 | 1 | O | bond | 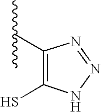 | N | 1,1-dimethylpropyl |
| 506 | 1 | O | bond | 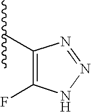 | S | 1,1-dimethylpropyl |
| 507 | 1 | O | bond | 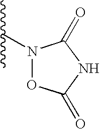 | O | 1,1-dimethylpropyl |
| 508 | 1 | O | bond | 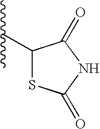 | S | 1,1-dimethylpropyl |
| 509 | 1 | O | bond | 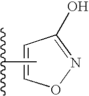 | S | 1,1-dimethylpropyl |
| 510 | 1 | O | bond | 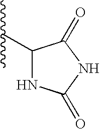 | O | 1,1-dimethylpropyl |
| 511 | 1 | O | bond | 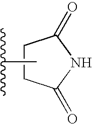 | S | 1,1-dimethylpropyl |
| 512 | 1 | O | bond | 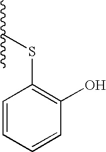 | O | 1,1-dimethylpropyl |
| 513 | 1 | O | bond | 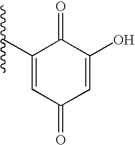 | S | 1,1-dimethylpropyl |
| 514 | 1 | O | bond | 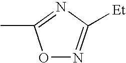 | N | 1,1-dimethylpropyl |
| 515 | 1 | O | bond | 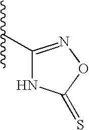 | O | 1,1-dimethylpropyl |
| 516 | 1 | O | bond | 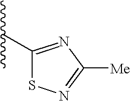 | S | 1,1-dimethylpropyl |

Compounds 517-610 are also exemplified for use in the present invention, and are defined as where Y is located at the 3-position of the heterocyclic ring for compounds 423-516, and n, A, D, Y, X, R₁, and R₂ remain the same as defined for compounds 423-516 in Tables XLIV, XLV, and XLVI.

Exemplary compound 611 is defined where S is located at the 3-position of the heterocyclic ring (3-thiazolidine), n is 1, R₁ is 1,1-dimethylpropyl, D is a bond, R₂ is COOH.

Exemplary compound 612 is defined where 0 is located at the 2-position of the heterocyclic ring (2-oxopentanoyl), n is 1, R₁ is 1,1-dimethylpropyl, D is a bond, R₂ is COOH (i.e. 3-(3,3-dimethyl-2-oxopentanoyl)-1,3-oxazolidine-4-carboxylic acid).

The present invention also contemplates other ring locations for the heteroatoms O, N, and S in sensorineurotrophic heterocyclic compounds. Also contemplated by the present invention are sensorineurotrophic heterocycles containing 3 or more heteroatoms chosen independently from O, N, and S.

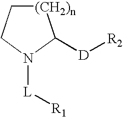

| No. | n | D | R₂ | L | R₁ |
|---|---|---|---|---|---|
| 613 | 1 | CH₂ | OH | 1,2-dioxoethyl | benzyl |
| 614 | 1 | bond | —CN | 1,2-dioxoethyl | 1,1-dimethylpropyl |
| 615 | 1 | bond | tetrazole | 1,2-dioxoethyl | 1,1-dimethylpropyl |
| 616 | 2 | bond | CONH₂ | 1,2-dioxoethyl | 1,1-dimethylpropyl |
| 617 | 1 | bond | COOH | 1,2-dioxoethyl | 1,1-dimethylpropyl |
| 618 | 2 | bond | COOH | 1,2-dioxoethyl | 1,1-dimethylpropyl |

In another embodiment of the invention, there is provided a compound for use in the treatment or prevention of sensorineural hearing loss embodiment of formula (LXVI):

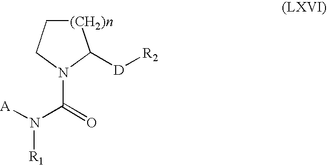

in which:

n is 1-3; R₁ and A are independently selected from the group consisting of hydrogen, C₁-C₉ straight or branched chain alkyl, C₂-C₉ straight or branched chain alkenyl, aryl, heteroaryl, carbocycle, and heterocycle;

D is a bond, or a C₁-C₁₀ straight or branched chain alkyl, C₂-C₁₀ alkenyl or C₂-C₁₀ alkynyl;

R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where

R³ is hydrogen, hydroxy, halo, halo(C₁-C₆)-alkyl, thiocarbonyl, (C₁-C₆)-alkoxy, (C₂-C₆)-alkenoxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl;

or a pharmaceutically acceptable salt, ester, or solvate thereof;

A preferred compound for use in this embodiment of this invention is (2S)-1-(cyclohexyl)carbamoyl-2-pyrrolidinecarboxylic acid.

Other preferred compounds for use in this embodiment of this invention are those in which R₂ is a carbocycle or heterocycle containing any combination of CH₂, O, S, or N in any chemically stable oxidation state, where any of the atoms of said ring structure are optionally substituted in one or more positions with R³.

Especially preferred embodiments of this aspect of the invention are those in which R₂ is selected from the group below:

(See figures on next page)

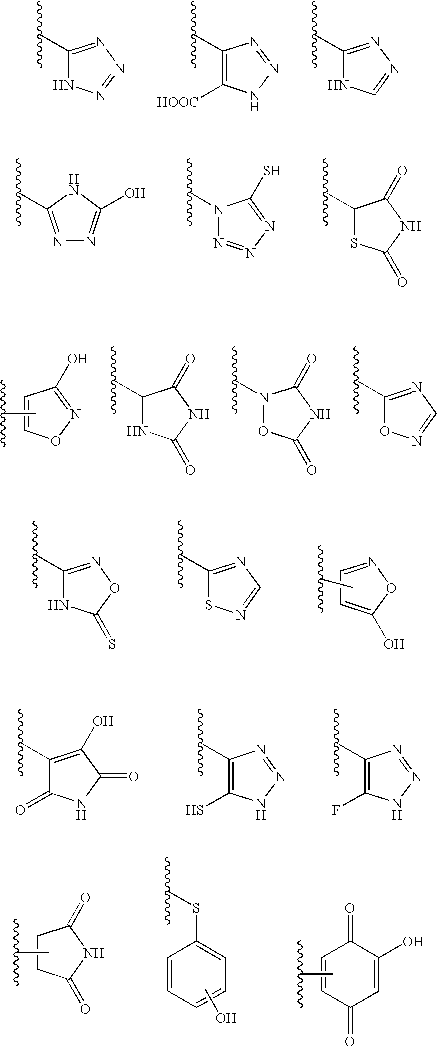

where the atoms of said ring structure may be optionally substituted at one or more positions with R³.

Another preferred embodiment of this invention is where R₂ is selected from the group consisting of —COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —COOH, —SO₃H, —SO₂HNR³, —PO₂ (R³)₂, —CN, —PO₃(R³)₂, —OR³, —SR³, —NHCOR³, —N(R³)₂, —CON(R³) 2, —CONH(O)R³—CONHNHSO₂R³, —COHNSO₂R³, and —CONR³CN wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl.

In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carbocyclic and heterocyclic isosteres contemplated by this aspect of the invention.

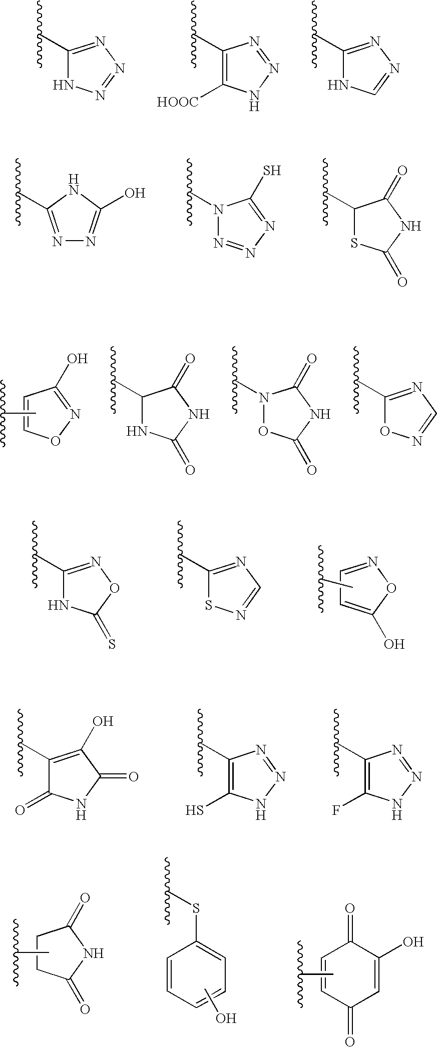

where the atoms of said ring structure may be optionally substituted at one or more positions with R³ wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl. The present invention contemplates that when chemical substituents are added to a carboxylic isostere then the inventive compound retains the properties of a carboxylic isostere.

The present invention contemplates that when a carboxylic isostere is optionally substituted with one or more moieties selected from R³, then the substitution cannot eliminate the carboxylic acid isosteric properties of the inventive compound. The present invention contemplates that the placement of one or more R³ substituents upon a carbocyclic or heterocyclic carboxylic acid isostere shall not be permitted at one or more atom(s) which maintain(s) or is/are integral to the carboxylic acid isosteric properties of the inventive compound, if such substituent(s) would destroy the carboxylic acid isosteric properties of the inventive compound.

A compound of the present invention, especially formula LXVI, wherein n is 1, X is O, D is a bond, R₁ is 1,1,dimethylpropyl, and R₂ is —CN, is named (2S)-1-(1,2-dioxo-3,3-dimethylpentyl)-2-pyrrolidinecarbonitrile.

Specific embodiments of the inventive compounds are presented in Table XLVII. The present invention contemplates employing the compounds of Table XLVII, below, for use in compositions and methods of the invention.

TABLE XLVII

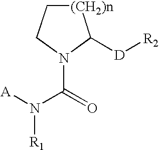

| No. | n | D | R₂ | A | R₁ |
|---|---|---|---|---|---|
| 619 | 1 | bond | COOH | H | cyclohexyl |
| 620 | 1 | bond | COOH | H | a-MethylBenzyl |
| 621 | 1 | bond | COOH | H | 4-MethylBenzyl |
| 622 | 1 | bond | Tetrazole | H | Benzyl |
| 623 | 1 | bond | SO₃H | H | a-MethylBenzyl |
| 624 | 1 | CH₂ | COOH | H | 4-MethylBenzyl |
| 625 | 1 | bond | SO₂HNMe | H | Benzyl |
| 626 | 1 | bond | CN | H | a-MethylBenzyl |
| 627 | 1 | bond | PO₃H₂ | H | 4-MethylBenzyl |
| 628 | 2 | bond | COOH | H | Benzyl |
| 629 | 2 | bond | COOH | H | a-MethylBenzyl |
| 630 | 2 | bond | COOH | H | 2-butyl |
| 631 | 2 | bond | COOH | H | 2-butyl |
| 632 | 2 | bond | COOH | H | Cyclohexyl |
| 633 | 2 | bond | PO₂HEt | H | i-propyl |
| 634 | 2 | bond | PO₃HPropyl | H | ethyl |
| 635 | 2 | bond | PO₃(Et)₂ | H | Methyl |
| 636 | 2 | bond | OMe | H | tert-butyl |
| 637 | 2 | bond | OEt | H | n-pentyl |
| 638 | 2 | bond | Opropyl | H | n-hexyl |
| 639 | 1 | bond | OButyl | H | Cyclohexyl |
| 639 | 1 | bond | OPentyl | H | cyclopentyl |
| 640 | 1 | bond | OHexyl | H | heptyl |
| 641 | 1 | bond | SMe | H | n-octyl |
| 642 | 1 | bond | SEt | H | n-hexyl |
| 643 | 2 | bond | SPropyl | H | n-hexyl |
| 644 | 2 | bond | SButyl | H | n-hexyl |
| 645 | 2 | bond | NHCOMe | H | n-hexyl |
| 646 | 2 | bond | NHCOEt | H | 2-thienyl |
| 647 | 1 | CH₂ | N(Me)₂ | H | adamantyl |
| 648 | 1 | (CH₂)₂ | N(Me)Et | H | adamantyl |
| 649 | 1 | (CH₂)₃ | CON(Me)₂ | H | adamantyl |
| 650 | 1 | (CH₂)₄ | CONHMe | H | adamantyl |
| 651 | 1 | (CH₂)₅ | CONHEt | H | adamantyl |
| 652 | 1 | (CH₂)₆ | CONHPropyl | H | adamantyl |
| 653 | 1 | bond | CONH(O)Me | H | Benzyl |
| 654 | 1 | bond | CONH(O)Et | H | α-methylphenyl |
| 655 | 1 | bond | CONH(O)Propyl | H | 4-Methylphenyl |
| 657 | 2 | bond | COOH | H | Benzyl |
| 658 | 2 | bond | COOH | H | α-Methylphenyl |
| 659 | 2 | bond | COOH | H | 4-Methylphenyl |
| 660 | 1 | CH₂ | COOH | Me | cyclohexyl |
| 661 | 1 | (CH₂)₂ | COOH | Et | cyclohexyl |
| 662 | 1 | (CH₂)₃ | COOH | Prop | cyclohexyl |
| 663 | 1 | (CH₂)₄ | COOH | But | cyclohexyl |
| 664 | 1 | (CH₂)₅ | COOH | H | cyclohexyl |
| 665 | 1 | (CH₂)₆ | COOH | H | cyclohexyl |
| 666 | 1 | (CH₂)₇ | COOH | H | cyclohexyl |
| 667 | 1 | (CH₂)₈ | COOH | H | cyclohexyl |
| 668 | 1 | (CH₂)₉ | COOH | H | cyclohexyl |
| 669 | 1 | (CH₂)₁₀ | COOH | H | cyclohexyl |
| 670 | 1 | C₂H₂ | COOH | H | cyclohexyl |
| 671 | 1 | 2-OH,Et | COOH | H | cyclohexyl |
| 672 | 1 | 2-butylene- | COOH | H | cyclohexyl |
| 673 | 1 | i-Pro | COOH | H | cyclohexyl |
| 674 | 1 | tert-Bu | COOH | H | cyclohexyl |
| 675 | 1 | 2-nitro Hexyl | COOH | H | cyclohexyl |
| 676 | 3 | (CH₂)₂ | CN | H | cyclohexyl |
| 677 | 1 | (CH₂)₃ | CN | H | cyclohexyl |
| 678 | 3 | bond | CONHNHSO₂Me | H | Benzyl |
| 679 | 3 | bond | CONHNHSO₂Et | H | α-Methylphenyl |
| 680 | 3 | bond | CONHSO₂Me | H | 4-Methylphenyl |
| 681 | 2 | bond | CONHNHSO₂Et | H | Phenyl |
| 682 | 2 | bond | CON(Me)CN | H | α-Methylphenyl |
| 683 | 2 | bond | CON(Et)CN | H | 4-Methylphenyl |
| 684 | 1 | (CH₂)₂ | COOH | H | methyl |
| 685 | 1 | (CH₂)₃ | COOH | H | ethyl |
| 686 | 1 | (CH₂)₄ | COOH | H | n-propyl |
| 687 | 1 | (CH₂)₅ | COOH | H | t-butyl |
| 688 | 1 | (CH₂)₆ | COOH | H | Pentyl |
| 689 | 1 | (CH₂)₇ | COOH | H | Hexyl |
| 690 | 1 | (CH₂)₈ | COOH | H | Heptyl |
| 691 | 1 | (CH₂)₉ | COOH | H | Octyl |
| 692 | 1 | (CH₂)₁₀ | COOH | H | Nonyl |
| 693 | 1 | C₂H₂ | COOH | H | Cyclohexyl |
| 694 | 1 | bond | 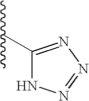 | H | cyclohexyl |
| 695 | 1 | bond | 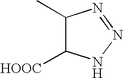 | H | cyclohexyl |
| 696 | 1 | bond | 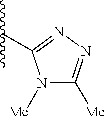 | H | cyclohexyl |
| 697 | 1 | bond | 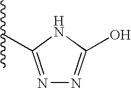 | H | cyclohexyl |
| 698 | 1 | bond | 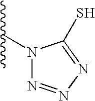 | H | cyclohexyl |
| 699 | 1 | bond | 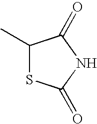 | H | cyclohexyl |
| 700 | 1 | bond | 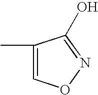 | H | cyclohexyl |
| 701 | 1 | bond | 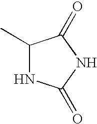 | H | cyclohexyl |
| 702 | 1 | bond | 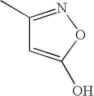 | H | cyclohexyl |
| 703 | 1 | bond | 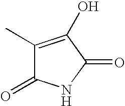 | H | cyclohexyl |
| 704 | 1 | bond | 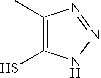 | H | cyclohexyl |
| 705 | 1 | bond | 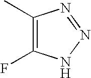 | H | cyclohexyl |
| 706 | 1 | bond | 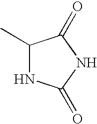 | H | cyclohexyl |
| 707 | 1 | bond | 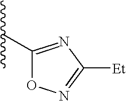 | H | cyclohexyl |
| 708 | 1 | bond | 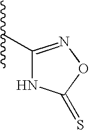 | H | cyclohexyl |
| 709 | 1 | bond | 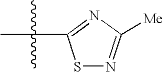 | H | cyclohexyl |
| 710 | 1 | bond | 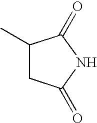 | H | cyclohexyl |
| 711 | 1 | bond | 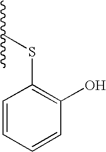 | H | cyclohexyl |
| 712 | 1 | bond | 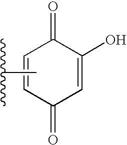 | H | cyclohexyl |

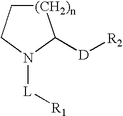

| No. | n | D | R₂ | L | R₁ |
|---|---|---|---|---|---|
| 713 | 1 | CH₂ | OH | 1,2-dioxoethyl | benzyl |
| 714 | 1 | bond | —CN | 1,2-dioxoethyl | 1,1-dimethylpropyl |
| 715 | 1 | bond | tetrazole | 1,2-dioxoethyl | 1,1-dimethylpropyl |
| 716 | 2 | bond | CONH₂ | 1,2-dioxoethyl | 1,1-dimethylpropyl |
| 717 | 1 | bond | COOH | 1,2-dioxoethyl | 1,1-dimethylpropyl |
| 718 | 2 | bond | COOH | 1,2-dioxoethyl | 1,1-dimethylpropyl |

A another preferred embodiment of the invention is the use for the treatment or prevention of sensorineural hearing loss with a compound of the formula (LXVII):

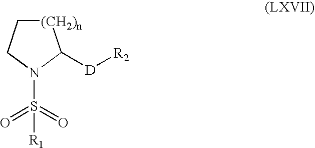

in which:

n is 1-3;

R₁ is selected from the group consisting of hydrogen, C₁-C₉ straight or branched chain alkyl, C₂-C₉ straight or branched chain alkenyl, aryl, heteroaryl, carbocycle, or heterocycle;

D is a bond, or a C₁-C₁₀ straight or branched chain alkyl, C₂-C₁₀ alkenyl or C₂-C₁₀ alkynyl;

R₂ is a carboxylic acid or a carboxylic acid isostere;

wherein said alkyl, alkenyl, alkynyl, aryl, heteroaryl, carbocycle, heterocycle, or carboxylic acid isostere is optionally substituted with one or more substituents selected from R³, where

R³ is hydrogen, hydroxy, halo, halo-(C₁-C₆)-alkoxy, thiocarbonyl, (C₁-C₆)-alkoxy, (C₂-C₆)-alkenyloxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;

or a pharmaceutically acceptable salt, ester or solvate thereof;

A preferred embodiment of this invention is the use of a compound in which R₂ is a carbocycle or heterocycle containing any combination of CH₂, O, S, or N in any chemically stable oxidation state, where any of the atoms of said ring structure are optionally substituted in one or more positions with R³.

Especially preferred embodiments of this aspect of the invention are the use of those compounds in which R₂ is selected from the group below:

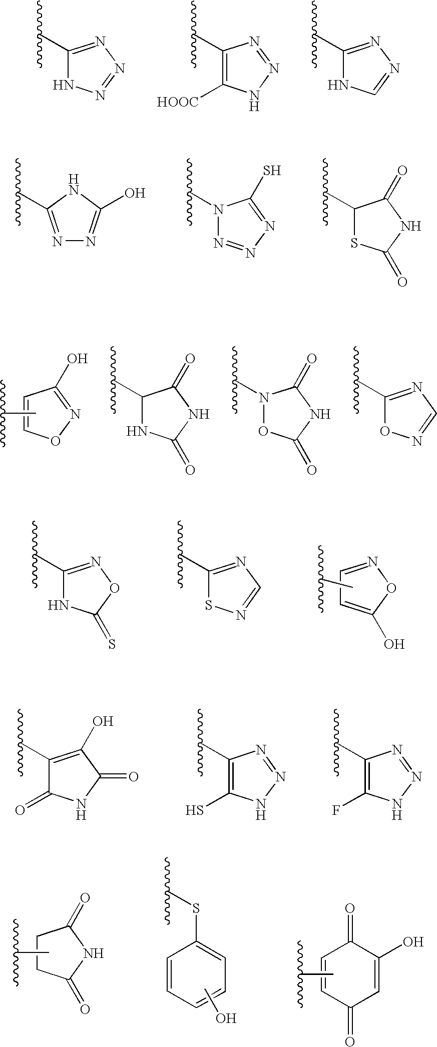

in which the atoms of said ring structure may be optionally substituted at one or more positions with R³.

Another preferred embodiment of this invention is where R₂ is selected from the group consisting of —COOH, —SO₃H, —SO₂HNR³, —PO₂(R³) 2, —CN, —PO₃(R³) 2, —OR³₁—SR³, —NHCOR³, —N(R³)₂, —CON(R³)₂, —CONH(O)R³, —CONHNHSO₂R³, —COHNSO₂R³, and —CONR³CN.

Preferred embodiments of this invention are the following compounds: (2S)-1-(phenylmethyl)sulfonyl-2-hydroxymethyl pyrrolidine; (2S)-1-(phenylmethyl)-sulfonyl-2-pyrrolidinetetrazole; (2S)-1-(phenyl-methyl)-sulfonyl-2-pyrrolidine carbonitrile; and compounds 719-821.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include

—COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³—CONHNHSO₂R³, —COHNSO₂R³, and —CONR³CN, wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl.

In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carbocyclic and heterocyclic isosteres contemplated by this aspect of the invention.

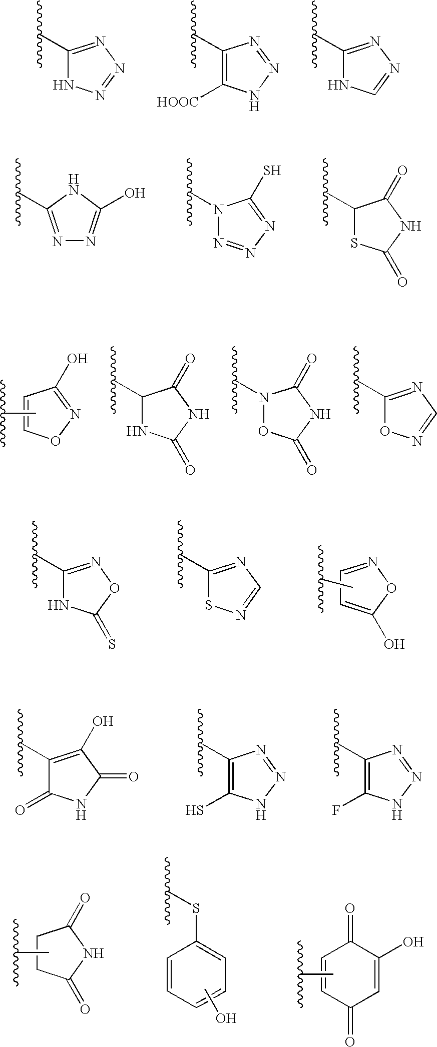

where the atoms of said ring structure may be optionally substituted at one or more positions with R³. The present invention contemplates that when chemical substituents are added to a carboxylic isostere then the inventive compound retains the properties of a carboxylic isostere. The present invention contemplates that when a carboxylic isostere is optionally substituted with one or more moieties selected from R³, then the substitution can not eliminate the carboxylic acid isosteric properties of the inventive compound. The present invention contemplates that the placement of one or more R³ substituents upon a carbocyclic or heterocyclic carboxylic acid isostere shall not be at an atom(s) which maintains or is integral to the carboxylic acid isosteric properties of the inventive compound if such a substituent(s) would destroy the carboxylic acid isosteric properties of the inventive compound.

Other carboxylic acid isosteres not specifically exemplified or described in this specification are also contemplated by the present invention.

A compound of the present invention, especially formula LXVII, wherein n is 1, D is a bond, R₁ is phenylmethyl, and R₂ is —CN, is named (2S)-1-(phenylmethyl) sulfonyl-2-pyrrolidine carbonitrile.

Specific embodiments of the inventive compounds are presented in Table XLVIII. The present invention contemplates employing the compounds of Table XLVIII, below, for use in compositions and methods of the invention.

TABLE XLVIII

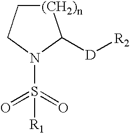

| No. | n | D | R₂ | R₁ |
|---|---|---|---|---|
| 719 | 1 | bond | COOH | Benzyl |
| 720 | 1 | bond | COOH | a-MethylBenzyl |
| 721 | 1 | bond | COOH | 4-MethylBenzyl |
| 722 | 1 | bond | Tetrazole | Benzyl |
| 723 | 1 | bond | SO₃H | a-MethylBenzyl |
| 724 | 1 | CH₂ | COOH | 4-MethylBenzyl |
| 725 | 1 | bond | SO₂HNMe | Benzyl |
| 726 | 1 | bond | CN | a-MethylBenzyl |
| 727 | 1 | bond | PO₃H₂ | 4-MethylBenzyl |
| 728 | 2 | bond | COOH | Benzyl |
| 729 | 2 | bond | COOH | a-MethylBenzyl |
| 730 | 2 | bond | COOH | 4-MethylBenzyl |
| 731 | 2 | bond | COOH | 3,4,5-trimethoxy- |
| | | | | phenyl |
| 732 | 2 | bond | COOH | Cyclohexyl |
| 733 | 2 | bond | PO₂HEt | i-propyl |
| 734 | 2 | bond | PO₃HPropyl | ethyl |
| 735 | 2 | bond | PO₃(Et)₂ | Methyl |
| 736 | 2 | bond | OMe | tert-butyl |
| 737 | 2 | bond | OEt | n-pentyl |
| 738 | 2 | bond | OPropyl | n-hexyl |
| 739 | 1 | bond | OButyl | Cyclohexyl |
| 740 | 1 | bond | OPentyl | cyclopentyl |
| 741 | 1 | bond | OHexyl | n-heptyl |
| 742 | 1 | bond | SMe | n-octyl |
| 743 | 1 | bond | SEt | n-nonyl |
| 744 | 2 | bond | SPropyl | 2-indolyl |
| 745 | 2 | bond | SButyl | 2-furyl |
| 746 | 2 | bond | NHCOMe | 2-thiazolyl |
| 747 | 2 | bond | NHCOEt | 2-thienyl |
| 748 | 1 | CH₂ | N(Me)₂ | 2-pyridyl |
| 749 | 1 | (CH₂)₂ | N(Me)Et | benzyl |
| 750 | 1 | (CH₂)₃ | CON(Me)₂ | benzyl |
| 751 | 1 | (CH₂)₄ | CONHMe | benzyl |
| 752 | 1 | (CH₂)₅ | CONHEt | benzyl |
| 753 | 1 | (CH₂)₆ | CONHPropyl | 1,1-dimethyl- |
| | | | | propyl |
| 754 | 1 | bond | CONH(O)Me | Benzyl |
| 755 | 1 | bond | CONH(O)Et | a-Methylphenyl |
| 756 | 1 | bond | CONH(O)Propyl | 4-Methylphenyl |
| 757 | 2 | bond | COOH | Benzyl |
| 758 | 2 | bond | COOH | a-Methylphenyl |
| 759 | 2 | bond | COOH | 4-Methylphenyl |
| 760 | 1 | CH₂ | COOH | benzyl |
| 761 | 1 | (CH₂)₂ | COOH | benzyl |
| 762 | 1 | (CH₂)₃ | COOH | benzyl |
| 763 | 1 | (CH₂)₄ | COOH | benzyl |
| 764 | 1 | (CH₂)₅ | COOH | benzyl |
| 765 | 1 | (CH₂)₆ | COOH | benzyl |
| 766 | 1 | (CH₂)₇ | COOH | benzyl |
| 767 | 1 | (CH₂)₈ | COOH | benzyl |
| 768 | 1 | (CH₂)₉ | COOH | benzyl |
| 769 | 1 | (CH₂)₁₀ | COOH | benzyl |
| 770 | 1 | C₂H₂ | COOH | benzyl |
| 771 | 1 | 2-hydroxyethyl | COOH | benzyl |
| 772 | 1 | 2-butylene | COOH | benzyl |
| 773 | 1 | i-Propyl | COOH | benzyl |
| 774 | 1 | tert-Butyl | COOH | benzyl |
| 775 | 1 | 2-nitrohexyl | COOH | benzyl |
| 776 | 3 | (CH₂)₂ | CN | benzyl |
| 777 | 1 | (CH₂)₃ | CN | benzyl |
| 778 | 3 | bond | CONHNHSO₂Me | Benzyl |
| 779 | 3 | bond | CONHNHSO₂Et | a-Methylphenyl |
| 780 | 3 | bond | CONHSO₂Me | 4-Methylphenyl |
| 781 | 2 | bond | CONHNHSO₂Et | Phenyl |
| 782 | 2 | bond | COH(Me)CN | a-Methylphenyl |
| 783 | 2 | bond | CON(Et)CN | 4-Methylphenyl |
| 784 | 1 | (CH₂)₂ | COOH | methyl |
| 785 | 1 | (CH₂)₃ | COOH | ethyl |
| 786 | 1 | (CH₂)₄ | COOH | n-propyl |
| 787 | 1 | (CH₂)₅ | COOH | t-butyl |
| 788 | 1 | (CH₂)₆ | COOH | Pentyl |
| 789 | 1 | (CH₂)₇ | COOH | Hexyl |
| 790 | 1 | (CH₂)₆ | COOH | Heptyl |
| 791 | 1 | (CH₂)₉ | COOH | Octyl |
| 792 | 1 | (CH₂)₁₀ | COOH | Nonyl |
| 793 | 1 | C₂H₂ | COOH | Cyclohexyl |
| 794 | 1 | bond | 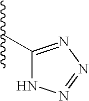 | benzyl |
| 795 | 1 | bond | 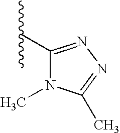 | benzyl |
| 796 | 1 | bond | 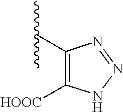 | benzyl |
| 797 | 1 | bond | 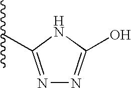 | benzyl |
| 798 | 1 | bond | 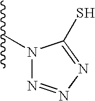 | benzyl |
| 799 | 1 | bond | 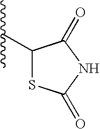 | benzyl |
| 800 | 1 | bond | 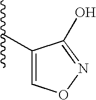 | benzyl |
| 801 | 1 | bond | 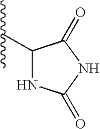 | benzyl |
| 802 | 1 | bond | 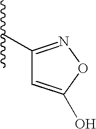 | benzyl |
| 803 | 1 | bond | 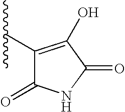 | benzyl |
| 804 | 1 | bond | 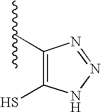 | benzyl |
| 805 | 1 | bond | 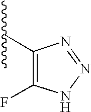 | benzyl |
| 806 | 1 | bond | 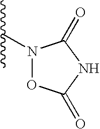 | benzyl |
| 807 | 1 | bond | 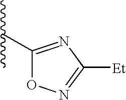 | benzyl |
| 808 | 1 | bond | 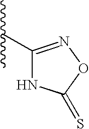 | benzyl |
| 809 | 1 | bond | 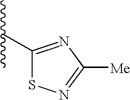 | benzyl |
| 810 | 1 | bond | 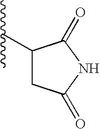 | benzyl |
| 811 | 1 | bond | 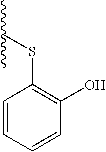 | benzyl |
| 812 | 1 | bond | 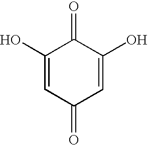 | benzyl |
| 813 | 1 | bond | CH₂OH | benzyl |
| 814 | 1 | bond | CONH₂ | benzyl |
| 815 | 1 | bond | CN | benzyl |

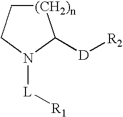

| No. | n | D | R₂ | L | R₁ |
|---|---|---|---|---|---|
| 816 | 1 | CH₂ | OH | 1,2-dioxoethyl | benzyl |
| 817 | 1 | bond | —CN | 1,2-dioxoethyl | 1,1-dimethylpropyl |
| 818 | 1 | bond | tetrazole | 1,2-dioxoethyl | 1,1-dimethylpropyl |
| 819 | 2 | bond | CONH₂ | 1,2-dioxoethyl | 1,1-dimethylpropyl |
| 820 | 1 | bond | COOH | 1,2-dioxoethyl | 1,1-dimethylpropyl |
| 821 | 2 | bond | COOH | 1,2-dioxoethyl | 1,1-dimethylpropyl |

### Synthesis of Sensorineurotrophic Compounds

The compounds for use in the methods and compositions of the invention may be readily prepared by standard techniques of organic chemistry, utilizing the general synthetic pathways depicted below.

In the preparation of the compounds of the invention, one skilled in the art will understand that one may need to protect or block various reactive functionalities on the starting compounds or intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to “Protective Groups in Organic Chemistry,” McOmie, ed., Plenum Press, New York, N.Y.; and “Protective Groups in Organic Synthesis,” Greene, ed., John Wiley & Sons, New York, N.Y. (1981) for the teaching of protective groups which may be useful in the preparation of compounds of the present invention.

The product and intermediates may be isolated or purified using one or more standard purification techniques, including, for example, one or more of simple solvent evaporation, recrystallization, distillation, sublimation, filtration, chromatography, including thin-layer chromatography, HPLC (e.g. reverse phase HPLC), column chromatography, flash chromatography, radial chromatography, trituration, and the like.

As described by Scheme I, cyclic amino acids 1 protected by suitable blocking groups P on the amino acid nitrogen may be reacted with thiols RSH to generate thioesters 2. After removal of the protecting group, the free amine 3 may be reacted with a variety of isocyanates or isothiocyanates to provide the final ureas or thioureas, respectively.

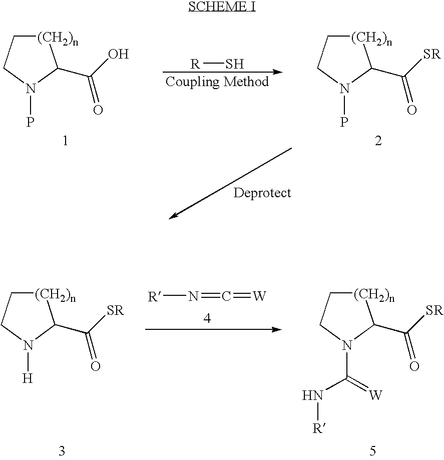

Isocyanates (R′NCO) or isothiocyanates (R′NCS) 4 may be conveniently prepared from the corresponding readily available amines by reaction with phosgene or thiophosgene, as depicted in Scheme II.

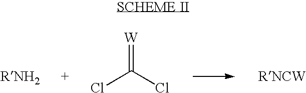

Thiols R—SH may be conveniently prepared from the corresponding readily available alcohols or halides via a two step replacement of halide by sulfur, as described in Scheme III. Halides may be reacted with thiourea, and the corresponding alkyl thiouronium salts hydrolyzed to provide thiols RSH. If alcohols are used as the starting materials, they may be first converted to the corresponding halides by standard methods.

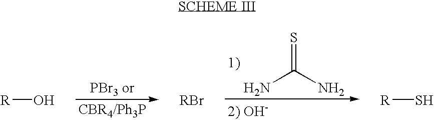

The compounds of formulas XX to XXIV may be readily prepared by standard techniques of organic chemistry, utilizing the general synthetic pathway depicted below. As described by Scheme IV, cyclic amino acids 1 protected by suitable blocking groups P on the amino acid nitrogen may be reacted with thiols RSH to generate thioesters 2. After removal of the protecting group, the free amine 3 may be reacted with various sulfonyl chlorides 4 to provide final products 5 in good to excellent yield.

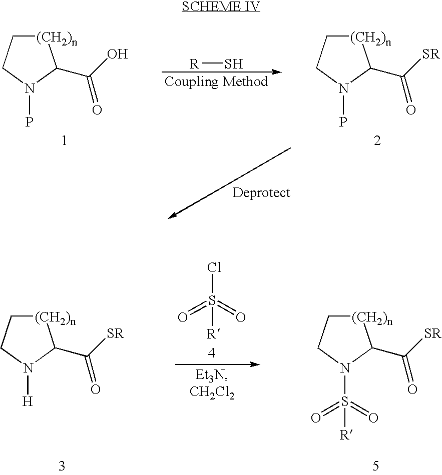

Thiols R—SH may be conveniently prepared from the corresponding readily available alcohols or halides via a two step replacement of halogen by sulfur, as described in Scheme V. Halides may be reacted with thiourea, and the corresponding alkyl thiouronium salts hydrolyzed to provide thiols RSH. If alcohols are used as the starting materials, they may be first converted to the corresponding halides by standard methods.

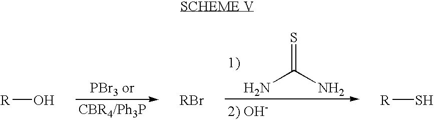

The compounds of formulas XXV to XXIX may be prepared by a variety of synthetic sequences that utilize established chemical transformations. The general pathway to the present compounds is described in Scheme VI. N-glyoxylproline derivatives may be prepared by reacting L-proline methyl ester with methyl oxalyl chloride as shown in Scheme VI. The resulting oxamates may be reacted with a variety of carbon nucleophiles to obtain intermediates compounds. These intermediates are then reacted with a variety of alcohols, amides, or protected amino acid residues to obtain the propyl esters and amides of the invention.

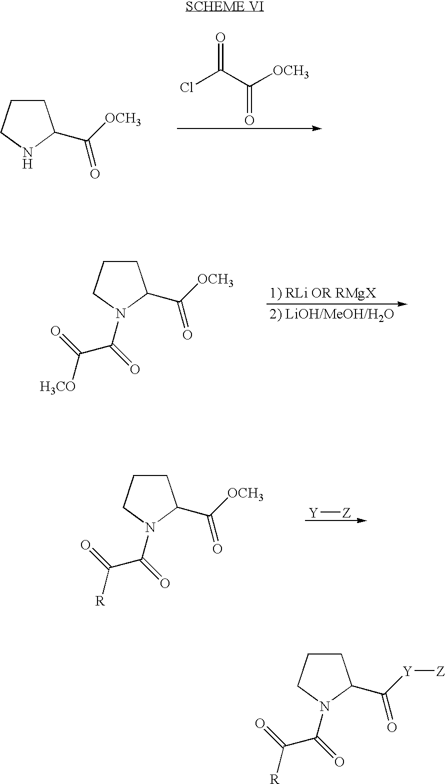

The substituted alcohols may be prepared by a number of methods known to those skilled in the art of organic synthesis. As described in Scheme VII, alkyl or aryl aldehydes may be homologated to phenyl propanols by reaction with methyl(triphenyl-phosphoranylidene)acetate to provide a variety of trans-cinnamates; these latter compounds may be reduced to the saturated alcohols by reaction with excess lithium aluminum hydride, or sequentially by reduction of the double bond by catalytic hydrogenation and reduction of the saturated ester by appropriate reducing agents. Alternatively, the trans-cinnamates may be reduced to (E)-allylic alcohols by the use of diisobutylaluminum hydride.

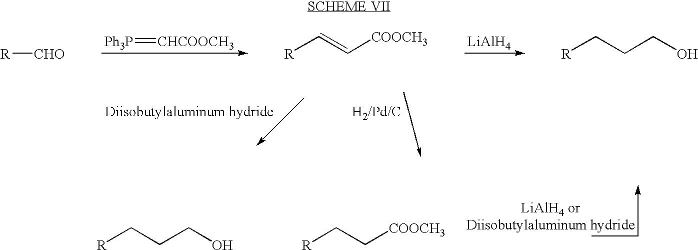

Longer chain alcohols may be prepared by homologation of benzylic and higher aldehydes. Alternatively, these aldehydes may be prepared by conversion of the corresponding phenylacetic and higher acids, and phenethyl and higher alcohols.

The general synthesis of the carboxylic acid isosteres of Formula LXV is outlined in Scheme VIII and IX:

N-glyoxylproline derivatives may be prepared by reacting L-proline methyl ester with methyl oxalyl chloride as shown in Scheme VIII. The resulting oxamates may be reacted with a variety of carbon nucleophiles to obtain compounds used in the present invention, as in Scheme IX.

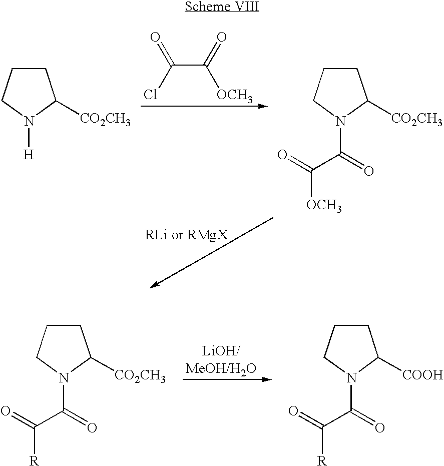

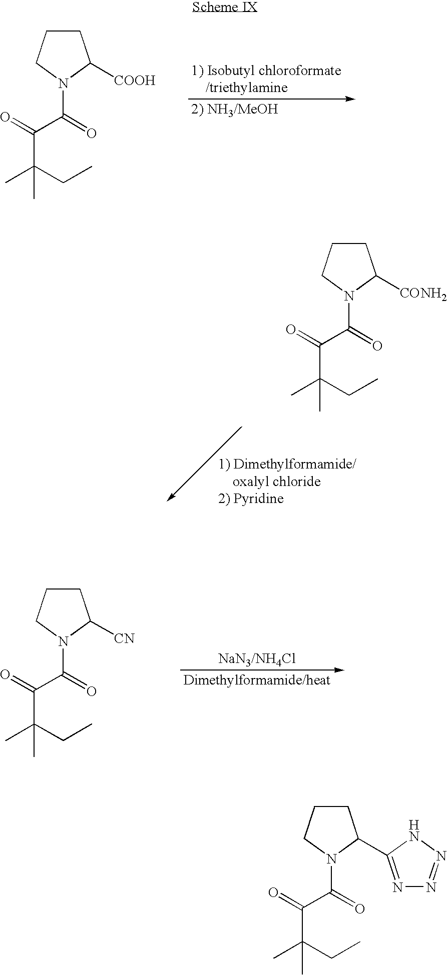

The compounds of formulae LXV may be readily prepared by standard techniques of organic chemistry, utilizing the general synthetic pathways depicted below for di-keto derivatives, sulfonamide derivatives, and urea or carbamate derivatives.

Cyclic amino acids 1 protected by suitable blocking groups P on the amino acid nitrogen may be reacted with thiols RSH to generate thioesters 2. After removal of the protecting group, the free amine 3 may be reacted with a variety of isocyanates or isothiocyanates to provide final ureas or thioureas, respectively.

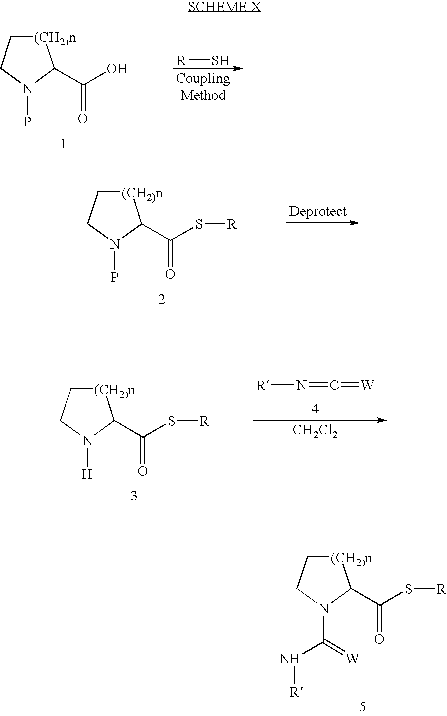

Another scheme for preparing ureas or carbamates is set forth below.

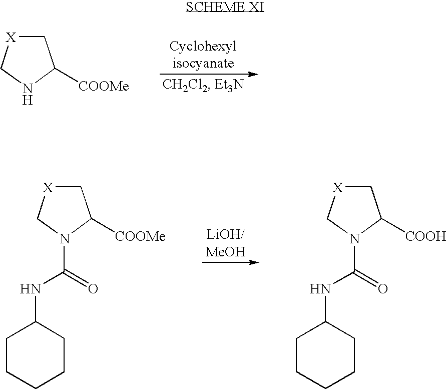

Isocyanates (R′NCO) or isothiocyanates (R′NCS) may be conveniently prepared from the corresponding readily available amines by reaction with phosgene or thiophosgene, as depicted below.

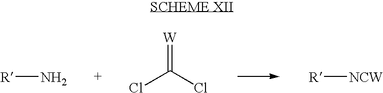

Thiols R—SH may be conveniently prepared from the corresponding readily available alcohols or halides via a two step replacement of halide by sulfur, as described below. Halides may be reacted with thiourea, and the corresponding alkyl thiouronium salts hydrolyzed to provide thiols RSH. If alcohols are used as the starting materials, they may be first converted to the corresponding halides by standard methods.

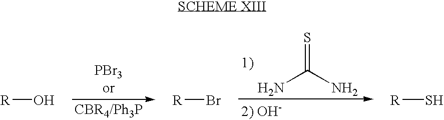

N-glyoxylproline derivatives may be prepared by reacting L-proline methyl ester with methyl oxalyl chloride as shown below. The resulting oxamates may be reacted with a variety of carbon nucleophiles to obtain compounds of the present invention or useful for preparing compounds of the present invention.

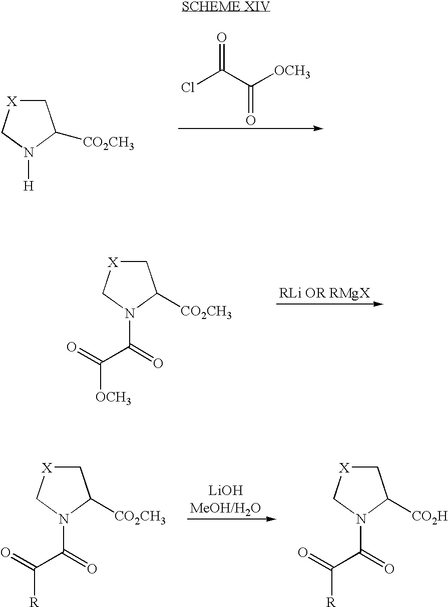

Synthetic schemes for preparing sulfonamide derivatives are known in the art and compounds of the present invention may be synthesized using schemes such as are set forth below.

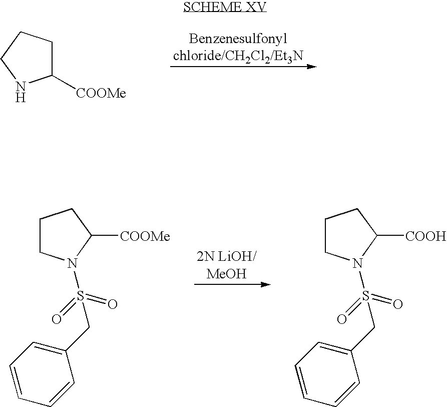

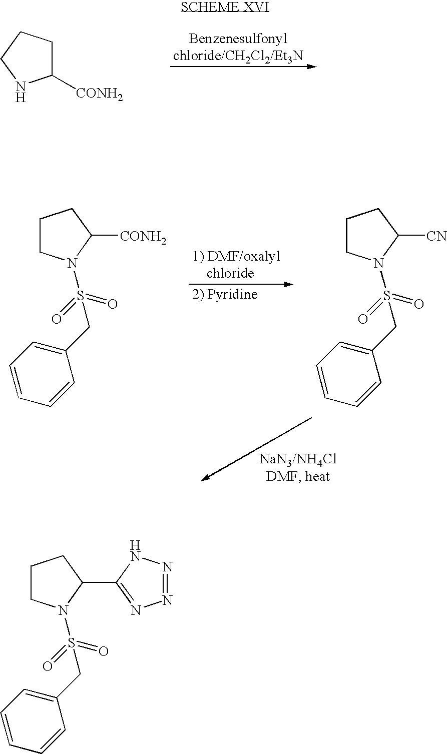

The general synthesis of the carboxylic acid isosteres of Formula LXVI may be prepared by a variety of synthetic sequences that utilize established chemical transformations. An exemplary general pathway to synthesize the present compounds is described in Scheme XVII.

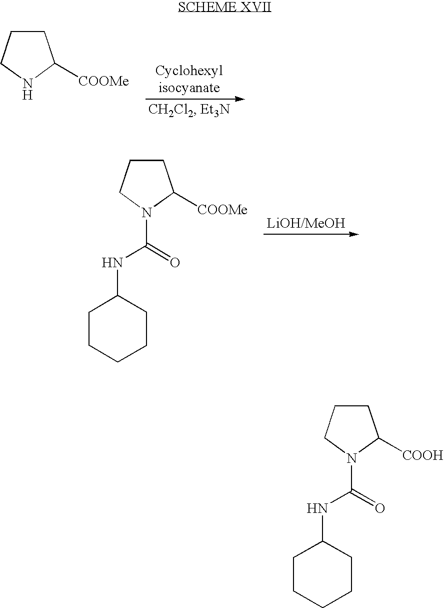

The compounds of formula LXVII may be prepared by a variety of synthetic sequences that utilize established chemical transformations. An exemplary general pathway to the present compounds is described in Schemes XVIII, XVI and XX.

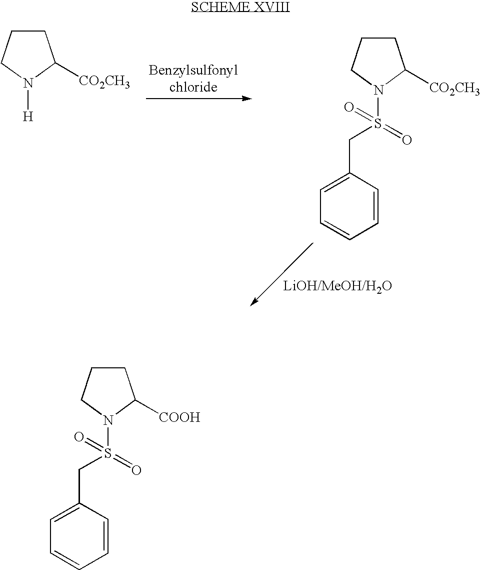

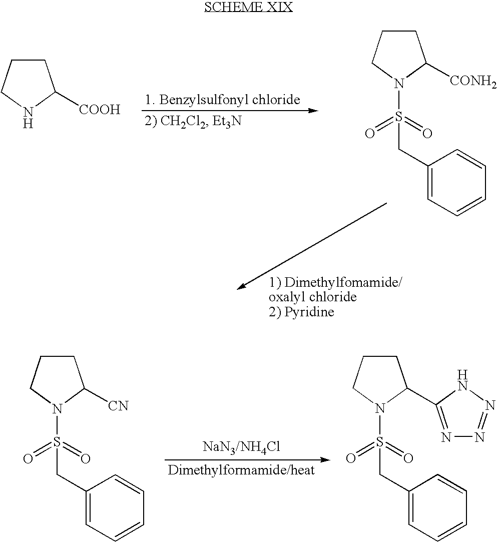

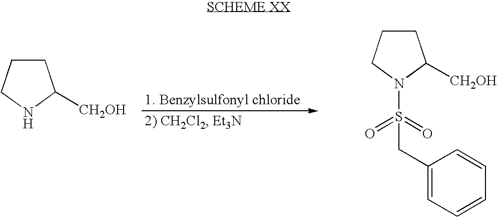

### Affinity for FKBP12

The compounds used in the inventive methods and pharmaceutical compositions may have an affinity for the FK506 binding protein, particularly FKBP12. The inhibition of the prolyl peptidyl cis-trans isomerase activity of FKBP may be measured as an indicator of this affinity.

### Kᵢ Test Procedure

The binding to FBKP12 and inhibition of the peptidyl-prolyl isomerase (rotamase) activity of the compounds used in the inventive methods and pharmaceutical compositions can be evaluated by known methods described in the literature (Harding et al., Nature, 1989, 341:758-760; Holt et al. J. Am. Chem. Soc., 115:9923-9938). These values are obtained as apparent Kᵢ's and are presented for representative compounds in TABLES IX to XVI.

The cis-trans isomerization of an alanine-proline bond in a model substrate, N-succinyl-Ala-Ala-Pro-Phe-p-nitroanilide, is monitored spectrophotometrically in a chymotrypsin-coupled assay, which releases para-nitroanilide from the trans form of the substrate. The inhibition of this reaction caused by the addition of different concentrations of inhibitor is determined, and the data is analyzed as a change in first-order rate constant as a function of inhibitor concentration to yield the apparent Kᵢ values.

In a plastic cuvette are added 950 mL of ice cold assay buffer (25 mM HEPES, pH 7.8, 100 mM NaCl), 10 mL of FKBP (2.5 mM in 10 mM Tris-Cl pH 7.5, 100 mM NaCl, 1 mM dithiothreitol), 25 mL of chymotrypsin (50 mg/ml in 1 mM HCl) and 10 mL of test compound at various concentrations in dimethyl sulfoxide. The reaction is initiated by the addition of 5 mL of substrate (succinyl-Ala-Phe-Pro-Phe-para-nitroanilide, 5 mg/mL in 2.35 mM LiCl in trifluoroethanol).

The absorbance at 390 nm versus time is monitored for 90 seconds using a spectrophotometer and the rate constants are determined from the absorbance versus time data files.

TABLE XLI
In Vitro Test Results - Formulas I to XIV

| Compound | Kᵢ (nM) |
|---|---|
| 1 | 31 |
| 2 | 210 |
| 3 | 85 |
| 9 | 104 |
| 10 | 12 |
| 11 | 299 |
| 12 | 442 |
| 14 | 313 |
| 28 | 108 |
| 29 | 59 |
| 30 | 11 |
| 31 | 8.7 |
| 32 | 362 |
| 33 | 1698 |
| 34 | 34 |
| 35 | 62 |
| 36 | 7 |
| 37 | 68 |
| 38 | 8.9 |
| 39 | 347 |
| 40 | 1226 |
| 41 | 366 |
| 42 | 28 |
| 43 | 259 |
| 44 | 188 |
| 45 | 31 |
| 46 | 757 |
| 47 | 21 |
| 48 | 127 |
| 49 | 1334 |
| 50 | 55 |
| 51 | 33 |
| 52 | 6 |
| 53 | 261 |
| 54 | 37 |
| 55 | 30 |
| 56 | 880 |
| 57 | 57 |
| 58 | 79 |
| 59 | 962 |
| 60 | 90 |
| 61 | 139 |
| 62 | 196 |
| 63 | 82 |
| 64 | 163 |
| 65 | 68 |
| 66 | 306 |
| 67 | 177 |
| 68 | 284 |
| 69 | 49 |
| 70 | 457 |
| 71 | 788 |
| 80 | 215 |
| 81 | 638 |
| Parent | 7.5 |
| (unoxidized) | |
| compound of | |
| Example 6 | |
| 95 (Example 6) | 225 |

TABLE XLII
In Vitro Test Results - Formulas XV to XXIV

| Compound | Kᵢ (nM) |
|---|---|
| 101 | +++ |
| 102 | ++ |
| 103 | ++ |
| 104 | ++ |
| 105 | ++ |
| 106 | + |
| 107 | ++ |
| 108 | +++ |
| 109 | +++ |
| 110 | +++ |
| 111 | ++ |
| 112 | +++ |
| 113 | +++ |
| 114 | +++ |
| 115 | +++ |
| 116 | ++ |
| 117 | +++ |
| 118 | ++ |
| 119 | ++ |
| 120 | ++ |
| 121 | ++ |
| 122 | + |
| 123 | ++ |
| 124 | +++ |
| 125 | +++ |
| 126 | +++ |
| 127 | ++ |
| 128 | +++ |
| 129 | +++ |
| 130 | +++ |
| 131 | +++ |
| 132 | ++ |

Relative potencies of compounds are ranked according to the following scale: ++++ denotes Kᵢ or ED50<1 nM; +++ denotes Kᵢ or ED50 of 1-50 nM; ++ denotes Kᵢ or ED 50 of 51-200 nM; + denotes Kᵢ or ED of 201-500 nM.

TABLE XLIII

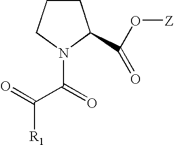

In Vitro Test Results - Formulas XXV to XXIX

| No. | Z | R′ | Kᵢ |
|---|---|---|---|
| 137 | 1,1-dimethylpropyl | 3-phenylpropyl | 42 |
| 138 | 1,1-dimethylpropyl | 3-phenyl-prop-2-(E)-enyl | 125 |
| 139 | 1,1-dimethylpropyl | 3-(3,4,5- | 200 |
| | | trimethoxyphenyl)propyl | |
| 140 | 1,1-dimethylpropyl | 3-(3,4,5-trimethoxyphenyl)- | 65 |
| | | prop-2-(E)-enyl | |
| 141 | 1,1-dimethylpropyl | 3-(4,5-methylenedioxy)- | 170 |
| | | phenylpropyl | |
| 142 | 1,1-dimethylpropyl | 3-(4,5- | 160 |
| | | methylenedioxy)phenylprop-2- | |
| | | (E)-enyl | |
| 143 | 1,1-dimethylpropyl | 3-cyclohexylpropyl | 200 |
| 144 | 1,1-dimethylpropyl | 3-cyclohexylprop-2-(E)-enyl | 600 |
| 145 | 1,1-dimethylpropyl | (1R)-1,3-diphenyl-1-propyl | 52 |
| 146 | 2-furanyl | 3-phenylpropyl | 4000 |
| 147 | 2-thienyl | 3-phenylpropyl | 92 |
| 148 | 2-thiazolyl | 3-phenylpropyl | 100 |
| 149 | phenyl | 3-phenylpropyl | 1970 |
| 150 | 1,1-dimethylpropyl | 3-(2,5- | 250 |
| | | dimethoxy)phenylpropyl | |
| 151 | 1,1-dimethylpropyl | 3-(2,5-dimethoxy)phenylprop- | 450 |
| | | 2-(E)-enyl | |
| 152 | 1,1-dimethylpropyl | 2-(3,4,5- | 120 |
| | | trimethoxyphenyl)ethyl | |
| 153 | 1,1-dimethylpropyl | 3-(3-pyridyl)propyl | 5 |
| 154 | 1,1-dimethylpropyl | 3-(2-pyridyl)propyl | 195 |
| 155 | 1,1-dimethylpropyl | 3-(4-pyridyl)propyl | 23 |
| 156 | cyclohexyl | 3-phenylpropyl | 82 |
| 157 | tert-butyl | 3-phenylpropyl | 95 |
| 158 | cyclohexylethyl | 3-phenylpropyl | 1025 |
| 159 | cyclohexylethyl | 3-(3-pyridyl)propyl | 1400 |
| 160 | tert-butyl | 3-(3-pyridyl)propyl | 3 |
| 161 | 1,1-dimethylpropyl | 3,3-diphenylpropyl | 5 |
| 162 | cyclohexyl | 3-(3-pyridyl)propyl | 9 |
| 163 | 2-thienyl | 3-(3-pyridyl)propyl | 1000 |
| 164 | tert-butyl | 3,3-diphenylpropyl | 5 |
| 165 | cyclohexyl | 3,3-diphenylpropyl | 20 |
| 166 | 2-thienyl | 3,3-diphenylpropyl | 150 |

TABLE XLIV
In Vitro Test Results

| Compound | Kᵢ (μM) |
|---|---|
| 172 | 140 |
| 175 | 13 |
| 177 | 170 |
| 178 | 250 |
| 179 | 25 |
| 181 | 17 |
| 185 | 12 |
| 202 | >10,000 |
| 207 | 1300 |
| 216 | >10,000 |
| 255 | 1800 |
| 256 | 28 |
| 257 | 39 |
| 258 | 75 |
| 259 | 70 |
| 260 | 165 |
| 261 | 740 |
| 262 | 725 |
| 263 | 130 |
| 264 | 30 |
| 265 | 60 |
| 266 | 15 |
| 267 | 12 |
| 268 | 120 |
| 269 | 20 |
| 270 | 103 |
| 271 | 760 |
| 272 | 210 |
| 273 | 32 |
| 274 | 2 |
| 275 | 24 |
| 276 | 5 |

### EXAMPLES

The following examples are illustrative of the present invention and are not intended to be limitations thereon. Unless otherwise indicated, all percentages are based upon 100% by weight of the final composition.

### Example 1

### Synthesis of (2S)-2-({1-oxo-5-phenyl}-pentyl-1-(3,3-dimethyl-1,2-dioxopentyl)pyrrolidine (1)

(2S)-2-(1-oxo-4-phenyl)butyl-N-benzylpyrrolidine

1-chloro-4-phenylbutane (1.78 g; 10.5 mmol) in 20 mL of THF was added to 0.24 g (10 mmol) of magnesium turnings in 50 mL of refluxing THF. After the addition was complete, the mixture was refluxed for an additional 5 hours, and then added slowly to a refluxing solution of N-benzyl-L-proline ethyl ester (2.30 g (10 mmol) in 100 mL of THF. After 2 hours of further reflux, the mixture was cooled and treated with 5 mL of 2 N HCl. The reaction mixture was diluted with ether (100 mL) and washed with saturated NaHCO₃, water and brine. The organic phase was dried, concentrated and chromatographed, eluting with 5:1 CH₂Cl₂:EtOAc to obtain 2.05 g (64%) of the ketone as an oil. ¹H NMR (CDCl₃; 300 MHz): δ 1.49-2.18 (m, 8H); 2.32-2.46 (m, 1H); 2.56-2.65 (m, 2H); 2.97-3.06 (m, 1H); 3.17-3.34 (m, 1H); 3.44-3.62 (m, 1H); 4.02-4.23 (m, 2H); 7.01-7.44 (m, 10H).

(2S)-2-(1-oxo-4-phenyl)butylpyrrolidine

The ketone compound (500 mg) and palladium hydroxide (20% on carbon, 50 mg) was hydrogenated at 40 psi in a Paar shaker overnight. The catalyst was removed by filtration and the solvent was removed in vacuo. The free amine was obtained as a yellow oil (230 mg; 100%). ¹H NMR (CDCl₃; 300 MHz): δ 1.75-2.34 (m, 10H); 2.55 (m, 2H); 2.95 (dm, 1H); 3.45-3.95 (m, 1H); 4.05 (m, 1H); 7.37 (m, 5H).

(2S)-2-(1-oxo-4-phenyl)butyl-1-(1,2-dioxo-2-methoxyethyl)pyrrolidine

To a solution of (2S)-2-(1-oxo-4-phenyl) butylpyrrolidine (230 mg; 1.0 mmol) in CH₂Cl₂ (20 mL) at 0° C. was added dropwise methyloxalyl chloride (135 mg; 1.1 mmol). After stirring at 0° C. for 3 hours, the reaction was quenched with saturated NH₄Cl and the organic phase was washed with water and brine and dried and concentrated. The crude residue was purified on a silica gel column, eluting with 20:1 CH₂Cl₂:EtOAc to obtain 300 mg of the oxamate as a clear oil (98%). ¹H NMR (CDCl₃; 300 MHz): δ 1.68 (m, 4H); 1.91-2.38 (m, 4H); 2.64 (t, 2H); 3.66-3.80 (m, 2H); 3.77, 3.85 (s, 3H total); 4.16 (m, 2H); 4.90 (m, 1H); 7.16 (m, 3H); 7.27 (m, 2H).

(2S)-2-({1-oxo-5-phenyl}-pentyl-1-(3,3-dimethyl-1,2-dioxopentyl)pyrrolidine (1)

To a solution of the oxamate above (250 mg; 0.79 mmol) in anhydrous ether (15 mL), cooled to −78° C., was added 1,1-dimethylpropyl-magnesium chloride (0.8 mL of a 1.0 M solution in ether; 0.8 mmol). After stirring the resulting mixture at −78° C. for 2 hours, the reaction was quenched by the addition of 2 mL of saturated NH₄Cl, followed by 100 mL of EtOAc. The organic phase was washed with brine, dried, concentrated, and purified on a silica gel column, eluting with 50:1 CH₂Cl₂:EtOAc. Compound 1 was obtained as a clear oil, 120 mg. ¹H NMR (CDCl₃, 300 MHz): δ0.87 (t, 3H, J=7.5); 1.22 (s, 3H) 1.25 (s, 3H); 1.67 (m, 4H); 1.70-2.33 (m, 6H); 2.61 (t, 2H, J=7.1); 3.52 (m, 2H); 4.17 (t, 2H, J=6.2); 4.52 (m, 1H); 7.16-7.49 (m, 5H). Analysis calculated for C₂₂H₃₁NO₃—H₂O: C, 70.37; H, 8.86; N, 3.73. Found: 70.48; H, 8.35; N, 3.69.

### Example 2

### Synthesis of 2-phenyl-1-ethyl 1-(3,3-dimethyl-1,2-dioxopentyl)-2-piperidinecarbothioate (10)

Methyl(2S)-1-(1,2-dioxo-2-methoxyethyl)-2-pyrrolidinecarboxylate

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 min, a solution of methyl oxalyl chloride (3.20 g; 26.12 mmol) in methylene chloride (45 mL) was added dropwise. The resulting mixture was stirred at 0° C. for 1.5 hour. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃): δ1.93 (dm, 2H); 2.17 (m, 2H); 3.62 (m, 2H); 3.71 (s, 3H); 3.79, 3.84 (s, 3H total); 4.86 (dd, 1H, J=8.4, 3.3).

Methyl(2S)-1-(1,2-dioxo-3,3-dimethylpentyl)-2-pyrrolidinecarboxylate

A solution of methyl (2S)-1-(1,2-dioxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil. ¹H NMR (CDCl₃): δ0.88 (t, 3H); 1.22, 1.26 (s, 3H each); 1.75 (dm, 2H); 1.87-2.10 (m, 3H); 2.23 (m, 1H); 3.54 (m, 2H); 3.76 (s, 3H); 4.52 (dm, 1H, J=8.4, 3.4).

(2S)-1-(1,2-dioxo-3,3-dimethylpentyl)-2-pyrrolidine-carboxylic acid

A mixture of methyl (2S)-1-(1,2-dioxo-3,3-dimethylpentyl)-2-pyrrolidinecarboxylate (2.10 g; 8.23 mmol), 1 N LiOH (15 mL), and methanol (50 mL) was stirred at 0° C. for 30 minutes and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-white solid which did not require further purification. ¹H NMR (CDCl₃): δ0.87 (t, 3H); 1.22, 1.25 (s, 3H each); 1.77 (dm, 2H); 2.02 (m, 2H); 2.17 (m, 1H); 2.25 (m, 1H); 3.53 (dd, 2H, J=10.4, 7.3); 4.55 (dd, 1H, J=8.6, 4.1).

2-phenyl-1-ethyl 1-(3,3-dimethyl-1,2-dioxopentyl)-2-piperidinecarbothioate (10)

To a solution of (2S)-1-(1,2-dioxo-3,3-dimethylpentyl)-2-pyrrolidinecarboxylic acid (241 mg; 1.0 mmol) in CH₂Cl₂ (10 mL) was added dicyclohexylcarbodiimide (226 mg; 1.1 mmol). After stirring the resulting mixture for 5 minutes, the solution was cooled to 0° C. and treated with a solution of phenyl mercaptan (138 mg; 1.0 mmol) and 4-dimethylaminopyridine (6 mg) in 5 ml of CH₂Cl₂. The mixture was allowed to warm to room temperature with stirring overnight. The solids were removed by filtration and the filtrate was concentrated in vacuo; the crude residue was purified by flash chromatography (10:1 hexane:EtOAc) to obtain 302 mg (84%) of compound 10 as an oil. ¹H NMR (CDCl₃, 300 MHz): 80.85 (t, 3H, J=7.5); 1.29 (s, 3H); 1.31 (s, 3H); 1.70-2.32 (m, 6H); 2.92 (t, 2H, J=7.4); 3.22 (t, 2H, J=7.4); 3.58 (m, 2H); 4.72 (m, 1H); 7.23-7.34 (m, 5H). Analysis calculated for C₂₀H₂₇NO₃S—0.4H₂O: C, 65.15; H, 7.60; N, 3.80. Found: C, 65.41; H, 7.49; N, 3.72.

### Example 3

### Synthesis of 2-phenyl-1-ethyl (2S)-1-(3,3-dimethyl-1,2-dioxopentyl)-2-pyrrolidinecarbothioate (9)

Methyl 1-(1,2-dioxo-2-methoxyethyl)-2-piperidine-carboxylate

A solution of methyl pipecolate hydrochloride (8.50 g; 47.31 mmol) in dry methylene chloride (100 mL) was cooled to 0° C. and treated with triethylamine (10.5 g; 103 mmol; 2.1 eq). After stirring the formed slurry under a nitrogen atmosphere for 15 minutes, a solution of methyl oxalyl chloride (8.50 g; 69.4 mmol) in methylene chloride (75 mL) was added dropwise. The resulting mixture was stirred at 0° C. for 1.5 hours. After filtering to remove solids, the organic phase was washed with water, dried over MgSO₄ and concentrated. The crude residue was purified on a silica gel column, eluting with 50% ethyl acetate in hexane, to obtain 9.34 g (86%) of the product as a reddish oil. Mixture of cis-trans amide rotamers; data for trans rotamer given. ¹H NMR (CDCl₃): δ1.22-1.45 (m, 2H); 1.67-1.78 (m, 3H); 2.29 (m, 1H); 3.33 (m, 1H); 3.55 (m, 1H); 3.76 (s, 3H); 3.85, 3.87 (s, 3H total); 4.52 (dd, 1H).

Methyl 1-(1,2-dioxo-3,3-dimethylpentyl)-2-piperidine-carboxylate

A solution of methyl 1-(1,2-dioxo-2-methoxyethyl)-2-piperidinecarboxylate (3.80 g; 16.57 mmol) in 75 mL of tetrahydrofuran (THF) was cooled to −78° C. and treated with 20.7 mL of a 1.0 M solution of 1,1-dimethyl-propylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 3.32 g (74%) of the oxamate as a colorless oil. ¹H NMR (CDCl₃): δ0.88 (t, 3H); 1.21, 1.25 (s, 3H each); 1.35-1.80 (m, 7H); 2.35 (m, 1H); 3.24 (m, 1H); 3.41 (m, 1H); 3.76 (s, 3H); 5.32 (d, 1H).

1-(1,2-dioxo-3,3-dimethylpentyl)-2-piperidine-carboxylic acid

A mixture of methyl 1-(1,2-dioxo-3,3-dimethylpentyl)-2-piperidinecarboxylate (3.30 g; 12.25 mmol), 1 N LiOH (15 mL), and methanol (60 mL) was stirred at 0° C. for 30 minutes and at room temperature overnight. The mixture was acidified to pH 1 with 1 N HCl, diluted with water, and extracted into 100 mL of methylene chloride. The organic extract was washed with brine and concentrated to deliver 2.80 g (87%) of snow-white solid which did not require further purification. ¹H NMR (CDCl₃): δ0.89 (t, 3H); 1.21, 1.24 (s, 3H each); 1.42-1.85 (m, 7H); 2.35 (m, 1H); 3.22 (d, 1H); 3.42 (m, 1H); 5.31 (d, 1H).

2-phenyl-1-ethyl (2S)-1-(3,3-dimethyl-1,2-dioxopentyl)-2-pyrrolidinecarbothioate (9)

To a solution of 1-(1,2-dioxo-3,3-dimethylpentyl)-2-piperidine-carboxylic acid (255 mg; 1.0 mmol) in CH₂Cl₂ (10 mL) was added dicyclohexylcarbodiimide (226 mg; 1.1 mmol). After stirring the resulting mixture for 5 minutes, the solution was cooled to 0° C. and treated with a solution of phenyl mercaptan (138 mg; 1.0 mmol) and 4-dimethylaminopyridine (6 mg) in 5 ml of CH₂Cl₂. The mixture was allowed to warm to room temperature with stirring overnight. The solids were removed by filtration and the filtrate was concentrated in vacuo; the crude residue was purified by flash chromatography (10:1 hexane:EtOAc) to obtain 300 mg (80%) of compound 9 as an oil. ¹H NMR (CDCl₃, 300 MHz): 80.94 (t, 3H, J=7.5); 1.27 (s, 3H); 1.30 (s, 3H); 1.34-1.88 (m, 7H); 2.45 (m, 1H); 2.90 (t, 2H, J=7.7); 3.26 (t, 2H, J=7.7); 3.27 (m, 1H); 3.38 (m, 1H); 5.34 (m, 1H); 7.24-7.36 (m, 5H). Analysis calculated for C₂₁H₂₉NO₃S: C, 67.17; H, 7.78; N, 3.73. Found: C, 67.02; H, 7.83; N, 3.78.

### Example 4

### Synthesis of 3-phenyl-1-propyl(2S)-1-(3,3-dimethyl-1,2-dioxopentyl)-2-(4-thiazolidine)carboxylate (80)

1-(1,2-dioxo-2-methoxyethyl)2-(4-thiazolidine)-carboxylate

A solution of L-thioproline (1.51 g; 11.34 mmol) in 40 mL of dry methylene chloride was cooled to 0° C. and treated with 3.3 mL (2.41 g; 23,81 mmol) of triethylamine. After stirring this mixture for 30 minutes, a solution of methyl oxalyl chloride (1.81 g; 14.74 mmol) was added dropwise. The resulting mixture was stirred at 0° C. for 1.5 hours, filtered through Celite to remove solids, dried and concentrated. The crude material was purified on a silica gel column, eluting with 10% MeOH in methylene chloride, to obtain 2.0 g of the oxamate as an orange-yellow solid.

3-phenyl-1-propyl(2S)-1-(1,2-dioxo-2-methoxyethyl)2-(4-thiazolidine)carboxylate

1-(1,2-dioxo-2-methoxyethyl)2-(4-thiazolidine)-carboxylate (500 mg; 2.25 mmol), 3-phenyl-1-propanol (465 mg; 3.42 mmol), dicyclohexylcarbodiimide (750 mg; 3.65 mmol), 4-dimethylaminopyridine (95 mg; 0.75 mmol) and camphorsulfonic acid (175 mg; 0.75 mmol) in 30 mL of methylene chloride were stirred together overnight. The mixture was filtered through Celite to remove solids and chromatographed (25% ethyl acetate/hexane) to obtain 690 mg of material. ¹H NMR (CDCl₃, 300 MHz): 81.92-2.01 (m, 2H); 2.61-2.69 (m, 2H); 3.34 (m, 1H); 4.11-4.25 (m, 2H); 4.73 (m, 1H); 5.34 (m, 1H); 7.12 (m, 3H); 7.23 (m, 2H).

3-phenyl-1-propyl(2S)-1-(3,3-dimethyl-1,2-dioxopentyl)-2-(4-thiazolidine)carboxylate (80)

A solution of 3-phenyl-1-propyl(2S)-1-(1,2-dioxo-2-methoxyethyl)2-(4-thiazolidine)carboxylate (670 mg; 1.98 mmol) in tetrahydrofuran (10 mL) was cooled to −78° C. and treated with 2.3 mL of a 1.0 M solution of 1,1-dimethylpropylmagnesium chloride in ether. After stirring the mixture for 3 hours, it was poured into saturated ammonium chloride, extracted into ethyl acetate, and the organic phase was washed with water, dried and concentrated. The crude material was purified on a silica gel column, eluting with 25% ethyl acetate in hexane, to obtain 380 mg of the compound of Example 4 as a yellow oil. ¹H NMR (CDCl₃, 300 MHz): δ0.86 (t, 3H); 1.21 (s, 3H); 1.26 (s, 3H); 1.62-1.91 (m, 3H); 2.01 (m, 2H); 2.71 (m, 2H); 3.26-3.33 (m, 2H); 4.19 (m, 2H); 4.58 (m, 1H); 7.19 (m, 3H); 7.30 (m, 2H). Analysis calculated for C₂₀H₂₇NO₄S: C, 63.63; H, 7.23; N, 3.71. Found: C, 64.29; H, 7.39; N, 3.46.

### Example 5

### Synthesis of 3-(3-pyridyl)-1-propyl(2S)-1-(3,3-dimethyl-1,2-dioxopentyl)-2-(4-thiazolidine) carboxylate (81)

The compound of Example 5 was prepared according to the procedure of Example 4, using 3-(3-pyridyl)-1-propanol in the final step, to yield 3-(3-pyridyl)-1-propyl(2S)-1-(3,3-dimethyl-1,2-dioxopentyl)-2-(4-thiazolidine)carboxylate. ¹H NMR (CDCl₃, 300 MHz): δ0.89 (t, 3H, J=7.3); 1.25 (s, 3H); 1.28 (s, 3H); 1.77 (q, 2H, J=7.3); 2.03 (tt, 2H, J=6.4, 7.5); 2.72 (t, 2H, J=7.5); 3.20 (dd, 1H, J=4.0, 11.8); 3.23 (dd, 1H, J=7.0, 11.8); 4.23 (t, 2H, J=6.4); 4.55 (d, 2H, J=8.9); 5.08 (dd, 1H, J=4.0, 7.0); 7.24 (m, 1H); 8.48 (m, 2H). Analysis calculated for C₁₉H₂₆N₂O₄S—0.5H₂O: C, 58.89; H, 7.02; N, 7.23. Found: C, 58.83; H, 7.05; N, 7.19.

### Example 6

### Synthesis of 3-(3-pyridyl)-1-propyl (2S)-1-(3,3-Dimethyl-1,2-dioxopentyl)-2-pyrrolidinecarboxylate, N-oxide (95)

Methyl (2S)-1-(1,2-dioxo-2-methoxyethyl)-2-pyrrolidinecarboxylate

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 minutes, a solution of methyl oxalyl chloride (3.20 g; 26.12 mmol) in methylene chloride (45 mL) was added dropwise. The resulting mixture was stirred at 0° C. for 1.5 hour. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃): δ1.93 (dm, 2H); 2.17 (m, 2H); 3.62 (m, 2H); 3.71 (s, 3H); 3.79, 3.84 (s, 3H total); 4.86 (dd, 1H, J=8.4, 3.3).

Methyl(2S)-1-(1,2-dioxo-3,3-dimethylpentyl)-2-pyrrolidinecarboxylate

A solution of methyl (2S)-1-(1,2-dioxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil. ¹H NMR (CDCl₃): δ0.88 (t, 3H); 1.22, 1.26 (s, 3H each); 1.75 (dm, 2H); 1.87-2.10 (m, 3H); 2.23 (m, 1H); 3.54 (m, 2H); 3.76 (s, 3H); 4.52 (dm, 1H, J=8.4, 3.4).

(2S)-1-(1,2-dioxo-3,3-dimethylpentyl)-2-pyrrolidinecarboxylic acid

A mixture of methyl (2S)-1-(1,2-dioxo-3,3-dimethylpentyl-2-pyrrolidine-carboxylate (2.10 g; 8.23 mmol), 1 N LiOH (15 mL), and methanol (50 mL) was stirred at 0° C. for 30 minutes and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-white solid which did not require further purification. ¹H NMR (CDCl₃): δ0.87 (t, 3H); 1.22, 1.25 (s, 3H each); 1.77 (dm, 2H); 2.02 (m, 2H); 2.17 (m, 1H); 2.25 (m, 1H); 3.53 (dd, 2H, J=10.4, 7.3); 4.55 (dd, 1H, J=8.6, 4.1).

3-(3-Pyridyl)-1-propyl(2S)-1-(3,3-dimethyl-1,2-dioxopentyl)-2-pyrrolidinecarboxylate

A mixture of (2S)-1-(1,2-dioxo-3,3-dimethylpentyl)-2-pyrrolidinecarboxylic acid (4.58 g; 19 mmol), 3-pyridinepropanol (3.91 g; 28.5 mmol), dicyclohexylcarbodiimide (6.27 g; 30.4 mmol), camphorsulfonic acid (1.47 g; 6.33 mmol) and 4-dimethyl aminopyridine (773 mg; 6.33 mmol) in methylene chloride (100 mL) was stirred overnight under a nitrogen atmosphere. The reaction mixture was filtered through Celite to remove solids and concentrated in vacuo. The crude material was triturated with several portions of ether, and the ether portions were filtered through Celite to remove solids and concentrated in vacuo. The concentrated filtrate was purified on a flash column (gradient elution, 25% ethyl acetate in hexane to pure ethyl acetate) to obtain 5.47 g (80%) of GPI 1046 as a colorless oil (partial hydrate). ¹H NMR (CDCl₃, 300 MHz): δ0.85 (t, 3H); 1.23, 1.26 (s, 3H each); 1.63-1.89 (m, 2H); 1.90-2.30 (m, 4H); 2.30-2.50 (m, 1H); 2.72 (t, 2H); 3.53 (m, 2H); 4.19 (m, 2H); 4.53 (m, 1H); 7.22 (m, 1H); 7.53 (dd, 1H); 8.45. Analysis calculated for C₂₀H₂₈NO₄.0.25H₂O: C, 65.82; H, 7.87; N, 7.68. Found: C, 66.01; H, 7.85; N, 7.64.

3-(3-Pyridyl)-1-propyl (2S)-1-(3,3-dimethyl-1,2-dioxopentyl)-2-pyrrolidinecarboxylate, N-oxide (95)

A solution of 3-(3-pyridyl)-1-propyl (2S)-1-(3,3-dimethyl-1,2-dioxopentyl)-2-pyrrolidinecarboxylate (190 mg; 0.52 mmol) and m-chloroperbenzoic acid (160 mg of 57%-86% material, 0.53 mmol) was stirred in methylene chloride (20 mL) at room temperature for 3 hours. The reaction mixture was diluted with methylene chloride and washed twice with 1 N NaOH. The organic extract was dried and concentrated, and the crude material was chromatographed, eluting with 10% methanol in ethyl acetate, to obtain 130 mg of the Compound 95 of Example 6. ¹H NMR (CDCl₃, 300 MHz): δ0.83 (t, 3H); 1.21 (s, 3H) 1.25 (s, 3H); 1.75-2.23 (m, 8H); 2.69 (t, 2H, J=7.5); 3.52 (t, 2H, J=6.3); 4.17 (dd, 2H, J=6.3); 4.51 (m, 1H); 7.16-7.22 (m, 2H); 8.06-8.11 (m, 2H). Analysis calculated for C₂₀H₂₈N₂O₅.0.75H₂O: C, 61.60; H, 7.63; N, 7.18. Found: C, 61.79; H, 7.58; N, 7.23.

### Example 7

### Synthesis of 3-(3-Pyridyl)-1-propylmercaptyl 2S-1-[(2-methylbutyl)carbamoyl]pyrrolidine-2-carboxylate (101)

3-(3-Pyridyl)-1-propylchloride

To a solution of 3-(3-pyridyl)-1-propanol (10 g; 72.4 mmol) in chloroform (100 mL) was added dropwise a solution of thionyl chloride (12.9 g; 108.6 mmol) in chloroform (50 mL). The resulting mixture was refluxed for 1 hour, then poured into ice-cold 50% aqueous potassium hydroxide (150 mL). The layers were separated, and the organic phase was dried, concentrated, and purified on a silica gel column, eluting with 40% ethylacetate in hexane, to obtain 10 g (65%) of the chloride as a clear oil. ¹H NMR (300 MHz, CDCl₃): δ2.02-2.11 (m, 2H); 2.77 (m, 2H); 3.51 (m, 2H); 7.20 (m, 1H); 7.49 (m, 1H); 8.45 (m, 2H).

3-(3-Pyridyl)-1-propylmercaptan

A mixture of 3-(3-pyridyl)-1-propylchloride (3 g; 19.4 mmol) and thiourea (1.48 g; 19.4 mmol) in ethanol (10 mL) was refluxed for 24 hours. Aqueous sodium hydroxide, 15 mL of a 0.75 N solution, was added, and the mixture was refluxed for an additional 2 hours. After cooling to room temperature, the solvent was removed in vacuo. Chromatographic purification of the crude thiol on a silica gel column eluting with 50% ethyl acetate in hexane delivered 1.2 g of 3-(3-Pyridyl)-1-propylmercaptan as a clear liquid. ¹H NMR (300 MHz, CDCl₃): δ1.34 (m, 1H); 1.90 (m, 2H); 2.52 (m, 2H); 2.71 (m, 2H); 7.81 (m, 1H); 7.47 (m, 1H); 8.42 (m, 2H).

3-(3-Pyridyl)-1-propylmercaptyl N-(tert-butyloxycarbonyl)pyrrolidine-2-carboxylate

A mixture of N-(tert-butyloxycarbonyl)-(S)-proline (3.0 g; 13.9 mmol); 3-(3-Pyridyl)-1-propylmercaptan (3.20 g; 20.9 mmol), dicyclohexylcarbodiimide (4.59 g; 22.24 mmol), camphorsulfonic acid (1.08 g; 4.63 mmol), and 4-dimethylaminopyridine (0.60 g; 4.63 mmol) in dry methylene chloride (100 mL) was stirred overnight. The reaction mixture was diluted with methylene chloride (50 mL) and water (100 mL), and the layers were separated. The organic phase was washed with water (3×100 mL), dried over magnesium sulfate, and concentrated, and the crude residue was purified on a silica gel column eluting with ethyl acetate to obtain 4.60 g (95%) of the thioester as a thick oil. ¹H NMR (300 MHz, CDCl₃): δ1.45 (s, 9H); 1.70-2.05 (m, 5H); 2.32 (m, 1H); 2.71 (t, 2H); 2.85 (m, 2H); 3.50 (m, 2H); 4.18 (m, 1H); 7.24 (m, 1H); 7.51 (m, 1H); 8.48 (m, 2H).

3-(3-Pyridyl)-1-propylmercaptyl pyrrolidine-2-carboxylate

A solution of 3-(3-Pyridyl)-1-mercaptyl N-(tert-butyloxycarbonyl)pyrrolidine-2-carboxylate (4.60 g; 13.1 mmol) in methylene chloride (60 mL) and trifluoroacetic acid (6 mL) was stirred at room temperature for three hours. Saturated potassium carbonate was added until the pH was basic, and the reaction mixture was extracted with methylene chloride (3×). The combined organic extracts were dried and concentrated to yield 2.36 g (75%) of the free amine as a thick oil. ¹H NMR (300 MHz, CDCl₃): δ1.87-2.20 (m, 6H); 2.79 (m, 2H); 3.03-3.15 (m, 4H total); 3.84 (m, 1H); 7.32 (m, 1H); 7.60 (m, 1H); 8.57 (m, 2H).

3-(3-Pyridyl)-1-propylmercaptyl 2S-1-[(2-methylbutyl)carbamoyl]pyrrolidine-2-carboxylate (101)

A solution of 2-methylbutylamine (113 mg; 1.3 mmol) and triethylamine (132 mg; 1.3 mmol) in methylene chloride (5 mL) was added to a solution of triphosgene (128 mg; 0.43 mmol) in methylene chloride (5 mL). The resulting mixture was refluxed for 1 hour and then cooled to room temperature. 3-(3-Pyridyl)-1-propylmercaptyl pyrrolidine-2-carboxylate (300 mg; 1.3 mmol) in 5 mL of methylene chloride was added and the resulting mixture was stirred for 1 hour and then partitioned between water and a 1:1 mixture of ethyl acetate and hexane. The organic phase was dried, concentrated and purified by column chromatography (50% ethyl acetate/hexane) to obtain 250 mg (55%) of the compound of Example 7 (Compound 101, Table VII) as an oil. ¹H NMR (CDCl₃, 300 MHz): δ0.89-0.93 (m, 6H); 1.10-1.20 (m, 1H); 1.27 (s, 1H); 1.36-1.60 (m, 2H); 1.72 (s, 2H); 1.97-2.28 (m, 6H); 2.70-2.75 (m, 2H); 2.92-3.54 (m, 6H); 4.45-4.47 (m, 1H); 7.21-7.29 (m, 1H); 7.53-7.56 (dd, 1H); 8.46-8.48 (s, 2H).

### Example 8

### Synthesis of 3-(3-Pyridyl)-1-propyl 2S-1-[(1′,1′-Dimethylpropyl)carbamoyl]pyrrolidine-2-carboxylate (102)

Reaction of 3-(3-pyridyl)-1-propylmercaptyl pyrrolidine-2-carboxylate with the isocyanate generated from tert-amylamine and triphosgene, as described for Example 7, provided the compound of Example 8 (Compound 102, Table VII) in 62% yield. ¹H NMR (CDCl₃, 300 MHz): δ0.83 (t, 3H); 1.27 (s, 6H); 1.64-1.71 (m, 2H); 1.91-2.02 (m, 7H); 2.66-2.71 (t, 2H); 2.85 (m, 2H); 3.29-3.42 (m, 2H); 4.11 (br, 1H); 4.37-4.41 (m, 1H).

### Example 9

### Synthesis of 3-(3-pyridyl)-1-propylmercaptyl 2S-1-[(cyclohexyl)thiocarbamoyl]-pyrrolidine-2-carboxylate (107)

A mixture of cyclohexylisothiocyanate (120 mg; 0.9 mmol), 3-(3-pyridyl)-1-propylmercaptyl pyrrolidine-2-carboxylate (200 mg; 0.9 mmol) and triethylamine (90 mg; 0.9 mmol) in 20 mL of methylene chloride was stirred for 1 hour and then partitioned between water and a 1:1 mixture of ethyl acetate and hexane. The organic phase was dried, concentrated and purified by column chromatography (50% ethyl acetate/hexane) to obtain 160 mg (47%) of the compound of Example 9 (Compound 107, Table VII). ¹H NMR (CDCl₃, 300 MHz): δ1.16-1.40 (m, 6H); 1.50-1.71 (m, 4H); 1.95-2.08 (m, 7H); 2.70-2.75 (t, 2H); 3.03 (m, 2H); 3.40-3.60 (m, 2H); 4.95-4.98 (d, 1H); 5.26-5.29 (d, 1H); 7.17-7.25 (m, 1H).

### Example 10

### Synthesis of 3-(para-Methoxyphenyl)-1-propylmercaptyl(2S)-N-(benzenesulfonyl)pyrrolidine-2-carboxylate (120)

3-(p-Methoxyphenyl)-1-propylbromide

To a solution of 3-(p-methoxyphenyl)-1-propanol (16.6 g; 0.1 mol) in 250 mL of toluene, cooled to 0° C., was added dropwise 26 mL of phosphorus tribromide (0.27 mol). Following completion of the addition, the reaction was stirred at room temperature for 1 hour, then refluxed for an additional hour. The reaction was cooled and poured onto ice, the layers were separated, and the organic phase washed with saturated sodium bicarbonate (3×) and brine (3×). The crude material obtained upon drying and evaporation of the solvent was chromatographed, eluting with 10% EtOAc/hexane, to obtain 14 g (61%) of 3-(p-methoxyphenyl)-1-propylbromide.

3-(p-Methoxyphenyl)-1-propylmercaptan

A mixture of 3-(p-methoxyphenyl)-1-propylbromide (14 g; 61 mmol) and thiourea (5.1 g; 67 mmol) in ethanol (150 mL) was refluxed for 48 hours. Evaporation of the solvent provided a clear glassy compound, which was dissolved in 50 mL of water and treated with 100 mL of 40% aqueous sodium hydroxide. After stirring the resulting mixture for two hours, the product was extracted into ether (3×), and the combined organic extracts were washed with sodium bicarbonate and brine, dried, and concentrated. Chromatographic purification of the crude thiol on a silica gel column eluting with 2% either in hexane delivered 10.2 g of 3-(p-methoxyphenyl)-1-propylmercaptan as a clear liquid. ¹H NMR (300 MHz, CDCl₃): δ1.34 (t, 1H); 1.88-1.92 (m, 2H); 2.49-2.53 (m, 2H); 2.64-2.69 (m, 2H); 3.77 (s, 3H); 6.80-6.84 (m, 2H); 7.06-7.24 (m, 2H).

3-(p-Methoxyphenyl)-1-mercaptyl N-(tert-butyloxycarbonyl)pyrrolidine-2-carboxylate

A mixture of N-(tert-butyloxycarbonyl)-(S)-proline (2.0 g; 9.29 mmol), 3-(p-methoxyphenyl)-1-propylmercaptan (1.86 g; 10.22 mmol), 1-(3-dimethylaminopropyl)-3-ethylcarbodiimide hydrochloride (1.96 g; 10.22 mmol), and 4-dimethylaminopyridine (catalytic) in dry methylene chloride (50 mL) was stirred overnight. The reaction mixture was diluted with methylene chloride (50 mL) and water 100 (mL), and the layers were separated. The organic phase was washed with water (3×100 mL), dried over magnesium sulfate, and concentrated to provide 3.05 g of the product (100%) as a thick oil. ¹H NMR (300 MHz, CDCl₃): δ1.15 (s, 9H); 1.84-2.31 (m, 6H); 2.61 (m, 2H); 2.83 (m, 2H); 3.51 (m, 2H); 3.75 (s, 3H); 6.79 (d, 2H, J=8.04); 7.05 (m, 2H).

3-(p-Methoxyphenyl)-1-mercaptyl pyrrolidine-2-carboxylate

A solution of 3-(p-methoxyphenyl)-mercaptyl N-(tert-butyloxycarbonyl)pyrrolidine-2-carboxylate (3.0 g; 8.94 mmol) in methylene chloride (60 mL) and trifluoroacetic acid (6 mL) was stirred at room temperature for three hours. Saturated potassium carbonate was added until the pH was basic, and the reaction mixture was extracted with methylene chloride (3×). The combined organic extracts were dried and concentrated to yield 1.73 g (69%) of the free amine as a thick oil. ¹H NMR (300 MHz, CDCl₃): δ1.80-2.23 (m, 6H); 2.62 (m, 2H); 2.81 (m, 2H); 3.01 (m, 2H); 3.75 (s, 3H); 3.89 (m, 1H); 6.81 (m, 2H); 7.06 (m, 2H).

3-(para-Methoxyphenyl)-1-propylmercaptyl (2S)-N-(benzenesulfonyl)pyrrolidine-2-carboxylate (120)

A solution of 3-(p-methoxyphenyl)-1-mercaptyl pyrrolidine-2-carboxylate (567 mg; 2.03 mmol) and benzenesulfonyl chloride (358 mg; 2.03 mmol) in methylene chloride (5 mL) was treated with diisopropylethylamine (290 mg; 2.23 mmol) and stirred overnight at room temperature. The reaction mixture was filtered to remove solids and applied directly to a silica gel column, eluting with 25% ethyl acetate in hexane, to obtain 540 mg of Compound 120 (Table VIII) as a clear oil. ¹H NMR (300 MHz, CDCl₃): δ1.65-1.89 (m, 6H); 2.61 (t, 2H, J=7.3); 2.87 (t, 2H, J=7.6); 3.26 (m, 1H); 3.54 (m, 1H); 3.76 (s, 3H); 4.34 (dd, 1H, J=2.7, 8.6); 6.79 (d, 2H, J=8.7); 7.06 (d, 2H, J=8.6); 7.49-7.59 (m, 3H); 7.86 (dd, 2H, J=1.5, 6.8).

### Example 11

### Synthesis of 3-(para-Methoxyphenyl)-1-propylmercaptyl(2S)-N-(a-toluenesulfonyl)pyrrolidine-2-carboxylate (121)

A solution of 3-(p-Methoxyphenyl)-1-mercaptyl pyrrolidine-2-carboxylate (645 mg; 2.30 mmol) and a-toluenesulfonyl chloride (440 mg; 2.30 mmol) in methylene chloride (5 mL) was treated with diisopropylethylamine (330 mg; 2.53 mmol) and stirred overnight at room temperature. Purification as described for Example 10 provided the compound of Example 11 (Compound 121, Table VIII) as a clear oil. ¹H NMR (300 MHz, CDCl₃): δ1.65-2.25 (m, 8H); 2.65 (t, 2H); 2.89-2.96 (m, 2H); 3.55-3.73 (m, 2H); 3.80 (s, 3H); 4.32 (s, 2H); 4.70-4.81 (m, 1H); 6.83 (d, 2H); 7.09 (d, 2H); 7.14 (m, 3H); 7.26 (m, 2H).

### Example 12

### Synthesis of 3-(para-Methoxyphenyl)-1-propylmercaptyl(2S)-N-(a-toluenesulfonyl)pyrrolidine-2-carboxylate (122)

A solution of 3-(p-methoxyphenyl)-1-mercaptyl pyrrolidine-2-carboxylate (567 mg; 2.30 mmol) and p-toluenesulfonyl chloride (425 mg; 2.23 mmol) in methylene chloride (5 mL) was stirred overnight at room temperature. Purification as described for Example 10 provided the compound of Example 12 (Compound 122, Table VIII) as a clear oil. ¹H NMR (300 MHz, CDCl₃): δ1.67-1.94 (m, 6H); 2.40 (s, 3H); 2.61 (t, 2H, J=7.3); 2.84 (m, 2H, J=7.2); 3.22 (m, 1H); 3.52 (m, 1H); 3.76 (s, 3H); 4.32 (dd, 1H, J-2.9, 8.5); 6.79 (d, 2H, J=6.5); 7.07 (d, 2H, J=6.5); 7.29 (d, 2H, J=6.5); 7.74 (d, 2H, J=6.5).

### Example 13

### Synthesis of 1,5-Diphenyl-3-pentylmercaptyl N-(para-toluenesulfonyl)pipecolate (134)

3-Phenyl-1-propanal

Oxalyl chloride (2.90 g; 2.29 mmol) in methylene chloride (50 mL), cooled to −78° C., was treated with dimethylsulfoxide (3.4 mL) in 10 mL of methylene chloride. After stirring for 5 min, 3-phenyl-1-propanol (2.72 g; 20 mmol) in 20 mL of methylene chloride was added, and the resulting mixture was stirred at −78° C. for 15 min, treated with 14 mL of triethylamine, stirred an additional 15 min, and poured into 100 mL of water. The layers were separated, the organic phase was dried and concentrated, and the crude residue was purified on a silica gel column, eluting with 10% ethyl acetate in hexane, to obtain 1.27 g (47%) of the aldehyde as a clear oil. ¹H NMR (300 MHz, CDCl₃): δ2.80 (m, 2H); 2.98 (m, 2H); 7.27 (m, 5H); 9.81 (2, 1H).

1,5-Diphenyl-3-pentanol

A solution of 2-(bromoethyl)benzene (1.73 g; 9.33 mmol) in diethylether (10 mL) was added to a stirred slurry of magnesium turnings (250 mg; 10.18 mmol) in 5 mL of ether. The reaction was initiated with a heat gun, and after the addition was complete the mixture was heated on an oil bath for 30 min. 3-Phenyl-1-propanal (1.25 g; 9.33 mmol) was added in 10 mL of ether, and reflux was continued for 1 hour. The reaction was cooled and quenched with saturated ammonium chloride, extracted into 2× ethyl acetate, and the combined organic portions were dried and concentrated. Chromatographic purification on a silica gel column (10% ethyl acetate in hexane) delivered 1.42 g (63%) of the diphenyl alcohol. ¹H NMR (300 MHz, CDCl₃): δ1.84 (m, 4H); 2.61-2.76 (m, 4H) 3.65 (m, 1H); 7.19-7.29 (m, 10H).

1,5-Diphenyl-3-bromopentane

To a solution of 1,5-diphenyl-3-pentanol (1.20 g (5 mmol) and carbon tetrabromide (1.67 g; 5 mmol) in methylene chloride (20 mL) was added triphenylphosphine (1.31 g; 5 mmol) portionwise, at 0° C. After stirring at room temperature for 18 hours, the mixture was concentrated, triturated with ether, and the solids removed by filtration. The filtrate was passed through a plug of silica gel, eluting with hexane:methylene chloride, 10:1, to give 1.35 g (90%) of the bromide as an oil which was used without further purification. ¹H NMR (300 MHz, CDCl₃): δ2.11-2.18 (m, 4H); 2.73 (m, 2H); 2.86 (m, 2H); 3.95 (m, 1H); 7.16-7.30 (m, 10H).

1,5-Diphenyl-3-pentylmercaptan

Using the procedure described in Example 10 for the conversion of bromides to thiols, 1,5-diphenyl-3-bromopentane was converted to 1,5-diphenyl-3-pentylmercaptan in 35% overall yield. ¹H NMR (300 MHz, CDCl₃): δ1.79 (m, 2H); 1.98 (m, 2H); 2.71 (m, 3H); 2.80 (m, 2H); 7.16-7.28 (m, 10H).

1,5-Diphenyl-3-pentylmercaptyl N-(tert-butyloxycarbonyl)pyrrolidine-2-carboxylate

A mixture of N-(tert-butyloxycarbonyl)-(S)-pipecolic acid (2.11 g; 9.29 mmol), 1,5-diphenyl-3-pentylmercaptan (2.58 g; 10.22 mmol), 1-(3-dimethylaminopropyl)-3-ethylcarbodiimide hydrochloride (1.96 g; 10.22 mmol) and 4-dimethylaminopyridine (catalytic) in dry methylene chloride (50 mL) was stirred overnight. the reaction mixture was diluted with methylene chloride (50 mL) and water (100 mL), and the layers were separated. The organic phase was washed with water (3×100 mL), dried over magnesium sulfate, and concentrated to provide 870 mg (20%) of the product as a thick oil, which was used without further purification.

1,5-Diphenyl-3-pentylmercaptyl pyrrolidine-2-carboxylate

A solution of 1,5-diphenyl-3-pentylmercaptyl N-(tert-butyloxycarbonyl)pyrrolidine-2-carboxylate (850 mg; 1.8 mmol) in methylene chloride (10 mL) and trifluoroacetic acid (1 mL) was stirred at room temperature for three hours. Saturated potassium carbonate was added until the pH was basic, and the reaction mixture was extracted with methylene chloride. The combined organic extracts were dried and concentrated to yield 480 mg (72%) of the free amine as a thick oil, which was used without further purification.

1,5-Diphenyl-3-pentylmercaptyl N-(para-toluenesulfonyl)pipecolate (134)

1,5-Diphenyl-3-pentylmercaptyl N-(para-toluenesulfonyl)pipecolate (18) was prepared from 1,5-diphenyl-3-pentylmercaptyl pyrrolidine-2-carboxylate and para-toluenesulfonyl chloride as described for Example 12, in 65% yield. ¹H NMR (CDCl₃, 300 MHz): δ0.80 (m, 4H); 1.23-1.97 (m, 5H); 2.15 (d, 1H); 2.61-2.69 (m, 4H); 3.23 (m, 1H); 3.44 (dm, 1H); 4.27 (s, 2H); 4.53 (d, 1H, J=4.5); 5.06 (m, 1H); 7.16-7.34 (m, 15H).

### Example 14

### Synthesis of 3-phenyl-1-propyl (2S)-1-(3,3-dimethyl-1,2-dioxopentyl)-2-pyrrolidinecarboxylate (137)

Methyl (2S)-1-(1,2-dioxo-2-methoxyethyl)-2-pyrrolidinecarboxylate

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 min, a solution of methyl oxalyl chloride (3.20 g; 26.12 mmol) in methylene chloride (45 mL) was added dropwise. The resulting mixture was stirred at 0° C. for 1.5 hour. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃) δ 1.93 (dm, 2H); 2.17 (m, 2H); 3.62 (m, 2H); 3.71 (s, 3H); 3.79, 3.84 (s, 3H total); 4.86 (dd, 1H, J=8.4, 3.3).

Methyl (2S)-1-(1,2-dioxo-3,3-dimethylpentyl)-2-pyrrolidinecarboxylate

A solution of methyl (2S)-1-(1,2-dioxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil. ¹H NMR (CDCl₃): δ0.88 (t, 3H); 1.22, 1.26 (s, 3H each); 1.75 (dm, 2H); 1.87-2.10 (m, 3H); 2.23 (m, 1H); 3.54 (m, 2H); 3.76 (s, 3H); 4.52 (dm, 1H, J=8.4, 3.4).

Synthesis of (2S)-1-(1,2-dioxo-3,3-dimethylpentyl)-2-pyrrolidinecarboxylic acid

A mixture of methyl (2S)-1-(1,2-dioxo-3,3-dimethylpentyl)-2-pyrrolidinecarboxylate (2.10 g; 8.23 mmol), 1 N LiOH (15 mL), and methanol (50 mL) was stirred at 0° C. for 30 minutes and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-white solid which did not require further purification. ¹H NMR (CDCl₃): δ0.87 (t, 3H); 1.22, 1.25 (s, 3H each); 1.77 (dm, 2H); 2.02 (m, 2H); 2.17 (m, 1H); 2.25 (m, 1H); 3.53 (dd, 2H, J=10.4, 7.3); 4.55 (dd, 1H, J=8.6, 4.1).

3-Phenyl-1-propyl (2S)-1-(3,3-dimethyl-1,2-dioxopentyl)-2-pyrrolidinecarboxylate (137)

A mixture of (2S)-1-(1,2-dioxo-3,3-dimethylpentyl)-2-pyrrolidine-carboxylic acid (600 mg; 2.49 mmol), 3-phenyl-1-propanol (508 mg; 3.73 mmol), dicyclohexylcarbodiimide (822 mg; 3.98 mmol), camphorsulfonic acid (190 mg; 0.8 mmol) and 4-dimethylaminopyridine (100 mg; 0.8 mmol) in methylene chloride (20 mL) was stirred overnight under a nitrogen atmosphere. The reaction mixture was filtered through Celite to remove solids and concentrated in vacuo, and the crude material was purified on a flash column (25% ethyl acetate in hexane) to obtain 720 mg (80%) of Example 14 as a colorless oil. ¹H NMR (CDCl₃): δ0.84 (t, 3H); 1.19 (s, 3H); 1.23 (s, 3H); 1.70 (dm, 2H); 1.98 (m, 5H); 2.22 (m, 1H); 2.64 (m, 2H); 3.47 (m, 2H); 4.14 (m, 2H); 4.51 (d, 1H); 7.16 (m, 3H); 7.26 (m, 2H).

### Example 15

### The method of Example 14 was utilized to prepare the following illustrative compounds.

Compound 138: 3-phenyl-1-prop-2-(E)-enyl (2S)-1-(3,3-dimethyl-1,2-dioxopentyl)-2-pyrrolidinecarboxylate, 80%. ¹H NMR (360 MHz, CDCl₃): δ0.86 (t, 3H); 1.21 (s, 3H); 1.25 (s, 3H); 1.54-2.10 (m, 5H); 2.10-2.37 (m, 1H); 3.52-3.55 (m, 2H); 4.56 (dd, 1H, J=3.8, 8.9); 4.78-4.83 (m, 2H); 6.27 (m, 1H); 6.67 (dd, 1H, J=15.9); 7.13-7.50 (m, 5H).

Compound 139: 3-(3,4,5-trimethoxyphenyl)-1-propyl (2S)-1-(3,3-dimethyl-1,2-dioxopentyl)-2-pyrrolidine-carboxylate, 61%. ¹H NMR (CDCl₃): δ0.84 (t, 3H); 1.15 (s, 3H); 1.24 (s, 3H); 1.71 (dm, 2H); 1.98 (m, 5H); 2.24 (m, 1H); 2.63 (m, 2H); 3.51 (t, 2H); 3.79 (s, 3H); 3.83 (s, 3H); 4.14 (m, 2H); 4.52 (m, 1H); 6.36 (s, 2H).

Compound 140: 3-(3,4,5-trimethoxyphenyl)-1-prop-2-(E)-enyl (2S)-1-(3,3-dimethyl-1,2-dioxopentyl)-2-pyrrolidine carboxylate, 66%. ¹H NMR (CDCl₃): δ0.85 (t, 3H); 1.22 (s, 3H); 1.25 (s, 3H); 1.50-2.11 (m, 5H); 2.11-2.40 (m, 1H); 3.55 (m, 2H); 3.85 (s, 3H); 3.88 (s, 6H); 4.56 (dd, 1H); 4.81 (m, 2H); 6.22 (m, 1H); 6.58 (d, 1H, J=16); 6.63 (s, 2H).

Compound 141: 3-(4,5-methylenedioxyphenyl)-1-propyl (2S)-1-(3,3-dimethyl-1,2-dioxopentyl)-2-pyrrolidine-carboxylate, 82%. ¹H NMR (360 MHz, CDCl₃): δ0.86 (t, 3H); 1.22 (s, 3H); 1.25 (s, 3H); 1.60-2.10 (m, 5H); 3.36-3.79 (m, 2H); 4.53 (dd, 1H, J=3.8, 8.6); 4.61-4.89 (m, 2H); 5.96 (s, 2H); 6.10 (m, 1H); 6.57 (dd, 1H, J=6.2, 15.8); 6.75 (d, 1H, J=8.0); 6.83 (dd, 1H, J=1.3, 8.0); 6.93 (s, 1H).

Compound 142: 3-(4,5-methylenedioxyphenyl)-1-prop-2-(E)-enyl (2S)-1-(3,3-dimethyl-1,2-dioxopentyl)-2-pyrrolidinecarboxylate, 82%. ¹H NMR (360 MHz, CDCl₃) δ0.86 (t, 3H); 1.22 (s, 3H); 1.25 (s, 3H); 1.60-2.10 (m, 5H); 2.10-2.39 (m, 1H); 3.36-3.79 (m, 2H); 4.53 (dd, 1H, J=3.8, 8.6); 4.61-4.89 (m, 2H); 5.96 (s, 2H); 6.10 (m, 1H); 6.57 (dd, 1H, J=6.2, 15.8); 6.75 (d, 1H, J=8.0); 6.83 (dd, 1H, J=1.3, 8.0); 6.93 (s, 1H).

Compound 144: 3-cyclohexyl-1-prop-2-(E)-enyl (2S)-1-(3,3-dimethyl-1,2-dioxopentyl)-2-pyrrolidine-carboxylate, 92%. ¹H NMR (360 MHz, CDCl₃): δ 0.86 (t, 3H); 1.13-1.40 (m+2 singlets, 9H total); 1.50-1.87 (m, 8H); 1.87-2.44 (m, 6H); 3.34-3.82 (m, 2H); 4.40-4.76 (m, 3H); 5.35-5.60 (m, 1H); 5.60-5.82 (dd, 1H, J=6.5, 16).

Compound 145: (1R)-1,3-Diphenyl-1-propyl (2S)-1-(3,3-dimethyl-1,2-dioxopentyl)-2-pyrrolidinecarboxylate, 90%. ¹H NMR (360 MHz, CDCl₃): δ0.85 (t, 3H); 1.20 (s, 3H); 1.23 (s, 3H); 1.49-2.39 (m, 7H); 2.46-2.86 (m, 2H); 3.25-3.80 (m, 2H); 4.42-4.82 (m, 1H); 5.82 (td, 1H, J=1.8, 6.7); 7.05-7.21 (m, 3H); 7.21-7.46 (m, 7H).

Compound 146: 3-phenyl-1-propyl (2S)-1-(1,2-dioxo-2-[2-furanyl])ethyl-2-pyrrolidinecarboxylate, 99%. ¹H NMR (300 MHz, CDCl₃): δ1.66-2.41 (m, 6H); 2.72 (t, 2H, J=7.5); 3.75 (m, 2H); 4.21 (m, 2H); 4.61 (m, 1H); 6.58 (m, 1H); 7.16-7.29 (m, 5H); 7.73 (m, 2H).

Compound 147: 3-phenyl-1-propyl (2S)-1-(1,2-dioxo-2-[2-thienyl])ethyl-2-pyrrolidinecarboxylate, 81%. ¹H NMR (300 MHz, CDCl₃): δ1.88-2.41 (m, 6H); 2.72 (dm, 2H); 3.72 (m, 2H); 4.05 (m, 1H); 4.22 (m, 1H); 4.64 (m, 1H); 7.13-7.29 (m, 6H); 7.75 (dm, 1H); 8.05 (m, 1H).

Compound 149: 3-phenyl-1-propyl (2S)-1-(1,2-dioxo-2-phenyl)ethyl-2-pyrrolidinecarboxylate, 99%. ¹H NMR (300 MHz, CDCl₃): δ1.97-2.32 (m, 6H); 2.74 (t, 2H, J=7.5); 3.57 (m, 2H); 4.24 (m, 2H); 4.67 (m, 1H); 6.95-7.28 (m, 5H); 7.51-7.64 (m, 3H); 8.03-8.09 (m, 2H).

Compound 150: 3-(2,5-dimethoxyphenyl)-1-propyl (2S)-1-(3,3-dimethyl-1,2-dioxopentyl)-2-pyrrolidine-carboxylate, 99%. ¹H NMR (300 MHz, CDCl₃): δ0.87 (t, 3H); 1.22 (s, 3H); 1.26 (s, 3H); 1.69 (m, 2H); 1.96 (m, 5H); 2.24 (m, 1H); 2.68 (m, 2H); 3.55 (m, 2H); 3.75 (s, 3H); 3.77 (s, 3H); 4.17 (m, 2H); 4.53 (d, 1H); 6.72 (m, 3H).

Compound 151: 3-(2,5-dimethoxyphenyl)-1-prop-2-(E)-enyl (2S)-1-(3,3-dimethyl-1,2-dioxopentyl)-2-pyrrolidine-carboxylate, 99%. ¹H NMR (300 MHz, CDCl₃): δ0.87 (t, 3H); 1.22 (s, 3H); 1.26 (s, 3H); 1.67 (m, 2H); 1.78 (m, 1H); 2.07 (m, 2H); 2.26 (m, 1H); 3.52 (m, 2H); 3.78 (s, 3H); 3.80 (s, 3H); 4.54 (m, 1H); 4.81 (m, 2H); 6.29 (dt, 1H, J=15.9); 6.98 (s, 1H).

Compound 152: 2-(3,4,5-trimethoxyphenyl)-1-ethyl (2S)-1-(3,3-dimethyl-1,2-dioxopentyl)-2-pyrrolidine-carboxylate, 97%. ¹H NMR (300 MHz, CDCl₃): δ0.84 (t, 3H); 1.15 (s, 3H); 1.24 (s, 3H); 1.71 (dm, 2H); 1.98 (m, 5H); 2.24 (m, 1H); 2.63 (m, 2H); 3.51 (t, 2H); 3.79 (s, 3H); 3.83 (s, 3H); 4.14 (m, 2H); 4.52 (m, 1H); 6.36 (s, 2H).

Compound 153: 3-(3-Pyridyl)-1-propyl (2S)-1-(3,3-dimethyl-1,2-dioxopentyl)-2-pyrrolidinecarboxylate, 80%. ¹H NMR (CDCl₃, 300 MHz): δ0.85 (t, 3H); 1.23, 1.26 (s, 3H each); 1.63-1.89 (m, 2H); 1.90-2.30 (m, 4H); 2.30-2.50 (m, 1H); 2.72 (t, 2H); 3.53 (m, 2H); 4.19 (m, 2H); 4.53 (m, 1H); 7.22 (m, 1H); 7.53 (dd, 1H); 8.45.

Compound 154: 3-(2-Pyridyl)-1-propyl (2S)-1-(3,3-dimethyl-1,2-dioxopentyl)-2-pyrrolidinecarboxylate, 88%. ¹H NMR (CDCl₃, 300 MHz): δ0.84 (t, 3H); 1.22, 1.27 (s, 3H each); 1.68-2.32 (m, 8H); 2.88 (t, 2H, J=7.5); 3.52 (m, 2H); 4.20 (m, 2H); 4.51 (m, 1H); 7.09-7.19 (m, 2H); 7.59 (m, 1H); 8.53 (d, 1H, J=4.9).

Compound 155: 3-(4-Pyridyl)-1-propyl (2S)-1-(3,3-dimethyl-1,2-dioxopentyl)-2-pyrrolidinecarboxylate, 91%. ¹H NMR (CDCl₃, 300 MHz): δ6.92-6.80 (m, 4H); 6.28 (m, 1H); 5.25 (d, 1H, J=5.7); 4.12 (m, 1H); 4.08 (s, 3H); 3.79 (s, 3H); 3.30 (m, 2H); 2.33 (m, 1H); 1.85-1.22 (m, 7H); 1.25 (s, 3H); 1.23 (s, 3H); 0.89 (t, 3H, J=7.5).

Compound 156: 3-phenyl-1-propyl (2S)-1-(2-cyclohexyl-1,2-dioxoethyl)-2-pyrrolidinecarboxylate, 91%. ¹H NMR (CDCl₃, 300 MHz): δ1.09-1.33 (m, 5H); 1.62-2.33 (m, 12H); 2.69 (t, 2H, J=7.5); 3.15 (dm, 1H); 3.68 (m, 2H); 4.16 (m, 2H); 4.53, 4.84 (d, 1H total); 7.19 (m, 3H); 7.29 (m, 2H).

Compound 157: 3-phenyl-1-propyl (2S)-1-(2-tert-butyl-1,2-dioxoethyl)-2-pyrrolidinecarboxylate, 92%. ¹H NMR (CDCl₃, 300 MHz): δ1.29 (s, 9H); 1.94-2.03 (m, 5H); 2.21 (m, 1H); 2.69 (m, 2H); 3.50-3.52 (m, 2H); 4.16 (m, 2H); 4.53 (m, 1H); 7.19 (m, 3H); 7.30 (m, 2H).

Compound 158: 3-phenyl-1-propyl (2S)-1-(2-cyclohexylethyl-1,2-dioxoethyl)-2-pyrrolidinecarboxylate, 97%. ¹H NMR (CDCl₃, 300 MHz): δ0.88 (m, 2H); 1.16 (m, 4H); 1.43-1.51 (m, 2H); 1.67 (m, 5H); 1.94-2.01 (m, 6H); 2.66-2.87 (m, 4H); 3.62-3.77 (m, 2H); 4.15 (m, 2H); 4.86 (m, 1H); 7.17-7.32 (m, 5H).

Compound 159: 3-(3-pyridyl)-1-propyl (2S)-1-(2-cyclohexylethyl-1,2-dioxoethyl)-2-pyrrolidinecarboxylate, 70%. ¹H NMR (CDCl₃, 300 MHz): δ0.87 (m, 2H); 1.16 (m, 4H); 1.49 (m, 2H); 1.68 (m, 4H); 1.95-2.32 (m, 7H); 2.71 (m, 2H); 2.85 (m, 2H); 3.63-3.78 (m, 2H); 4.19 (m, 2H); 5.30 (m, 1H); 7.23 (m, 1H); 7.53 (m, 1H); 8.46 (m, 2H).

Compound 160: 3-(3-pyridyl)-1-propyl (2S)-1-(2-tert-butyl-1,2-dioxoethyl)-2-pyrrolidinecarboxylate, 83%. ¹H NMR (CDCl₃, 300 MHz): δ1.29 (s, 9H); 1.95-2.04 (m, 5H); 2.31 (m, 1H); 2.72 (t, 2H, J=7.5); 3.52 (m, 2H); 4.18 (m, 2H); 4.52 (m, 1H); 7.19-7.25 (m, 1H); 7.53 (m, 1H); 8.46 (m, 2H).

Compound 161: 3,3-diphenyl-1-propyl (2S)-1-(3,3-dimethyl-1,2-dioxopentyl)-2-pyrrolidinecarboxylate, 99%. ¹H NMR (CDCl₃, 300 MHz): δ0.85 (t, 3H); 1.21, 1.26 (s, 3H each); 1.68-2.04 (m, 5H); 2.31 (m, 1H); 2.40 (m, 2H); 3.51 (m, 2H); 4.08 (m, 3H); 4.52 (m, 1H); 7.18-7.31 (m, 10H).

Compound 162: 3-(3-pyridyl)-1-propyl (2S)-1-(2-cyclohexyl-1,2-dioxoethyl)-2-pyrrolidinecarboxylate, 88%. ¹H NMR (CDCl₃, 300 MHz): δ1.24-1.28 (m, 5H); 1.88-2.35 (m, 11H); 2.72 (t, 2H, J=7.5); 3.00-3.33 (dm, 1H); 3.69 (m, 2H); 4.19 (m, 2H); 4.55 (m, 1H); 7.20-7.24 (m, 1H); 7.53 (m, 1H); 8.47 (m, 2H).

Compound 163: 3-(3-Pyridyl)-1-propyl (2S)-N-([2-thienyl]glyoxyl)pyrrolidinecarboxylate, 49%. ¹H NMR (CDCl₃, 300 MHz): δ1.81-2.39 (m, 6H); 2.72 (dm, 2H); 3.73 (m, 2H); 4.21 (m, 2H); 4.95 (m, 1H); 7.19 (m, 2H); 7.61 (m, 1H); 7.80 (d, 1H); 8.04 (d, 1H); 8.46 (m, 2H).

Compound 164: 3,3-Diphenyl-1-propyl (2S)-1-(3,3-dimethyl-1,2-dioxobutyl)-2-pyrrolidinecarboxylate, 99%. ¹H NMR (CDCl₃, 300 MHz): δ1.27 (s, 9H); 1.96 (m, 2H); 2.44 (m, 4H); 3.49 (m, 1H); 3.64 (m, 1H); 4.08 (m, 4H); 4.53 (dd, 1H); 7.24 (m, 10H).

Compound 165: 3,3-Diphenyl-1-propyl (2S)-1-cyclohexyl glyoxyl-2-pyrrolidinecarboxylate, 91%. ¹H NMR (CDCl₃, 300 MHz): δ1.32 (m, 6H); 1.54-2.41 (m, 10H); 3.20 (dm, 1H); 3.69 (m, 2H); 4.12 (m, 4H); 4.52 (d, 1H); 7.28 (m, 10H).

Compound 166: 3,3-Diphenyl-1-propyl (2S)-1-(2-thienyl) glyoxyl-2-pyrrolidinecarboxylate, 75%. ¹H NMR (CDCl₃, 300 MHz): δ2.04 (m, 3H); 2.26 (m, 2H); 2.48 (m, 1H); 3.70 (m, 2H); 3.82-4.18 (m, 3H total); 4.64 (m, 1H); 7.25 (m, 11H); 7.76 (dd, 1H); 8.03 (m, 1H).

### Example 16

General procedure for the synthesis of acrylic esters, exemplified for methyl (3,3,5-trimethoxy)-trans-cinnamate.

A solution of 3,4,5-trimethoxybenzaldehyde (5.0 g; 25.48 mmol) and methyl (triphenyl-phosphoranylidene)acetate (10.0 g; 29.91 mmol) in tetrahydrofuran (250 mL) was refluxed overnight. After cooling, the reaction mixture was diluted with 200 mL of ethyl acetate and washed with 2×200 mL of water, dried, and concentrated in vacuo. The crude residue was chromatographed on a silica gel column, eluting with 25% ethyl acetate in hexane, to obtain 5.63 g (88%) of the cinnamate as a white crystalline solid. ¹H NMR (300 MHz; CDCl₃): δ3.78 (s, 3H); 3.85 (s, 6H); 6.32 (d, 1H, J=16); 6.72 (s, 2H); 7.59 (d, 1H, J=16).

### Example 17

General procedure for the synthesis of saturated alcohols from acrylic esters, exemplified for (3,4,5-trimethoxy) phenylpropanol.

A solution of methyl (3,3,5-trimethoxy)-trans-cinnamate (1.81 g; 7.17 mmol) in tetrahydrofuran (30 mL) was added in a dropwise manner to a solution of lithium aluminum hydride (14 mmol) in THF (35 mL), with stirring and under an argon atmosphere. After the addition was complete, the mixture was heated to 75° C. for 4 hours. After cooling, it was quenched by the careful addition of 15 mL of 2 N NaOH followed by 50 mL of water. The resulting mixture was filtered through Celite to remove solids, and the filter cake was washed with ethyl acetate. The combined organic fractions were washed with water, dried, concentrated in vacuo, and purified on a silica gel column, eluting with ethyl acetate to obtain 0.86 g (53%) of the alcohol as a clear oil. ¹H NMR (300 MHz; CDCl₃): δ1.23 (br, 1H); 1.87 (m, 2H); 2.61 (t, 2H, J=7.1); 3.66 (t, 2H); 3.80 (s, 3H); 3.83 (s, 6H); 6.40 (s, 2H).

### Example 18

General procedure for the synthesis of trans-allylic alcohols from acrylic esters, exemplified for (3,4,5-trimethoxy)phenylprop-2-(E)-enol.

A solution of methyl (3,3,5-trimethoxy)-trans-cinnamate (1.35 g; 5.35 mmol) in toluene (25 mL) was cooled to −10° C. and treated with a solution of diisobutylaluminum hydride in toluene (11.25 mL of a 1.0 M solution; 11.25 mmol). The reaction mixture was stirred for 3 hours at 0° C. and then quenched with 3 mL of methanol followed by 1 N HCl until the pH was 1. The reaction mixture was extracted into ethyl acetate and the organic phase was washed with water, dried and concentrated. Purification on a silica gel column eluting with 25% ethyl acetate in hexane furnished 0.96 g (80%) of a thick oil. ¹H NMR (360 MHz; CDCl₃): δ3.85 (s, 3H); 3.87 (s, 6H); 4.32 (d, 2H, J=5.6); 6.29 (dt, 1H, J=15.8, 5.7), 6.54 (d, 1H, J=15.8); 6.61 (s, 2H).

### Example 19

### Synthesis of (2S)-1-(3,3-dimethyl-1,2-dioxopentyl)-2-pyrrolidinecarboxylate (421)

Synthesis of (2S)-1-(1,2-dioxo-2-methoxyethyl)-2-pyrrolidinecarboxylate.

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 min, a solution of methyl oxalyl chloride (3.20 g; 26.12 mmol) in methylene chloride (45 mL) was added dropwise. The resulting mixture was stirred at 0° C. for 1.5 hr. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃): δ 1.93 (dm, 2H); 2.17 (m, 2H); 3.62 (m, 2H); 3.71 (s, 3H); 3.79, 3.84 s, 3H total); 4.86 (dd, 1H, J=8.4, 3.3).

Synthesis of methyl (2S)-1-(1,2-dioxo-3,3-dimethylpentyl)-2-pyrrolidinecarboxylate.

A solution of methyl (2S)-1-(1,2-dioxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil. ¹H NMR (CDCl₃):δ 0.88 (t, 3H); 1.22, 1.26 (s, 3H each); 1.75 (dm, 2H); 1.87-2.10 (m, 3H); 2.23 (m, 1H); 3.54 (m, 2H); 3.76 (s, 3H); 4.52 (dm, 1H, J=8.4, 3.4).

Synthesis of (2S)-1-(1,2-dioxo-3,3-dimethylpentyl)-2-pyrrolidinecarboxylic acid

A mixture of methyl (2S)-1-(1,2-dioxo-3,3-dimethylpentyl)-2-pyrrolidinecarboxylate (2.10 g; 8.23 mmol), 1 N LiOH (15 mL), and methanol (50 mL) was stirred at 0° C. for 30 min and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-white solid which did not require further purification. ¹H NMR (CDCl₃):δ 0.87 (t, 3H); 1.22, 1.25 (s, 3H each); 1.77 (dm, 2H); 2.02 (m, 2H); 2.17 (m, 1H); 2.25 (m, 1H); 3.53 (dd, 2H, J=10.4, 7.3); 4.55 (dd, 1H, J=8.6, 4.1).

### Example 20

### Synthesis of (2S)-1-(1,2-dioxo-3,3-dimethylpentyl)-2-pyrrolidinecarboxamide (318)

Isobutyl chloroformate (20 mmol, 2.7 mL) was added to a solution containing (2S)-1-(1,2-dioxo-3,3-dimethylpentyl)-2-pyrrolidinecarboxylic acid (4.89 g, 20 mmol)(from Example 19) in 50 mL methylene chloride at −10° C. with stirring. After 5 minutes, ammonia was added dropwise (20 mmol, 10 mL of 2 M ethyl alcohol solution). The reaction was warmed up to room temperature after stirring at −10° C. for 30 minutes. The mixture was diluted with water, and extracted into 200 mL methylene chloride. The organic extract was concentrated and further purified by silica gel to give 4.0 g of product as a white solid (81.8% yield). ¹H NMR (CDCl₃): δ0.91 (t, 3H, J=7.5); 1.28 (s, 6H, each); 1.63-1.84 (m, 2H); 1.95-2.22 (m, 3H); 2.46 (m, 1H); 3.55-3.67 (m, 2H); 4.67 (t, 1H, J=7.8); 5.51-5.53 (br, 1H, NH); 6.80 (br, 1H, NH).

### Example 21

### Synthesis of (2S)-1-(1,2-dioxo-3,3-dimethylpentyl)-2-pyrrolidinecarbonitrile (313)

To a solution of 0.465 mL DMF (6 mmol) in 10 mL acetonitrile at 0° C. was added 0.48 mL (5.5 mmol) of oxalyl chloride. A white precipitate formed immediately and was accompanied by gas evolution. When complete, a solution of 1.2 g (5 mmol) of (2S)-1-(1,2-dioxo-3,3-dimethylpentyl)-2-pyrrolidinecarboxamide (from Example 20) in 2.5 mL acetonitrile was added. When the mixture became homogeneous, 0.9 mL (11 mmol) pyridine was added. After 5 min., the mixture was diluted into water and extracted by 200 mL ethyl acetate. The organic layer was concentrated and further purified by silica gel to give 0.8 g product as a white solid (72% yield). ¹H NMR (CDCl₃): δ0.87 (t, 3H, J=7.5); 1.22 (s, 3H); 1.24 (s, 3H); 1.80 (m, 2H); 2.03-2.23 (m, 4H); 3.55 (m, 2H); 4.73 (m, 1H).

### Example 22

### Synthesis of (2S)-1-(1,2-dioxo-3,3-dimethylpentyl)-2-pyrrolidinetetrazole (314)

A mixture of (2S)-1-(1,2-dioxo-3,3-dimethylpentyl)-2-pyrrolidinecarbonitrile (222 mg, 1 mmol)(from Example 21), NaN₃ (81 mg, 1.3 mmol) and NH₄Cl (70 mg, 1.3 mmol) in 3 mL DMF was stirred at 130° C. for 16 hours. The mixture was concentrated and purified by silica gel to afford 200 mg product as white solid (75.5% yield). ¹H NMR (CDCl₃): δ 0.88 (t, 3H, J=7.5); 1.22 (s, 6H); 1.68 (m, 2H); 2.05-2.36 (m, 3H); 2.85 (m, 1H); 3.54 (m, 1H); 3.75 (m, 1H); 5.40 (m, 1H).

### Example 23

### Synthesis of 3-(3,3-dimethyl-2-oxopentanoyl)-1,3-oxazolidine-4-carboxylic acid (612)

Methyl 1,3-oxazolidine-4-carboxylate

This compound was synthesized according to the procedure found in J. Med. Chem. (1990) 33:1459-1469.

Methyl 2-[4-(methoxycarbonyl)(1,3-oxazolidin-3-yl)]-2-oxoacetate

To an ice cooled solution of methyl 1,3-oxazolidine-4-carboxylate (0.65 g, 4.98 mM) were added triethylamine (0.76 ml, 5.45 mM) and methyl oxalyl chloride (0.5 ml, 5.45 mM). This mixture was stirred at 0° C. for 2 hours. After this time the mixture was washed with water, then brine, dried with anhydrous magnesium sulfate, filtered and evaporated. The resulting pale yellow oil was flash chromatographed eluting with 30% EtOAc/hexane, 50% EtOAc/hexane, and finally 75% EtOAc/hexane. A clear oil of product (0.52 g, 48%) was obtained. Anal. (C₈H₁₁NO₆)C,H,N; ¹H NMR (CDCl₃, 400 MHz) δ (2 rotamers 1:1) 3.78 (s, 1.5H); 3.79 (s, 1.5H); 3.87 (s, 1.5H); 3.91 (s, 1.5H); 4.14-4.36 (m, 2H); 4.70 (dd, 0.5H, J=4.1, 6.8); 5.08 (dd, 0.5H, J=3.1, 6.7); 5.10 (d, 0.5H, J=5.9); 5.27 (d, 0.5H, J=5.8); 5.36 (dd, 1H, J=5.3, 17.8).

Methyl 3-(3,3-dimethyl-2-oxopentanoyl)-1,3-oxazolidine-4-carboxylate

To a solution of methyl 2-[4-(methoxycarbonyl)-(1,3-oxazolidin-3-yl)]-2-oxoacetate (0.84 g, 3.87 mM) in THF (50 ml) cooled to −78° C. was added 1,1-dimethylpropyl-magnesium chloride (1M in THF, 8 ml, 8 mM). After 3 hrs. at −78° C. the mixture was quenched with saturated NH₄Cl (50 ml) and extracted with ethyl acetate (100 ml). The organic layer separated, washed with brine (100 ml), dried with anhydrous magnesium sulfate, filtered and evaporated. The resulting pale yellow oil was flash chromatographed eluting with 20% EtOAc/hexane. A clear oil (3) (0.61 g, 61%) was obtained. ¹H NMR (CDCl₃, 400 MHz): δ 0.85 (t, 3H, J=7.5); 1.25 (s, 3H); 1.26 (s, 3H); 1.67-1.94 (m, 2H); 3.79 (s, 3H); 4.12-4.31 (m, 2H); 4.64 (dd, 1H, J=4.1, 6.8); 5.04 (dd, 2H, J=4.9, 9.4).

3-(3,3-dimethyl-2-oxopentanoyl)-1,3-oxazolidine-4-carboxylic acid (612)

Methyl 3-(3,3-dimethyl-2-oxopentanoyl)-1,3-oxazolidine-4-carboxylate (3) (0.6 g, 2.33 mM) was dissolved in MeOH (25 ml) and added LiOH (1M in water, 10 ml, 10 mM). This mixture was stirred overnight at room temperature. The residues were evaporated and partitioned between EtOAc (50 ml) and 2N HCl (50 mL). The aqueous layer was extracted twice more with EtOAc (2×25 ml). The extracts were washed with brine (50 ml), dried with anhydrous magnesium sulfate, filtered and evaporated. A clear oil product (0.49 g, 86%) was obtained. Anal. (C₁₁H₁₇NO₅) C, H, N; ¹H NMR (CDCl₃, 400 MHz): δ 0.84 (t, 3H, J=7.5); 1.25 (s, 6H); 1.70-1.95 (m, 2H); 4.22-4.29 (m, 2H); 4.66 (dd, 1H, J=4.6, 6.5); 5.04 (dd, 2H, J=5.0, 8.9); 7.67 (bs, 1H).

### Example 24

### Synthesis of (2S)-1-(N-cyclohexylcarbamoyl) pyrrolidine-2-carboxylic acid (619)

Methyl (2S)-1-(N-cyclohexylcarbamoyl)pyrrolidine-2-carboxylate.

A mixture of cyclohexyl isocyanate (3.88 g; 31 mmol), L-proline ester hydrochloride (5.0 g; 30.19 mmol), and triethylamine (9 mL) in methylene chloride (150 ml) was stirred overnight at room temperature. The reaction mixture was washed with 2×100 ml of 1 N HCL and 1×100 ml of water. The organic phase was dried, concentrated and purified on a silica gel column (50% EtOAc/hexane) to yield the urea as a thick oil, ¹H NMR (CDCl₃, 400 MHz): δ 1.09-1.15 (m, 3H); 1.33 (m, 2H); 1.68 (m, 3H); 1.93-2.05 (m, 6H); 3.33 (m, 1H); 3.43 (m, 1H); 3.46 (m, 1H); 3.73 (s, 3H); 4.39 (m, 1H); 4.41 (m, 1H).

(2S)-1-(N-cyclohexylcarbamoyl)pyrrolidine-2-carboxylic acid (619)

Methyl (2S)-1-(N-cyclohexylcarbamoyl)pyrrolidine-2-carboxylate (3.50 g) was dissolved in methanol (60 ml), cooled to 0° C., and treated with 2N LiOH (20 ml). After stirring overnight, the mixture was partitioned between ether and water. The ether layer was discarded and the aqueous layer was made acidic (pH 1) with 1N HCl and extracted with methylene chloride. Drying and removal of the solvent provided 2.20 g of the product as a white solid, ¹H NMR (CDCl₃, 400 MHz): δ 1.14-1.18 (m, 3H); 1.36-1.38 (m, 2H); 1.71-1.75 (m, 3H); 1.95-2.04 (m, 5H); 2.62 (m, 1H); 3.16 (m, 1H); 3.30-3.33 (m, 1H); 3.67 (m, 1H); 4.38 (br, 1H); 4.46 (m, 1H).

### Example 25

### Synthesis of (2S)-N-(benzylsulfonyl)-2-pyrrolidinecarboxylic acid (719)

To a cooled (0° C.) solution of proline methyl ester hydrochloride salt (5.0 g; 30.19 mmol) in 200 mL of methylene chloride was added triethylamine (35 mL) and benzenesulfonyl chloride (5.75 g; 30.19 mmol). The mixture was stirred for one hour at 0° C. and then washed with 2×100 mL of water. The organic phase was dried and concentrated. Chromatography eluting with 50% EtOAc/hexane delivered 8.14 g (5%) of the N-sulfonamide methyl ester, which was dissolved in 120 mL of methanol, cooled to 0° C., and treated with 40 mL of 1 N lithium hydroxide. The mixture was stirred for 1 hour at 0° C. and then overnight at room temperature. After making the reaction mixture acidic (pH 1) with 1 N HCl, the product was extracted into methylene chloride and dried and concentrated to yield 4.25 g of (2S)-N-(benzylsulfonyl)-2-pyrrolidinecarboxylic acid (A) as a white solid, ¹H NMR (CDCl₃, 400 MHz): δ 1.85-1.90 (m, 2H); 2.08 (m, 1H); 2.18 (m, 1H); 3.04 (m, 1H); 3.27 (m, 1H); 4.32-4.35 (m, 2H); 4.45 (m, 1H); 4.45 (m, 2H); 7.36 (m, 3H); 7.48 (m, 2H); 10.98 (br, 1H).

### Example 26

### Synthesis of (2S)-1-(phenylmethylsulfonyl)-2-hydroxymethylpyrrolidine (813)

To a solution of (S)-(+)-2-pyrrolidinemethanol (1.01 g, 10 mmol) and triethylamine (1.5 ml, 11 mmol) in 30 ml methylene chloride was added 1.9 g (10 mmol) α-toluenesulfonyl chloride at 0° C. with stirring. The reaction was gradually warmed up to room temperature and stirred overnight. The mixture was diluted with water, and extracted into 200 ml methylene chloride. The organic extract was concentrated and further purified by silica gel to give 1.5 g product as a white solid (58.9% yield). ¹H NMR (CDCl₃) δ 01.71-1.88 (m, 4H); 2.05 (br, 1H, OH); 3.22 (m, 2H); 3.47 (m, 2H); 3.67 (m, 1H); 4.35 (s, 2H); 7.26-7.44 (m, 5H, aromatic).

### Example 27

### Synthesis of (2S)-1-(phenylmethyl)sulfonyl-2-pyrrolidinecarboxamide (814)

To a solution of L-prolinamide (2.28 g, 20 mmol) and triethylamine (5.76 ml, 42 mmol) in 40 ml methylene chloride was added 3.92 g (20 mmol) α-toluenesulfonyl chloride at 0° C. with stirring. The reaction was gradually warmed up to room temperature and stirred overnight. The mixture was diluted with water, and extracted into 200 ml methylene chloride. The organic extract was concentrated and further purified by silica gel to give 3.0 g product as a white solid (55.7% yield). ¹H NMR (CDCl₃): δ 01.89 (m, 3H); 2.25 (m, 1H); 3.40 (m, 1H); 3.50 (m, 1H); 3.96 (m, 1H); 4.35 (s, 2H); 7.39-7.45 (m, 5H, aromatic).

### Example 28

### Synthesis of (2S)-1-(phenylmethyl)sulfonyl-2-pyrrolidinecarbonitrile (815)

To a solution of 0.67 ml DMF (8.7 mmol) in 10 ml acetonitrile at 0° C. was added 0.70 ml (8.0 mmol) oxalyl chloride. A white precipitate was formed immediately and was accompanied by gas evolution. When complete, a solution of 2.0 g (7.5 mmol) of (2S)-1-(phenylmethyl)sulfonyl-2-pyrrolidine-carboxamide in 5.0 ml acetonitrile was added. When the mixture became homogeneous, 1.35 ml (16.5 mmol) pyridine was added. After 5 min., the mixture was diluted with water, and extracted by 200 ml ethyl acetate. The organic layer was concentrated and further purified by silica gel to give 1.5 g product as a white solid (80% yield). ¹H NMR (CDCl₃): δ 1.92 (m, 2H); 2.01 (m, 1H); 2.11 (m, 1H); 3.45 (m, 2H); 4.35 (s, 2H); 4.65 (m, 1H); 7.26-7.45 (m, 5H, aromatic).

### Example 29

### Synthesis of (2S)-1-(phenylmethyl)sulfonyl-2-pyrrolidinetetrazole (722).

A mixture of (2S)-1-(phenylmethyl)sulfonyl-2-pyrrolidinecarbonitrile (250 mg, 1 mmol), NaN₃ (81 mg, 1.3 mmol) and NH₄Cl (70 mg, 1.3 mmol) in 3 ml DMF was stirred at 130° C. for 16 hours. The mixture was concentrated and purified by silica gel to give 120 mg product as a white solid (41.1% yield). ¹H NMR (CDCl₃): δ 01.95 (m, 2H); 2.21 (m, 1H); 2.90 (m, 1H); 3.40 (m, 2H); 4.27 (s, 2H); 5.04 (m, 1H); 7.36-7.41 (m, 5H, aromatic); 8.05 (s, 1H, NH).

The following sensorineurotrophic compounds (referenced by Compound No.) were used in the following non-limiting examples to demonstrate the efficacy of the compounds of the invention in the treatment and prevention of sensorineural degeneration:

| Com- | |
|---|---|
| pound | |
| No. | Structure |
| I | 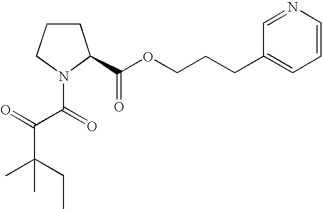 |
| II | 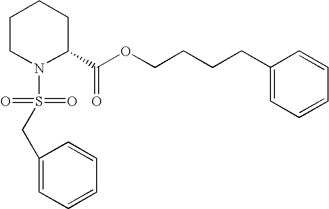 |
| III | 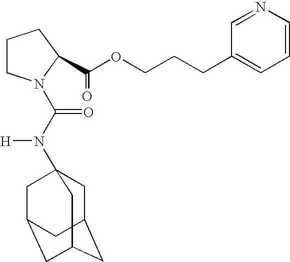 |
| IV | 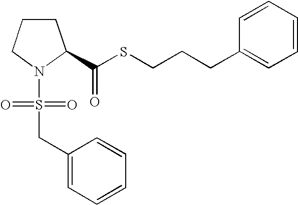 |
| V | 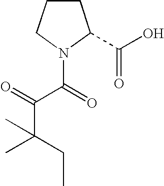 |
| VI | 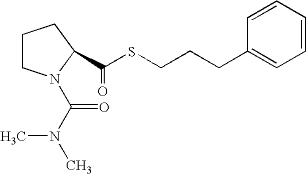 |
| VII | 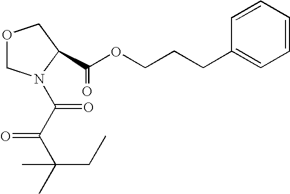 |
| VIII | 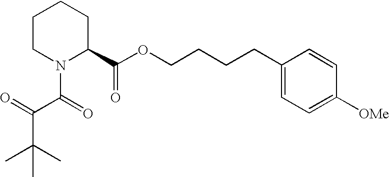 |
| IX | 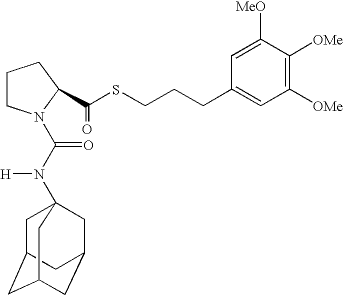 |
| X | 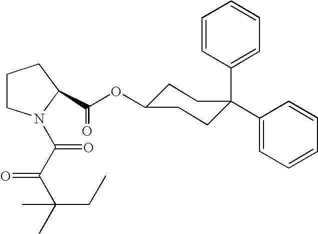 |
| XI | 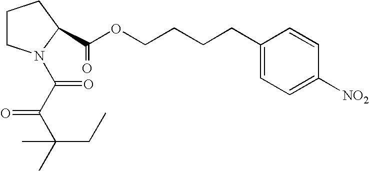 |
| XII | 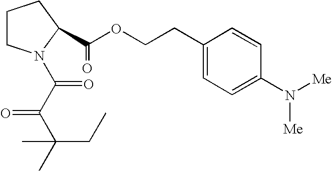 |
| XIII | 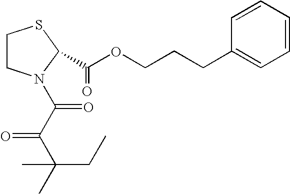 |
| XIV | 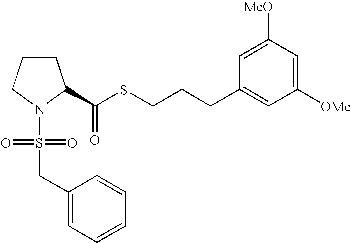 |
| XV | 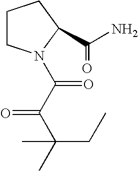 |
| XVI | 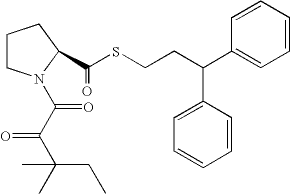 |
| XVII | 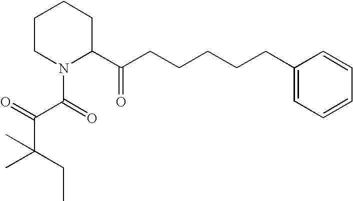 |
| XVIII | 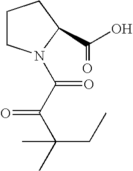 |
| XIX | 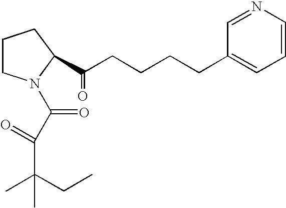 |
| XX | 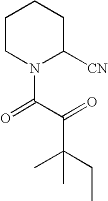 |
| XXI | 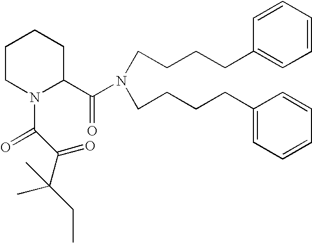 |
| XXII | 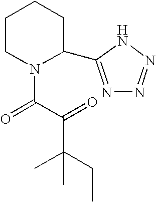 |
| XXIII | 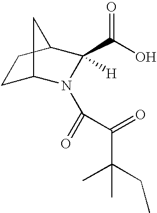 |
| XXIV | 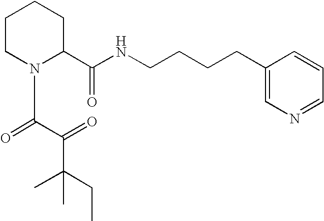 |
| XXV | 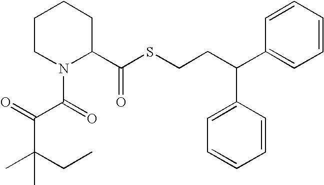 |

Example 30 addresses the effect of Compound I administration on hair cells in a cochlear explant culture system. Examples 31 and 32 address the effects of administration of Compound I on hair cells in the cochlea of guinea pigs treated with clinically relevant ototoxic therapeutic agents such as neomycin and cisplatin. The organ of Corti explant culture studies and those of the animal model of deafness clearly demonstrate that the sensorineurotrophic compound protects the hair cells of the organ of Corti against ototoxin-induced degeneration and loss of hearing.

### Example 30

### Materials

The following materials and methods were used in the Examples:

Organ of Corti Dissecting Solution:

Dulbecco's Phosphate Buffered Saline (“D-PBS”; 1×, without calcium chloride, without magnesium chloride. Cat. #14190-136, Life Technologies, Inc., Gibco BRL, Rockville, Md. 20850), containing 1.5 g/L D-Glucose (Dextrose. Cat. #15023-021, Life Technologies, Inc., Gibco BRL, Rockville, Md. 20850).

Organ of Corti Explant Culture Medium

1. High glucose Dulbecco's Modified Eagle Medium (“DMEM”; 1×, with L-glutamine, without sodium pyruvate. Cat. #11965-084, Life Technologies, Inc., Gibco BRL, Rockville, Md. 20850)

2. 0.15 g/100 ml of D-Glucose (Dextrose. Cat. #15023-021, Life Technologies, Inc., Gibco BRL, Rockville, Md. 20850)

3. 1% N-2 Supplement (100×, Cat. #17502-030, Life Technologies, Inc., Gibco BRL, Rockville, Md. 20850)

4. 100 Units/ml of Penicillin G, Potassium (Penicillin; Cat. #21840-020, Life Technologies, Inc., Gibco BRL, Rockville, Md. 20850)

### Methods

### Preparation of Medium

DMEM was supplemented with 1% N-2 supplement, and D-glucose was added to a final concentration of 1.5 g/L. Penicillin was added at 100 Units/ml. After mixing, the medium was filtered and kept at 4° C. The medium was prepared fresh just before use to minimize inter-experimental variations. Plastic pipettes and containers were used throughout to minimize protein adsorption.

### Dissecting tools and culture dishes

1. The 4″ and 5″ dissecting forceps and 4″ dissecting scissors were from Roboz Surgical, Washington, D.C.

2. Falcon sterile 96-well microplates (Flat Bottom. Cat. #3072), tissue culture plasticware and polypropylene centrifuge tubes were from Becton-Dickinson, Lincoln Park, N.J.

### Product Solutions

The sensorineurotrophic compound stock solution was stored at room temperature and prepared fresh for each culture. The stock solution was diluted in 10 μl of 100% EtOH for every milligram of sensorineurotrophic compound in the stock solution (approximately 250 mM). This solution of 250 mM sensorineurotrophic compound in 100% EtOH was diluted in normal culture medium to working concentrations of 50000 nM, 5000 nM, 500 nM, 50 nM, 5000 pM, 500 pM, 50 pM, 10 pM, 5 pM, 1 pM, 0.5 pM, 0.1 pM, and 0.01 pM. Ten microliters of ten-fold concentrated sensorineurotrophic compound product solutions were added to Organ of Corti explant cultures containing ototoxin medium (90 μl), so that the final sensorineurotrophic compound concentrations were 5000 nM, 500 nM, 50 nM, 5 nM, 500 pM, 50 pM, 5 pM, 1 pM, 0.5 pM, 0.1 pM, 0.05 pM, 0.01 pM, and 0.001 pM. Control cultures received normal medium (10 μl). The sensorineurotrophic compound treatments were initiated at first day culture (one day before ototoxin treatment), and repeated with ototoxin treatment at second day.

### Ototoxins and Related Reagents

1. Neomycin solution (Cat. #N1142, Sigma, St. Louis, Mo.) was used at final concentration of 0.6 mM. A fresh solution was made for each experiment by adding 90 μl of 1 mg/ml neomycin to 1410 μl medium.

2. Cisplatin (Platinol-AQ., Cat. #NDC 0015-3220-22, Bristol-Myers Squibb Laboratories, Princeton, N.J.) was used at a final concentration of 35 μg/ml. A fresh solution was prepared for each experiment by adding 52.5 μl of 1 mg/ml cisplatin to 1447.5 μl medium.

3. Triton X-100 (t-Octylphenoxypoly-ethoxyethanol. Cat. #X-100, Sigma., St. Louis, Mo.)

4. Phalloidin (FITC Labeled., Cat. #P-5282, Sigma, St. Louis, Mo.)

5. Vectashield (Mounting Medium, Cat. #H-1000, Vector Laboratories, Inc., Burlingame, Calif.)

### Preparation of Rat Organ of Corti Explant

Organ of Corti explants were obtained from P3-P4 Wistar rats. Rats were decapitated, the lower jaw was cut out and skin removed. The temporal bone was collected in dissection solution, the otic capsule exposed and the bony-cartilaginous cochlear capsule was carefully separated from the temporal bone. Freed cochlea were transferred to another Petri dish with dissection solution for further dissection. Intact organs of Corti were obtained by using a fine forceps to hold central VIII nerve tissue and remove it out, then the stria vascular membrane was carefully stripped off, starting from the apex or base. The organ of Corti then was transferred to a 35-mm diameter Petri dish containing cold PBS supplemented with glucose and was ready to be cultured.

### Cochlea Explant Culture Procedure

Cochlea explants were cultured in uncoated 96 microplates. A single organ of Corti was placed in a well and was kept floating in the medium. Explants were kept in normal medium for 24 hours (90 μl/well). The sensorineurotrophic compound solution (10 μl) was added to the “treated” cultures and 10 μl medium was added to controls cultures. After 24 hours of incubation, the media were changed and the explants were exposed to ototoxin-containing medium (90 μl), with sensorineurotrophic compound solution (10 μl) or without (control). The cultures were incubated for an additional 3 days. The explants were then fixed with 4% paraformaldehyde in 0.1 M D-PBS for 30 minutes at room temperature and processed for immunostaining.

### FITC-Phalloidin Staining of Hair Cells

To identify and count hair cells in the organ of Corti, a direct immunostaining method was used to label the actin present naturally in the stereocilia bundles of the hair cells. The explants were washed three times with D-PBS (200 μl per well) and permeabilized with 1% Triton X-100 in D-PBS for 15 minutes at room temperature. After three washes in D-PBS, the explants were incubated with FITC-labeled Phalloidin (1:60 from stock, 50 μl/well) for 45 minutes at room temperature. The plates were covered with aluminum foil because the Phalloidin is light sensitive. After three more washes with D-PBS, the labeled explants were placed in a drop of glycerol on a microscope slide, covered with a glass coverslip and sealed with nail polish. The explants were observed under a Nikon Diaphot-300 inverted fluorescence microscope, using FITC filters and fluorescence optics.

### Determination of Hair Cell Number

For each experiment, 2 to 4 cochlea were used. In each cochlea, the number of hair cells was counted in 2-3 sections, 175 μm in length each. Only the sections in the middle turn of the cochlea were analyzed. Each experiment was repeated several times. The number of hair cells in control and cisplatin- or neomycin-treated cultures was generated from analyzing 40 cochlea experiments.

### Results

Hair cells in the floating explant cultures did not die during the experiment period of four days. Thus, the number of phalloidin-stained cells present at the end of the 4 days experiment period, in the absence of ototoxins and treatments, was 105.4±6.9 (n=28). Ototoxins added to the explants on the second day post-plating caused a very significant loss in hair cell number found after 4 days in vitro. Exposure to 35 g/ml cisplatin 24 hours after plating caused a loss of more than 80 percent of the hair cells: only 17.6%±5.1 (n=20) of the initial number of hair cells survived and after exposure to 0.6 mM neomycin, only 5.0%±3.8 (n=26) of the hair cells survived. There was a marked difference in the morphology of the organs of Corti between this two treatments: while the treatment with neomycin resulted in almost-complete loss of hair cells, those that were spared were still organized in the typical four row structure (3 rows of outer hair cells and one row of inner hair cells). Cisplatin treatment, on the other hand, caused a marked disruption of the four-row-structure and the surviving cells were randomly located, indicating a damage caused also to the supporting cells underlying the hair cells.

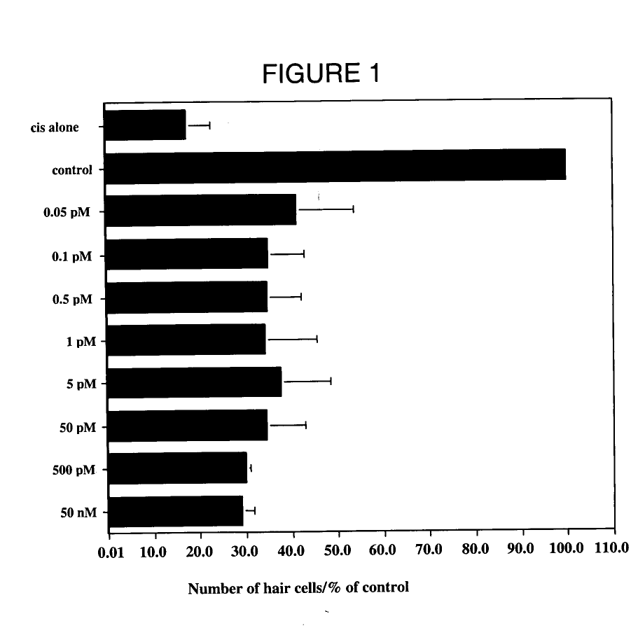

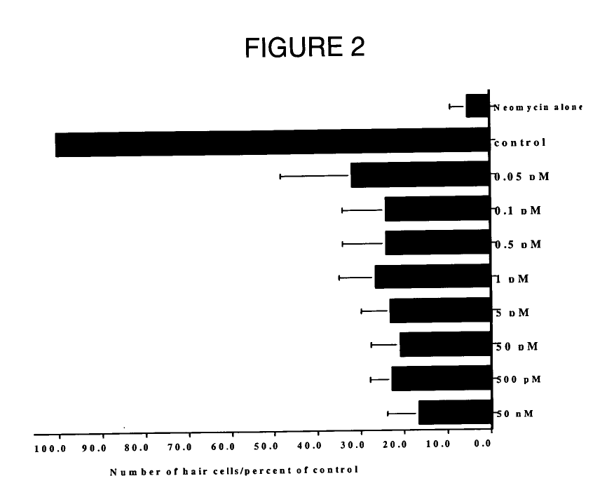

In cultures that received Compound I at the time of plating (pretreatment), a significantly higher number of hair cells survived the 3-day exposure to ototoxins (from day 2 to day 4) compared to cultures containing the ototoxin alone. In cultures exposed to cisplatin (FIG. 1), treatment with Compound I at concentrations as low as 0.05 pM resulted in an increase in surviving hair cells from the 17% of the untreated to 41.4%. This, however, was already the maximal activity of Compound I as the effect did not titrate out along the range of concentration tested (0.05 pM-50 nM). Cultures that received neomycin showed a reduction of 95% in hair cells compared to controls. Treatment with Compound I together with the neomycin reduced this loss to around 70% (31.8%±16.4 surviving hair cells) at a concentration of 0.05 pM, an effect which again, did not titrate out nor was increased with higher concentrations of Compound I.

### Example 31

### Protection by Compound I of Hair Cells Against Intramiddle Ear Neomycin-Induced Ototoxicity

### Materials

Ototoxins—Neomycin sulfate: (Cat. #N-1876, Sigma, St. Louis, Mo.)

Vehicle—20% Intralipid: Intralipid is a 20% I.V. fat emulsion (Cat. #NDC 0338-0491-O₂, Pharmacia Inc., Clayton, N.C.). Each 100 ml contains: Soybean oil 20.0 g, Phospholipids (from powdered egg yolk) 1.2 g, Glycerin, USP 2.25 g, Water for injection qs, and pH 8.0 (6.0-8.9), adjusted with sodium hydroxide.

Ethyl alcohol: 200 proof dehydrated alcohol, USP (Quantum Chemical Company, Tuscola, Ill.)

Saline solution: 0.9% sterile sodium chloride aqueous solution (Cat #NDC 57319-077-06, Phoenix Pharmaceutical, Inc., St. Joseph, Mo.)

Gelfoam: absorbable gelatin sponge, USP (Cat. #NDC 0009-0396-01, Upjohn, Kalamazoo, Mich.)

Guinea pigs: Female pigmented guinea pigs (more sensitive than albino to ototoxicity induced by aminoglycoside antibiotics) from NIH, body weight: 300-400 g

Phalloidin: FITC Labeled. (Cat. #P-5282, Sigma, St. Louis, Mo.)

Vectashield: Mounting Medium. (Cat. #H-1000, Vector Laboratories, Inc., Burlingame, Calif.)

### Methods

### The First Middle Ear Administration of Sensorineurotrophic Compound

Twenty guinea pigs used in this study were divided into two groups: 10 animals received 10 ng and 10 received 1 ng of the sensorineurotrophic compound.

Preparation of the Sensorineurotrophic compound: On the day of use, sensorineurotrophic compound stock solutions were prepared fresh as follows:

Sensorineurotrophic compound stock A: A stock solution of sensorineurotrophic compound at 1 mg/10 ml in 100% ethanol was prepared and then diluted and mixed in Intralipid at 10 ng/100 μl.

Sensorineurotrophic compound stock B: A stock solution of sensorineurotrophic compound at 1 mg/100 ml in 100% ethanol was prepared and then diluted and mixed in 20% Intralipid at 1 ng/100 μl.

### The Middle Ear Administration

Animals were anesthetized with an intramuscular injection of a mixture of ketamine (80 mg/kg) and xylazine (4 mg/kg). Through a post-auricular incision, the right bulla was identified. A hole was drilled to open the middle ear cavity (care was taken not to injure the tympanic annulus or ossicles). A piece of gelfoam (˜2 mm³) soaked with the sensorineurotrophic compound solution was inserted into the round window niche. The remaining sensorineurotrophic compound solution (˜100 μl) then was injected into the middle ear cavity. In the 10 ng dose group, 100 μl of sensorineurotrophic compound stock A was administered; and in the 1 ng dose group, 100 μl of sensorineurotrophic compound stock B was administered to the middle ear cavity. The hole was covered with a piece of clear plastic sheet which was stuck on the skull with a superglue. The incision was closed with clips. The same procedure was performed at the left bulla, but 100 μl of vehicle solution instead of sensorineurotrophic compound solution was administered. The animals were maintained in the prone position until they woke up to ensure filling of the middle ear cavity.

### The Second Middle Ear Administration of Sensorineurotrophic Compound and Neomycin Ototoxin

After two days, the animals received a second middle ear administration of sensorineurotrophic compound or vehicle together with neomycin. Solutions were prepared for the two groups of animals as follows:

Solution A: Neomycin was dissolved in sensorineurotrophic compound stock A described above. The final concentration of neomycin was 5 mg and the sensorineurotrophic compound concentration was 10 ng in a 100 μl vehicle solution.

Solution B: Neomycin was dissolved in sensorineurotrophic compound stock B described above. The final concentration of neomycin was 5 mg and the concentration of sensorineurotrophic compound was 1 ng in a 100 μl vehicle solution.

Solution C: Neomycin was dissolved in 20% Intralipid to a final concentration of 5 mg in 100 μl vehicle.

The plastic cover sheet on the bulla window was removed and the middle ear cavity was exposed. The old sensorineurotrophic compound or vehicle was sucked off and the old gelfoam was removed from the round window niche. A piece of gelfoam with fresh stock solution containing sensorineurotrophic compound and neomycin was administered to the round window niche, and the remaining solution (˜100 μl, solution A for the 10 ng dose group and solution B for the 1 ng dose group, respectively) was injected into the middle ear cavity of the right bulla.

Solution C (100 μl) was administered to the left ear for both groups in the same way.

The animals were maintained in the prone position until waking up to ensure filling of the middle ear cavity.

### Perfusion And Fixation

Fourteen days after the second surgery, animals were perfused transcardially with a PBS flush followed by a fixative of 4% paraformaldehyde in 0.1M PBS. Immediately following the perfusion, the temporal bone was removed from the head. The bulla was opened and the cochlea was exposed. The apex was opened and the membrane of the round and oval windows was punched. The fixative solution was gently infused into the perilymphatic space through the apex hole and then allowed to flow out from windows. Then the cochleae were post-fixed in the same fixative solution for at least one day.

### FITC-Phalloidin Staining of Hair Cells

To identify and count hair cells in the organ of Corti, a direct immunostaining method was used to label the actin present naturally in the stereocilia bundles of the hair cells. The cochlea was dissected and the perilymphatic space was fully exposed. The samples were washed three times with PBS (1 ml per well) and permeabilized with 1% Triton X-100 in PBS for 10 min minutes at room temperature. After three washes in PBS, the cochlea samples were incubated with FITC-labeled Phalloidin (1:60 from stock, i.e. 1.67 μg/ml in concentration, 1 ml/well) for 45 minutes at room temperature. The plates were covered with aluminum foil because the Phalloidin is light sensitive. After three more washes with PBS, the labeled cochleas were then bisected and all four turns were removed by microdissection, preserving the hook portion of the basal turn. The turns were mounted on a coverslip (24×60 mm) with Vectashield mounting medium, covered with a glass coverslip and sealed with nail polish. The cochlea turns were observed under a Nikon Diaphot-300 inverted fluorescence microscope, using FITC filters and fluorescence optics.

### Determination of Hair Cell Number

The cochlea turns were observed under a Nikon Diaphot-300 inverted fluorescence microscope, using FITC filters and fluorescence optics. In each cochlea, the number of missing outer hair cells (“OHC”) was counted in each 175 μm segment (containing 20 OHCs in each row of OHC) beginning from the apex and continuing toward the base. The numbers were filled in a cochleogram form for analysis of the percentage of OHC loss in each row, each turn and in whole cochlea of left and right ears. There are four turns per cochlea, the apex called turn 1 is counted from the top 3.5 mm in length, middle turns including turns 2 (counted 3.5 mm-7.0 mm from apex) and turn 3 (7.0 mm-10.5 mm from apex), and the basal turn called turn 4 (10.5 mm-14.0 mm).

### Results

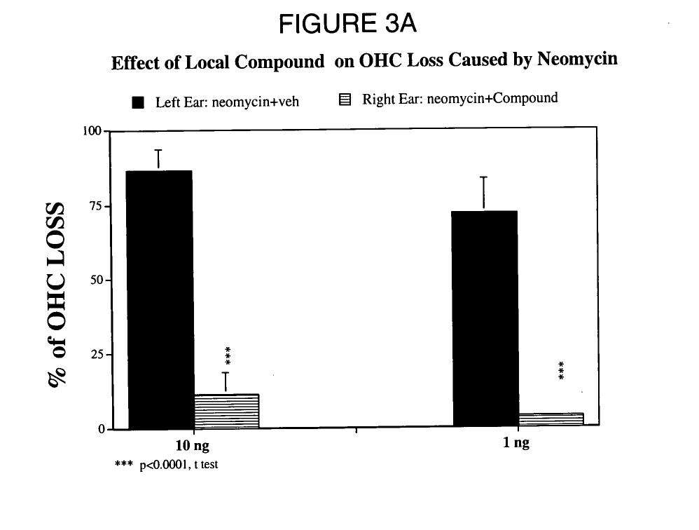

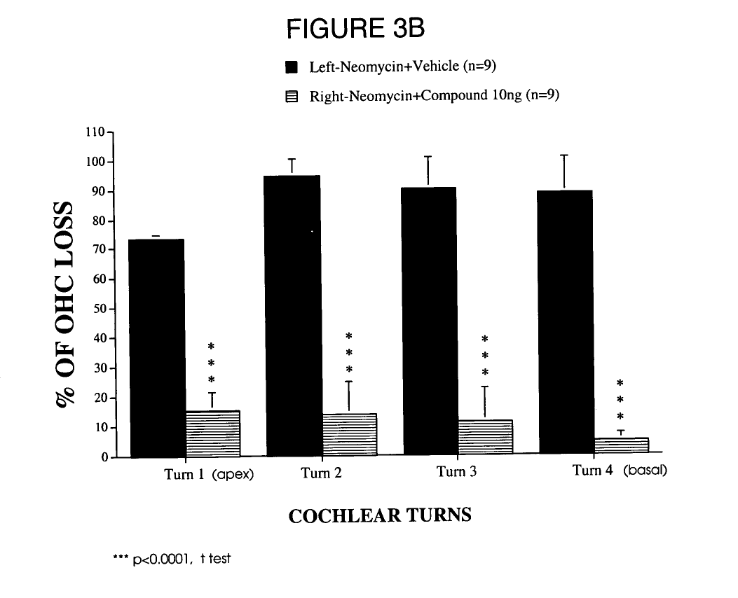

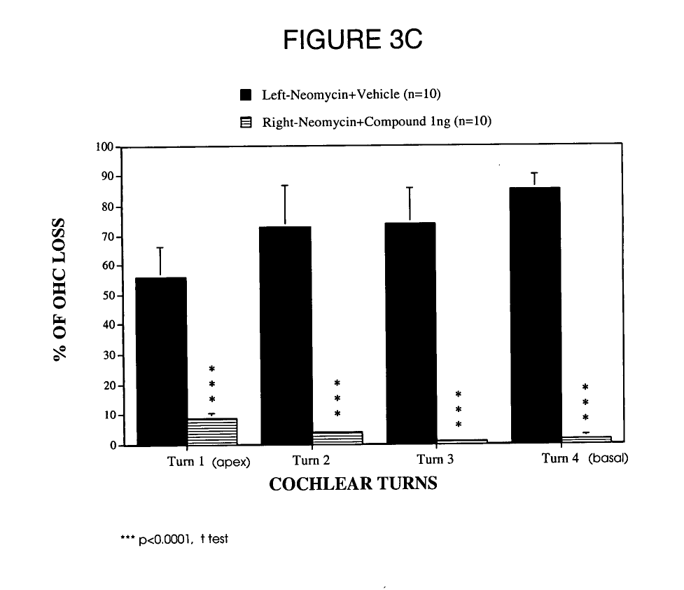

Table XLVI and FIG. 3A show that there was a large and significant (p<0.0001, t-test) difference in the number of OHCs lost betweem vehicle and sensorineurotrophic compound treated animals after exposure to ototoxins. Treatment with either 10 ng or 1 ng of sensorineurotrophic compound are around 75% and 70% of hair cells respectively. Maximal protective activity was on the basal turns (FIGS. 3B and 3C). The results indicate that under this experimental paradigm the sensorineurotrophic compound was able to protect completely hair cells against ototoxicity.

TABLE XLVI
Protection against Neomycin-induced OHC Loss (%) in
Intramiddle Ear Administered Models

| | Left - | Right - | |
|---|---|---|---|
| | vehicle | (Treated) | |
| Treatment | mean ± SEM | mean ± SEM | t-test |
| 10 ng (n = 9) | 86.78 ± 6.81 | 11.44 ± 7.27 | p < 0.0001 |
| turn-1 | 73.36 ± 1.12 | 15.47 ± 6.05 | p < 0.0001 |
| turn-2 | 94.72 ± 5.59 | 13.93 ± 10.75 | p < 0.0001 |
| turn-3 | 90.10 ± 10.50 | 11.64 ± 10.85 | p < 0.0001 |
| turn-4 | 88.94 ± 11.73 | 4.69 ± 2.85 | p < 0.0001 |
| 1 ng (n = 7) | 72.14 ± 11.19 | 3.86 ± 0.37 | p < 0.0001 |
| turn-1 | 56.11 ± 9.90 | 8.85 ± 1.47 | p < 0.0001 |
| turn-2 | 72.96 ± 13.97 | 4.01 ± 0.53 | p < 0.0001 |
| turn-3 | 74.07 ± 11.59 | 0.92 ± 0.10 | p < 0.0001 |
| turn-4 | 85.43 ± 4.82 | 1.67 ± 1.25 | p < 0.0001 |

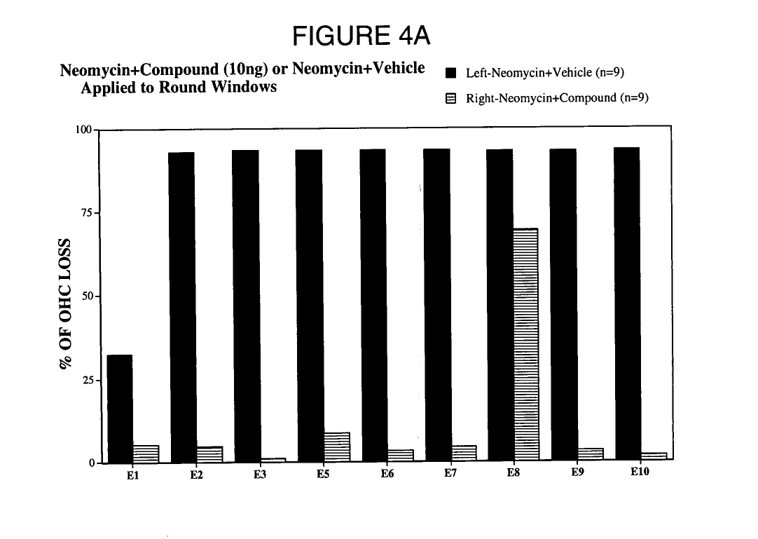

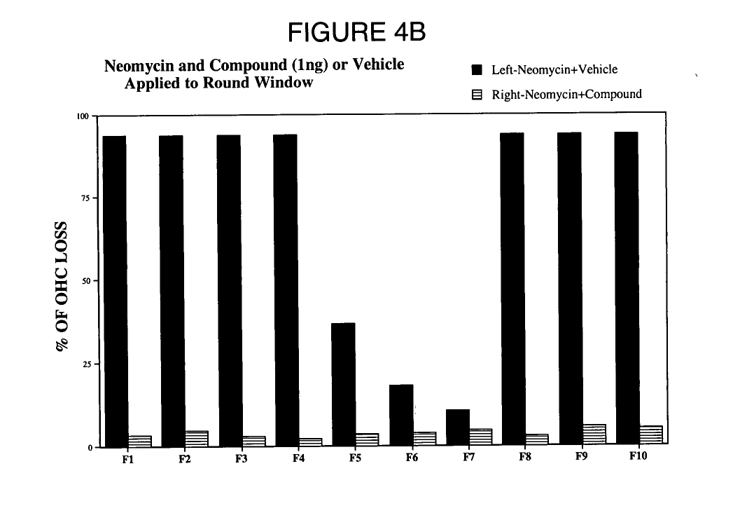

Intramiddle ear administered neomycin caused a marked disruption of the four-row-structure and the surviving cells were randomly located. Treatment with neomycin and vehicle resulted in almost complete loss of hair cells in most animals. There was a very minimal loss of hair cells in all the animals treated with sensorineurotrophic compound at 1 ng and all but one, in the group treated with 10 ng. (FIGS. 4A and 4B).

### Example 32

### Protection of Hair Cells Against Ototoxicity Induced by Intramiddle Ear Administration of Neomycin by Systemically Administered Sensorineurotrophic Compound I

### Methods and Materials

The materials used are those described in Example 1.

### Systemic Administration of Sensorineurotrophic Compound

Twenty guinea pigs were treated either with sensorineurotrophic compound or vehicle prior to administration of the ototoxin. Ten of the guinea pigs were subcutaneously injected with freshly made sensorineurotrophic compound solution. On the day of injection, 100 mg of the sensorineurotrophic compound was dissolved in 1 ml of ethanol, then 20% of the Intralipids solution was added to make a final volume of 3 ml. The final sensorineurotrophic compound concentration was 10 mg/0.3 ml. Each animal was subcutaneously injected with 0.3 ml of the sensorineurotrophic compound solution at day 0, day 2 and day 7. Another 10 animals were subcutaneously injected with 0.3 ml of the vehicle (20% Intralipids), individually at day 0, day 2 and day 7.

### Middle Ear Administration of Neomycin

At day 2, guinea pigs used in this study were administered neomycin or neomycin vehicle in the middle ear.

Animals were anesthetized with intramuscular injection of a mixture of ketamine (80 mg/kg) and xylazine (4 mg/kg). Through a post-auricular incision, the right bulla was identified. A hole was drilled to open the middle ear cavity (care was taken not to injure the tympanic annulus or ossicles). A piece of gelfoam (˜2 mm³) was soaked with neomycin solution (fresh made at a concentration of 50 mg/ml) and was inserted into the round window niche. The remaining neomycin solution (˜100 μl) was then injected into the middle ear cavity. A total of 5 mg of neomycin was applied to the right middle ear. The hole was covered with a clear plastic sheet and stuck on the skull with superglue. The incision was closed with clips. The same procedure was performed at the left ear, but vehicle solution (100 μl of 0.9% saline) was administered instead of neomycin. To ensure filling of the middle ear cavity, the animals were maintained in the prone position until they woke up.

### Perfusion and Fixation

On the 16th day, animals were perfused transcardially with a PBS flush following by a fixative of 4% paraformaldehyde in 0.1M PBS. Immediately following the perfusion, the temporal bone was removed from the head. The bulla was opened and the cochlea was exposed. The apex was opened and the membrane of the round and oval windows was broken. The fixative solution was infused into the perilymphatic space of the cochlea, and the fixative solution was gently irrigated through the apex hole and then allowed to flow out from the windows. The cochleae then were post-fixed in the same fixative solution for at least one day.

The staining and counting of hair cells was performed in the same manner as described in Example 2.

### Results

### Protective Effects of Systemically Administered Sensorineurotrophic Compound Against Neomycin-Induced Hair Cell Loss

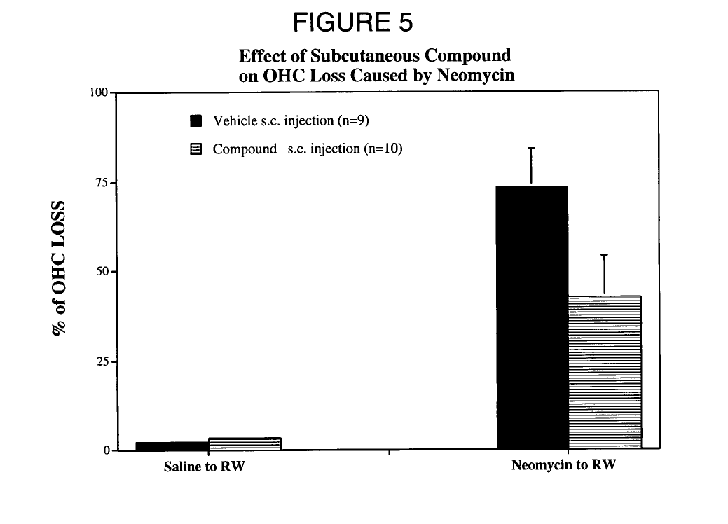

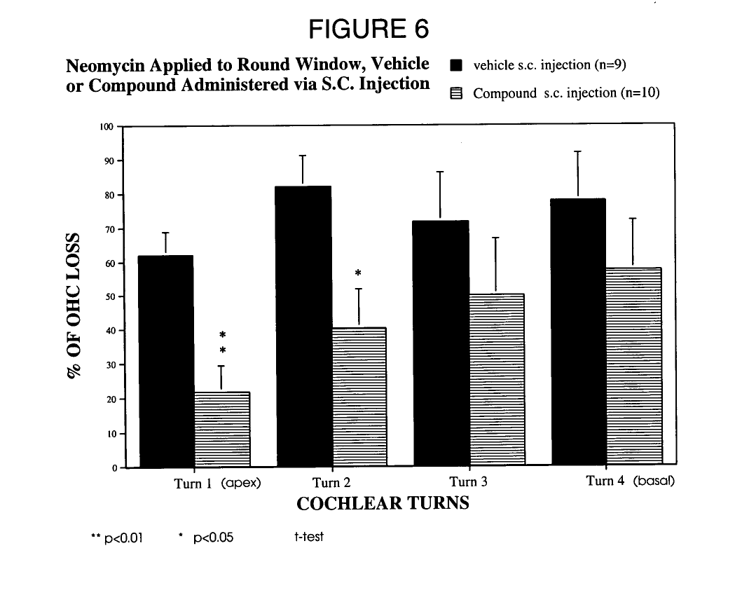

There was a significant difference in the loss of hair cells between vehicle and sensorineurotrophic compound treated animals (˜31%, FIG. 5). While neomycin alone in the vehicle treated animals induced about 75% of hair cell loss, treatment with the sensorineurotrophic compound resulted in a loss of only about 45%. This significant protection was observed on the apex turns and top middle turns (FIG. 6).

### Example 33

### Compound XVI Protects Hair Cells Against Intramiddle Ear Neomycin-Induced Ototoxicity

### Materials

The materials used in the following Example were obtained as follows:

Ototoxins—Neomycin sulfate: (Cat. #N-1876, Sigma, St. Louis, Mo.)

Vehicle—20% Intralipid: Intralipid is a 20% I.V. fat emulsion (Cat. #NDC 0338-0491-O₂, Pharmacia Inc., Clayton, N.C.). Each 100 ml contains: Soybean oil 20.0 g, Phospholipids (from powdered egg yolk) 1.2 g, Glycerin, USP 2.25 g, Water or injection qs, and Calories 200 kcal. pH 8.0 (6.0-8.9), adjusted with sodium hydroxide.

Ethyl alcohol: 200 proof Dehydrated alcohol, USP (Quantum Chemical Company, Tuscola, Ill.)

Saline solution: 0.9% sterile sodium chloride aqueous solution (Cat #NDC 57319-077-06, Phoenix Pharmaceutical, Inc., St. Joseph, Mo.)

Gelfoam: absorbable gelatin sponge, USP (Cat. #NDC 0009-0396-01, Upjohn, Kalamazoo, Mich.)

Guinea pigs: Female pigmented guinea pigs (more sensitive than albino to the ototoxicity induced by aminoglycoside antibiotics) from NIH, body weight: 300-400 g

Phalloidin: FITC Labeled. Louis, Mo.) (Cat. #P-5282, Sigma. St.

Vectashield: mounting Medium. (Cat. #H-1000, Vector, Burlingame, Calif.)

### Methods

### The First Middle Ear Administration of Compound XVI

Ten guinea pigs were used in this study. Each animal received 10 ng of Compound XVI in one ear and vehicle in the other.

Preparation of Compound XVI:

Compound XVI Stock A Solution: A solution of Compound XVI at 1 mg/10 ml in 100% ethanol was firstly prepared and then it was diluted and mixed in Intralipid at 10 ng/100 μl.

This stock solution was made fresh daily, and discarded after use.

The vehicle was 20% Intralipid.

### Middle Ear Administration

Animals were anesthetized with intramuscular injection of a mixture of ketamine (80 mg/kg) and xylazine (4 mg/kg). Through a post-auricular incision, the right bulla was identified. A hole was drilled to open the middle ear cavity (care was taken not to injure the tympanic annulus or ossicles). A piece of gelfoam (˜2 mm³) was soaked with Compound XVI solution and was inserted into the round window niche. The remaining Compound XVI solution (˜100 μl) was then injected into the middle ear cavity. The hole was covered with a piece of clear plastic sheet which was glued to the skull with a superglue. The incision was closed with clips. The same procedure was performed at the left bulla, but administered with 100 μl of vehicle solution instead of Compound XVI. The animals were maintained in the prone position until they woke up to ensure filling of the middle ear cavity.

### The Second Middle Ear Administration of Compound XVI and Neomycin Ototoxin

After two days, the animals received the second administration of Compound XVI or vehicle together with neomycin in the middle ear. Solutions were prepared for the two groups of animals as following:

Solution A: Neomycin was dissolved in Compound XVI stock A solution, described above. The final concentration of neomycin was 5 mg and the Compound XVI was 10 ng in a 100 μl vehicle solution.

Solution B: Neomycin was dissolved in 20% Intralipids to a final concentration of 5 mg in 100 μl vehicle.

When the incision was reopened, the plastic cover sheet on the bulla window was removed. The old Compound XVI or vehicle was sucked off with a vacuum device and the old gelfoam was removed from the round window niche. A piece of gelfoam with fresh stock solution containing Compound XVI and neomycin was administered to the round window niche, and the remaining solution (˜100 μl), was injected to the middle ear cavity of the right bulla.

Solution B (100 μl) was administered to the left ear for both groups in the same way.

The animals were maintained in the prone position until waking up to ensure filling of the middle ear cavity.

### Perfusion and Fixation

Fourteen days after the second surgery, animals were perfused transcardially with a PBS flush following by a fixative of 4% paraformaldehyde in 0.1M PBS. Immediately following the perfusion, the temporal bone was removed from the head. The bulla was opened and the cochlea was exposed. The apex was opened and the membrane of the round and oval windows was punched. The fixative solution was gently infused into the perilymphatic space through the apex hole and then allowed to flow out from the windows. Then the cochleae were post-fixed in the same fixative solution for at least one day.

### FITC-Phalloidin Staining of Hair Cells

To identify and count hair cells in the organ of Corti, a direct immunostaining method was used to label the actin present naturally in the stereocilia bundles of the hair cells. The cochlea was dissected and the perilymphatic space was fully exposed. The samples were washed three times with PBS (1 ml per well) and permeabilized with 1% Triton X-100 in PBS for 10 min minutes at room temperature. After three washes in PBS, The cochlea samples were incubated with FITC-labeled Phalloidin (1:60 from stock, i.e. 1.67 μl/ml in concentration, 1 ml/well) for 45 minutes at room temperature. The plates were covered with aluminum foil as the Phalloidin is light sensitive. After three more washes with PBS, the labeled cochleas were then bisected and all four turns were removed by microdissection, preserving the hook portion of the basal turn. The turns were mounted on a coverslip (24×60 mm) with Vectashield mounting medium, covered with a glass coverslip and sealed with nail polish. The cochlea turns were observed under a Nikon Diaphot-300 inverted fluorescence microscope, using FITC filters and fluorescence optics.

### Determination of Hair Cell Number

The cochlea turns were observed under a Nikon Diaphot-300 inverted fluorescence microscope, using FITC filters and fluorescence optics. In each cochlea, the number of missed outer hair cells (OHC) was counted in each 175 μm segment (containing 20 OHCs in each row of OHC) beginning from the apex and continuing toward the base. The numbers were filled in a cochleogram form for analysis of the percentage of OHC loss in each row, each turn and in whole cochlea of left and right ears. There are four turns per cochlea, the apex called turn 1 is counted top 3.5 mm in length, middle turns including turns 2 (counted 3.5 mm-7.0 mm from apex) and turn 3 (7.0 mm-10.5 mm from apex), and the basal turn called turn 4 (10.5 mm-14.0 mm).

### Results

Compound XVI Protects OHC Loss (%) in Intramiddle Ear Administered Neomycin-Induced Hearing Loss Models

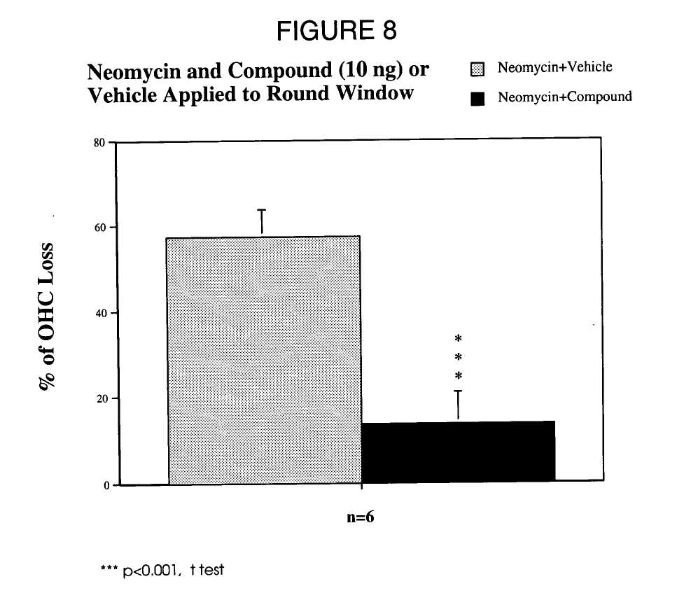

FIG. 8: Comparison between hair cell number in ears treated with neomycin and vehicle and ears treated with neomycin and Compound XVI—mean of a group.

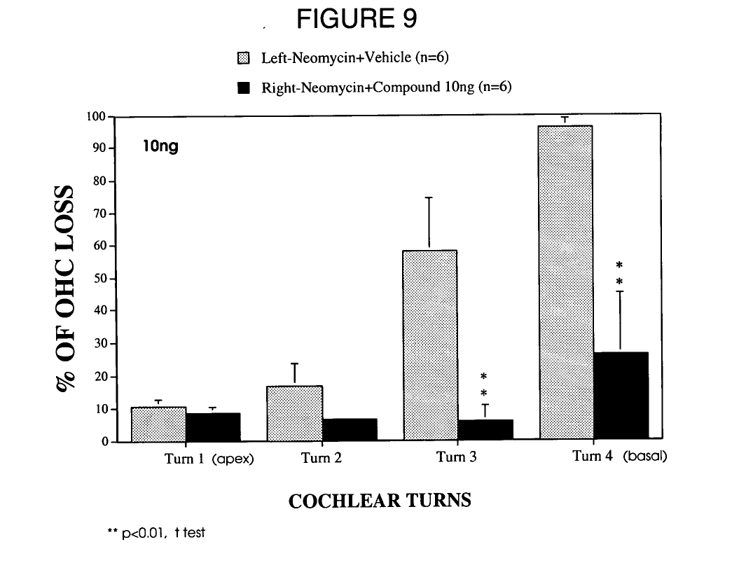

FIG. 9: comparison between hair cell number in ear treated with neomycin and vehicle and ear treated with neomycin and Compound XVI—separation into the four turns of the cochlea

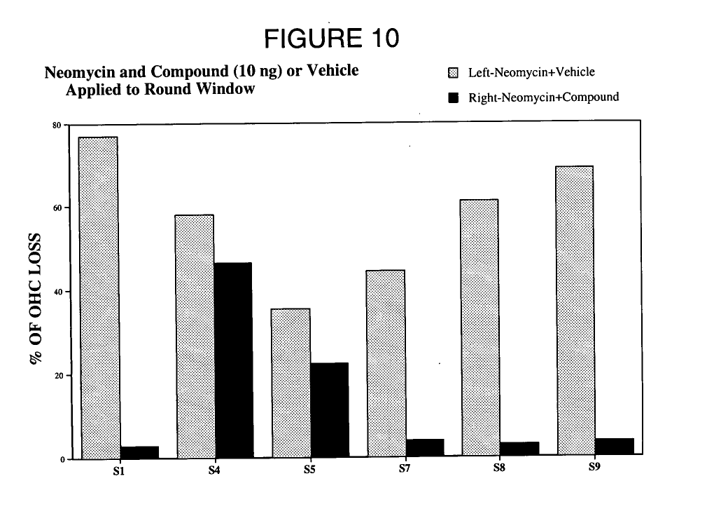

FIG. 10: comparison between hair cell number in ear treated with neomycin and vehicle and ear treated with neomycin and Compound XVI—individual animals

FIG. 8 demonstrates that there was a marked difference (over 50%, p<0.0001, t-test) in the number of OHCs lost in animals treated with vehicle and Compound XVI when exposed to neomycin. FIG. 10 demonstrates the variability between individual animals in the group regarding both the ototoxicity and protection. In 4 out of the 6 animals, there was a complete loss of outer hair cells in the cochlea (S1; S7; S8 and S9)[note—in the figures, “S”, “E” or “F”, or any other letter, followed by a number is a code designation for a particular animal]. The two others had smaller losses: around 50% (S4) and around 25% (S5). In each one of these animals, however, there were more hair cells found in the GPI treated ear than the one treated with vehicle. This protection effect ranged between a minimum of 10% (S5) and maximum of 85% (S1). FIG. 9 demonstrates that the biggest loss of hair cells was found in the basal turn and the second turn (the adjacent turn) of the cochlea, as previously known for the effect of ototoxins in the inner ear. Even in those turns, where hair cells are the most vulnerable, Compound XVI was able to completely prevent the loss in the second turn (turn 3) and to reduce it from almost 100% to around 30%, in the basal turn (turn 4).

### Example 34

### Systemic Administered Compound XXV Protects Hair Cells Against Ototoxicity Induced by Cisplatin

### Materials

The materials used in the this Example were as follows:

Vehicle—20% Intralipid: Intralipid is a 20% I.V. fat emulsion (Cat. #NDC 0338-0491-O₂, Pharmacia Inc., Clayton, N.C.). Each 100 ml contains: Soybean oil 20.0 g, Phospholipids (from powdered egg yolk) 1.2 g, Glycerin, USP 2.25 g, water for injection qs, and Calories 200 kcal. pH 8.0 (6.0-8.9), adjusted with sodium hydroxide.

Ethyl alcohol: 200 proof Dehydrated alcohol, USP (Quantum Chemical Company, Tuscola, Ill.)

Saline solution: 0.9% sterile sodium chloride aqueous solution (Cat #NDC 57319-077-06, Phoenix Pharmaceutical, Inc., St. Joseph, Mo.)

Guinea pigs: Male pigmented guinea pigs (more sensitive than albino to the ototoxicity induced by aminoglycoside antibiotics) from NIH, body weight: 150-200 g

Phalloidin: FITC Labeled. (Cat. #P-5282, Sigma, St. Louis, Mo.)

Vectashield: Mounting medium. (cat. #H-1000, Vector, Burlingame, Calif.)

Cisplatin: Platinol-AQ, in a solution of 1 mg cisplatin and 9 mg sodium chloride in water from Bristol Laboratories (Bristol-Myers. Squibb Co. Princeton, N.J. 08543).

### Methods

### Systemic Administration of Compound XXV and Cisplatin

Twenty male pigmented guinea pigs were divided into two groups (10 in each). One group was treated with Compound XXV while the other with vehicle—2 days prior to the first cisplatin injection. The test compound or vehicle was delivered by daily sub-cutaneous injection. On the day of injection, 100 mg of Compound XXV was dissolved in 1 ml of ethanol, then added to 20% Intralipid solution to a final volume of 3 ml and final Compound XXV concentration of 10 mg/0.3 ml. Each animal was subcutaneously injected with 30 mg/kg of Compound XXV solution Two days after the beginning of test compound injections (d2), cisplatin intraperitoneal injection was given to all animals at 4 mg/kg. After 3 days, a second cisplatin injection was given to all animals (d5). After another 3 days, a third injection (d8) and after 3 more days the fourth and last injection of cisplatin was given (d11). Test compound injections continued daily until day 21 (10 days after the last cisplatin injection).

### Preyers' Reflex Monitoring

Preyers' reflex is a rough indication of hearing function in rodents. In response to a noise stimuli, created by clapping hands or knocking two pieces of metal together, the pina of the ear near which the noise was created, twitches backward and than returns to its regular position. If hearing function of a ear is compromised, the twitch of the pina will be delayed and small. If the ear is deafened, the pina will not move at all in response to the sound stimuli created. The animals in this experiment were monitored daily for their Preyer's reflex.

### Perfusion and Fixation

On the 21st day, animals were perfused transcardially with a PBS flush following by a fixative of 4% paraformaldehyde in 0.1 M PBS. Immediately following the perfusion, the temporal bone was removed from the head. The bulla was opened and the cochlea was exposed. The apex was opened and the membrane of the round and oval windows was broken. The fixative solution was infused into the perilymphatic space of the cochlea, and the fixative solution was gently irrigated through the apex hole and then allowed to flow out from windows. Then the cochleae were post-fixed in the same fixative solution for at least one day.

### FITC-Phalloidin Staining of Hair Cells

Staining was performed in the same manner as in Example 4

### Determination of Hair Cell Number

Determination of hair cell numbers was determined in the same manner as in Example 4.

### Results

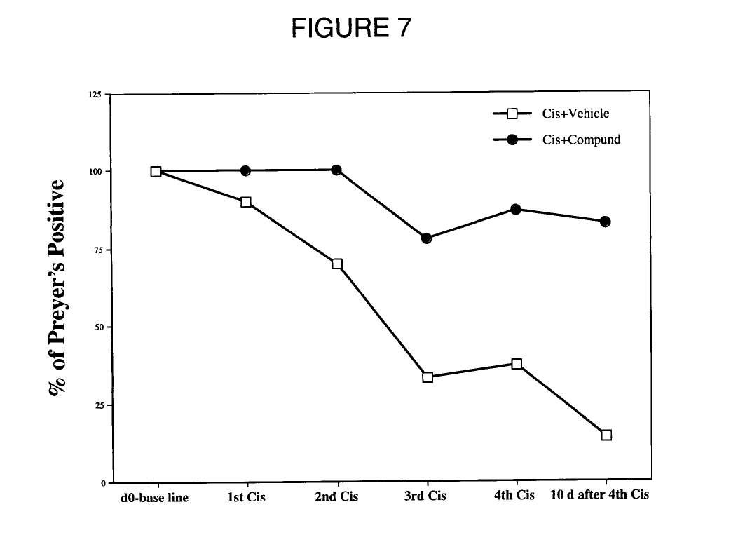

FIG. 7: Percent of animals per group responding with Preyer's reflex.

FIG. 7 demonstrates the protective effect of Compound XXV on hearing function. Already after the first cisplatin injection, there are a few animals in the vehicle treated group that lose their Preyer's reflex. The proportion of animals losing hearing increases significantly after every cisplatin injection in the vehicle treated group. In the group of animals receiving Compound XXV, on the other hand, there is some loss only after the second injection of cisplatin but it stays at that level (about 20% of the animals) even 10 days after the 4th injection while at that time, in the vehicle treated group, more than 80% of the animals have lost their Preyer's reflex.

### Example 35

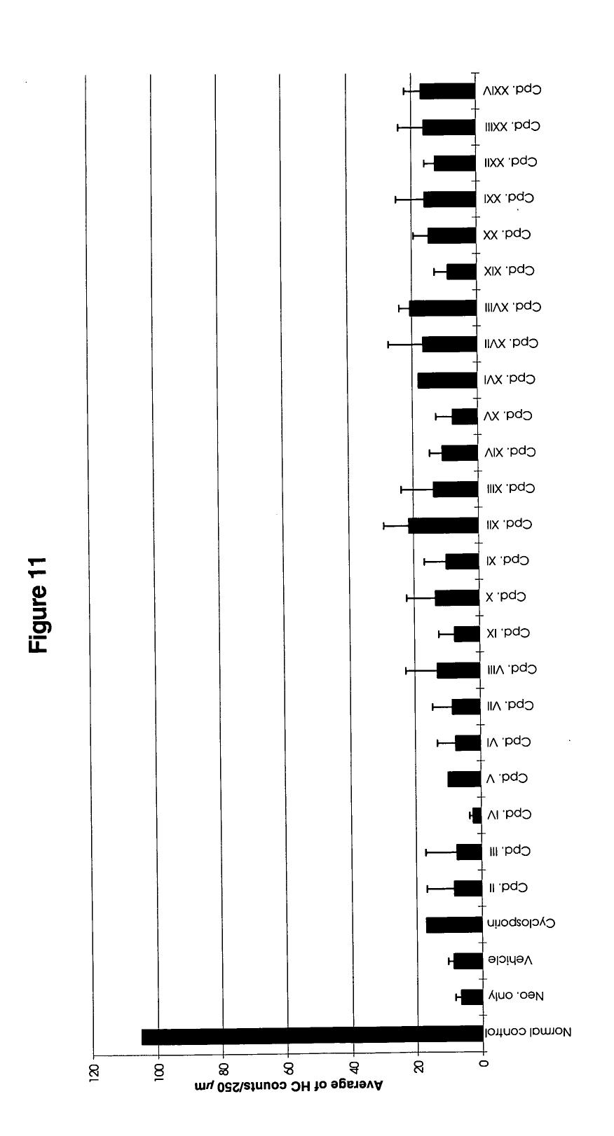

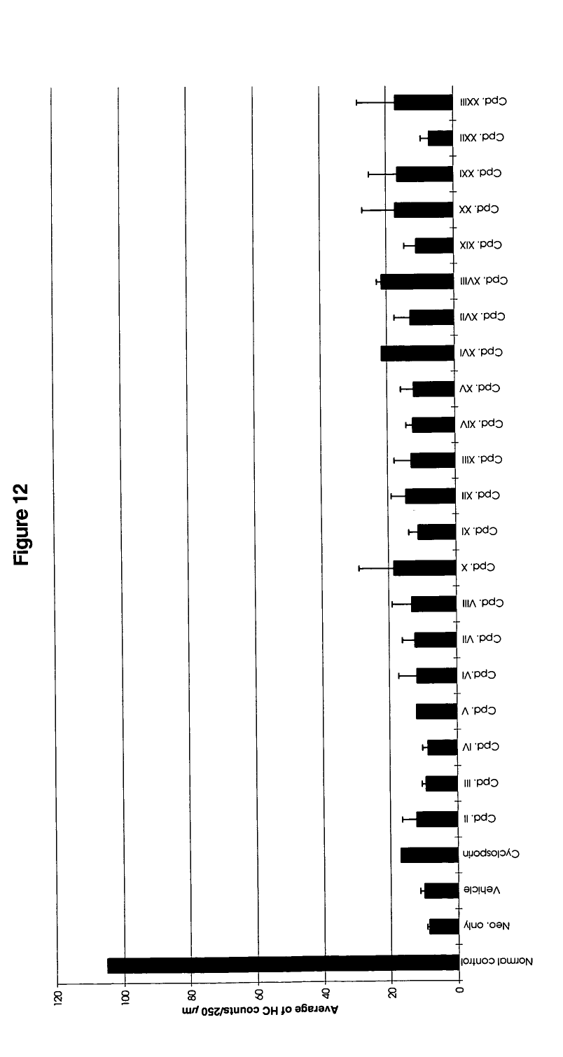

A variety of other sensorineurotrophic compounds, described in Table XLV above, were tested using the cochlear explant procedure outlined in Example 30. The compounds showed a significant enhancement in survival of hair cells relative to neomycin treated explants without the benefit of treatment of the sensorineurotrophic compound of the invention. The results of these studies are provided in FIGS. 11 (1 pM therapeutic drug concentration) and 12 (10 pM therapeutic drug concentration).

I claim:

1. A method for the prevention or treatment of sensorineural hearing loss which comprises administering to a warm-blooded animal a sensorineurotrophic compound of the formula (I′): 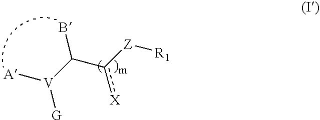
wherein
A′ is hydrogen, C₁ or C₂ alkyl, or benzyl;
B′ is C₁-C₄ straight or branched chain alkyl, benzyl or cyclohexylmethyl; or,
A′ and B′, taken together with the atoms to which they are attached, form a 5-7 membered saturated, unsaturated or aromatic heterocylic or carbocyclic ring which contains one or more additional O, C(R₁)₂, S(O)ₚ, N, NR₁, or NR₅ atoms;
V is CH, S, or N;
G is 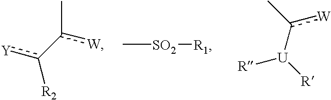
each R₁, independently, is hydrogen, C₁-C₉ straight or branched chain alkyl, or C₂-C₉ straight or branched chain alkenyl or alkynyl, C₃-C₉ cycloalkyl, C₅-C₇ cycloalkenyl, a carboxylic acid or carboxylic acid isostere, N(R₄), Ar₁, Ar₄ or K-L wherein said alkyl, cycloalkyl, cycloalkenyl, alkynyl, alkenyl, Ar₁ or Ar₄ is optionally substituted with one or more substituent(s) independently selected from the group consisting of:
2-furyl, 2-thienyl, pyridyl, phenyl, C₃-C₆ cycloalkyl wherein said furyl, thienyl, pyridyl, phenyl or cycloalkyl group optionally is substituted with C₁-C₄ alkoxy, (Ar₁)ₙ, halo, halo-C₁-C₆-alkyl, carbonyl, thiocarbonyl, C₁-C₆ thioester, cyano, imino, COOR₆ in which R₆ is C₁-C₉ straight or branched chain alkyl or alkenyl, hydroxy, nitro, trifluoromethyl, C₁-C₆ alkoxy, C₂-C₄ alkenyloxy, C₁-C₆ alkylaryloxy C₁-C₆ aryloxy, aryl-(C₁-C₆)-alkyloxy, phenoxy, benzyloxy, thio-(C₁-C₆)-alkyl, C₁-C₆-alkylthio, sulfhydryl, sulfonyl, amino, (C₁-C₆)-mono- or di-alkylamino, amino-(C₁-C₆)-alkyl, aminocarboxy, C₃-C₈ cycloalkyl, C₁-C₆ straight or branched chain alkyl, C₂-C₆ straight or branched chain alkenyl optionally substituted with (Ar₁)ₙ, C₃-C₈ cycloalkyl, C₁-C₆ straight or branched chain alkyl, C₂-C₆ straight or branched chain alkenyl substituted with C₃-C₈ cycloalkyl, C₃-C₈ cycloalkyl, and Ar₂, and, wherein any carbon atom of an alkyl or alkenyl group may optionally replaced with O, NR₅, or S(O)_{P}; or,
R₁ is a moiety of the formula: 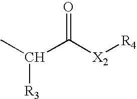
wherein:
R₃ is C₁-C₉ straight or branched chain alkyl which is optionally substituted with C₃-C₈ cycloalkyl or Ar₁;
X₂ is O or NR₆, wherein R₆ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;
R₂ is C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl, or cycloalkenyl is optionally substituted with one or more substituents selected from the group consisting of C₁-C₆ straight or branched chain alkyl, C₂-C₆ straight or branched chain alkenyl, C₃-C₈ cycloalkyl, C₅-C₇ cycloalkenyl, (Ar₁)ₙ and hydroxy; or,
R₂ is either hydrogen or P; Y is either oxygen or CH-P, provided that if R₂ is hydrogen, then Y is CH-P, or if Y is oxygen then R₂ is P;
P is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar₅, or Ar₅
Ar₁ or Ar₂, independently, is an alicyclic or aromatic, mono-, bi- or tricyclic, carbo- or heterocyclic ring, wherein the ring is optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₃-C₈ cycloalkyl, C₅-C₇ cycloalkenyl, C₁-C₄ alkoxy, C₂-C₄ alkenyloxy, phenoxy, benzyloxy, and amino; wherein the individual ring contains 5-8 members; and wherein the heterocyclic ring contains 1-6 heteroatom(s) independently selected from the group consisting of O, N, and S, and, wherein any aromatic or tertiary alkylamine is optionally oxidized to a corresponding N-oxide;
m is 0 or 1
n is 1 or 2;
p is 0, 1, or 2;
t is 0, 1, 2, 3, or 4;
X is O, CH₂ or S;
W and Y, independently, are O, S, CH₂ or H₂;
Z is C(R₁)₂, O, S, a direct bond or NR₁; or, Z-R₁ is J-K-L, 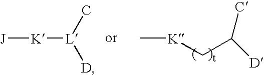
wherein:
C and D are, independently, hydrogen, Ar₄, Ar₁, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, Ar₁ and Ar₄; wherein said alkyl, alkenyl, cycloalkyl or cycloalkenyl is optionally substituted with C₁-C₆ alkyl, C₂-C₆ alkenyl, hydroxy, amino, halo, haloalkyl, thiocarbonyl, C₁-C₆ ester; C₁-C₆ thioester, C₁-C₆ alkoxy, C₁-C₆ alkenoxy, cyano, nitro, imino, C₁-C₆ alkylamino, amino-(C₁-C₆)alkyl, sulfhydryl, thio-(C₁-C₆)alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NR₅, or (SO)ₚ;
C′ and D′ are independently hydrogen, Ar₅, C₁-C₆ straight or branched chain alkyl, or C₂-C₆ straight or branched chain alkenyl, wherein said alkyl or alkenyl is optionally substituted with C₅-C₇ cycloalkyl, C₅-C₇ cycloalkenyl, or Ar₅, wherein, one or two carbon atom(s) of said alkyl or alkenyl may be substituted with one or two heteroatom(s) independently selected from the group consisting of oxygen, sulfur, SO, and SO₂ in chemically reasonable substitution patterns, or 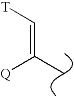
wherein Q is hydrogen, C₁-C₆ straight or branched chain alkyl, or C₂-C₆ straight or branched chain alkenyl; and
T is Ar₅ or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl J is O, NR₁, S, or (CR₁)₂;
K is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl or Ar₃₁ is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl or Ar₃₁ is optionally replaced with O, NR′″, or S(O)ₚ;
K′is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, haloalkyl, thiocarbonyl, ester, thioester, alkoxy, alkenoxy, cyano, nitro, imino, alkylamino, aminoalkyl, sulfhydryl, thioalkyl, sulfonyl, or oxygen to form a carbonyl, or wherein any carbon atom of said alkyl or alkenyl is optionally replaced with O, NR₅, S(O);
K″ is C(R₁)₂, O, S, a direct bond or NR₁;
R′″ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₃ group;
L is an aromatic amine or a tertiary amine oxidized to a corresponding N-oxide;
said aromatic amine being selected from the group consisting of pyridyl, pyrimidyl, quinolinyl, and isoquinolinyl, said aromatic amine being optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; and wherein
said tertiary amine is NRₓR_{y}R_{z}, wherein Rₓ, R_{y}, and R_{z} are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl, or Ar₃ is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar₃ is optionally replaced with O, NR′, S(O)ₚ;
L′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, haloalkyl, thiocarbonyl, ester, thioester, alkoxy, alkenoxy, cyano, nitro, imino, alkylamino, aminoalkyl, sulfhydryl, thioalkyl, sulfonyl, or oxygen to form a carbonyl, or wherein any carbon atom of said alkyl or alkenyl is optionally replaced with O, NR₅, S(O)ₚ
Ar₃ is selected from the group consisting of pyrrolidinyl, pyridyl, pyrimidyl, pyrazyl, pyridazyl, quinolinyl, and isoquinolinyl; or,
Ar₄ is an alicyclic or aromatic, mono-, bi- or tricyclic, carbo- or heterocyclic ring, wherein the ring is optionally substituted with one or more substituent(s) independently selected from the group consisting of alkylamino, amido, amino, aminoalkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, ester, formanilido, halo, haloalkyl, hydroxy, imino, isocyano, isonitrilo, nitrilo, nitro, nitroso, phenoxy, sulfhydryl, sulfonylsulfoxy, thio, thioalkyl, thiocarbonyl, thiocyano, thioester, thioformamido, trifluoromethyl, and carboxylic and heterocyclic moieties; wherein the individual alicyclic or aromatic ring contains 5-8 members and wherein said heterocyclic ring contains 1-6 heteroatom(s) independently selected from the group consisting of O, N, and S; and wherein any aromatic or tertiary alkyl amine is optionally oxidized to a corresponding N-oxide;
Ar₅ is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar₅ optionally contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
R₅ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₄ or Ar₁ group;
U is either O or N, provided that:
when U is O, then R′ is a lone pair of electrons and R″ is selected from the group consisting of Ar₄, C₃-C₈ cycloalkyl, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; and
when U is N, then R′ and R″ are, independently, selected from the group consisting of hydrogen, Ar₄, C₃-C₁₀ cycloalkyl, a C₇-C₁₂ bi- or tri-cyclic carbocycle, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; or R′ and R″ are taken together to form a heterocyclic 5- or 6-membered ring selected from the group consisting of pyrrolidine, imidazolidine, pyrazolidine, piperidine, and piperazine; or,
a pharmaceutically acceptable salt, ester or solvate thereof.

2. A method as claimed in claim 1 in which the sensorineurotrophic compound is a compound of formula I: 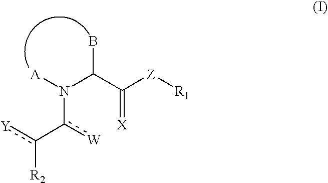
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing one or more heteroatom(s) independently selected from the group consisting of O, S, SO, SO₂, N, NH, and NR₂;
X is either O or S;
Z is either S, CH₂, CHR₁ or CR₁R₃;
W and Y are independently O, S, CH₂ or H₂;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxy; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

3. A method as claimed in claim 2 in which the sensorineurotrophic compound is a compound of formula II: 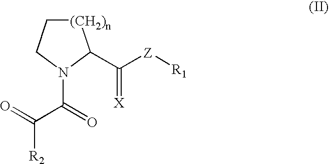
or a pharmaceutically acceptable salt, ester, or solvate thereof, wherein:
n is 1 or 2;
X is O or S;
Z is selected from the group consisting of S, CH₂, CHR₁, and CR₁R₃;
R₁ and R₃ are independently selected from the group consisting of C₁-C₅ straight or branched chain alkyl, C₂-C₅ straight or branched chain alkenyl, and Ar₁, wherein said alkyl, alkenyl or Ar₁ is unsubstituted or substituted with one or more substituent(s) independently selected from the group consisting of halo, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, and Ar₁;
R₂ is selected from the group consisting of C₁-C₉ straight or branched chain alkyl, C₂-C₉ straight or branched chain alkenyl, C₃-C₈ cycloalkyl, C₅-C₇ cycloalkenyl, and Ar₁; and
Ar₁ is phenyl, benzyl, pyridyl, fluorenyl, thioindolyl or naphthyl, wherein said Ar₁ is unsubstituted or substituted with one or more substituent(s) independently selected from the group consisting of halo, trifluoromethyl, hydroxy, nitro, C₁-C₆ straight or branched chain alkyl, C₂-C₆ straight or branched chain alkenyl, C₁-C₄ alkoxy, C₂-C₄ alkenyloxy, phenoxy, benzyloxy, and amino.

4. A method as claimed in claim 2 in which the sensorineurotrophic compound is a compound of formula III: 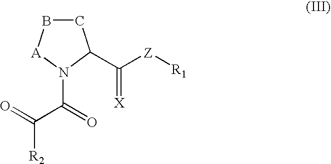
or a pharmaceutically acceptable salt, ester, or solvate thereof, wherein:
A, B, and C are independently CH₂, O, S, SO, SO₂, NH or NR₂;
X is O or S;
Z is S, CH₂, CHR₁ or CR₁R₃;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

5. A method as claimed in claim 2 in which the sensorineurotrophic compound is a compound of formula IV: 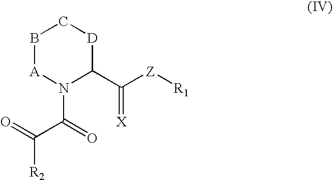
or a pharmaceutically acceptable salt, ester, or solvate thereof, wherein:
A, B, C and D are independently CH₂, O, S, SO, SO₂, NH or NR₂;
X is O or S;
Z is S, CH₂, CHR₁ or CR₁R₃;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ᵣ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₃-C₈ cycloalkyl, C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoro-methyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

6. A method as claimed in claim 1 in which the sensorineurotrophic agent may be a compound of formula VI: 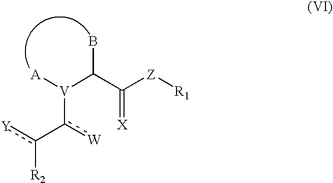
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₁;
X is O or S;
Z is O, NH or NR₁;
W and Y are independently O, S, CH₂ or H₂;
R₁ is C₁-C₆ straight or branched chain alkyl or C₂-C₆ straight or branched chain alkenyl, which is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain or alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

7. The method of claim 6 in which the sensorineurotrophic compound is a compound of formula VII: 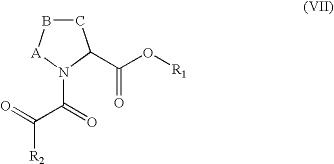
or a pharmaceutically acceptable salt, ester, or solvate thereof, wherein:
A, B and C are independently CH₂, O, S, SO, SO₂, NH or NR₁;
R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁; and
Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

8. The method of claim 7 in which the sensorineurotrophic compound is:

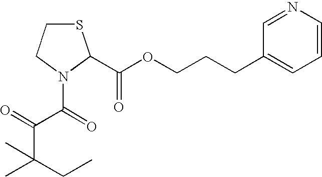

9. A method as claimed in claim 7 in which:
A is CH₂;
B is CH₂ or S;
C is CH₂ or NH;
R₁ is selected from the group consisting of 3-phenylpropyl and 3-(3-pyridyl)propyl; and
R₂ is selected from the group consisting of 1,1-dimethylpropyl, cyclohexyl, and tert-butyl.

10. A method as claimed in claim 6 in which the sensorineurotrophic compound is a compound of formula VIII: 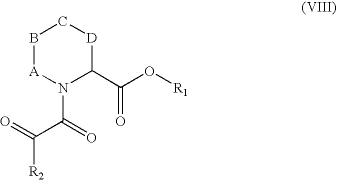
or a pharmaceutically acceptable salt, ester, or solvate thereof, wherein:
A, B, C and D are independently CH₂, O, S, SO, SO₂, NH or NR₁;
R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁; and
Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

11. A method of claim 10 in which:
A is CH₂;
B is CH₂;
C is S, O or NH;
D is CH₂;
R₁ is selected from the group consisting of 3-phenylpropyl and (3,4,5-trimethoxy)phenylpropyl; and
R₂ is selected from the group consisting of 1,1-dimethylpropyl, cyclohexyl, tert-butyl, phenyl, and 3,4,5-trimethoxyphenyl.

12. A method as claimed in claim 1 in which the sensorineurotrophic agent may be a compound of formula IX: 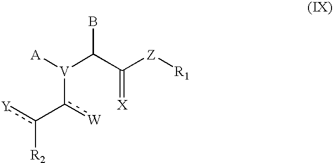
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₃, wherein R is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;
Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and
X is O or S;
Z is O, NH or NR₁;
W and Y are independently O, S, CH₂ or H₂;
R₁ is C₁-C₆ straight or branched chain alkyl or C₂-C₆ straight or branched chain alkenyl, which is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain or alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

13. A method as claimed in claim 1 in which the sensorineurotrophic compound is a compound of formula X: 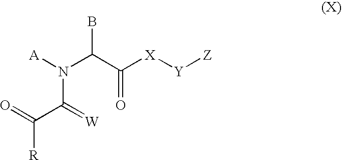
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing one or more heteroatom(s) independently selected from the group consisting of CH, CH₂, O, S, SO, SO₂, N, NH, and NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₂;
Ar₁ and Ar₂ are independently selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is selected from the group consisting of pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl or C₂-C₆ straight or branched chain alkenyl optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

14. A method as claimed in claim 13 in which the sensorineurotrophic compound is a compound of formula XI: 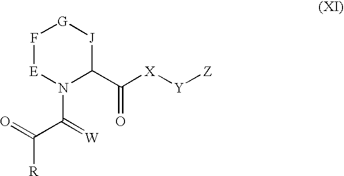
or a pharmaceutically acceptable salt, ester, or solvate thereof, wherein:
E, F, G and J are independently CH₂, O, S, SO, SO₂, NH or NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl, and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, and isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

15. A method as claimed in claim 13 in which the sensorineurotrophic compound is a compound of formula XII: 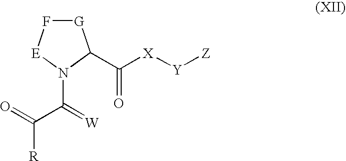
or a pharmaceutically acceptable salt, ester, or solvate thereof, wherein:
E, F, and G are independently CH₂, O, S, SO, SO₂, NH or NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

16. A method as claimed in claim 13 in which the sensorineurotrophic compound is a compound of formula XIII: 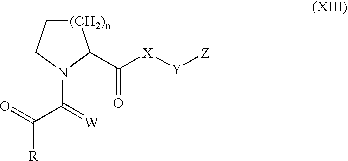
or a pharmaceutically acceptable salt, ester, or solvate thereof, wherein:
n is 1, 2, or 3, forming a 5-7 member heterocyclic ring;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃, independently, are hydrogen, C₁-C₄ straight or branched chain alkyl, C₃-C₄ straight or branched chain alkenyl or alkynyl, or Y-Z.

17. A method as claimed in claim 1 in which the sensorineurotrophic agent may be a compound of formula XIV: 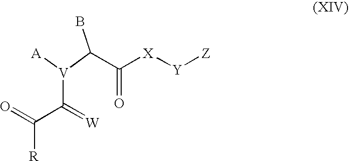
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR₇;
R₇ is either C₁-C₉ straight or branched chain alkyl, C₂-C₉ straight or branched chain alkenyl, C₃-C₉ cycloalkyl, C₅-C₇ cycloalkenyl, or Ar₃, wherein R₇ is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;
Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₂;
Ar₁ and Ar₂ are independently selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen;
wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is selected from the group consisting of pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl or C₂-C₆ straight or branched chain alkenyl optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

18. A method as claimed in claim 1 in which the sensorineurotrophic compound is a compound of formula XV: 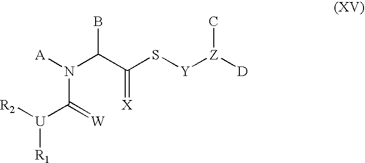
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more additional heteroatom(s) independently selected from the group consisting of O, S, SO, SO₂, N, NH, and NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

19. A method as claimed in claim 18 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

20. A method as claimed in claim 18 in which the sensorineurotrophic compound is a compound of formula XVI: 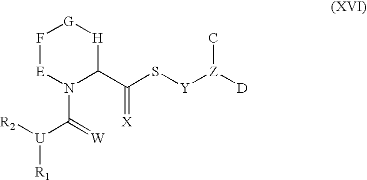
or a pharmaceutically acceptable salt, ester, or solvate thereof, wherein:
E, F, G and J are independently CH₂, O, S, SO, SO₂, NH, or NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

21. A method as claimed in claim 20 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

22. A method as claimed in claim 18 in which the sensorineurotrophic compound is a compound of formula XVII: 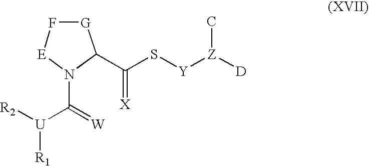
or a pharmaceutically acceptable salt, ester, or solvate thereof, wherein:
E, F, and G are independently CH₂, O, S, SO, SO₂, NH, and NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₈ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

23. A method as claimed in claim 22 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

24. A method as claimed in claim 1 in which the sensorineurotrophic compound is a compound of formula XVIII: 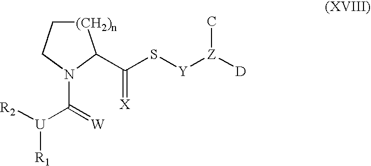
or a pharmaceutically acceptable salt, ester, or solvate thereof, wherein:
n is 1, 2 or 3;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain or alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

25. A method as claimed in claim 24 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

26. A method as claimed in claim 1 in which the sensorineurotrophic compound is a compound of formula XIX: 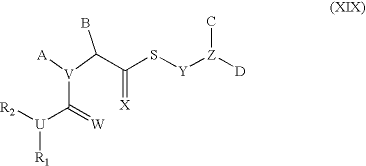
or a pharmaceutically acceptable salt, ester, or solvate thereof, wherein:
V is CH, N, or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂; and
A and B, together with the nitrogen and carbon atoms to which they are respectively attached, form a 5-7 membered saturated or unsaturated heterocyclic ring containing, in addition to the nitrogen atom, one or more additional heteroatom(s) independently selected from the group consisting of O, S, SO, SO₂, N, NH, and NR₃;
X is either O or S;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

27. A method as claimed in claim 1 in which the sensorineurotrophic compound is a compound of formula XX: 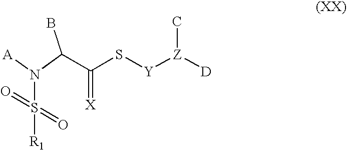
a pharmaceutically acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₃, S, SO, or SO₂.

28. A method as claimed in claim 27 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

29. A method as claimed in claim 28 in which A and B, together with the nitrogen and carbon atoms to which they are respectfully attached, form a 6 membered saturated or unsaturated heterocyclic ring; and R₂ is C₄-C₇ branched chain alkyl, C₄-C₇ cycloalkyl, phenyl, or 3,4,5-trimethoxyphenyl.

30. A method as claimed in claim 27 in which the sensorineurotrophic compound is selected from the group consisting of:
3-(para-Methoxyphenyl)-1-propylmercaptyl(2S)-N-(benzenesulfonyl)pyrrolidine-2-carboxylate;
3-(para-Methoxyphenyl)-1-propylmercaptyl(2S)-N-(α-toluenesulfonyl)pyrrolidine-2-carboxylate;
3-(para-Methoxyphenyl)-1-propylmercaptyl(2S)-N-(α-toluenesulfonyl)pyrrolidine-2-carboxylate;
1,5-Diphenyl-3-pentylmercaptyl-N-(para-toluenesulfonyl)pipecolate; and
pharmaceutically acceptable salts and solvates thereof.

31. A method as claimed in claim 27 in which the sensorineurotrophic compound is a compound of formula XXI: 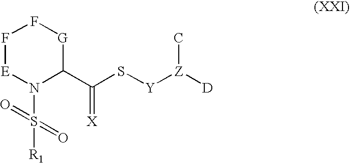
or a pharmaceutically acceptable salt, ester, or solvate thereof, wherein:
E, F, G and J are independently CH₂, O, S, SO, SO₂, NH or NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₃, S, SO, or SO₂.

32. A method as claimed in claim 31 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

33. A method as claimed in claim 27 in which the sensorineurotrophic agent is a compound of formula XXII: 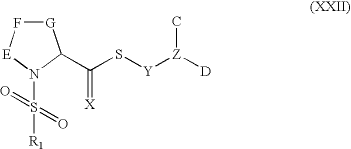
or a pharmaceutically acceptable salt, ester, or solvate thereof, wherein:
E, F, and G are independently CH₂, O, S, SO, SO₂, NH or NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₃, S, SO, or SO₂.

34. A method as claimed in claim 33 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

35. A method as claimed in claim 27 in which the sensorineurotrophic compound is a compound of formula XXIII: 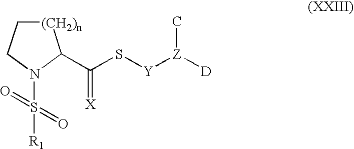
or a pharmaceutically acceptable salt, ester, or solvate thereof, wherein:
n is 1, 2 or 3;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₃, S, SO, or SO₂.

36. A method as claimed in claim 35 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

37. A method as claimed in claim 1 in which the sensorineurotrophic compound is a compound of formula XXIV: 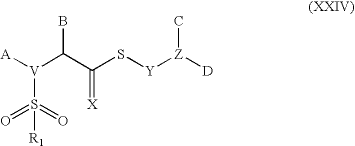
or a pharmaceutically acceptable salt, ester, or solvate thereof, wherein:
V is CH, N, or S;
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₂, S, SO, or SO₂.

38. A method as claimed in claim 1 in which the sensorineurotrophic compound is a compound of formula XXV: 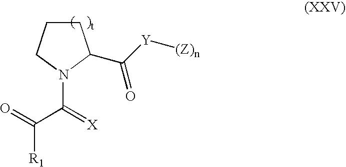
or a pharmaceutically acceptable salt, ester, or solvate thereof, wherein:
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
X is O, S, CH₂ or H₂;
Y is O or NR₂, wherein R₂ is a direct bond to a Z, hydrogen or C₁-C₆ alkyl; and
each Z, independently, is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 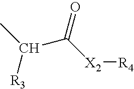
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;
n is 1 or 2, and;
t is 1, 2 or 3.

39. A method as claimed in claim 38 in which the compound is selected from the group consisting of:
3-phenyl-1-propyl (2S)-1-(3,3-dimethyl-1,2-dioxopentyl)-2-pyrrolidinecarboxylate;
3-phenyl-1-prop-2-(E)-enyl (2S)-1-(3,3-dimethyl-1,2-dioxopentyl)-2-pyrrolidinecarboxylate;
3-(3,4,5-trimethoxyphenyl)-1-propyl (2S)-1-(3,3-dimethyl-1,2-dioxopentyl)-2-pyrrolidine-carboxylate;
3-(3,4,5-trimethoxyphenyl)-1-prop-2-(E)-enyl (2S)-1-(3,3-dimethyl-1,2-dioxopentyl)-2-pyrrolidinecarboxylate;
3-(4,5-dichlorophenyl)-1-propyl (2S)-1-(3,3-dimethyl-1,2-dioxopentyl)-2-pyrrolidinecarboxylate;
3-(4,5-dichlorophenyl)-1-prop-2-(E)-enyl (2S)-1-(3,3-dimethyl-1,2-dioxopentyl)-2-pyrrolidine-carboxylate;
3-(4,5-methylenedioxyphenyl)-1-propyl (2S)-1-(3,3-dimethyl-1,2-dioxopentyl)-2-pyrrolidine-carboxylate;
3-(4,5-methylenedioxyphenyl)-1-prop-2-(E)-enyl (2S)-1-(3,3-dimethyl-1,2-dioxopentyl)-2-pyrrolidinecarboxylate;
3-cyclohexyl-1-propyl (2S)-1-(3,3-dimethyl-1,2-dioxopentyl)-2-pyrrolidinecarboxylate;
3-cyclohexyl-1-prop-2-(E)-enyl (2S)-1-(3,3-dimethyl-1,2-dioxopentyl)-2-pyrrolidinecarboxylate;
(1R)-1,3-diphenyl-1-propyl (2S)-1-(3,3-dimethyl-1,2-dioxopentyl)-2-pyrrolidinecarboxylate;
(1R)-1,3-diphenyl-1-prop-2-(E)-enyl (2S)-1-(3,3-dimethyl-1,2-dioxopentyl)-2-pyrrolidine-carboxylate;
(1R)-1-cyclohexyl-3-phenyl-1-propyl (2S)-1-(3,3-dimethyl-1,2-dioxopentyl)-2-pyrrolidine-carboxylate;
(1R)-1-cyclohexyl-3-phenyl-1-prop-2-(E)-enyl (2S)-1-(3,3-dimethyl-1,2-dioxopentyl)-2-pyrrolidinecarboxylate;
(1R)-1-(4,5-dichlorophenyl)-3-phenyl-1-propyl (2S)-1-(3,3-dimethyl-1,2-dioxopentyl)-2-pyrrolidine-carboxylate;
3-phenyl-1-propyl (2S)-1-(1,2-dioxo-2-cyclohexyl)ethyl-2-pyrrolidinecarboxylate;
3-phenyl-1-propyl (2S)-1-(1,2-dioxo-4-cyclohexyl)butyl-2-pyrrolidinecarboxylate;
3-phenyl-1-propyl (2S)-1-(1,2-dioxo-2-[2-furanyl])ethyl-2-pyrrolidinecarboxylate;
3-phenyl-1-propyl (2S)-1-(1,2-dioxo-2-[2-thienyl])ethyl-2-pyrrolidinecarboxylate;
3-phenyl-1-propyl (2S)-1-(1,2-dioxo-2-[2-thiazolyl])ethyl-2-pyrrolidinecarboxylate;
3-phenyl-1-propyl (2S)-1-(1,2-dioxo-2-phenyl)ethyl-2-pyrrolidinecarboxylate;
1,7-diphenyl-4-heptyl (2S)-1-(3,3-dimethyl-1,2-dioxopentyl)-2-pyrrolidinecarboxylate;
3-phenyl-1-propyl (2S)-1-(3,3-dimethyl-1,2-dioxo-4-hydroxybutyl)-2-pyrrolidinecarboxylate;
3-phenyl-1-propyl (2S)-1-(3,3-dimethyl-1,2-dioxopentyl)-2-pyrrolidinecarboxamide;
1-[1-(3,3-dimethyl-1,2-dioxopentyl)-L-proline]-L-phenylalanine ethyl ester;
1-[1-(3,3-dimethyl-1,2-dioxopentyl)-L-proline]-L-leucine ethyl ester;
1-[1-(3,3-dimethyl-1,2-dioxopentyl)-L-proline]-L-phenylglycine ethyl ester;
1-[1-(3,3-dimethyl-1,2-dioxopentyl)-L-proline]-L-phenylalanine phenyl ester;
1-[1-(3,3-dimethyl-1,2-dioxopentyl)-L-proline]-L-phenylalanine benzyl ester;
1-[1-(3,3-dimethyl-1,2-dioxopentyl)-L-proline]-L-isoleucine ethyl ester; and
pharmaceutically acceptable salts, esters, and solvates thereof.

40. A method as claimed in claim 38 in which the sensorineurotrophic compound is a compound of formula XXVI: 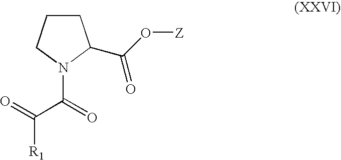
or a pharmaceutically acceptable salt, ester, or solvate thereof, wherein:
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
Z is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 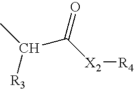
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl; and
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl.

41. A method as claimed in claim 1 in which the sensorineurotrophic agent may be a compound of formula XXVII: 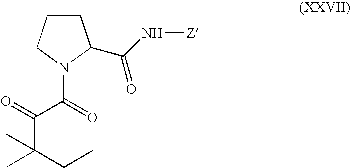
or a pharmaceutically acceptable salt, ester, or solvate thereof, wherein:
Z′ is the fragment 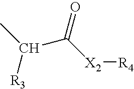
wherein:
R₃ is C₁-C₉ straight or branched chain alkyl or unsubstituted Ar₁, wherein said alkyl is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and
Ar₁ is selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino.

42. A method as claimed in claim 38 in which the sensorineurotrophic agent may also be a compound of formula XXVIII: 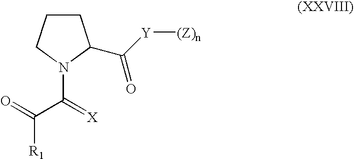
wherein:
R₁ is C₁-C₆ straight or branched chain alkyl, C₂-C₆ straight or branched chain alkenyl, C₃-C₆ cycloalkyl or Ar₁, wherein said alkyl or alkenyl is unsubstituted or substituted with C₃-C₆ cycloalkyl or Ar₂;
Ar₁ and Ar₂ are independently selected from the group consisting of 2-furyl, 2-thienyl, and phenyl;
X is selected from the group consisting of oxygen and sulfur;
Y is oxygen or NR₂, wherein R₂ is a direct bond to a Z, hydrogen or C₁-C₆ alkyl;
each Z, independently, is hydrogen, C₁-C₆ straight or branched chain alkyl, or C₂-C₆ straight or branched chain alkenyl, wherein said Z is substituted with one or more substituent(s) independently selected from the group consisting of 2-furyl, 2-thienyl, C₃-C₆ cycloalkyl, pyridyl, and phenyl, each having one or more substituent(s) independently selected from the group consisting of hydrogen and C₁-C₄ alkoxy; and
n is 1 or 2.

43. A method as claimed in claim 42 in which the compound is selected from the group consisting of:
3-(2,5-dimethoxyphenyl)-1-propyl (2S)-1-(3,3-dimethyl-1,2-dioxopentyl)-2-pyrrolidinecarboxylate;
3-(2,5-dimethoxyphenyl)-1-prop-2-(E)-enyl (2S)-1-(3,3-dimethyl-1,2-dioxopentyl)-2-pyrrolidine-carboxylate;
2-(3,4,5-trimethoxyphenyl)-1-ethyl (2S)-1-(3,3-dimethyl-1,2-dioxopentyl)-2-pyrrolidinecarboxylate;
3-(3-pyridyl)-1-propyl (2S)-1-(3,3-dimethyl-1,2-dioxopentyl)-2-pyrrolidinecarboxylate;
3-(2-pyridyl)-1-propyl (2S)-1-(3,3-dimethyl-1,2-dioxopentyl)-2-pyrrolidinecarboxylate;
3-(4-pyridyl)-1-propyl (2S)-1-(3,3-dimethyl-1,2-dioxopentyl)-2-pyrrolidinecarboxylate;
3-phenyl-1-propyl (2S)-1-(2-tert-butyl-1,2-dioxoethyl)-2-pyrrolidinecarboxylate;
3-phenyl-1-propyl (2S)-1-(2-cyclohexylethyl-1,2-dioxoethyl)-2-pyrrolidinecarboxylate;
3-(3-pyridyl)-1-propyl (2S)-1-(2-cyclohexylethyl-1,2-dioxoethyl)-2-pyrrolidine-carboxylate;
3-(3-pyridyl)-1-propyl (2S)-1-(2-tert-butyl-1,2-dioxoethyl)-2-pyrrolidinecarboxylate;
3,3-diphenyl-1-propyl (2S)-1-(3,3-dimethyl-1,2-dioxopentyl)-2-pyrrolidinecarboxylate;
3-(3-pyridyl)-1-propyl (2S)-1-(2-cyclohexyl-1,2-dioxoethyl)-2-pyrrolidinecarboxylate;
3-(3-pyridyl)-1-propyl (2S)-N-([2-thienyl]glyoxyl)pyrrolidinecarboxylate;
3,3-diphenyl-1-propyl (2S)-1-(3,3-dimethyl-1,2-dioxobutyl)-2-pyrrolidinecarboxylate;
3,3-diphenyl-1-propyl (2S)-1-cyclohexylglyoxyl-2-pyrrolidinecarboxylate;
3,3-diphenyl-1-propyl (2S)-1-(2-thienyl)glyoxyl-2-pyrrolidinecarboxylate; and
pharmaceutically acceptable salts, esters, and solvates thereof.

44. A method as claimed in claim 1 in which the sensorineurotrophic compound is a compound of formula XXIX: 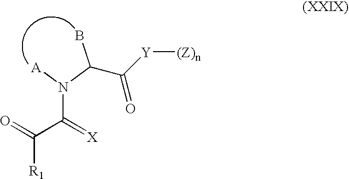
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo-(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, alkylthio, sulfhydryl, amino, (C₁-C₆)-alkylamino, amino-(C₁-C₆)-alkyl, aminocarboxyl, and Ar₂;
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S;
X is O, S, CH₂ or H₂;
Y is O or NR₂, wherein R₂ is a direct bond to a Z, hydrogen or C₁-C₆ alkyl; and
each Z, independently, is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 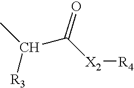
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl; and
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and,
n is 1 or 2.

45. A method as claimed in claim 1 in which the sensorineurotrophic compound is a compound of formula (LV): 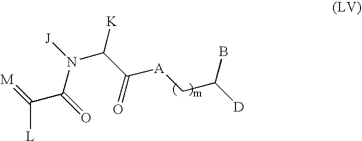
or a pharmaceutically acceptable salt, ester, or solvate thereof, wherein:
m is 0-3;
A is CH₂, O, NH, or N—(C₁-C₄ alkyl);
B and D are independently hydrogen, Ar, C₅-C₇ cycloalkyl substituted C₁-C₆ straight or branched chain alkyl or C₂-C₆ straight or branched chain alkenyl, C₅-C₇ cycloalkenyl substituted C₁-C₆ straight or branched chain alkyl or C₂-C₆ straight or branched chain alkenyl, or Ar substituted C₁-C₆ straight or branched chain alkyl or C₂-C₆ straight or branched chain alkenyl, wherein in each case, one or two carbon atom(s) of said alkyl or alkenyl may be substituted with one or two heteroatom(s) independently selected from the group consisting of oxygen, sulfur, SO, and SO₂ in chemically reasonable substitution patterns, or 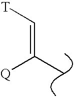
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
L is either hydrogen or U; M is either oxygen or CH-U, provided that if L is hydrogen, then M is CH-U, or if M is oxygen then L is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylmethyl; or J and K are taken together to form a 5-7 membered heterocyclic ring which is substituted with oxygen, sulfur, SO, or SO₂.

46. A method as claimed in claim 1 in which the sensorineurotrophic compound is a compound of formula (LVI): 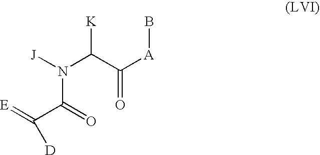
or a pharmaceutically acceptable salt, ester, or solvate thereof, wherein:
A is O, NH, or N—(C₁-C₄ alkyl);
B is hydrogen, CHL-Ar, C₁-C₆ straight or branched chain alkyl, C₂-C₆ straight or branched chain alkenyl, C₅-C₇ cycloalkyl, C₅-C₇ cycloalkenyl, Ar substituted C₁-C₆ alkyl or C₂-C₆ alkenyl, or 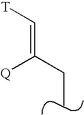
wherein L and Q are independently hydrogen, C₁-C₆ straight or branched chain alkyl, or C₂-C₆ straight or branched chain alkenyl; and
T is Ar or C₅-C₇ cyclohexyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 2-pyridyl, 3-pyridyl, 4-pyridyl and phenyl having 1-3 substituent(s) independently selected from the group consisting of hydrogen, halo, hydroxy, nitro, CF₃, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, and phenyl.
D is hydrogen or U; E is oxygen or CH-U, provided that if D is hydrogen, then E is CH-U, or if E is oxygen, then D is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇-cycloalkyl, C₅-C₇ cycloalkenyl substituted with C₁-C₄ straight or branched chain alkyl or C₂-C₄ straight or branched chain alkenyl, 2-indolyl, 3-indolyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylethyl; or J and K are taken together to form a 5-7 membered heterocyclic ring which is substituted with oxygen, sulfur, SO, or SO₂.

47. A method as claimed in claim 1 in which the sensorineurotrophic compound is a compound of formula LVIII: 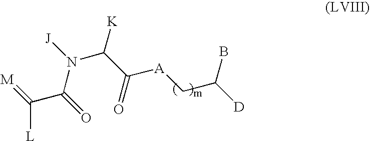
or a pharmaceutically acceptable salt, ester, or solvate thereof, wherein:
V is CH, N, or S;
J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;
Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;
A is CH₂, O, NH, or N—(C₁-C₄ alkyl);
B and D are independently hydrogen, Ar, C₅-C₇ cycloalkyl substituted C₁-C₆ straight or branched chain alkyl or C₂-C₆ straight or branched chain alkenyl, C₅-C₇ cycloalkenyl substituted C₁-C₆ straight or branched chain alkyl or C₂-C₆ straight or branched chain alkenyl, or Ar substituted C₁-C₆ straight or branched chain alkyl or C₂-C₆ straight or branched chain alkenyl, wherein in each case, one or two carbon atom(s) of said alkyl or alkenyl may be substituted with one or two heteroatom(s) independently selected from the group consisting of oxygen, sulfur, SO, and SO₂ in chemically reasonable substitution patterns, or 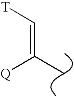
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
L is either hydrogen or U; M is either oxygen or CH-U, provided that if L is hydrogen, then M is CH-U, or if M is oxygen then L is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylmethyl; or J and K are taken together to form a 5-7 membered heterocyclic ring which is substituted with oxygen, sulfur, SO, or SO₂.

48. A method as claimed in claim 1 in which the sensorineurotrophic compound is a compound of the formula (LIX): 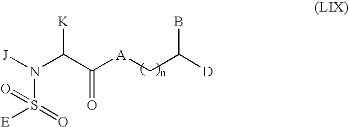
or a pharmaceutically acceptable salt, ester or solvate thereof, wherein:
A is CH₂, O, NH, or N—(C₁-C₄ alkyl);
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 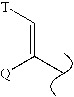
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar;
J is hydrogen, C₁ or C₂ alkyl, or benzyl; K is C₁-C₄ straight or branched chain alkyl, benzyl, or cyclohexylmethyl; or J and K are taken together to form a 5-7 membered heterocyclic ring which is substituted with O, S, SO, or SO₂;
n is 0 to 3.

49. A method as claimed in claim 1 in which the sensorineurotrophic compound is a compound of Formula LXI: 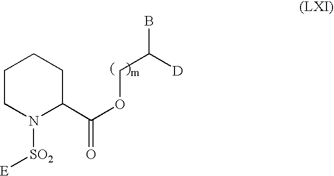
or a pharmaceutically acceptable salt, ester or solvate thereof, wherein:
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 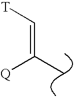
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and
m is 0 to 3.

50. A method as claimed in claim 1 in which the sensorineurotrophic compound is a compound of Formula (LXII): 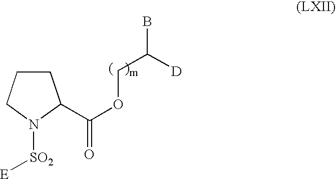
or a pharmaceutically acceptable salt thereof, wherein:
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl, or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 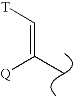
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and
m is 0 to 3.

51. A method as claimed in claim 1 in which the sensorineurotrophic compound is a compound of Formula LXIII: 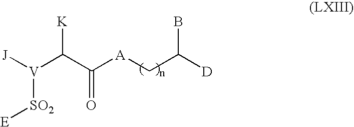
or a pharmaceutically acceptable salt, ester, or solvate thereof, wherein:
V is CH, N, or S;
J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;
Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;
A is CH₂, O, NH, or N—(C₁-C₄ alkyl);
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 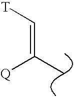
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar;
J is hydrogen, C₁ or C₂ alkyl, or benzyl; K is C₁-C₄ straight or branched chain alkyl, benzyl, or cyclohexylmethyl; or J and K are taken together to form a 5-7 membered heterocyclic ring which is substituted with O, S, SO, or SO₂;
n is 0 to 3.

52. A method as claimed in claim 1 in which the sensorineurotrophic compound is a compound of formula (LXIV): 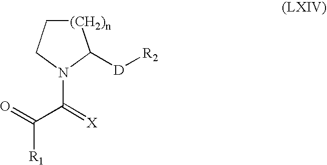
in which:
n is 1-3;
X is either O or S;
R₁ is selected from the group consisting of C₁-C₉ straight or branched chain alkyl, C₂-C₉ straight or branched chain alkenyl, aryl, heteroaryl, carbocycle, or heterocycle;
D is a bond, or a C₁-C₁₀ straight or branched chain alkyl, C₂-C₁₀ alkenyl or C₂-C₁₀ alkynyl; and
R₂ is a carboxylic acid or a carboxylic acid isostere;
or a pharmaceutically acceptable salt, ester, or solvate thereof.

53. A method as claimed in claim 52 in which
R₂ is selected from the group: 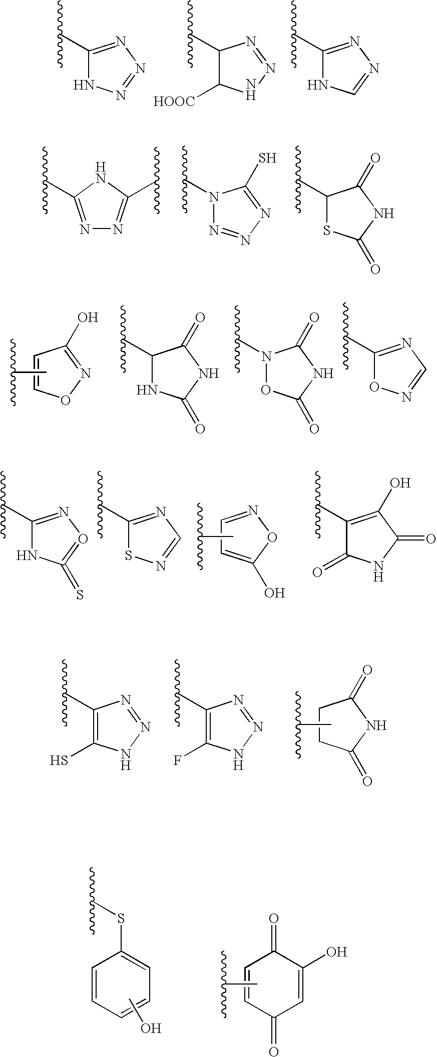
—COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl.

54. A method as claimed in claim 1 in which the sensorineurotrophic compound is a compound of formula (LXV): 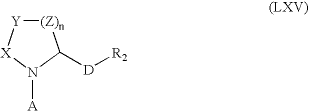
in which
X, Y, and Z are independently selected from the group consisting of C, O, S, or N, provided that X, Y, and Z are not all C;
n is 1-3;
A is selected from the group consisting of L₁, L₂, L₃, or L₄, in which 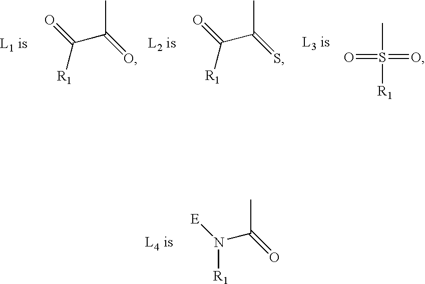
and R₁ and E, independently, are selected from the group consisting of hydrogen, C₁-C₉ straight or branched chain alkyl, C₂-C₉ straight or branched chain alkenyl, aryl, heteroaryl, carbocycle, and heterocycle;
R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where
R³ is hydrogen, hydroxy, halo, halo(C₁-C₆)-alkyl, thiocarbonyl, (C₁-C₆)-alkoxy, (C₂-C₆)-alkenoxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;
or a pharmaceutically acceptable salt, ester, or solvate thereof.

55. A method as claimed in claim 1 in which the sensorineurotrophic compound is a compound of formula (LXVI): 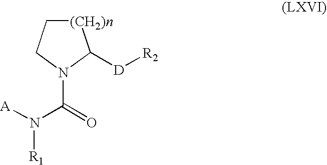
in which:
n is 1-3;
R₁ and A are independently selected from the group consisting of hydrogen, C₁-C₉ straight or branched chain alkyl, C₂-C₉ straight or branched chain alkenyl, aryl, heteroaryl, carbocycle, and heterocycle;
D is a bond, or a C₁-C₁₀ straight or branched chain alkyl, C₂-C₁₀ alkenyl or C₂-C₁₀ alkynyl;
R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where
R³ is hydrogen, hydroxy, halo, halo(C₁-C₆)-alkyl, thiocarbonyl, (C₁-C₆)-alkoxy, (C₂-C₆)-alkenoxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl;
or a pharmaceutically acceptable salt, ester, or solvate thereof.

56. A method as claimed in claim 1 in which the sensorineurotrophic compound is a compound of formula (LXVII): 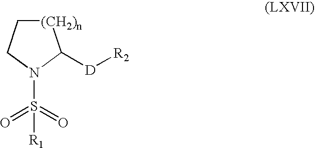
in which:
n is 1-3;
R₁ is selected from the group consisting of hydrogen, C₁-C₉ straight or branched chain alkyl, C₂-C₉ straight or branched chain alkenyl, aryl, heteroaryl, carbocycle, or heterocycle;
D is a bond, or a C₁-C₁₀ straight or branched chain alkyl, C₂-C₁₀ alkenyl or C₂-C₁₀ alkynyl;
R₂ is a carboxylic acid or a carboxylic acid isostere;
wherein said alkyl, alkenyl, alkynyl, aryl, heteroaryl, carbocycle, heterocycle, or carboxylic acid isostere is optionally substituted with one or more substituents selected from R³, where
R³ is hydrogen, hydroxy, halo, halo-(C₁-C₆)-alkoxy, thiocarbonyl, (C₁-C₆)-alkoxy, (C₂-C₆)-alkenyloxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;
or a pharmaceutically acceptable salt, ester or solvate thereof.

57. A method for treating or preventing hearing loss which comprises administering to a warm-blooded animal a compound selected from the group comprising: 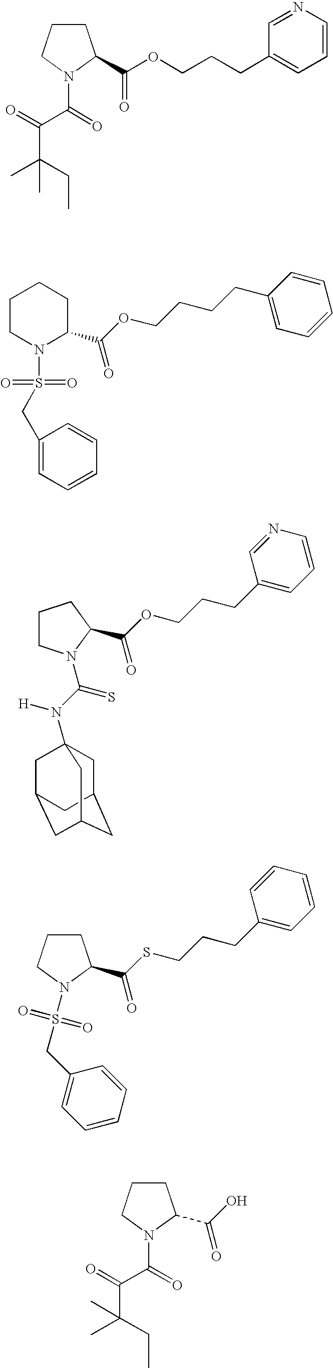 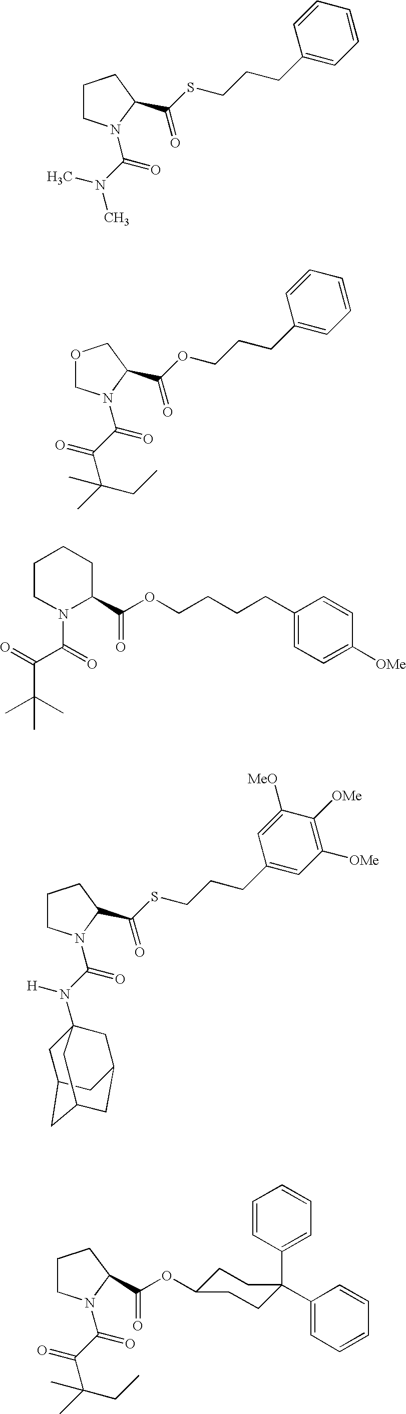 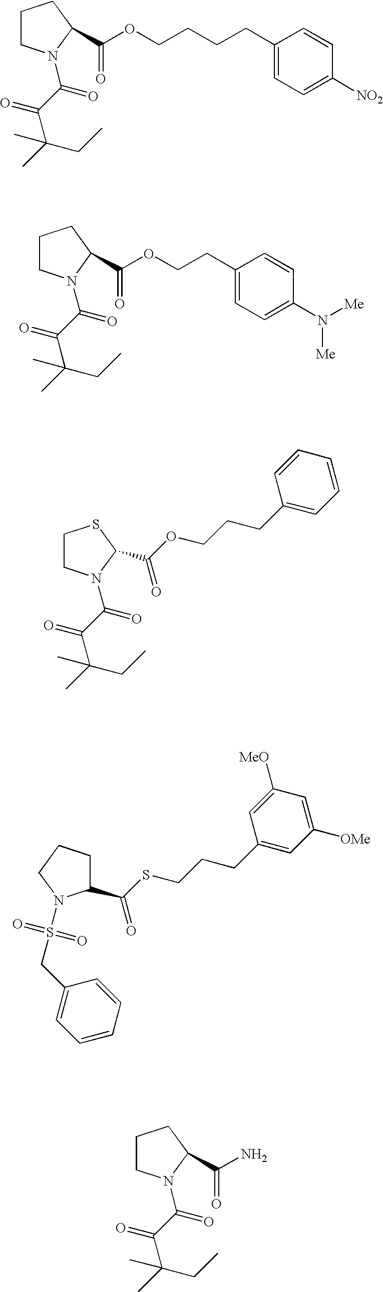 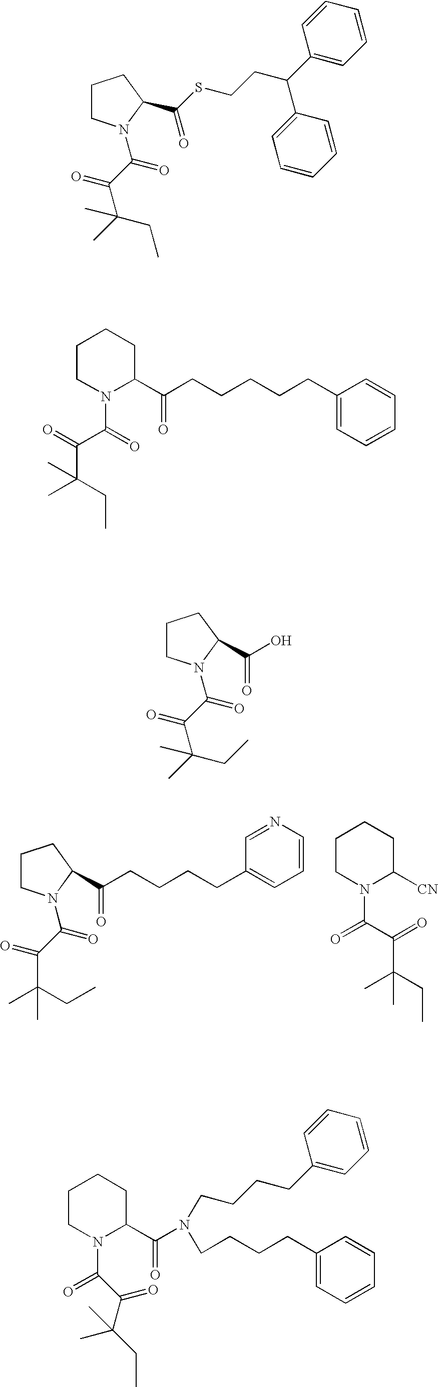 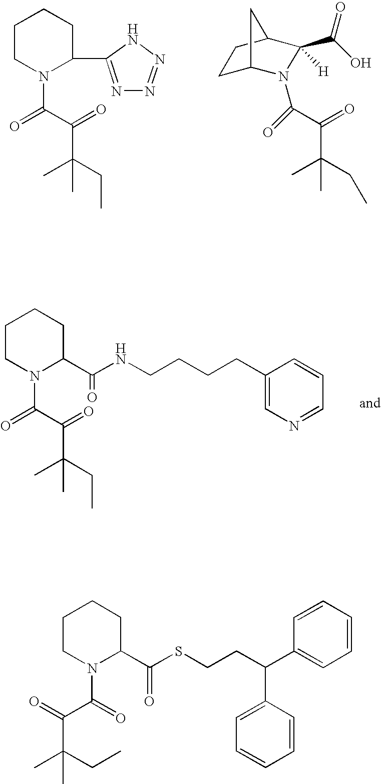
or a pharmaceutically acceptable salt, ester or solvate thereof.

58. A method for the prevention or treatment of injury or degeneration of inner ear sensory cells which comprises administering to a warm-blooded animal a sensorineurotrophic compound of the formula (I′): 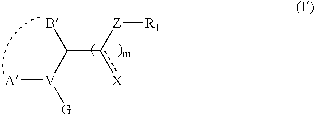
wherein
A′ is hydrogen, C₁ or C₂ alkyl, or benzyl;
B′ is C₁-C₄ straight or branched chain alkyl, benzyl or cyclohexylmethyl; or,
A′ and B′, taken together with the atoms to which they are attached, form a 5-7 membered saturated, unsaturated or aromatic heterocylic or carbocyclic ring which contains one or more additional O, C(R₁)₂, S(O)ₚ, N, NR₁, or NR₅ atoms;
V is CH, S, or N;
G is 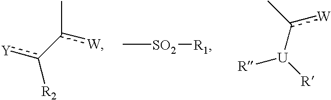
each R₁, independently, is hydrogen, C₁-C₉ straight or branched chain alkyl, or C₂-C₉ straight or branched chain alkenyl or alkynyl, C₃-C₉ cycloalkyl, C₅-C₇ cycloalkenyl, a carboxylic acid or carboxylic acid isostere, N(R₄)ₙ, Ar₁, Ar₄ or K-L wherein said alkyl, cycloalkyl, cycloalkenyl, alkynyl, alkenyl, Ar₁ or Ar₄ is optionally substituted with one or more substituent(s) independently selected from the group consisting of:
2-furyl, 2-thienyl, pyridyl, phenyl, C₃-C₆ cycloalkyl wherein said furyl, thienyl, pyridyl, phenyl or cycloalkyl group optionally is substituted with C₁-C₄ alkoxy, (Ar₁)ₙ, halo, halo-C₁-C₆-alkyl, carbonyl, thiocarbonyl, C₁-C₆ thioester, cyano, imino, COOR₆ in which R₆ is C₁-C₉ straight or branched chain alkyl or alkenyl, hydroxy, nitro, trifluoromethyl, C₁-C₆ alkoxy, C₂-C₄ alkenyloxy, C₁-C₆ alkylaryloxy C₁-C₆ aryloxy, aryl-(C₁-C₆)-alkyloxy, phenoxy, benzyloxy, thio-(C₁-C₆)-alkyl, C₁-C₆-alkylthio, sulfhydryl, sulfonyl, amino, (C₁-C₆)-mono- or di-alkylamino, amino-(C₁-C₆)-alkyl, aminocarboxy, C₃-C₈ cycloalkyl, C₁-C₆ straight or branched chain alkyl, C₂-C₆ straight or branched chain alkenyl optionally substituted with (Ar₁)ₙ, C₃-C₈ cycloalkyl, C₁-C₆ straight or branched chain alkyl, C₂-C₆ straight or branched chain alkenyl substituted with C₃-C₈ cycloalkyl, C₃-C₈ cycloalkyl, and Ar₂, and, wherein any carbon atom of an alkyl or alkenyl group may optionally replaced with O, NR₅, or S(O)ₚ; or,
R₁ is a moiety of the formula: 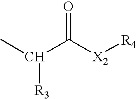
wherein:
R₃ is C₁-C₉ straight or branched chain alkyl which is optionally substituted with C₃-C₈ cycloalkyl or Ar₁;
X₂ is O or NR₆, wherein R₆ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;
R₂ is C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl, or cycloalkenyl is optionally substituted with one or more substituents selected from the group consisting of C₁-C₆ straight or branched chain alkyl, C₂-C₆ straight or branched chain alkenyl, C₃-C₈ cycloalkyl, C₅-C₇ cycloalkenyl, (Ar₁)ₙ and hydroxy; or,
R₂ is either hydrogen or P; Y is either oxygen or CH-P, provided that if R₂ is hydrogen, then Y is CH-P, or if Y is oxygen then R₂ is P;
P is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar₅, or Ar₅
Ar₁ or Ar₂, independently, is an alicyclic or aromatic, mono-, bi- or tricyclic, carbo- or heterocyclic ring, wherein the ring is optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₃-C₈ cycloalkyl, C₅-C₇ cycloalkenyl, C₁-C₄ alkoxy, C₂-C₄ alkenyloxy, phenoxy, benzyloxy, and amino; wherein the individual ring contains 5-8 members; and wherein the heterocyclic ring contains 1-6 heteroatom(s) independently selected from the group consisting of O, N, and S, and, wherein any aromatic or tertiary alkylamine is optionally oxidized to a corresponding N-oxide;
m is 0 or 1 n is 1 or 2;
p is 0, 1, or 2;
t is 0, 1, 2, 3, or 4;
X is O, CH₂ or S;
W and Y, independently, are O, S, CH₂ or H₂;
Z is C(R₁)₂, O, S, a direct bond or NR₁; or, Z-R₁ is J-K-L, 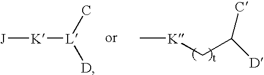
wherein:
C and D are, independently, hydrogen, Ar₄, Ar₁, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, Ar₁ and Ar₄; wherein said alkyl, alkenyl, cycloalkyl or cycloalkenyl is optionally substituted with C₁-C₆ alkyl, C₂-C₆ alkenyl, hydroxy, amino, halo, haloalkyl, thiocarbonyl, C₁-C₆ ester, C₁-C₆ thioester, C₁-C₆ alkoxy, C₁-C₆ alkenoxy, cyano, nitro, imino, C₁-C₆ alkylamino, amino-(C₁-C₆)alkyl, sulfhydryl, thio-(C₁-C₆)alkyl, or sulfonyl; wherein any carbon atom of said alkyl or alkenyl is optionally substituted in one or more position(s) with oxygen to form a carbonyl; or
wherein any carbon atom of said alkyl or alkenyl is optionally replaced with O, NR₅, or (SO)ₚ;
C′ and D′ are independently hydrogen, Ar₅, C₁-C₆ straight or branched chain alkyl, or C₂-C₆ straight or branched chain alkenyl, wherein said alkyl or alkenyl is optionally substituted with C₅-C₇ cycloalkyl, C₅-C₇ cycloalkenyl, or Ar₅, wherein, one or two carbon atom(s) of said alkyl or alkenyl may be substituted with one or two heteroatom(s) independently selected from the group consisting of oxygen, sulfur, SO, and SO₂ in chemically reasonable substitution patterns, or 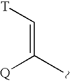
wherein Q is hydrogen, C₁-C₆ straight or branched chain alkyl, or C₂-C₆ straight or branched chain alkenyl; and
T is Ar₅ or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl J is O, NR₁, S, or (CR₁)₂;
K is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl or Ar₃, is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl or Ar₃, is optionally replaced with O, NR′″, or S(O)ₚ;
K′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, haloalkyl, thiocarbonyl, ester, thioester, alkoxy, alkenoxy, cyano, nitro, imino, alkylamino, aminoalkyl, sulfhydryl, thioalkyl, sulfonyl, or oxygen to form a carbonyl, or wherein any carbon atom of said alkyl or alkenyl is optionally replaced with O, NR₅, S(O)ₚ;
K″ is C(R₁)₂′ O, S, a direct bond or NR₁;
R′″ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₃ group;
L is an aromatic amine or a tertiary amine oxidized to a corresponding N-oxide;
said aromatic amine being selected from the group consisting of pyridyl, pyrimidyl, quinolinyl, and isoquinolinyl, said aromatic amine being optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; and wherein
said tertiary amine is NRₓR_{y}R_{z}, wherein Rₓ, R_{y}, and R_{z} are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl, or Ar₃ is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar₃ is optionally replaced with O, NR′, S(O)ₚ;
L′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, haloalkyl, thiocarbonyl, ester, thioester, alkoxy, alkenoxy, cyano, nitro, imino, alkylamino, aminoalkyl, sulfhydryl, thioalkyl, sulfonyl, or oxygen to form a carbonyl, or wherein any carbon atom of said alkyl or alkenyl is optionally replaced with O, NR₅, S(O)ₚ
Ar₃ is selected from the group consisting of pyrrolidinyl, pyridyl, pyrimidyl, pyrazyl, pyridazyl, quinolinyl, and isoquinolinyl; or,
Ar₄ is an alicyclic or aromatic, mono-, bi- or tricyclic, carbo- or heterocyclic ring, wherein the ring is optionally substituted with one or more substituent(s) independently selected from the group consisting of alkylamino, amido, amino, aminoalkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, ester, formanilido, halo, haloalkyl, hydroxy, imino, isocyano, isonitrilo, nitrilo, nitro, nitroso, phenoxy, sulfhydryl, sulfonylsulfoxy, thio, thioalkyl, thiocarbonyl, thiocyano, thioester, thioformamido, trifluoromethyl, and carboxylic and heterocyclic moieties; wherein the individual alicyclic or aromatic ring contains 5-8 members and wherein said heterocyclic ring contains 1-6 heteroatom(s) independently selected from the group consisting of O, N, and S; and wherein any aromatic or tertiary alkyl amine is optionally oxidized to a corresponding N-oxide;
Ar₅ is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar₅ optionally contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
R₅ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₄ or Ar₁ group;
U is either O or N, provided that:
when U is O, then R′ is a lone pair of electrons and R″ is selected from the group consisting of Ar₄, C₃-C₈ cycloalkyl, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; and
when U is N, then R′ and R″ are, independently, selected from the group consisting of hydrogen, Ar₄, C₃-C₁₀ cycloalkyl, a C₇-C₁₂ bi- or tri-cyclic carbocycle, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; or R′ and R″ are taken together to form a heterocyclic 5- or 6-membered ring selected from the group consisting of pyrrolidine, imidazolidine, pyrazolidine, piperidine, and piperazine; or,
a pharmaceutically acceptable salt, ester or solvate thereof.

59. A method as claimed in claim 58 in which the sensorineurotrophic compound is a compound of formula I: 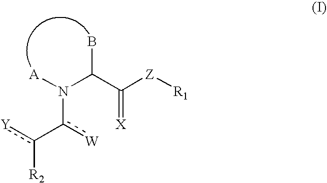
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing one or more heteroatom(s) independently selected from the group consisting of O, S, SO, SO₂, N, NH, and NR₂;
X is either O or S;
Z is either S, CH₂, CHR₁ or CR₁R₃;
W and Y are independently O, S, CH₂ or H₂;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ᵣ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxy; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

60. A method as claimed in claim 59 in which the sensorineurotrophic compound is a compound of formula II: 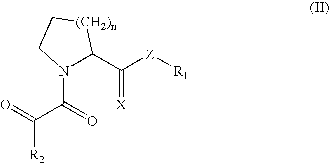
or a pharmaceutically acceptable salt, ester, or solvate thereof, wherein:
n is 1 or 2;
X is O or S;
Z is selected from the group consisting of S, CH₂, CHR₁, and CR₁R₃;
R₁ and R₃ are independently selected from the group consisting of C₁-C₅ straight or branched chain alkyl, C₂-C₅ straight or branched chain alkenyl, and Ar₁, wherein said alkyl, alkenyl or Ar₁ is unsubstituted or substituted with one or more substituent(s) independently selected from the group consisting of halo, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, and Ar₁;
R₂ is selected from the group consisting of C₁-C₉ straight or branched chain alkyl, C₂-C₉ straight or branched chain alkenyl, C₃-C₈ cycloalkyl, C₅-C₇ cycloalkenyl, and Ar₁; and
Ar₁ is phenyl, benzyl, pyridyl, fluorenyl, thioindolyl or naphthyl, wherein said Ar₁ is unsubstituted or substituted with one or more substituent(s) independently selected from the group consisting of halo, trifluoromethyl, hydroxy, nitro, C₁-C₆ straight or branched chain alkyl, C₂-C₆ straight or branched chain alkenyl, C₁-C₄ alkoxy, C₂-C₄ alkenyloxy, phenoxy, benzyloxy, and amino.

61. A method as claimed in claim 59 in which the sensorineurotrophic compound is a compound of formula III: 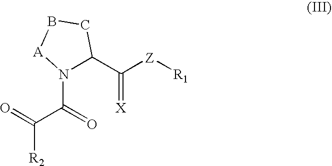
or a pharmaceutically acceptable salt, ester, or solvate thereof, wherein:
A, B, and C are independently CH₂, O, S, SO, SO₂, NH or NR₂;
X is O or S;
Z is S, CH₂, CHR₁ or CR₁R₃;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

62. A method as claimed in claim 59 in which the sensorineurotrophic compound is a compound of formula IV: 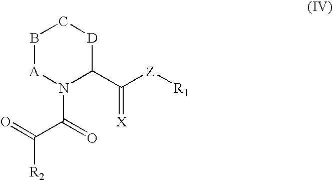
or a pharmaceutically acceptable salt, ester, or solvate thereof, wherein:
A, B, C and D are independently CH₂, O, S, SO, SO₂, NH or NR₂;
X is O or S;
Z is S, CH₂, CHR₁ or CR₁R₃;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ᵣ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₃-C₈ cycloalkyl, C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoro-methyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

63. A method as claimed in claim 58 in which the sensorineurotrophic agent may be a compound of formula VI: 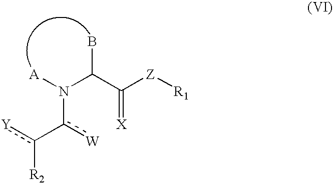
or a pharmaceutically-acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₁;
X is O or S;
Z is O, NH or NR₁;
W and Y are independently O, S, CH₂ or H₂;
R₁ is C₁-C₆ straight or branched chain alkyl or C₂-C₆ straight or branched chain alkenyl, which is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain or alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

64. The method of claim 63 in which the sensorineurotrophic compound is a compound of formula VII: 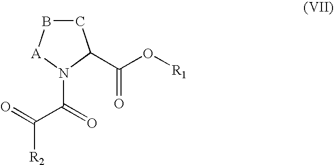
or a pharmaceutically acceptable salt, ester, or solvate thereof, wherein:
A, B and C are independently CH₂, O, S, SO, SO₂, NH or NR₁;
R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁; and
Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

65. The method of claim 64 in which the sensorineurotrophic compound is:

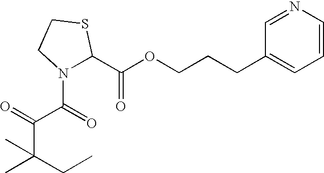

66. A method as claimed in claim 64 in which:
A is CH₂;
B is CH₂ or S;
C is CH₂ or NH;
R₁ is selected from the group consisting of 3-phenylpropyl and 3-(3-pyridyl)propyl; and
R₂ is selected from the group consisting of 1,1-dimethylpropyl, cyclohexyl, and tert-butyl.

67. A method as claimed in claim 63 in which the sensorineurotrophic compound is a compound of formula VIII: 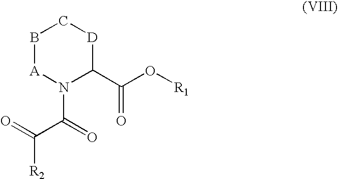
or a pharmaceutically acceptable salt, ester, or solvate thereof, wherein:
A, B, C and D are independently CH₂, O, S, SO, SO₂, NH or NR₁;
R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁; and
Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

68. A method of claim 67 in which:
A is CH₂;
B is CH₂;
C is S, O or NH;
D is CH₂;
R₁ is selected from the group consisting of 3-phenylpropyl and (3,4,5-trimethoxy)phenylpropyl; and
R₂ is selected from the group consisting of 1,1-dimethylpropyl, cyclohexyl, tert-butyl, phenyl, and 3,4,5-trimethoxyphenyl.

69. A method as claimed in claim 58 in which the sensorineurotrophic agent may be a compound of formula IX: 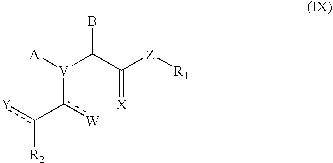
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₃, wherein R is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;
Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and
X is O or S;
Z is O, NH or NR₁;
W and Y are independently O, S, CH₂ or H₂;
R₁ is C₁-C₆ straight or branched chain alkyl or C₂-C₆ straight or branched chain alkenyl, which is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain or alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

70. A method as claimed in claim 58 in which the sensorineurotrophic compound is a compound of formula X: 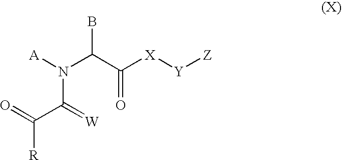
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing one or more heteroatom(s) independently selected from the group consisting of CH, CH₂, O, S, SO, SO₂, N, NH, and NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₂;
Ar₁ and Ar₂ are independently selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is selected from the group consisting of pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl or C₂-C₆ straight or branched chain alkenyl optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

71. A method as claimed in claim 70 in which the sensorineurotrophic compound is a compound of formula XI: 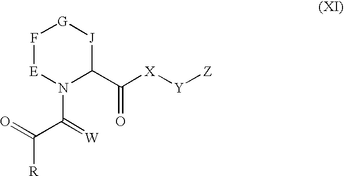
or a pharmaceutically acceptable salt, ester, or solvate thereof, wherein:
E, F, G and J are independently CH₂, O, S, SO, SO₂, NH or NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl, and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, and isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

72. A method as claimed in claim 70 in which the sensorineurotrophic compound is a compound of formula XII: 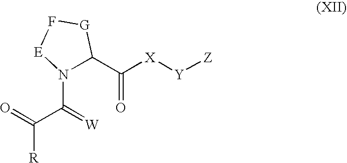
or a pharmaceutically acceptable salt, ester, or solvate thereof, wherein:
E, F, and G are independently CH₂, O, S, SO, SO₂, NH or NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

73. A method as claimed in claim 70 in which the sensorineurotrophic compound is a compound of formula XIII: 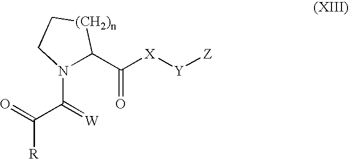
or a pharmaceutically acceptable salt, ester, or solvate thereof, wherein:
n is 1, 2, or 3, forming a 5-7 member heterocyclic ring;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃, independently, are hydrogen, C₁-C₄ straight or branched chain alkyl, C₃-C₄ straight or branched chain alkenyl or alkynyl, or Y-Z.

74. A method as claimed in claim 58 in which the sensorineurotrophic agent may be a compound of formula XIV: 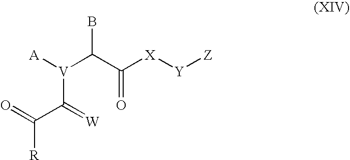
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, —SO₂, N, NH, and NR₇;
R₇ is either C₁-C₉ straight or branched chain alkyl, C₂-C₉ straight or branched chain alkenyl, C₃-C₉ cycloalkyl, C₅-C₇ cycloalkenyl, or Ar₃, wherein R₇ is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;
Ar₃ and Ar₄ are independently an alicyclic or aromatic, mono-, bi- or tricyclic, carbo- or heterocyclic ring; wherein the individual ring size is 5-8 members;
wherein said heterocyclic ring contains 1-6 heteroatom(s) independently selected from the group consisting of O, N, and S; and
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₂;
Ar₁ and Ar₂ are independently selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is selected from the group consisting of pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl or C₂-C₆ straight or branched chain alkenyl optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with 0. NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

75. A method as claimed in claim 58 in which the sensorineurotrophic compound is a compound of formula XV: 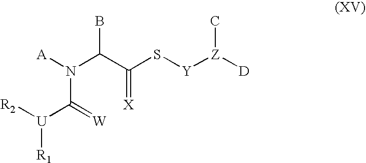
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more additional heteroatom(s) independently selected from the group consisting of O, S, SO, SO₂, N, NH, and NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

76. A method as claimed in claim 75 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

77. A method as claimed in claim 75 in which the sensorineurotrophic compound is a compound of formula XVI: 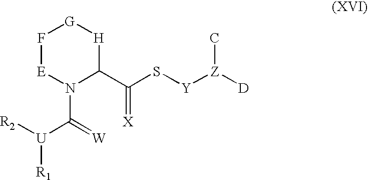
or a pharmaceutically acceptable salt, ester, or solvate thereof, wherein:
E, F, G and J are independently CH₂, O, S, SO, SO₂, NH, or NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

78. A method as claimed in claim 77 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

79. A method as claimed in claim 75 in which the sensorineurotrophic compound is a compound of formula XVII: 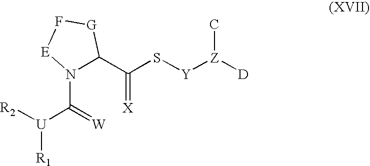
or a pharmaceutically acceptable salt, ester, or solvate thereof, wherein:
E, F, and G are independently CH₂, O, S, SO, SO₂, NH, and NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₈ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

80. A method as claimed in claim 79 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

81. A method as claimed in claim 58 in which the sensorineurotrophic compound is a compound of formula XVIII: 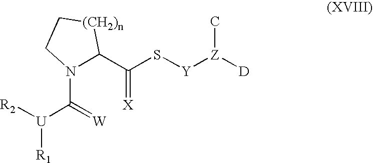
or a pharmaceutically acceptable salt, ester, or solvate thereof, wherein:
n is 1, 2 or 3;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain or alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

82. A method as claimed in claim 81 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

83. A method as claimed in claim 58 in which the sensorineurotrophic compound is a compound of formula XIX: 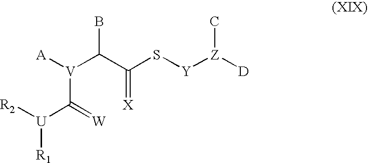
or a pharmaceutically acceptable salt, ester, or solvate thereof, wherein:
V is CH, N, or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂; and
A and B, together with the nitrogen and carbon atoms to which they are respectively attached, form a 5-7 membered saturated or unsaturated heterocyclic ring containing, in addition to the nitrogen atom, one or more additional heteroatom(s) independently selected from the group consisting of O, S, SO, SO₂, N, NH, and NR₃;
X is either O or S;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

84. A method as claimed in claim 58 in which the sensorineurotrophic compound is a compound of formula XX: 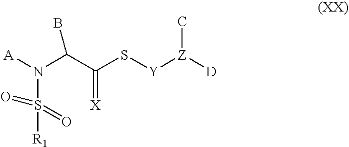
a pharmaceutically acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₃, S, SO, or SO₂.

85. A method as claimed in claim 84 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

86. A method as claimed in claim 85 in which A and B, together with the nitrogen and carbon atoms to which they are respectfully attached, form a 6 membered saturated or unsaturated heterocyclic ring; and R₂ is C₄-C₇ branched chain alkyl, C₄-C₇ cycloalkyl, phenyl, or 3,4,5-trimethoxyphenyl.

87. A method as claimed in claim 84 in which the sensorineurotrophic compound is selected from the group consisting of:
3-(para-Methoxyphenyl)-1-propylmercaptyl(2S)-N-(benzenesulfonyl)pyrrolidine-2-carboxylate;
3-(para-Methoxyphenyl)-1-propylmercaptyl(2S)-N-(α-toluenesulfonyl)pyrrolidine-2-carboxylate;
3-(para-Methoxyphenyl)-1-propylmercaptyl(2S)-N-(α-toluenesulfonyl)pyrrolidine-2-carboxylate;
1,5-Diphenyl-3-pentylmercaptyl-N-(para-toluenesulfonyl)pipecolate; and
pharmaceutically acceptable salts and solvates thereof.

88. A method as claimed in claim 84 in which the sensorineurotrophic compound is a compound of formula XXI: 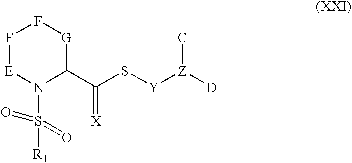
or a pharmaceutically acceptable salt, ester, or solvate thereof, wherein:
E, F, G and J are independently CH₂, O, S, SO, SO₂, NH or NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₃, S, SO, or SO₂.

89. A method as claimed in claim 88 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

90. A method as claimed in claim 84 in which the sensorineurotrophic agent is a compound of formula XXII: 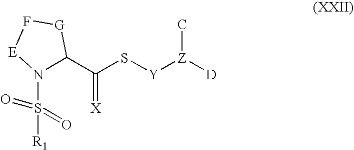
or a pharmaceutically acceptable salt, ester, or solvate thereof, wherein:
E, F, and G are independently CH₂, O, S, SO, SO₂, NH or NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₃, S, SO, or SO₂.

91. A method as claimed in claim 90 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

92. A method as claimed in claim 84 in which the sensorineurotrophic compound is a compound of formula XXIII: 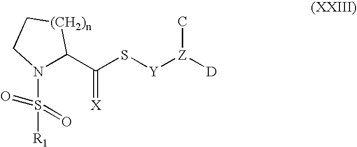
or a pharmaceutically acceptable salt, ester, or solvate thereof, wherein:
n is 1, 2 or 3;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₃, S, SO, or SO₂.

93. A method as claimed in claim 92 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

94. A method as claimed in claim 58 in which the sensorineurotrophic compound is a compound of formula XXIV: 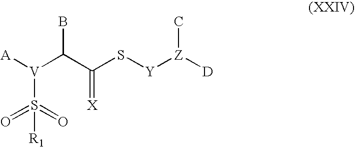
or a pharmaceutically acceptable salt, ester, or solvate thereof, wherein:
V is CH, N, or S;
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₂, S, SO, or SO₂.

95. A method as claimed in claim 58 in which the sensorineurotrophic compound is a compound of formula XXV: 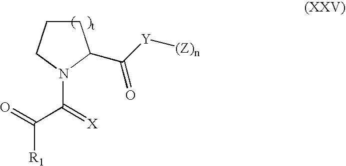
or a pharmaceutically acceptable salt, ester, or solvate thereof, wherein:
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
X is O, S, CH₂ or H₂;
Y is O or NR₂, wherein R₂ is a direct bond to a Z, hydrogen or C₁-C₆ alkyl; and
each Z, independently, is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 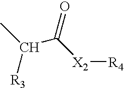
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;
n is 1 or 2, and;
t is 1, 2 or 3.

96. A method as claimed in claim 95 in which the compound is selected from the group consisting of:
3-phenyl-1-propyl (2S)-1-(3,3-dimethyl-1,2-dioxopentyl)-2-pyrrolidinecarboxylate;
3-phenyl-1-prop-2-(E)-enyl (2S)-1-(3,3-dimethyl-1,2-dioxopentyl)-2-pyrrolidinecarboxylate;
3-(3,4,5-trimethoxyphenyl)-1-propyl (2S)-1-(3,3-dimethyl-1,2-dioxopentyl)-2-pyrrolidine-carboxylate;
3-(3,4,5-trimethoxyphenyl)-1-prop-2-(E)-enyl (2S)-1-(3,3-dimethyl-1,2-dioxopentyl)-2-pyrrolidinecarboxylate;
3-(4,5-dichlorophenyl)-1-propyl (2S)-1-(3,3-dimethyl-1,2-dioxopentyl)-2-pyrrolidinecarboxylate;
3-(4,5-dichlorophenyl)-1-prop-2-(E)-enyl (2S)-1-(3,3-dimethyl-1,2-dioxopentyl)-2-pyrrolidine-carboxylate;
3-(4,5-methylenedioxyphenyl)-1-propyl (2S)-1-(3,3-dimethyl-1,2-dioxopentyl)-2-pyrrolidine-carboxylate;
3-(4,5-methylenedioxyphenyl)-1-prop-2-(E)-enyl (2S)-1-(3,3-dimethyl-1,2-dioxopentyl)-2-pyrrolidinecarboxylate;
3-cyclohexyl-1-propyl (2S)-1-(3,3-dimethyl-1,2-dioxopentyl)-2-pyrrolidinecarboxylate;
3-cyclohexyl-1-prop-2-(E)-enyl (2S)-1-(3,3-dimethyl-1,2-dioxopentyl)-2-pyrrolidinecarboxylate;
(1R)-1,3-diphenyl-1-propyl (2S)-1-(3,3-dimethyl-1,2-dioxopentyl)-2-pyrrolidinecarboxylate;
(1R)-1,3-diphenyl-1-prop-2-(E)-enyl (2S)-1-(3,3-dimethyl-1,2-dioxopentyl)-2-pyrrolidine-carboxylate;
(1R)-1-cyclohexyl-3-phenyl-1-propyl (2S)-1-(3,3-dimethyl-1,2-dioxopentyl)-2-pyrrolidine-carboxylate;
(1R)-1-cyclohexyl-3-phenyl-1-prop-2-(E)-enyl (2S)-1-(3,3-dimethyl-1,2-dioxopentyl)-2-pyrrolidinecarboxylate;
(1R)-1-(4,5-dichlorophenyl)-3-phenyl-1-propyl (2S)-1-(3,3-dimethyl-1,2-dioxopentyl)-2-pyrrolidine-carboxylate;
3-phenyl-1-propyl (2S)-1-(1,2-dioxo-2-cyclohexyl)ethyl-2-pyrrolidinecarboxylate;
3-phenyl-1-propyl (2S)-1-(1,2-dioxo-4-cyclohexyl)butyl-2-pyrrolidinecarboxylate;
3-phenyl-1-propyl (2S)-1-(1,2-dioxo-2-[2-furanyl])ethyl-2-pyrrolidinecarboxylate;
3-phenyl-1-propyl (2S)-1-(1,2-dioxo-2-[2-thienyl])ethyl-2-pyrrolidinecarboxylate;
3-phenyl-1-propyl (2S)-1-(1,2-dioxo-2-[2-thiazolyl])ethyl-2-pyrrolidinecarboxylate;
3-phenyl-1-propyl (2S)-1-(1,2-dioxo-2-phenyl)ethyl-2-pyrrolidinecarboxylate;
1,7-diphenyl-4-heptyl (2S)-1-(3,3-dimethyl-1,2-dioxopentyl)-2-pyrrolidinecarboxylate;
3-phenyl-1-propyl (2S)-1-(3,3-dimethyl-1,2-dioxo-4-hydroxybutyl)-2-pyrrolidinecarboxylate;
3-phenyl-1-propyl (2S)-1-(3,3-dimethyl-1,2-dioxopentyl)-2-pyrrolidinecarboxamide;
1-[1-(3,3-dimethyl-1,2-dioxopentyl)-L-proline]-L-phenylalanine ethyl ester;
1-[1-(3,3-dimethyl-1,2-dioxopentyl)-L-proline]-L-leucine ethyl ester;
1-[1-(3,3-dimethyl-1,2-dioxopentyl)-L-proline]-L-phenylglycine ethyl ester;
1-[1-(3,3-dimethyl-1,2-dioxopentyl)-L-proline]-L-phenylalanine phenyl ester;
1-[1-(3,3-dimethyl-1,2-dioxopentyl)-L-proline]-L-phenylalanine benzyl ester;
1-[1-(3,3-dimethyl-1,2-dioxopentyl)-L-proline]-L-isoleucine ethyl ester; and
pharmaceutically acceptable salts, esters, and solvates thereof.

97. A method as claimed in claim 95 in which the sensorineurotrophic compound is a compound of formula XXVI: 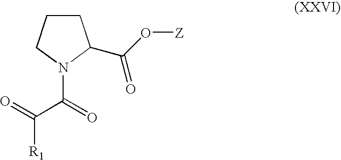
or a pharmaceutically acceptable salt, ester, or solvate thereof, wherein:
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
Z is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 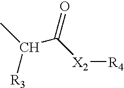
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl; and
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl.

98. A method as claimed in claim 58 in which the sensorineurotrophic agent may be a compound of formula XXVII: 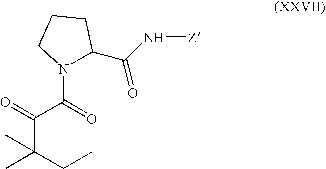
or a pharmaceutically acceptable salt, ester, or solvate thereof, wherein:
Z′ is the fragment 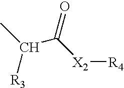
wherein:
R₃ is C₁-C₉ straight or branched chain alkyl or unsubstituted Ar₁, wherein said alkyl is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and
Ar₁ is selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino.

99. A method as claimed in claim 95 in which the sensorineurotrophic agent may also be a compound of formula XXVIII: 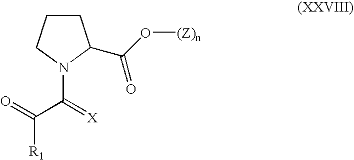
wherein:
R₁ is C₁-C₆ straight or branched chain alkyl, C₂-C₆ straight or branched chain alkenyl, C₃-C₆ cycloalkyl or Ar₁, wherein said alkyl or alkenyl is unsubstituted or substituted with C₃-C₆ cycloalkyl or Ar₂;
Ar₁ and Ar₂ are independently selected from the group consisting of 2-furyl, 2-thienyl, and phenyl;
X is selected from the group consisting of oxygen and sulfur;
Y is oxygen or NR₂, wherein R₂ is a direct bond to a Z, hydrogen or C₁-C₆ alkyl;
each Z, independently, is hydrogen, C₁-C₆ straight or branched chain alkyl, or C₂-C₆ straight or branched chain alkenyl, wherein said Z is substituted with one or more substituent(s) independently selected from the group consisting of 2-furyl, 2-thienyl, C₃-C₆ cycloalkyl, pyridyl, and phenyl, each having one or more substituent(s) independently selected from the group consisting of hydrogen and C₁-C₄ alkoxy; and
n is 1 or 2.

100. A method as claimed in claim 99 in which the compound is selected from the group consisting of:
3-(2,5-dimethoxyphenyl)-1-propyl (2S)-1-(3,3-dimethyl-1,2-dioxopentyl)-2-pyrrolidinecarboxylate;
3-(2,5-dimethoxyphenyl)-1-prop-2-(E)-enyl (2S)-1-(3,3-dimethyl-1,2-dioxopentyl)-2-pyrrolidine-carboxylate;
2-(3,4,5-trimethoxyphenyl)-1-ethyl (2S)-1-(3,3-dimethyl-1,2-dioxopentyl)-2-pyrrolidinecarboxylate;
3-(3-pyridyl)-1-propyl (2S)-1-(3,3-dimethyl-1,2-dioxopentyl)-2-pyrrolidinecarboxylate;
3-(2-pyridyl)-1-propyl (2S)-1-(3,3-dimethyl-1,2-dioxopentyl)-2-pyrrolidinecarboxylate;
3-(4-pyridyl)-1-propyl (2S)-1-(3,3-dimethyl-1,2-dioxopentyl)-2-pyrrolidinecarboxylate;
3-phenyl-1-propyl (2S)-1-(2-tert-butyl-1,2-dioxoethyl)-2-pyrrolidinecarboxylate;
3-phenyl-1-propyl (2S)-1-(2-cyclohexylethyl-1,2-dioxoethyl)-2-pyrrolidinecarboxylate;
3-(3-pyridyl)-1-propyl (2S)-1-(2-cyclohexylethyl-1,2-dioxoethyl)-2-pyrrolidine-carboxylate;
3-(3-pyridyl)-1-propyl (2S)-1-(2-tert-butyl-1,2-dioxoethyl)-2-pyrrolidinecarboxylate;
3,3-diphenyl-1-propyl (2S)-1-(3,3-dimethyl-1,2-dioxopentyl)-2-pyrrolidinecarboxylate;
3-(3-pyridyl)-1-propyl (2S)-1-(2-cyclohexyl-1,2-dioxoethyl)-2-pyrrolidinecarboxylate;
3-(3-pyridyl)-1-propyl (2S)-N-([2-thienyl]glyoxyl)pyrrolidinecarboxylate;
3,3-diphenyl-1-propyl (2S)-1-(3,3-dimethyl-1,2-dioxobutyl)-2-pyrrolidinecarboxylate;
3,3-diphenyl-1-propyl (2S)-1-cyclohexylglyoxyl-2-pyrrolidinecarboxylate;
3,3-diphenyl-1-propyl (2S)-1-(2-thienyl)glyoxyl-2-pyrrolidinecarboxylate; and
pharmaceutically acceptable salts, esters, and solvates thereof.

101. A method as claimed in claim 58 in which the sensorineurotrophic compound is a compound of formula XXIX: 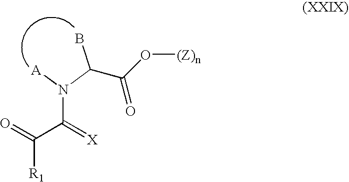
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo-(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, alkylthio, sulfhydryl, amino, (C₁-C₆)-alkylamino, amino-(C₁-C₆)-alkyl, aminocarboxyl, and Ar₂;
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S;
X is O, S, CH₂ or H₂;
Y is O or NR₂, wherein R₂ is a direct bond to a Z, hydrogen or C₁-C₆ alkyl; and
each Z, independently, is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 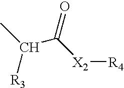
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl; and
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and,
n is 1 or 2.

102. A method as claimed in claim 58 in which the sensorineurotrophic compound is a compound of formula (LV): 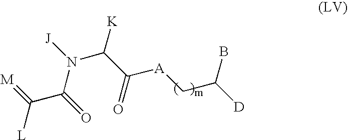
or a pharmaceutically acceptable salt, ester, or solvate thereof, wherein:
m is 0-3;
A is CH₂, O, NH, or N—(C₁-C₄ alkyl);
B and D are independently hydrogen, Ar, C₅-C₇ cycloalkyl substituted C₁-C₆ straight or branched chain alkyl or C₂-C₆ straight or branched chain alkenyl, C₅-C₇ cycloalkenyl substituted C₁-C₆ straight or branched chain alkyl or C₂-C₆ straight or branched chain alkenyl, or Ar substituted C₁-C₆ straight or branched chain alkyl or C₂-C₆ straight or branched chain alkenyl, wherein in each case, one or two carbon atom(s) of said alkyl or alkenyl may be substituted with one or two heteroatom(s) independently selected from the group consisting of oxygen, sulfur, SO, and SO₂ in chemically reasonable substitution patterns, or 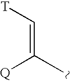
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
L is either hydrogen or U; M is either oxygen or CH-U, provided that if L is hydrogen, then M is CH-U, or if M is oxygen then L is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylmethyl; or J and K are taken together to form a 5-7 membered heterocyclic ring which is substituted with oxygen, sulfur, SO, or SO₂.

103. A method as claimed in claim 58 in which the sensorineurotrophic compound is a compound of formula (LVI): 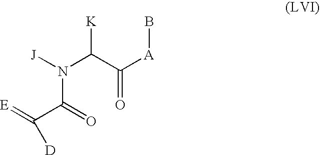
or a pharmaceutically acceptable salt, ester, or solvate thereof, wherein:
A is O, NH, or N—(C₁-C₄ alkyl);
B is hydrogen, CHL-Ar, C₁-C₆ straight or branched chain alkyl, C₂-C₆ straight or branched chain alkenyl, C₅-C₇ cycloalkyl, C₅-C₇ cycloalkenyl, Ar substituted C₁-C₆ alkyl or C₂-C₆ alkenyl, or 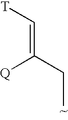
wherein L and Q are independently hydrogen, C₁-C₆ straight or branched chain alkyl, or C₂-C₆ straight or branched chain alkenyl; and
T is Ar or C₅-C₇ cyclohexyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 2-pyridyl, 3-pyridyl, 4-pyridyl and phenyl having 1-3 substituent(s) independently selected from the group consisting of hydrogen, halo, hydroxy, nitro, CF₃, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, and phenyl.
D is hydrogen or U; E is oxygen or CH-U, provided that if D is hydrogen, then E is CH-U, or if E is oxygen, then D is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇-cycloalkyl, C₅-C₇ cycloalkenyl substituted with C₁-C₄ straight or branched chain alkyl or C₂-C₄ straight or branched chain alkenyl, 2-indolyl, 3-indolyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylethyl; or J and K are taken together to form a. 5-7 membered heterocyclic ring which is substituted with oxygen, sulfur, SO, or SO₂.

104. A method as claimed in claim 58 in which the sensorineurotrophic compound is a compound of formula LVIII: 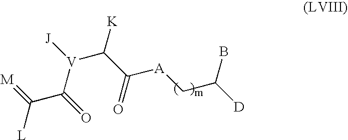
or a pharmaceutically acceptable salt, ester, or solvate thereof, wherein:
V is CH, N, or S;
J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;
Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;
A is CH₂, O, NH, or N—(C₁-C₄ alkyl);
B and D are independently hydrogen, Ar, C₅-C₇ cycloalkyl substituted C₁-C₆ straight or branched chain alkyl or C₂-C₆ straight or branched chain alkenyl, C₅-C₇ cycloalkenyl substituted C₁-C₆ straight or branched chain alkyl or C₂-C₆ straight or branched chain alkenyl, or Ar substituted C₁-C₆ straight or branched chain alkyl or C₂-C₆ straight or branched chain alkenyl, wherein in each case, one or two carbon atom(s) of said alkyl or alkenyl may be substituted with one or two heteroatom(s) independently selected from the group consisting of oxygen, sulfur, SO, and SO₂ in chemically reasonable substitution patterns, or 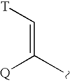
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
L is either hydrogen or U; M is either oxygen or CH-U, provided that if L is hydrogen, then M is CH-U, or if M is oxygen then L is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylmethyl; or J and K are taken together to form a 5-7 membered heterocyclic ring which is substituted with oxygen, sulfur, SO, or SO₂.

105. A method as claimed in claim 58 in which the sensorineurotrophic compound is a compound of the formula (LIX): 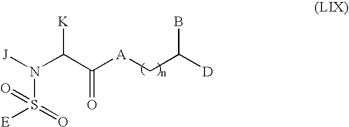
or a pharmaceutically acceptable salt, ester or solvate thereof, wherein:
A is CH₂, O, NH, or N—(C₁-C₄ alkyl);
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 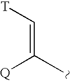
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar;
J is hydrogen, C₁ or C₂ alkyl, or benzyl; K is C₁-C₄ straight or branched chain alkyl, benzyl, or cyclohexylmethyl; or J and K are taken together to form a 5-7 membered heterocyclic ring which is substituted with O, S, SO, or SO₂;
n is 0 to 3.

106. A method as claimed in claim 58 in which the sensorineurotrophic compound is a compound of Formula LXI: 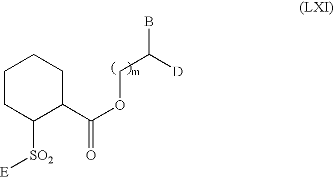
or a pharmaceutically acceptable salt, ester or solvate thereof, wherein:
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 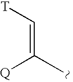
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and
m is 0 to 3.

107. A method as claimed in claim 58 in which the sensorineurotrophic compound is a compound of Formula (LXII): 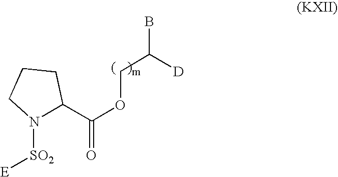
or a pharmaceutically acceptable salt thereof, wherein:
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl, or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 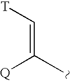
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and
m is 0 to 3.

108. A method as claimed in claim 58 in which the sensorineurotrophic compound is a compound of Formula LXIII: 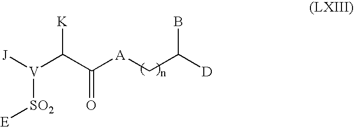
or a pharmaceutically acceptable salt, ester, or solvate thereof, wherein:
V is CH, N, or S;
J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;
Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;
A is CH₂, O, NH, or N—(C₁-C₄ alkyl);
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 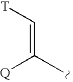
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar;
J is hydrogen, C₁ or C₂ alkyl, or benzyl; K is C₁-C₄ straight or branched chain alkyl, benzyl, or cyclohexylmethyl; or J and K are taken together to form a 5-7 membered heterocyclic ring which is substituted with O, S, SO, or SO₂;
n is 0 to 3.

109. A method as claimed in claim 58 in which the sensorineurotrophic compound is a compound of formula (LXIV): 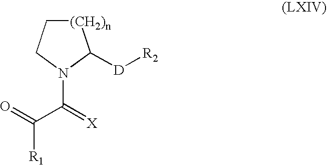
in which:
n is 1-3;
X is either O or S;
R₁ is selected from the group consisting of C₁-C₉ straight or branched chain alkyl, C₂-C₉ straight or branched chain alkenyl, aryl, heteroaryl, carbocycle, or heterocycle;
D is a bond, or a C₁-C₁₀ straight or branched chain alkyl, C₂-C₁₀ alkenyl or C₂-C₁₀ alkynyl; and
R₂ is a carboxylic acid or a carboxylic acid isostere;
or a pharmaceutically acceptable salt, ester, or solvate thereof.

110. A method as claimed in claim 109 in which.
R₂ is selected from the group: 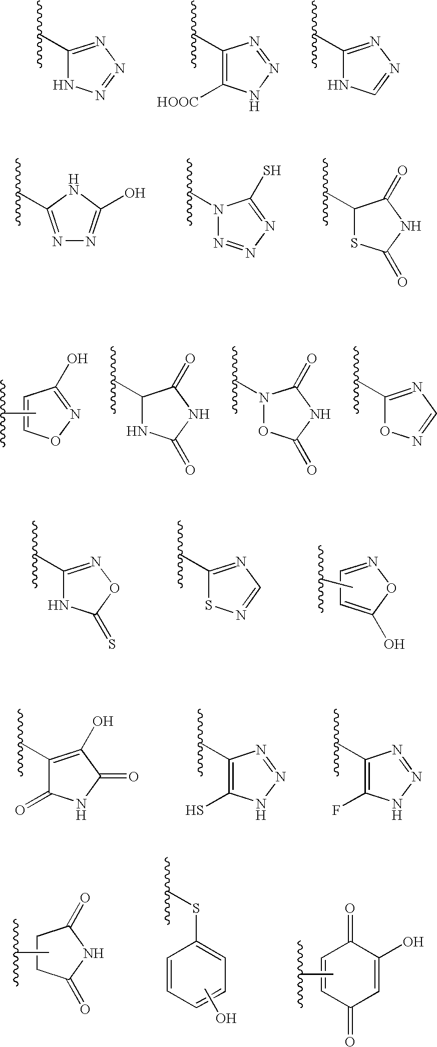
—COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl.

111. A method as claimed in claim 58 in which the sensorineurotrophic compound is a compound of formula (LXV): 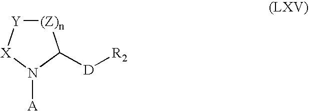
in which
X, Y, and Z are independently selected from the group consisting of C, O, S, or N, provided that X, Y, and Z are not all C;
n is 1-3;
A is selected from the group consisting of L₁, L₂, L₃, or L₄, in which 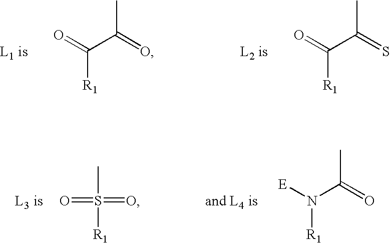
and R₁ and E, independently, are selected from the group consisting of hydrogen, C₁-C₉ straight or branched chain alkyl, C₂-C₉ straight or branched chain alkenyl, aryl, heteroaryl, carbocycle, and heterocycle;
R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where
R³ is hydrogen, hydroxy, halo, halo(C₁-C₆)-alkyl, thiocarbonyl, (C₁-C₆)-alkoxy, (C₂-C₆)-alkenoxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;
or a pharmaceutically acceptable salt, ester, or solvate thereof.

112. A method as claimed in claim 58 in which the sensorineurotrophic compound is a compound of formula (LXVI): 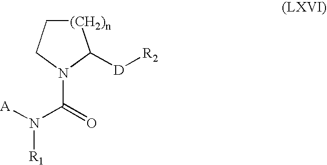
in which:
n is 1-3;
R₁ and A are independently selected from the group consisting of hydrogen, C₁-C₉ straight or branched chain alkyl, C₂-C₉ straight or branched chain alkenyl, aryl, heteroaryl, carbocycle, and heterocycle;
D is a bond, or a C₁-C₁₀ straight or branched chain alkyl, C₂-C₁₀ alkenyl or C₂-C₁₀ alkynyl;
R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where R³ is hydrogen, hydroxy, halo, halo(C₁-C₆)-alkyl, thiocarbonyl, (C₁-C₆)-alkoxy, (C₂-C₆)-alkenoxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl;
or a pharmaceutically acceptable salt, ester, or solvate thereof.

113. A method as claimed in claim 58 in which the sensorineurotrophic compound is a compound of formula (LXVII): 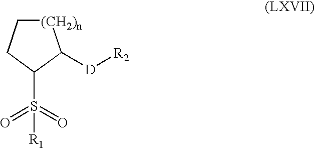
in which:
n is 1-3;
R₁ is selected from the group consisting of hydrogen, C₁-C₉ straight or branched chain alkyl, C₂-C₉ straight or branched chain alkenyl, aryl, heteroaryl, carbocycle, or heterocycle;
D is a bond, or a C₁-C₁₀ straight or branched chain alkyl, C₂-C₁₀ alkenyl or C₂-C₁₀ alkynyl;
R₂ is a carboxylic acid or a carboxylic acid isostere;
wherein said alkyl, alkenyl, alkynyl, aryl, heteroaryl, carbocycle, heterocycle, or carboxylic acid isostere is optionally substituted with one or more substituents selected from R³, where
R³ is hydrogen, hydroxy, halo, halo-(C₁-C₆)-alkoxy, thiocarbonyl, (C₁-C₆)-alkoxy, (C₂-C₆)-alkenyloxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;
or a pharmaceutically acceptable salt, ester or solvate thereof.

114. A method for the prevention or treatment of injury or degeneration of inner ear sensory cells which comprises administering to a warm-blooded animal a compound selected from the group comprising: 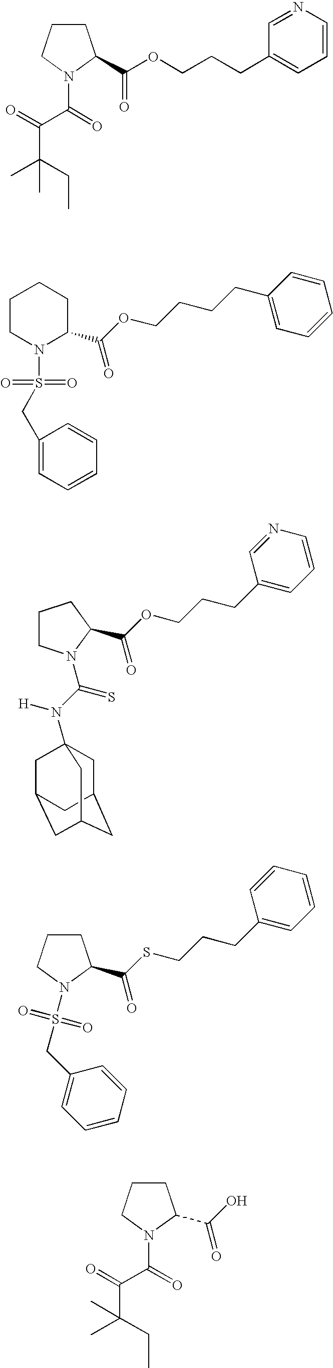 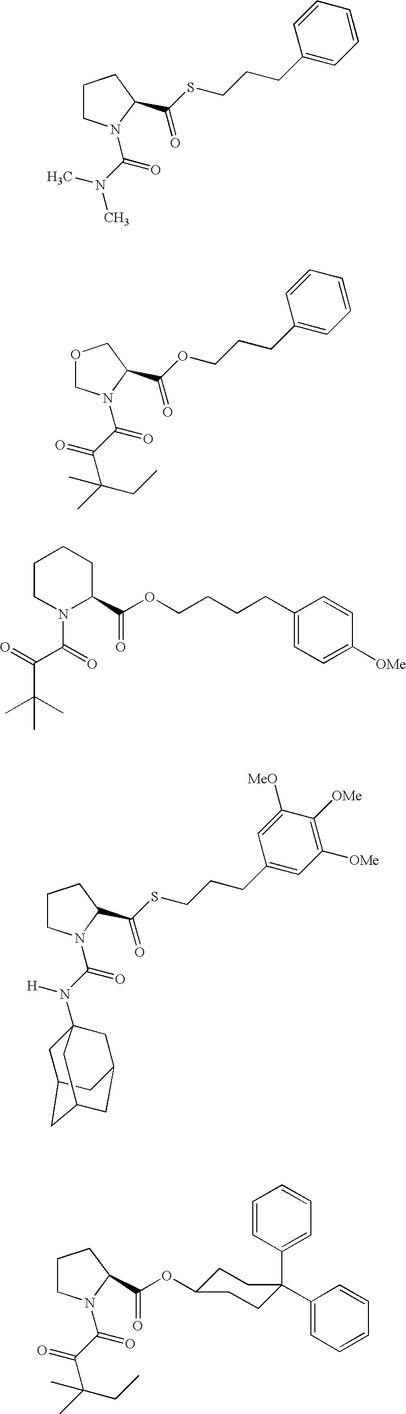 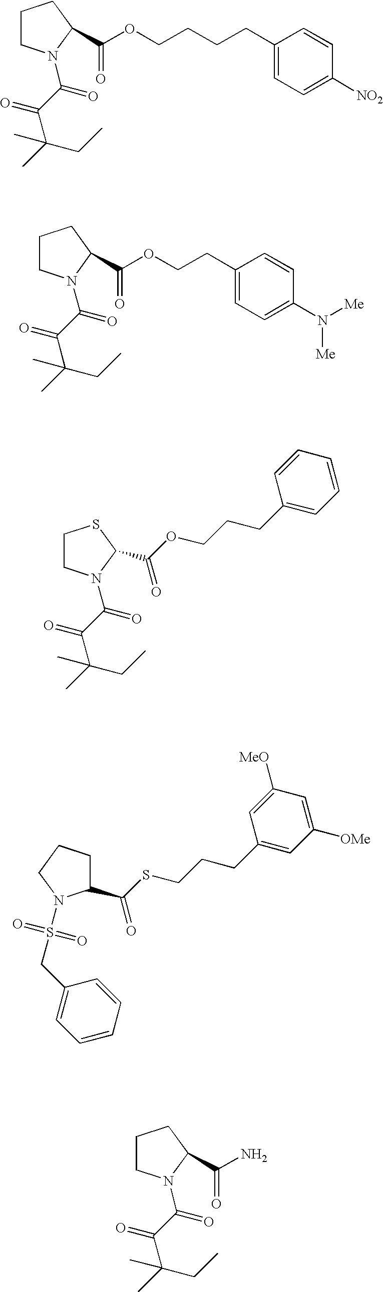 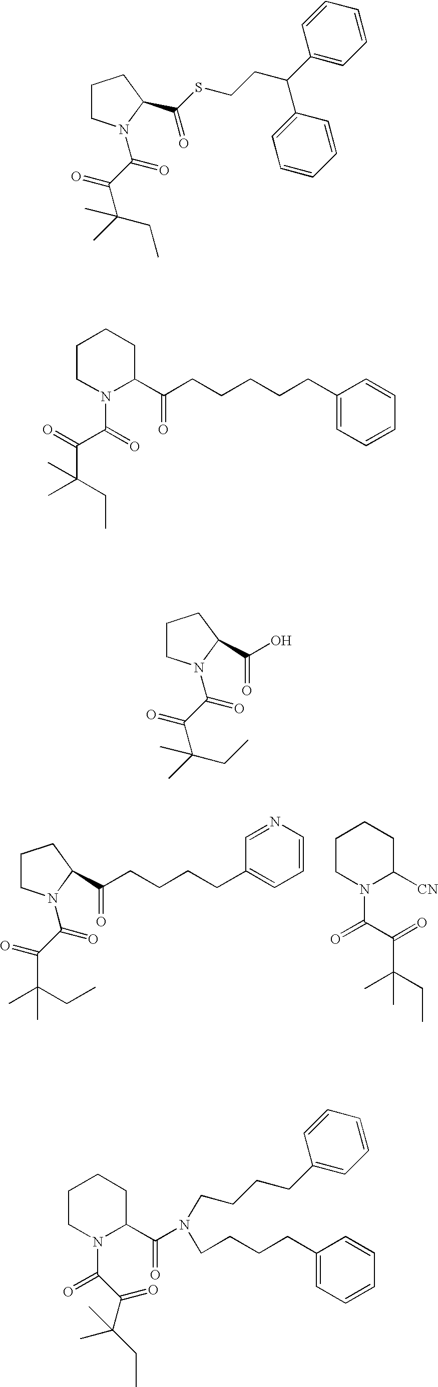 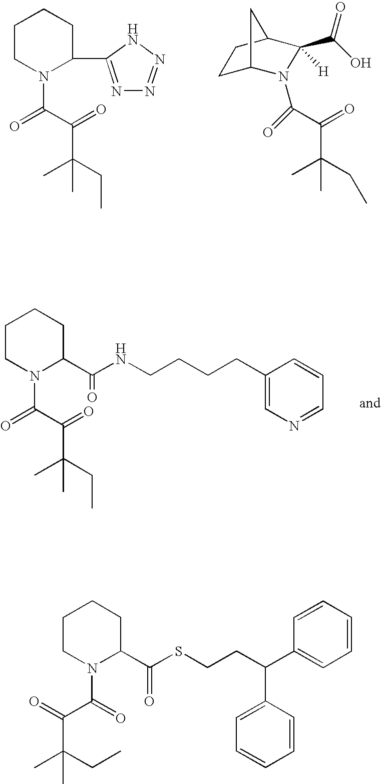
or a pharmaceutically acceptable salt, ester of solvate thereof.

115. A method for the prevention or treatment of a vestibular disorder which comprises administering to a warm-blooded animal a sensorineurotrophic compound of the formula (I′): 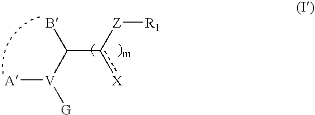
wherein
A′ is hydrogen, C₁ or C₂ alkyl, or benzyl;
B′ is C₁-C₄ straight or branched chain alkyl, benzyl or cyclohexylmethyl; or,
A′ and B′, taken together with the atoms to which they are attached, form a 5-7 membered saturated, unsaturated or aromatic heterocylic or carbocyclic ring which contains one or more additional O, C(R₁)₂, S(O)ₚ, N, NR₁, or NR₅ atoms;
V is CH, S, or N;
G is 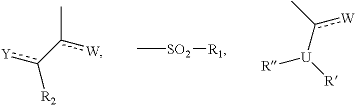
each R₁, independently, is hydrogen, C₁-C₉ straight or branched chain alkyl, or C₂-C₉ straight or branched chain alkenyl or alkynyl, C₃-C₉ cycloalkyl, C₅-C₇ cycloalkenyl, a carboxylic acid or carboxylic acid isostere, N(R₄)ₙ, Ar₁, Ar₄ or K-L wherein said alkyl, cycloalkyl, cycloalkenyl, alkynyl, alkenyl, Ar₁ or Ar₄ is optionally substituted with one or more substituent(s) independently selected from the group consisting of:
2-furyl, 2-thienyl, pyridyl, phenyl, C₃-C₆ cycloalkyl wherein said furyl, thienyl, pyridyl, phenyl or cycloalkyl group optionally is substituted with C₁-C₄ alkoxy, (Ar₁)ₙ, halo, halo-C₁-C₆-alkyl, carbonyl, thiocarbonyl, C₁-C₆ thioester, cyano, imino, COOR₆ in which R₆ is C₁-C₉ straight or branched chain alkyl or alkenyl, hydroxy, nitro, trifluoromethyl, C₁-C₆ alkoxy, C₂-C₄ alkenyloxy, C₁-C₆ alkylaryloxy C₁-C₆ aryloxy, aryl-(C₁-C₆)-alkyloxy, phenoxy, benzyloxy, thio-(C₁-C₆)-alkyl, C₁-C₆-alkylthio, sulfhydryl, sulfonyl, amino, (C₁-C₆)-mono- or di-alkylamino, amino-(C₁-C₆)-alkyl, aminocarboxy, C₃-C₈ cycloalkyl, C₁-C₆ straight or branched chain alkyl, C₂-C₆ straight or branched chain alkenyl optionally substituted with (Ar₁)ₙ, C₃-C₈ cycloalkyl, C₁-C₆ straight or branched chain alkyl, C₂-C₆ straight or branched chain alkenyl substituted with C₃-C₈ cycloalkyl, C₃-C₈ cycloalkyl, and Ar₂₁ and, wherein any carbon atom of an alkyl or alkenyl group may optionally replaced with O, NR₅ or S(O)_{P}; or,
R₁ is a moiety of the formula: 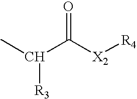
wherein:
R₃ is C₁-C₉ straight or branched chain alkyl which is optionally substituted with C₃-C₈ cycloalkyl or Ar₁;
X₂ is O or NR₆, wherein R₆ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;
R₂ is C₀-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl, or cycloalkenyl is optionally substituted with one or more substituents selected from the group consisting of C₁-C₆ straight or branched chain alkyl, C₂-C₆ straight or branched chain alkenyl, C₃-C₈ cycloalkyl, C₅-C₇ cycloalkenyl, (Ar₁)ₙ and hydroxy; or,
R₂ is either hydrogen or P; Y is either oxygen or CH-P, provided that if R₂ is hydrogen, then Y is CH-P, or if Y is oxygen then R₂ is P;
P is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar₅, or Ar₅
Ar₁ or Ar₂, independently, is an alicyclic or aromatic, mono-, bi- or tricyclic, carbo- or heterocyclic ring, wherein the ring is optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₃-C₈ cycloalkyl, C₅-C₇ cycloalkenyl, C₁-C₄ alkoxy, C₂-C₄ alkenyloxy, phenoxy, benzyloxy, and amino; wherein the individual ring contains 5-8 members; and wherein the heterocyclic ring contains 1-6 heteroatom(s) independently selected from the group consisting of O, N, and S, and, wherein any aromatic or tertiary alkylamine is optionally oxidized to a corresponding N-oxide;
m is 0 or 1
n is 1 or 2;
p is 0, 1, or 2;
t is 0, 1, 2, 3, or 4;
X is O, CH₂ or S;
W and Y, independently, are O, S, CH₂ or H₂;
Z is C(R₁)₂, O, S, a direct bond or NR₁; or, Z-R₁ is J-K-L, 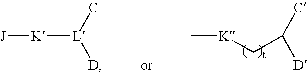
wherein:
C and D are, independently, hydrogen, Ar₄, Ar₁, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, Ar₁ and Ar₄; wherein said alkyl, alkenyl, cycloalkyl or cycloalkenyl is optionally substituted with C₁-C₆ alkyl, C₂-C₆ alkenyl, hydroxy, amino, halo, haloalkyl, thiocarbonyl, C₁-C₆ ester, C₁-C₆ thioester, C₁-C₆ alkoxy, C₁-C₆ alkenoxy, cyano, nitro, imino, C₁-C₆ alkylamino, amino-(C₁-C₆)alkyl, sulfhydryl, thio-(C₁-C₆)alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NR₅, or (SO)ₚ;
C′ and D′ are independently hydrogen, Ar₅, C₁-C₆ straight or branched chain alkyl, or C₂-C₆ straight or branched chain alkenyl, wherein said alkyl or alkenyl is optionally substituted with C₅-C₇ cycloalkyl, C₅-C₇ cycloalkenyl, or Ar₅, wherein, one or two carbon atom(s) of said alkyl or alkenyl may be substituted with one or two heteroatom(s) independently selected from the group consisting of oxygen, sulfur, SO, and SO₂ in chemically reasonable substitution patterns, or 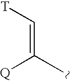
wherein Q is hydrogen, C₁-C₆ straight or branched chain alkyl, or C₂-C₆ straight or branched chain alkenyl; and
T is Ar₅ or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl J is O, NR₁, S, or (CR₁)₂;
K is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl or Ar₃, is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl or Ar₃₁ is optionally replaced with O, NR′″, or S(O)ₚ;
K′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, haloalkyl, thiocarbonyl, ester, thioester, alkoxy, alkenoxy, cyano, nitro, imino, alkylamino, aminoalkyl, sulfhydryl, thioalkyl, sulfonyl, or oxygen to form a carbonyl, or wherein any carbon atom of said alkyl or alkenyl is optionally replaced with O, NR₅, S(O)ₚ;
K″ is C(R₁)₂, O, S, a direct bond or NR₁;
R′″ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₃ group;
L is an aromatic amine or a tertiary amine oxidized to a corresponding N-oxide;
said aromatic amine being selected from the group consisting of pyridyl, pyrimidyl, quinolinyl, and isoquinolinyl, said aromatic amine being optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; and wherein
said tertiary amine is NRₓR_{y}R_{z}, wherein Rₓ, R_{y}, and R_{z} are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl, or Ar₃ is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar₃ is optionally replaced with O, NR′, S(O)ₚ;
L′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, haloalkyl, thiocarbonyl, ester, thioester, alkoxy, alkenoxy, cyano, nitro, imino, alkylamino, aminoalkyl, sulfhydryl, thioalkyl, sulfonyl, or oxygen to form a carbonyl, or wherein any carbon atom of said alkyl or alkenyl is optionally replaced with O, NR₅, S(O)ₚ
Ar₃ is selected from the group consisting of pyrrolidinyl, pyridyl, pyrimidyl, pyrazyl, pyridazyl, quinolinyl, and isoquinolinyl; or,
Ar₄ is an alicyclic or aromatic, mono-, bi- or tricyclic, carbo- or heterocyclic ring, wherein the ring is optionally substituted with one or more substituent(s) independently selected from the group consisting of alkylamino, amido, amino, aminoalkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, ester, formanilido, halo, haloalkyl, hydroxy, imino, isocyano, isonitrilo, nitrilo, nitro, nitroso, phenoxy, sulfhydryl, sulfonylsulfoxy, thio, thioalkyl, thiocarbonyl, thiocyano, thioester, thioformamido, trifluoromethyl, and carboxylic and heterocyclic moieties; wherein the individual alicyclic or aromatic ring contains 5-8 members and wherein said heterocyclic ring contains 1-6 heteroatom(s) independently selected from the group consisting of O, N, and S; and wherein any aromatic or tertiary alkyl amine is optionally oxidized to a corresponding N-oxide;
Ar₅ is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar₅ optionally contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
R₅ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₄ or Ar₁ group;
U is either O or N, provided that:
when U is O, then R′ is a lone pair of electrons and R″ is selected from the group consisting of Ar₄, C₃-C₈ cycloalkyl, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; and
when U is N, then R′ and R″ are, independently, selected from the group consisting of hydrogen, Ar₄, C₃-C₁₀ cycloalkyl, a C₇-C₁₂ bi- or tri-cyclic carbocycle, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; or R′ and R″ are taken together to form a heterocyclic 5- or 6-membered ring selected from the group consisting of pyrrolidine, imidazolidine, pyrazolidine, piperidine, and piperazine; or,
a pharmaceutically acceptable salt, ester or solvate thereof.

116. A method as claimed in claim 115 in which the sensorineurotrophic compound is a compound of formula I: 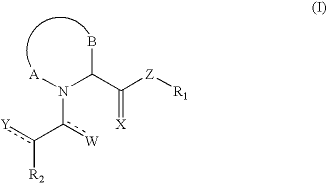
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing one or more heteroatom(s) independently selected from the group consisting of O, S, SO, SO₂, N, NH, and NR₂;
X is either O or S;
Z is either S, CH₂, CHR₁ or CR₁R₃;
W and Y are independently O, S, CH₂ or H₂;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxy; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

117. A method as claimed in claim 116 in which the sensorineurotrophic compound is a compound of formula II: 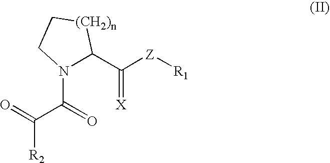
or a pharmaceutically acceptable salt, ester, or solvate thereof, wherein:
n is 1 or 2;
X is O or S;
Z is selected from the group consisting of S, CH₂, CHR₁, and CR₁R₃;
R₁ and R₃ are independently selected from the group consisting of C₁-C₅ straight or branched chain alkyl, C₂-C₅ straight or branched chain alkenyl, and Ar₁, wherein said alkyl, alkenyl or Ar₁ is unsubstituted or substituted with one or more substituent(s) independently selected from the group consisting of halo, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, and Ar₁;
R₂ is selected from the group consisting of C₁-C₉ straight or branched chain alkyl, C₂-C₉ straight or branched chain alkenyl, C₃-C₈ cycloalkyl, C₅-C₇ cycloalkenyl, and Ar₁; and
Ar₁ is phenyl, benzyl, pyridyl, fluorenyl, thioindolyl or naphthyl, wherein said Ar₁ is unsubstituted or substituted with one or more substituent(s) independently selected from the group consisting of halo, trifluoromethyl, hydroxy, nitro, C₁-C₆ straight or branched chain alkyl, C₂-C₆ straight or branched chain alkenyl, C₁-C₄ alkoxy, C₂-C₄ alkenyloxy, phenoxy, benzyloxy, and amino.

118. A method as claimed in claim 116 in which the sensorineurotrophic compound is a compound of formula III: 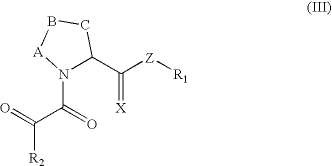
or a pharmaceutically acceptable salt, ester, or solvate thereof, wherein:
A, B, and C are independently CH₂, O, S, SO, SO₂, NH or NR₂;
X is O or S;
Z is S, CH₂, CHR₁ or CR₁R₃;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

119. A method as claimed in claim 116 in which the sensorineurotrophic compound is a compound of formula IV: 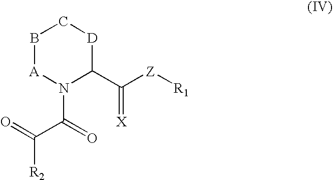
or a pharmaceutically acceptable salt, ester, or solvate thereof, wherein:
A, B, C and D are independently CH₂, O, S, SO, SO₂, NH or NR₂;
X is O or S;
Z is S, CH₂, CHR₁ or CR₁R₃;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₃-C₈ cycloalkyl, C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoro-methyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

120. A method as claimed in claim 115 in which the sensorineurotrophic agent may be a compound of formula VI: 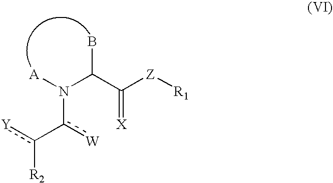
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₁;
X is O or S;
Z is O, NH or NR₁;
W and Y are independently O, S, CH₂ or H₂;
R₁ is C₁-C₆ straight or branched chain alkyl or C₂-C₆ straight or branched chain alkenyl, which is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain or alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

121. The method of claim 120 in which the sensorineurotrophic compound is a compound of formula VII: 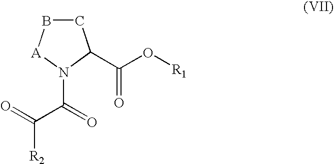
or a pharmaceutically acceptable salt, ester, or solvate thereof, wherein:
A, B and C are independently CH₂, O, S, SO, SO₂, NH or NR₁;
R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁; and
Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

122. The method of claim 121 in which the sensorineurotrophic compound is:

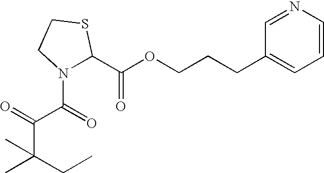

123. A method as claimed in claim 121 in which:
A is CH₂;
B is CH₂ or S;
C is CH₂ or NH;
R₁ is selected from the group consisting of 3-phenylpropyl and 3-(3-pyridyl)propyl; and
R₂ is selected from the group consisting of 1,1-dimethylpropyl, cyclohexyl, and tert-butyl.

124. A method as claimed in claim 120 in which the sensorineurotrophic compound is a compound of formula VIII: 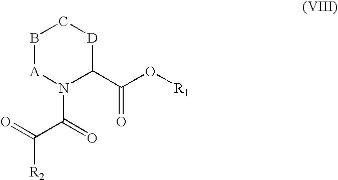
or a pharmaceutically acceptable salt, ester, or solvate thereof, wherein:
A, B, C and D are independently CH₂, O, S, SO, SO₂, NH or NR₁;
R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;
n is 1 or 2;
R₂ is either C₁-C₉ straight or branched chain alkyl, C₂-C₉ straight or branched chain alkenyl, C₃-C₈ cycloalkyl, C₅-C₇ cycloalkenyl, or Ar₁; and
Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

125. A method of claim 124 in which:
A is CH₂;
B is CH₂;
C is S, O or NH;
D is CH₂;
R₁ is selected from the group consisting of 3-phenylpropyl and (3,4,5-trimethoxy)phenylpropyl; and
R₂ is selected from the group consisting of 1,1-dimethylpropyl, cyclohexyl, tert-butyl, phenyl, and 3,4,5-trimethoxyphenyl.

126. A method as claimed in claim 115 in which the sensorineurotrophic agent may be a compound of formula IX: 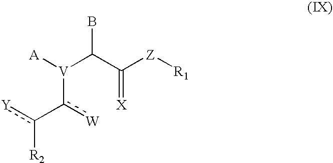
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₃, wherein R is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;
Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and
X is O or S;
Z is O, NH or NR₁;
W and Y are independently O, S, CH₂ or H₂;
R₁ is C₁-C₆ straight or branched chain alkyl or C₂-C₆ straight or branched chain alkenyl, which is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is either C₁-C₉ straight or branched chain alkyl, C₂-C₉ straight or branched chain or alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and
Ar₁ and Ar₂ are independently an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

127. A method as claimed in claim 115 in which the sensorineurotrophic compound is a compound of formula X: 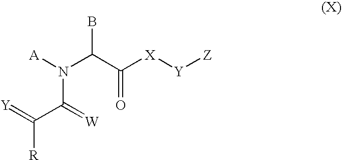
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing one or more heteroatom(s) independently selected from the group consisting of CH, CH₂, O, S, SO, SO₂, N, NH, and NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₂;
Ar₁ and Ar₂ are independently selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is selected from the group consisting of pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl or C₂-C₆ straight or branched chain alkenyl optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

128. A method as claimed in claim 127 in which the sensorineurotrophic compound is a compound of formula XI: 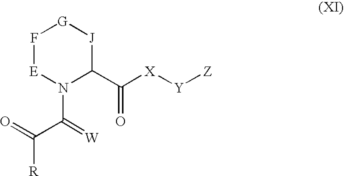
or a pharmaceutically acceptable salt, ester, or solvate thereof, wherein:
E, F, G and J are independently CH₂, O, S, SO, SO₂, NH or NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl, and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, and isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

129. A method as claimed in claim 127 in which the sensorineurotrophic compound is a compound of formula XII: 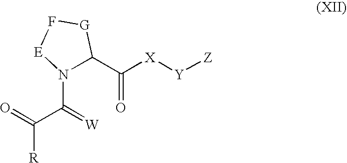
or a pharmaceutically acceptable salt, ester, or solvate thereof, wherein:
E, F, and G are independently CH₂, O, S, SO, SO₂, NH or NR₁;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

130. A method as claimed in claim 127 in which the sensorineurotrophic compound is a compound of formula XIII: 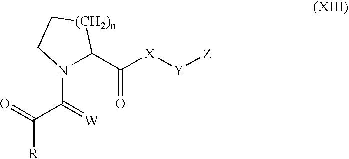
or a pharmaceutically acceptable salt, ester, or solvate thereof, wherein:
n is 1, 2, or 3, forming a 5-7 member heterocyclic ring;
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;
Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃, independently, are hydrogen, C₁-C₄ straight or branched chain alkyl, C₃-C₄ straight or branched chain alkenyl or alkynyl, or Y-Z.

131. A method as claimed in claim 115 in which the sensorineurotrophic agent may be a compound of formula XIV: 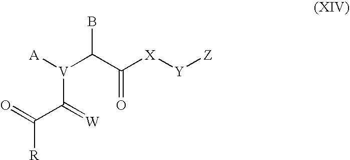
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR₇;
R₇ is either C₁-C₉ straight or branched chain alkyl, C₂-C₉ straight or branched chain alkenyl, C₃-C₉ cycloalkyl, C₅-C₇ cycloalkenyl, or Ar₃, wherein R₇ is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;
Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and
W is O, S, CH₂, or H₂;
R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₂;
Ar₁ and Ar₂ are independently selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;
X is O, NH, NR₁, S, CH, CR₁, or CR₁R₃;
Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is an aromatic amine or a tertiary amine oxidized to a corresponding N-oxide;
said aromatic amine is selected from the group consisting of pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
said tertiary amine is NR₄R₅R₆, wherein R₄, R₅, and R₆ are independently selected from the group consisting of C₁-C₆ straight or branched chain alkyl or C₂-C₆ straight or branched chain alkenyl optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;
Ar is selected from the group consisting of pyrrolidinyl, pyridyl, pyrimidyl, pyrazyl, pyridazyl, quinolinyl, and isoquinolinyl; and
R₁ and R₃ are independently hydrogen, C₁-C₄ straight or branched chain alkyl, C₃-C₄ straight or branched chain alkenyl or alkynyl, or Y-Z.

132. A method as claimed in claim 115 in which the sensorineurotrophic compound is a compound of formula XV: 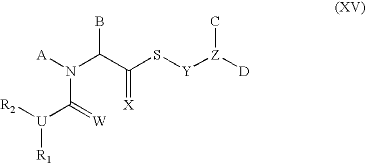
or a pharmaceutically acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more additional heteroatom(s) independently selected from the group consisting of O, S, SO, SO₂, N, NH, and NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

133. A method as claimed in claim 132 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

134. A method as claimed in claim 132 in which the sensorineurotrophic compound is a compound of formula XVI: 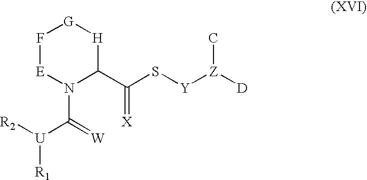
or a pharmaceutically acceptable salt, ester, or solvate thereof, wherein:
E, F, G and J are independently CH₂, O, S, SO, SO₂, NH, or NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

135. A method as claimed in claim 134 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

136. A method as claimed in claim 132 in which the sensorineurotrophic compound is a compound of formula XVII: 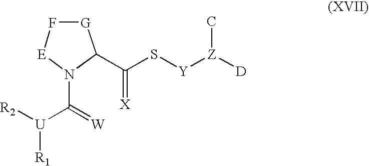
or a pharmaceutically acceptable salt, ester, or solvate thereof, wherein:
E, F, and G are independently CH₂, O, S, SO, SO₂, NH, and NR₃;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₈ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

137. A method as claimed in claim 136 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

138. A method as claimed in claim 115 in which the sensorineurotrophic compound is a compound of formula XVIII: 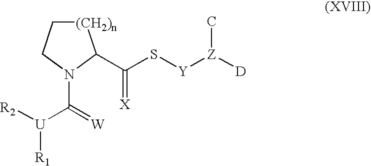
or a pharmaceutically acceptable salt, ester, or solvate thereof, wherein:
n is 1, 2 or 3;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain or alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

139. A method as claimed in claim 138 in which Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

140. A method as claimed in claim 116 in which the sensorineurotrophic compound is a compound of formula XIX: 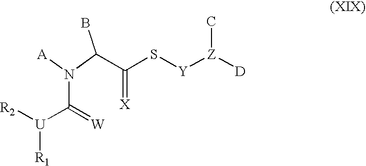
or a pharmaceutically acceptable salt, ester, or solvate thereof, wherein:
V is CH, N, or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂; and
A and B, together with the nitrogen and carbon atoms to which they are respectively attached, form a 5-7 membered saturated or unsaturated heterocyclic ring containing, in addition to the nitrogen atom, one or more additional heteroatom(s) independently selected from the group consisting of O, S, SO, SO₂, N, NH, and NR₃;
X is either O or S;
Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;
W is O or S; and
U is either O or N, provided that:
when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and
when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

141. A method as claimed in claim 115 in which the sensorineurotrophic compound is a compound of formula XX: 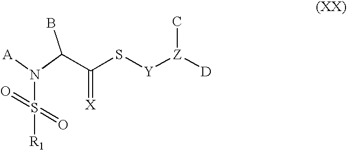
a pharmaceutically acceptable salt, ester, or solvate thereof, wherein:
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₃, S, SO, or SO₂.

142. A method as claimed in claim 141 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

143. A method as claimed in claim 142 in which A and B, together with the nitrogen and carbon atoms to which they are respectfully attached, form a 6 membered saturated or unsaturated heterocyclic ring; and R₂ is C₄-C₇ branched chain alkyl, C₄-C₇ cycloalkyl, phenyl, or 3,4,5-trimethoxyphenyl.

144. A method as claimed in claim 141 in which the sensorineurotrophic compound is selected from the group consisting of:
3-(para-Methoxyphenyl)-1-propylmercaptyl(2S)-N-(benzenesulfonyl)pyrrolidine-2-carboxylate;
3-(para-Methoxyphenyl)-1-propylmercaptyl(2S)-N-α-toluenesulfonyl)pyrrolidine-2-carboxylate;
3-(para-Methoxyphenyl)-1-propylmercaptyl(2S)-N-α-toluenesulfonyl)pyrrolidine-2-carboxylate;
1,5-Diphenyl-3-pentylmercaptyl-N-(para-toluenesulfonyl)pipecolate; and
pharmaceutically acceptable salts and solvates thereof.

145. A method as claimed in claim 141 in which the sensorineurotrophic compound is a compound of formula XXI: 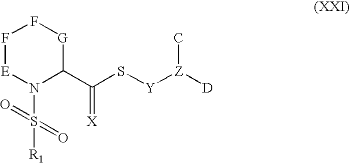
or a pharmaceutically acceptable salt, ester, or solvate thereof, wherein:
E, F, G and J are independently CH₂, O, S, SO, SO₂, NH or NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₃, S, SO, or SO₂.

146. A method as claimed in claim 145 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

147. A method as claimed in claim 141 in which the sensorineurotrophic agent is a compound of formula XXII: 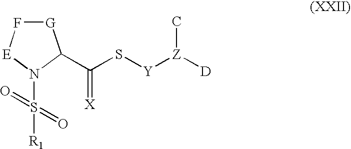
or a pharmaceutically acceptable salt, ester, or solvate thereof, wherein:
E, F, and G are independently CH₂, O, S, SO, SO₂, NH or NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₃, S, SO, or SO₂.

148. A method as claimed in claim 147 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

149. A method as claimed in claim 141 in which the sensorineurotrophic compound is a compound of formula XXIII: 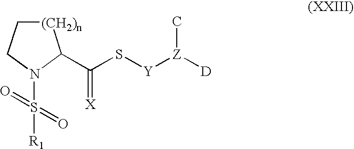
or a pharmaceutically acceptable salt, ester, or solvate thereof, wherein:
n is 1, 2 or 3;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₃, S, SO, or SO₂.

150. A method as claimed in claim 149 in which Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

151. A method as claimed in claim 115 in which the sensorineurotrophic compound is a compound of formula XXIV: 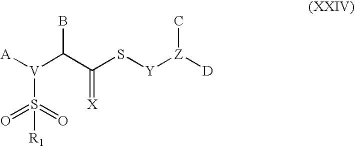
or a pharmaceutically acceptable salt, ester, or solvate thereof, wherein:
V is CH, N, or S;
A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₂;
X is either O or S;
Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₂, S, SO, or SO₂;
R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;
Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;
Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;
C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and
R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₂, S, SO, or SO₂.

152. A method as claimed in claim 115 in which the sensorineurotrophic compound is a compound of formula XXV: 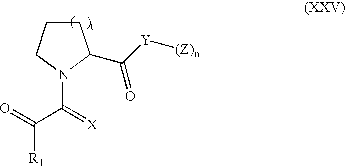
or a pharmaceutically acceptable salt, ester, or solvate thereof, wherein:
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
X is O, S, CH₂ or H₂;
Y is O or NR₂, wherein R₂ is a direct bond to a Z, hydrogen or C₁-C₆ alkyl; and
each Z, independently, is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 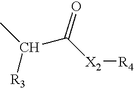
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;
n is 1 or 2, and;
t is 1, 2 or 3.

153. A method as claimed in claim 152 in which the compound is selected from the group consisting of:
3-phenyl-1-propyl (2S)-1-(3,3-dimethyl-1,2-dioxopentyl)-2-pyrrolidinecarboxylate;
3-phenyl-1-prop-2-(E)-enyl (2S)-1-(3,3-dimethyl-1,2-dioxopentyl)-2-pyrrolidinecarboxylate;
3-(3,4,5-trimethoxyphenyl)-1-propyl (2S)-1-(3,3-dimethyl-1,2-dioxopentyl)-2-pyrrolidine-carboxylate;
3-(3,4,5-trimethoxyphenyl)-1-prop-2-(E)-enyl (2S)-1-(3,3-dimethyl-1,2-dioxopentyl)-2-pyrrolidinecarboxylate;
3-(4,5-dichlorophenyl)-1-propyl (2S)-1-(3,3-dimethyl-1,2-dioxopentyl)-2-pyrrolidinecarboxylate;
3-(4,5-dichlorophenyl)-1-prop-2-(E)-enyl (2S)-1-(3,3-dimethyl-1,2-dioxopentyl)-2-pyrrolidine-carboxylate;
3-(4,5-methylenedioxyphenyl)-1-propyl (2S)-1-(3,3-dimethyl-1,2-dioxopentyl)-2-pyrrolidine-carboxylate;
3-(4,5-methylenedioxyphenyl)-1-prop-2-(E)-enyl (2S)-1-(3,3-dimethyl-1,2-dioxopentyl)-2-pyrrolidinecarboxylate;
3-cyclohexyl-1-propyl (2S)-1-(3,3-dimethyl-1,2-dioxopentyl)-2-pyrrolidinecarboxylate;
3-cyclohexyl-1-prop-2-(E)-enyl (2S)-1-(3,3-dimethyl-1,2-dioxopentyl)-2-pyrrolidinecarboxylate;
(1R)-1,3-diphenyl-1-propyl (2S)-1-(3,3-dimethyl-1,2-dioxopentyl)-2-pyrrolidinecarboxylate;
(1R)-1,3-diphenyl-1-prop-2-(E)-enyl (2S)-1-(3,3-dimethyl-1,2-dioxopentyl)-2-pyrrolidine-carboxylate;
(1R)-1-cyclohexyl-3-phenyl-1-propyl (2S)-1-(3,3-dimethyl-1,2-dioxopentyl)-2-pyrrolidine-carboxylate;
(1R)-1-cyclohexyl-3-phenyl-1-prop-2-(E)-enyl (2S)-1-(3,3-dimethyl-1,2-dioxopentyl)-2-pyrrolidinecarboxylate;
(1R)-1-(4,5-dichlorophenyl)-3-phenyl-1-propyl (2S)-1-(3,3-dimethyl-1,2-dioxopentyl)-2-pyrrolidine-carboxylate;
3-phenyl-1-propyl (2S)-1-(1,2-dioxo-2-cyclohexyl)ethyl-2-pyrrolidinecarboxylate;
3-phenyl-1-propyl (2S)-1-(1,2-dioxo-4-cyclohexyl)butyl-2-pyrrolidinecarboxylate;
3-phenyl-1-propyl (2S)-1-(1,2-dioxo-2-[2-furanyl])ethyl-2-pyrrolidinecarboxylate;
3-phenyl-1-propyl (2S)-1-(1,2-dioxo-2-[2-thienyl])ethyl-2-pyrrolidinecarboxylate;
3-phenyl-1-propyl (2S)-1-(1,2-dioxo-2-[2-thiazolyl])ethyl-2-pyrrolidinecarboxylate;
3-phenyl-1-propyl (2S)-1-(1,2-dioxo-2-phenyl)ethyl-2-pyrrolidinecarboxylate;
1,7-diphenyl-4-heptyl (2S)-1-(3,3-dimethyl-1,2-dioxopentyl)-2-pyrrolidinecarboxylate;
3-phenyl-1-propyl (2S)-1-(3,3-dimethyl-1,2-dioxo-4-hydroxybutyl)-2-pyrrolidinecarboxylate;
3-phenyl-1-propyl (2S)-1-(3,3-dimethyl-1,2-dioxopentyl)-2-pyrrolidinecarboxamide;
1-[1-(3,3-dimethyl-1,2-dioxopentyl)-L-proline]-L-phenylalanine ethyl ester;
1-[1-(3,3-dimethyl-1,2-dioxopentyl)-L-proline]-L-leucine ethyl ester;
1-[1-(3,3-dimethyl-1,2-dioxopentyl)-L-proline]-L-phenylglycine ethyl ester;
1-[1-(3,3-dimethyl-1,2-dioxopentyl)-L-proline]-L-phenylalanine phenyl ester;
1-[1-(3,3-dimethyl-1,2-dioxopentyl)-L-proline]-L-phenylalanine benzyl ester;
1-[1-(3,3-dimethyl-1,2-dioxopentyl)-L-proline]-L-isoleucine ethyl ester; and
pharmaceutically acceptable salts, esters, and solvates thereof.

154. A method as claimed in claim 152 in which the sensorineurotrophic compound is a compound of formula XXVI: 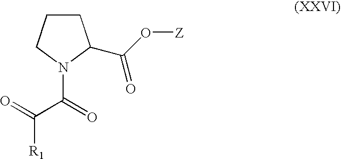
or a pharmaceutically acceptable salt, ester, or solvate thereof, wherein:
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;
Z is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 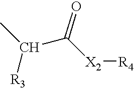
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl; and
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl.

155. A method as claimed in claim 115 in which the sensorineurotrophic agent may be a compound of formula XXVII: 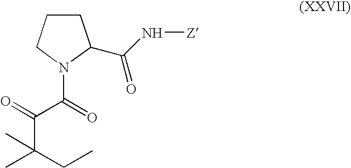
or a pharmaceutically acceptable salt, ester, or solvate thereof, wherein:
Z′ is the fragment 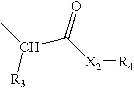
wherein:
R₃ is C₁-C₉ straight or branched chain alkyl or unsubstituted Ar₁, wherein said alkyl is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl;
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and
Ar₁ is selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino.

156. A method as claimed in claim 152 in which the sensorineurotrophic agent may also be a compound of formula XXVIII: 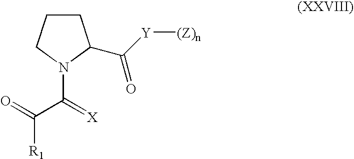
wherein:
R₁ is C₁-C₆ straight or branched chain alkyl, C₂-C₆ straight or branched chain alkenyl, C₃-C₆ cycloalkyl or Ar₁, wherein said alkyl or alkenyl is unsubstituted or substituted with C₃-C₆ cycloalkyl or Ar₂;
Ar₁ and Ar₂ are independently selected from the group consisting of 2-furyl, 2-thienyl, and phenyl;
X is selected from the group consisting of oxygen and sulfur;
Y is oxygen or NR₂, wherein R₂ is a direct bond to a Z, hydrogen or C₁-C₆ alkyl;
each Z, independently, is hydrogen, C₁-C₆ straight or branched chain alkyl, or C₂-C₆ straight or branched chain alkenyl, wherein said Z is substituted with one or more substituent(s) independently selected from the group consisting of 2-furyl, 2-thienyl, C₃-C₆ cycloalkyl, pyridyl, and phenyl, each having one or more substituent(s) independently selected from the group consisting of hydrogen and C₁-C₄ alkoxy; and
n is 1 or 2.

157. A method as claimed in claim 156 in which the compound is selected from the group consisting of:
3-(2,5-dimethoxyphenyl)-1-propyl (2S)-1-(3,3-dimethyl-1,2-dioxopentyl)-2-pyrrolidinecarboxylate;
3-(2,5-dimethoxyphenyl)-1-prop-2-(E)-enyl (2S)-1-(3,3-dimethyl-1,2-dioxopentyl)-2-pyrrolidine-carboxylate;
2-(3,4,5-trimethoxyphenyl)-1-ethyl (2S)-1-(3,3-dimethyl-1,2-dioxopentyl)-2-pyrrolidinecarboxylate;
3-(3-pyridyl)-1-propyl (2S)-1-(3,3-dimethyl-1,2-dioxopentyl)-2-pyrrolidinecarboxylate;
3-(2-pyridyl)-1-propyl (2S)-1-(3,3-dimethyl-1,2-dioxopentyl)-2-pyrrolidinecarboxylate;
3-(4-pyridyl)-1-propyl (2S)-1-(3,3-dimethyl-1,2-dioxopentyl)-2-pyrrolidinecarboxylate;
3-phenyl-1-propyl (2S)-1-(2-tert-butyl-1,2-dioxoethyl)-2-pyrrolidinecarboxylate;
3-phenyl-1-propyl (2S)-1-(2-cyclohexylethyl-1,2-dioxoethyl)-2-pyrrolidinecarboxylate;
3-(3-pyridyl)-1-propyl (2S)-1-(2-cyclohexylethyl-1,2-dioxoethyl)-2-pyrrolidine-carboxylate;
3-(3-pyridyl)-1-propyl (2S)-1-(2-tert-butyl-1,2-dioxoethyl)-2-pyrrolidinecarboxylate;
3,3-diphenyl-1-propyl (2S)-1-(3,3-dimethyl-1,2-dioxopentyl)-2-pyrrolidinecarboxylate;
3-(3-pyridyl)-1-propyl (2S)-1-(2-cyclohexyl-1,2-dioxoethyl)-2-pyrrolidinecarboxylate;
3-(3-pyridyl)-1-propyl (2S)-N-([2-thienyl]glyoxyl)pyrrolidinecarboxylate;
3,3-diphenyl-1-propyl (2S)-1-(3,3-dimethyl-1,2-dioxobutyl)-2-pyrrolidinecarboxylate;
3,3-diphenyl-1-propyl (2S)-1-cyclohexylglyoxyl-2-pyrrolidinecarboxylate;
3,3-diphenyl-1-propyl (2S)-1-(2-thienyl)glyoxyl-2-pyrrolidinecarboxylate; and
pharmaceutically acceptable salts, esters, and solvates thereof.

158. A method as claimed in claim 115 in which the sensorineurotrophic compound is a compound of formula XXIX: 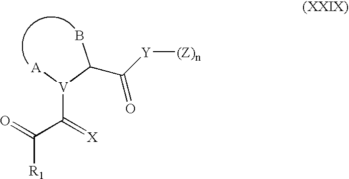
or a pharmaceutically acceptable salt, ester, or solvate thereof, wherein:
V is CH, N, or S;
A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo-(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, alkylthio, sulfhydryl, amino, (C₁-C₆)-alkylamino, amino-(C₁-C₆)-alkyl, aminocarboxyl, and Ar₂;
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;
Ar₁ and Ar₂ are independently an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S;
X is O, S, CH₂ or H₂;
Y is O or NR₂, wherein R₂ is a direct bond to a Z, hydrogen or C₁-C₆ alkyl; and
each Z, independently, is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment 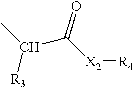
wherein:
R₃ is C₁-C₉ straight or branched chain alkyl which is unsubstituted or substituted with C₃-C₈ cycloalkyl or Ar₁;
X₂ is O or NR₅, wherein R₅ is selected from the group consisting of hydrogen, C₁-C₆ straight or branched chain alkyl, and C₂-C₆ straight or branched chain alkenyl; and
R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and,
n is 1 or 2.

159. A method as claimed in claim 115 in which the sensorineurotrophic compound is a compound of formula (LV): 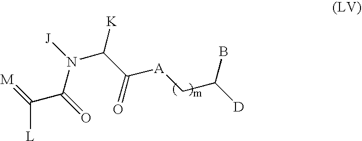
or a pharmaceutically acceptable salt, ester, or solvate thereof, wherein:
m is 0-3;
A is CH₂, O, NH, or N—(C₁-C₄ alkyl);
B and D are independently hydrogen, Ar, C₅-C₇ cycloalkyl substituted C₁-C₆ straight or branched chain alkyl or C₂-C₆ straight or branched chain alkenyl, C₅-C₇ cycloalkenyl substituted C₁-C₆ straight or branched chain alkyl or C₂-C₆ straight or branched chain alkenyl, or Ar substituted C₁-C₆ straight or branched chain alkyl or C₂-C₆ straight or branched chain alkenyl, wherein in each case, one or two carbon atom(s) of said alkyl or alkenyl may be substituted with one or two heteroatom(s) independently selected from the group consisting of oxygen, sulfur, SO, and SO₂ in chemically reasonable substitution patterns, or 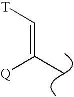
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
L is either hydrogen or U; M is either oxygen or CH-U, provided that if L is hydrogen, then M is CH-U, or if M is oxygen then L is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylmethyl; or J and K are taken together to form a 5-7 membered heterocyclic ring which is substituted with oxygen, sulfur, SO, or SO₂.

160. A method as claimed in claim 115 in which the sensorineurotrophic compound is a compound of formula (LVI): 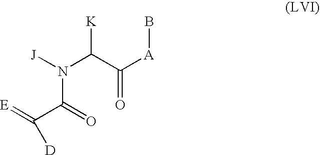
or a pharmaceutically acceptable salt, ester, or solvate thereof, wherein:
A is O, NH, or N—(C₁-C₄ alkyl);
B is hydrogen, CHL-Ar, C₁-C₆ straight or branched chain alkyl, C₂-C₆ straight or branched chain alkenyl, C₅-C₇ cycloalkyl, C₅-C₇ cycloalkenyl, Ar substituted C₁-C₆ alkyl or C₂-C₆ alkenyl, or 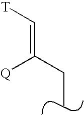
wherein L and Q are independently hydrogen, C₁-C₆ straight or branched chain alkyl, or C₂-C₆ straight or branched chain alkenyl; and
T is Ar or C₅-C₇ cyclohexyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 2-pyridyl, 3-pyridyl, 4-pyridyl and phenyl having 1-3 substituent(s) independently selected from the group consisting of hydrogen, halo, hydroxy, nitro, CF₃, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, and phenyl.
D is hydrogen or U; E is oxygen or CH-U, provided that if D is hydrogen, then E is CH-U, or if E is oxygen, then D is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇-cycloalkyl, C₅-C₇ cycloalkenyl substituted with C₁-C₄ straight or branched chain alkyl or C₂-C₄ straight or branched chain alkenyl, 2-indolyl, 3-indolyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylethyl; or J and K are taken together to form a 5-7 membered heterocyclic ring which is substituted with oxygen, sulfur, SO, or SO₂.

161. A method as claimed in claim 115 in which the sensorineurotrophic compound is a compound of formula LVIII: 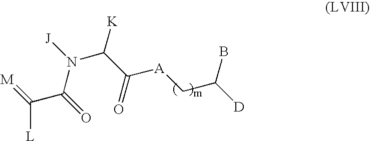
or a pharmaceutically acceptable salt, ester, or solvate thereof, wherein:
V is CH, N, or S;
J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;
Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;
A is CH₂, O, NH, or N—(C₁-C₄ alkyl);
B and D are independently hydrogen, Ar, C₅-C₇ cycloalkyl substituted C₁-C₆ straight or branched chain alkyl or C₂-C₆ straight or branched chain alkenyl, C₅-C₇ cycloalkenyl substituted C₁-C₆ straight or branched chain alkyl or C₂-C₆ straight or branched chain alkenyl, or Ar substituted C₁-C₆ straight or branched chain alkyl or C₂-C₆ straight or branched chain alkenyl, wherein in each case, one or two carbon atom(s) of said alkyl or alkenyl may be substituted with one or two heteroatom(s) independently selected from the group consisting of oxygen, sulfur, SO, and SO₂ in chemically reasonable substitution patterns, or 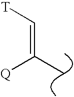
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl;
Ar is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;
L is either hydrogen or U; M is either oxygen or CH-U, provided that if L is hydrogen, then M is CH-U, or if M is oxygen then L is U;
U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar, or Ar;
J is hydrogen, C₁ or C₂ alkyl, or benzyl; K is C₁-C₄ straight or branched chain alkyl, benzyl or cyclohexylmethyl; or J and K are taken together to form a 5-7 membered heterocyclic ring which is substituted with oxygen, sulfur, SO, or SO₂.

162. A method as claimed in claim 115 in which the sensorineurotrophic compound is a compound of the formula (LIX): 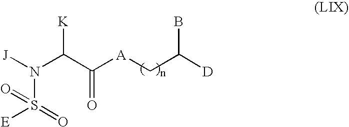
or a pharmaceutically acceptable salt, ester or solvate thereof, wherein:
A is CH₂, O, NH, or N—(C₁-C₄ alkyl);
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 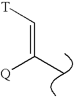
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar;
J is hydrogen, C₁ or C₂ alkyl, or benzyl; K is C₁-C₄ straight or branched chain alkyl, benzyl, or cyclohexylmethyl; or J and K are taken together to form a 5-7 membered heterocyclic ring which is substituted with O, S, SO, or SO₂;
n is 0 to 3.

163. A method as claimed in claim 115 in which the sensorineurotrophic compound is a compound of Formula LXI: 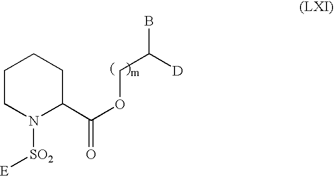
or a pharmaceutically acceptable salt, ester or solvate thereof, wherein:
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 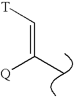
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and
m is 0 to 3.

164. A method as claimed in claim 115 in which the sensorineurotrophic compound is a compound of Formula (LXII): 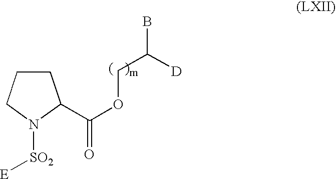
or a pharmaceutically acceptable salt thereof, wherein:
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl, or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 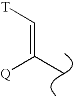
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄-straight or branched chain alkenyl, (C₂-C₄ alkyl or C₂-C₄ alkenyl)-Ar, or Ar; and
m is 0 to 3.

165. A method as claimed in claim 115 in which the sensorineurotrophic compound is a compound of Formula LXIII: 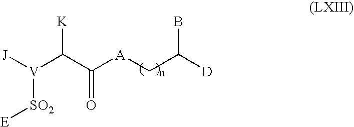
or a pharmaceutically acceptable salt, ester, or solvate thereof, wherein:
V is CH, N, or S;
J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;
R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;
Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;
A is CH₂, O, NH, or N—(C₁-C₄ alkyl);
B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or 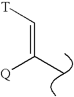
wherein Q is hydrogen, C₁-C₆ straight or branched chain alkyl, or C₂-C₆ straight or branched chain alkenyl; and
T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl;
provided that both B and D are not hydrogen;
Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;
E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar;
J is hydrogen, C₁ or C₂ alkyl, or benzyl; K is C₁-C₄ straight or branched chain alkyl, benzyl, or cyclohexylmethyl; or J and K are taken together to form a 5-7 membered heterocyclic ring which is substituted with O, S, SO, or SO₂;
n is 0 to 3.

166. A method as claimed in claim 115 in which the sensorineurotrophic compound is a compound of formula (LXIV): 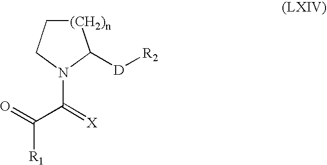
in which:
n is 1-3;
X is either O or S;
R₁ is selected from the group consisting of C₁-C₉ straight or branched chain alkyl, C₂-C₉ straight or branched chain alkenyl, aryl, heteroaryl, carbocycle, or heterocycle;
D is a bond, or a C₁-C₁₀ straight or branched chain alkyl, C₂-C₁₀ alkenyl or C₂-C₁₀ alkynyl; and
R₂ is a carboxylic acid or a carboxylic acid isostere;
or a pharmaceutically acceptable salt, ester, or solvate thereof.

167. A method as claimed in claim 166 in which:
R₂ is selected from the group: 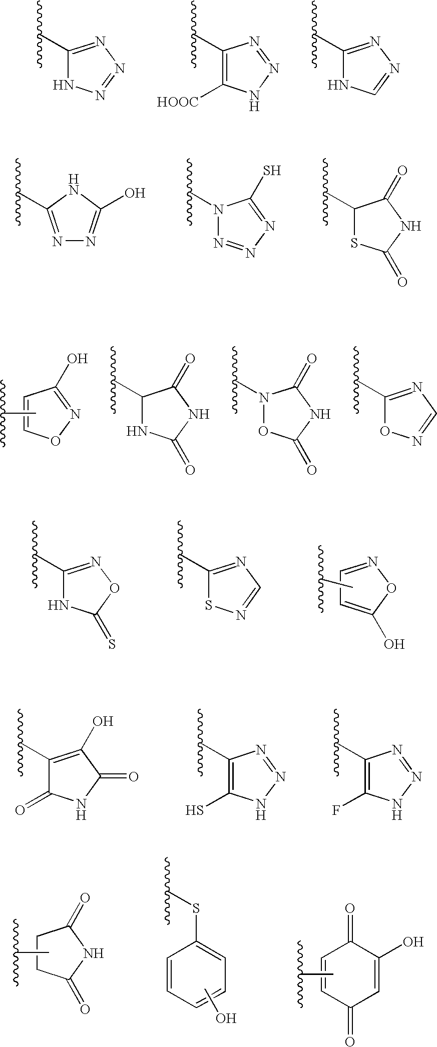
—COOH, —SO₃H, —SO₂HNR³, —PO₂ (R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl.

168. A method as claimed in claim 115 in which the sensorineurotrophic compound is a compound of formula (LXV): 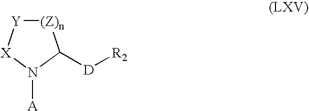
in which
X, Y, and Z are independently selected from the group consisting of C, O, S, or N, provided that X, Y, and Z are not all C;
n is 1-3;
A is selected from the group consisting of L₁, L₂, L₃, or L₄, in which 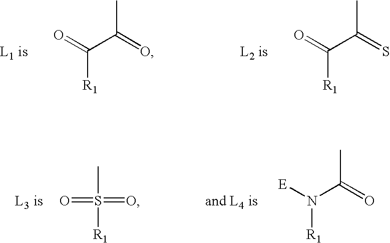
and R₁ and E, independently, are selected from the group consisting of hydrogen, C₁-C₉ straight or branched chain alkyl, C₂-C₉ straight or branched chain alkenyl, aryl, heteroaryl, carbocycle, and heterocycle;
R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where R³ is hydrogen, hydroxy, halo, halo(C₁-C₆)-alkyl, thiocarbonyl, (C₁-C₆)-alkoxy, (C₂-C₆)-alkenoxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;
or a pharmaceutically acceptable salt, ester, or solvate thereof.

169. A method as claimed in claim 115 in which the sensorineurotrophic compound is a compound of formula (LXVI): 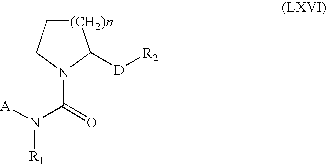
in which:
n is 1-3;
R₁ and A are independently selected from the group consisting of hydrogen, C₁-C₉ straight or branched chain alkyl, C₂-C₉ straight or branched chain alkenyl, aryl, heteroaryl, carbocycle, and heterocycle;
D is a bond, or a C₁-C₁₀ straight or branched chain alkyl, C₂-C₁₀ alkenyl or C₂-C₁₀ alkynyl;
R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where
R³ is hydrogen, hydroxy, halo, halo(C₁-C₆)-alkyl, thiocarbonyl, (C₁-C₆)-alkoxy, (C₂-C₆)-alkenoxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl;
or a pharmaceutically acceptable salt, ester, or solvate thereof.

170. A method as claimed in claim 115 in which the sensorineurotrophic compound is a compound of formula (LXVII): 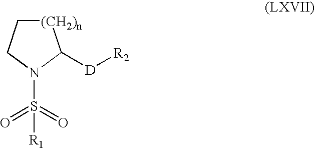
in which:
n is 1-3;
R₁ is selected from the group consisting of hydrogen, C₁-C₉ straight or branched chain alkyl, C₂-C₉ straight or branched chain alkenyl, aryl, heteroaryl, carbocycle, or heterocycle;
D is a bond, or a C₁-C₁₀ straight or branched chain alkyl, C₂-C₁₀ alkenyl or C₂-C₁₀ alkynyl;
R₂ is a carboxylic acid or a carboxylic acid isostere;
wherein said alkyl, alkenyl, alkynyl, aryl, heteroaryl, carbocycle, heterocycle, or carboxylic acid isostere is optionally substituted with one or more substituents selected from R³, where
R³ is hydrogen, hydroxy, halo, halo-(C₁-C₆)-alkoxy, thiocarbonyl, (C₁-C₆)-alkoxy, (C₂-C₆)-alkenyloxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;
or a pharmaceutically acceptable salt, ester or solvate thereof.

171. A method for the prevention or treatment of a vestibular disorder which comprises administering to a warm-blooded animal a sensorineurotrophic compound selected from the group comprising: 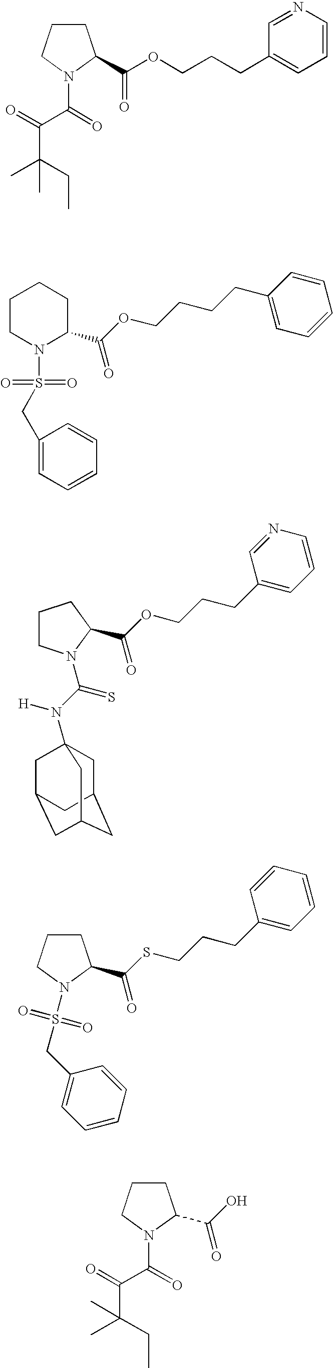 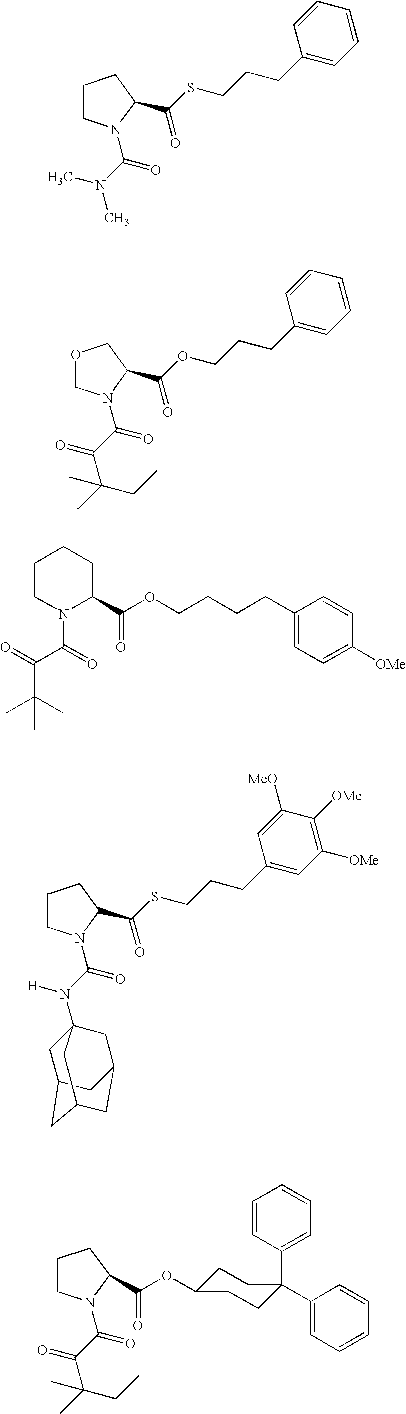 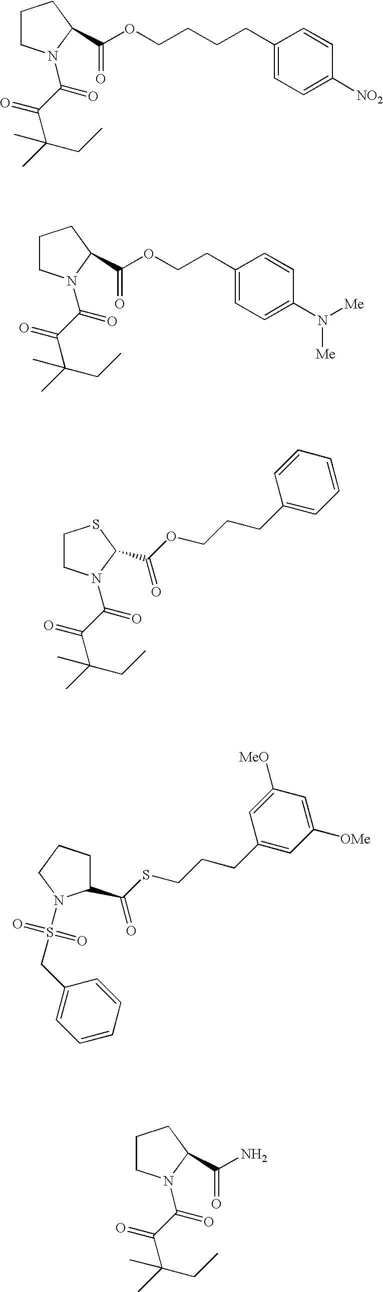 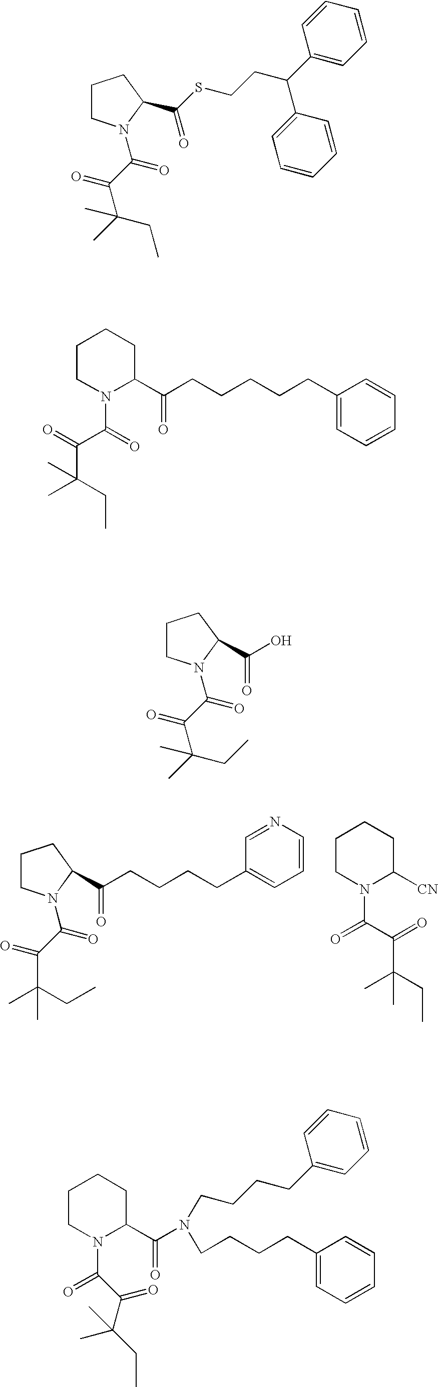 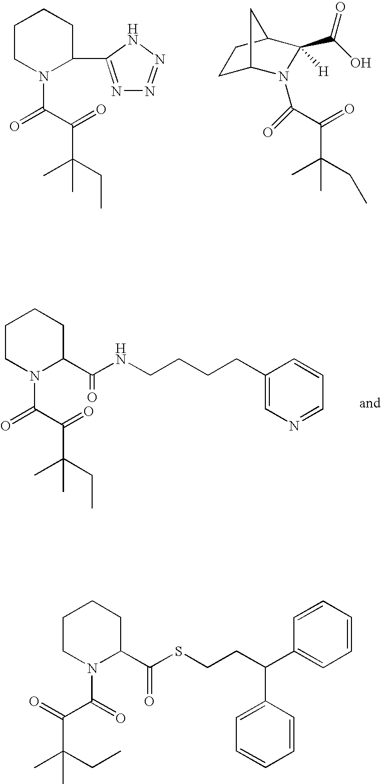
or a pharmaceutically acceptable salt, solvate or ester thereof.
